(19) United States
(12) Patent Application Publication
(10) Pub. No.: US 2026/0310075 A1
(43) Pub. Date: Oct. 8, 2026

(54) ONIUM SALT MONOMER, POLYMER, CHEMICALLY AMPLIFIED RESIST COMPOSITION, AND PATTERN FORMING PROCESS

(71) Applicant: Shin-Etsu Chemical Co., Ltd., Tokyo (JP)

(72) Inventors: Ryo Sudo, Joetsu-shi (JP); Gentaro Hida, Joetsu-shi (JP); Miyu Yoshida, Joetsu-shi (JP)

(73) Assignee: Shin-Etsu Chemical Co., Ltd., Tokyo (JP)

(21) Appl. No.: 19/568,947

(22) Filed: Mar. 17, 2026

(30) Foreign Application Priority Data
Mar. 26, 2025 (JP) 2025-051348

(51) Int. Cl. G03F 7/004; C07C 309/58; C07C 309/59; C07C 309/60; C07C 381/12; C08F 22/24; G03F 7/00; G03F 7/039; G03F 7/20

(52) U.S. Cl. CPC G03F 7/0045; C07C 309/58; C07C 309/59; C07C 309/60; C07C 381/12; C08F 22/24; G03F 7/0397; G03F 7/2004; G03F 7/70033

(57) ABSTRACT

The onium salt monomer can be used for a chemically amplified resist composition which is processed by photolithography using, in particular, high-energy radiation such as a KrF excimer laser beam, an ArF excimer laser beam, an electron beam (EB) or EUV. The onium salt monomer having the following formula (a).

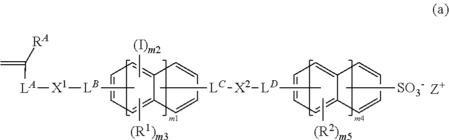

### CROSS-REFERENCE TO RELATED APPLICATION

This non-provisional application claims priority under 35 U.S.C. § 119(a) on Patent Application No. 2025-051348 filed in Japan on Mar. 26, 2025, the entire contents of which are hereby incorporated by reference.

### TECHNICAL FIELD

The present invention relates to an onium salt monomer, a polymer, a chemically amplified resist composition, and a pattern forming process.

### BACKGROUND ART

To meet the demand for higher integration density and operating speed of LSIs, the effort to reduce the pattern rule is in rapid progress. The wide-spreading flash memory market and the demand for increased storage capacities drive forward the miniaturization technology. As the advanced miniaturization technology, manufacturing of microelectronic 65-nm mode devices by the ArF lithography has been implemented in a mass scale. Manufacturing of 45-nm node devices by the next generation ArF immersion lithography is approaching to the verge of high-volume application. The candidates for the next generation 32-nm node include ultra-high NA lens immersion lithography using a liquid having a higher refractive index than water in combination with a high refractive index lens and a high refractive index resist film, EUV lithography of wavelength 13.5 nm, and double patterning version of the ArF lithography, on which active research efforts have been made.

As the pattern feature size is reduced, the line width roughness (LWR) of line patterns and the critical dimension uniformity (CDU) of hole patterns and dot patterns are regarded significant. It is pointed out that these factors are affected by the segregation and agglomeration of a base polymer and acid generator and the diffusion of generated acid. There is a tendency that as the resist film becomes thinner, LWR or CDU becomes greater. A film thickness reduction to comply with the progress of size reduction causes a degradation of LWR and CDU, which becomes a serious problem.

The EUV lithography resist must meet high sensitivity, high resolution and low LWR at the same time. As the acid diffusion distance is reduced, LWR is reduced, but sensitivity becomes lower. For example, as the PEB temperature is lowered, the outcome is a reduced LWR, but a lower sensitivity. As the amount of quencher added is increased, the outcome is a reduced LWR, but a lower sensitivity. It is necessary to overcome the tradeoff relation between sensitivity and LWR.

Patent Document 1 discloses a resist compound comprising repeat units derived from an onium salt of a polymerizable unsaturated bond-containing sulfonic acid. The so called polymer-bound acid generator is capable of generating a polymer type sulfonic acid upon exposure and is characterized by a very short distance of acid diffusion. Sensitivity may be enhanced by increasing a proportion of the acid generator. In the case of addition type acid generators, as the amount of acid generator added is increased, a higher sensitivity is achievable, but the acid diffusion distance is also increased. Since the acid diffusion is non-uniform, increased acid diffusion leads to degraded LWR and CDU. With respect to a balance of sensitivity, LWR and CDU, the polymer-bound acid generator has a high capability.

Since iodine atoms are highly absorptive to EUV of wavelength 13.5 nm, they generate secondary electrons upon light exposure. This effect is noteworthy in the EUV lithography. Patent Document 2 describes a photoacid generator having an iodized anion. Patent Document 3 describes a photoacid generator having an iodized anion and containing a polymerizable group.

An impact of a perfluoroalkyl compound (PFAS) on the health has been pointed out, and there are moves in European REACH to place a restriction on the production and sale of PFAS compounds. In the context of semiconductor lithography, many compounds containing PFAS are currently used. Materials containing PFAS are used for surfactants and acid generators, for example. The photoacid generators disclosed in Patent Documents 1, 2 and 3 are based on fluoroalkanesulfonic acid units, and have been pointed out to have significant impacts on the health and environment.

Patent Document 4 discloses a fluorobenzenesulfonic acid onium salt as an alternative to PFAS. Photoacid generators having a fluorobenzenesulfonic acid structure do not have fluoroalkanesulfonic acid in the structure, and thus have reduced impacts on the health and environment. Electron withdrawing groups, one of which is fluorine, can increase the acid strength of the sulfonic acid. However, replacement of hydrogen on the aromatic ring of the benzenesulfonic acid by fluorine cannot give sufficient acid strength because of the resonance effect of the aromatic group.

### CITATION LIST

Patent Document 1: JP 4425776
Patent Document 2: JP 6720926
Patent Document 3: JP 6973274
Patent Document 4: JP-A 2018-159744

### SUMMARY OF THE INVENTION

In the field of acid-catalyzed chemically amplified resist compositions, it is desired to develop a resist composition having a higher sensitivity, and improved lithographic performance such as improved LWR, CDU, exposure latitude (EL) and depth of focus (DOF), and excellent etch resistance after pattern formation.

The present invention has been made in view of the above-described circumstances, and an object of the present invention is to provide an onium salt monomer for a chemically amplified resist composition which is processed by photolithography using, in particular, high-energy radiation such as a KrF excimer laser beam, an ArF excimer laser beam, an electron beam (EB) or EUV, has excellent solvent solubility and a high sensitivity and contrast, and is excellent in lithographic performance such as LWR, CDU, EL and DOF, and excellent in resistance to pattern collapse even in fine pattern formation, a polymer comprising repeat units derived from the onium salt monomer, a chemically amplified resist composition containing the polymer, and a pattern forming process using the chemically amplified resist composition.

The inventors have found that by using, as a polymer-bound photoacid generator, a polymer comprising repeat units derived from an onium salt monomer having a polymerizable group, an aromatic ring, and a benzenesulfonic acid structure substituted with an electron withdrawing group, a chemically amplified resist composition having a high sensitivity, high contrast, high resolution, and improved lithographic performance such as improved EL, LWR, CDU and DOF can be obtained.

The present invention provides the following onium salt monomer, polymer, chemically amplified resist composition, and pattern forming process.

1. An onium salt monomer having the following formula (a):

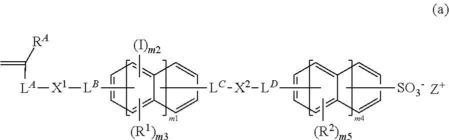

wherein m1 is 0 or 1, m2 is 0, 1, 2, 3 or 4 in case of m1=0, and 0, 1, 2, 3, 4, 5 or 6 in case of m1=1, m3 is 0, 1, 2, 3 or 4 in case of m1=0, and 0, 1, 2, 3, 4, 5 or 6 in case of m1=1, m2+m3 is from 0 to 4 when m1=0 and m2+m3 is from 0 to 6 when m1=1, m4 is 0 or 1, m5 is 1, 2, 3 or 4 in case of m4=0, and 1, 2, 3, 4, 5 or 6 in case of m4=1,
R^{A} is hydrogen atom or methyl,
R¹ is halogen exclusive of iodine, nitro group, hydroxy group, cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, a plurality of R¹ may be identical or different and a plurality of R¹ may bond together to form a ring with the carbon atoms to which they are attached, when m3 is 2 or more,
R² is halogen exclusive of iodine, or an electron withdrawing group, a plurality of R² may be identical or different and a plurality of R² may bond together to form a ring with the carbon atoms to which they are attached, when m5 is 2 or more, excluding that all R² are fluorine,
L^{A}, L^{B}, L^{C} and L^{D} are each independently a single bond, ester bond, ether bond, amide bond, sulfonate ester bond, sulfonamide bond, carbonate bond or carbamate bond,
each of X¹ and X² is a single bond, or a C₁-C₄₀ hydrocarbylene group which may contain a heteroatom, and
Z⁺ is a sulfonium cation having the following formula (Z-1) or an iodonium cation having the following formula (Z-2);

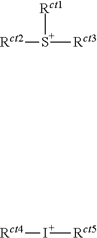

wherein R^{ct1} to R^{ct5} are each independently halogen, or a C₁-C₃₀ hydrocarbyl group which may contain a heteroatom, and R^{ct1} and R^{ct2} may bond together to form a ring with the sulfur atom to which they are attached.
2. The onium salt monomer of 1 which has the following formula (a1):

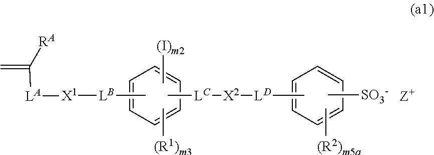

wherein m2, m3, R^{A}, R¹, R², X¹, X², L^{A} to L^{D} and Z⁺ are as defined above, and m5a is 1, 2, 3 or 4.
3. The onium salt monomer of 2 which has the following formula (a2):

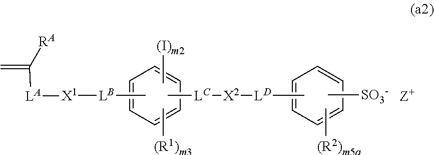

wherein m2, m3, m5a, R^{A}, R¹, X¹, X², L^{A} to L^{D} and Z⁺ are as defined above, and R^{2A} is an electron withdrawing group exclusive of halogen.
4. A monomeric photoacid generator comprising the onium salt monomer of any one of 1 to 3.
5. The polymer comprising repeat units derived from the monomeric photoacid generator of 4.
6. The polymer of 5 comprising repeat units of at least one type selected from repeat units of the formulae (b1) and (b2):

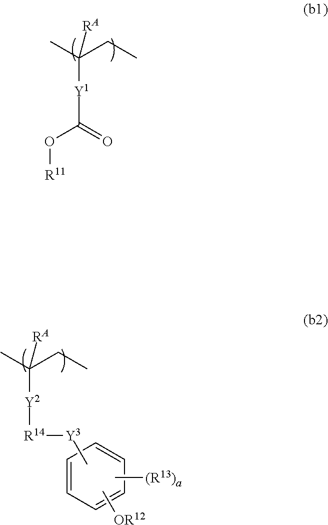

wherein R^{A} is each independently hydrogen or methyl,
Y¹ is a single bond, a phenylene group, a naphthylene group, or a C₁-C₁₂ linking group containing at least one selected from an ester bond, an ether bond and a lactone ring, the phenylene group, the naphthylene group and the linking group may have at least one selected from a hydroxy group, a C₁-C₈ saturated hydrocarbyloxy group and a C₂-C₈ saturated hydrocarbylcarbonyloxy group,
Y² is a single bond or an ester bond.

Y³ is a single bond, an ether bond or an ester bond.

R¹¹ and R¹² are each independently an acid labile group.

R¹³ is a C₁-C₄ saturated hydrocarbyl group, a halogen atom, a C₂-C₅ saturated hydrocarbylcarbonyl group, a cyano group, or C₂-C₅ saturated hydrocarbyloxycarbonyl group.

R¹⁴ is a single bond or C₁-C₆ alkanediyl group, and the alkanediyl group may contain at least one selected from a hydroxy group, a C₁-C₈ saturated hydrocarbyloxy group, a C₂-C₈ saturated hydrocarbylcarbonyloxy group, an ether bond and an ester bond, and
a is 0, 1, 2, 3 or 4.
   7. A chemically amplified resist composition comprising a base polymer containing the polymer of 6.
   8. The chemically amplified resist composition of 7, further comprising an organic solvent.
   9. The chemically amplified resist composition of 7 or 8, further comprising a quencher.
   10. The chemically amplified resist composition of any of 7 to 9, further comprising an acid generator.
   11. The chemically amplified resist composition of any of 7 to 10 further comprising a surfactant.
   12. A pattern forming process comprising the steps of applying the chemically amplified resist composition of any of 7 to 11 onto a substrate to form a resist film thereon, exposing the resist film to high-energy radiation, and developing the exposed resist film in a developer.
   13. The pattern forming process of 12, wherein the high-energy radiation is ArF excimer laser having a wavelength 193 nm, KrF excimer laser having a wavelength 248 nm, EB, or EUV having a wavelength 3 to 15 nm.

### Advantageous Effects of the Invention

In a polymer comprising repeat units derived from the inventive onium salt monomer and generating an acid upon exposure, the acid strength of the benzenesulfonic acid is increased by an electron withdrawing group to achieve a high sensitivity, and the low acid strength of a benzenesulfonic acid substituted with only fluorine is improved. Further, since the electron withdrawing group has an atomic weight larger than that of fluorine, ease of movement of the benzenesulfonic acid is reduced, and acid diffusion can be suppressed, so that it is possible to improve lithography performance such as LWR, CDU, EL and DOF.

### DESCRIPTION OF THE PREFERRED EMBODIMENT

### [Onium Salt Monomer]

The inventive onium salt monomer has the following formula (a).

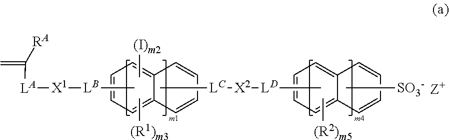

In the formula (a), m1 is 0 or 1. The relevant structure is a benzene ring when m1=0, and a naphthalene ring when m1=1. From the aspect of solvent solubility, the benzene ring corresponding to m1=0 is preferred. m2 is 0, 1, 2, 3 or 4 in case of m1=0, and 0, 1, 2, 3, 4, 5 or 6 in case of m1=1, From the aspect of reactant availability, m2 is preferably 0, 1 or 2, more preferably 1 or 2. m3 is 0, 1, 2, 3 or 4 in case of m1=0, and 0, 1, 2, 3, 4, 5 or 6 in case of m1=1, m2+m3 is from 0 to 4 when m1=0 and m2+m3 is from 0 to 6 when m1=1,

In the formula (a), m4 is 0 or 1. The sulfonium salt monomer has a benzene ring when m4 is 0, and a naphthalene ring when m4 is 1, and m4 is preferably 0 from the aspect of solvent solubility. m5 is 1, 2, 3 or 4 in case of m4=0, and 1, 2, 3, 4, 5 or 6 in case of m4=1, By substituting benzenesulfonic acid with an electron withdrawing group, the acid strength of the sulfonic acid is increased, and the sensitivity is improved, but since it is difficult to procure raw materials for the benzenesulfonic acid substituted with a plurality of electron withdrawing groups, m5 is preferably 1, 2, or 3.

In the formula (a), R^{A} is hydrogen atom or methyl.

In the formula (a), R¹ is halogen exclusive of iodine, nitro group, hydroxy group, cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. The hydrocarbyl group and hydrocarbyl moiety in the hydrocarbyloxy and hydrocarbylthio groups may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icosyl groups; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl and adamantyl groups; C₂-C₂₀ alkenyl groups such as vinyl, allyl, propenyl, butenyl and hexenyl groups; C₃-C₂₀ cyclic unsaturated hydrocarbyl groups such as a cyclohexenyl group; C₂-C₂₀ aryl groups such as phenyl and naphthyl groups; C₇-C₂₀ aralkyl groups such as benzyl, 1-phenylethyl and 2-phenylethyl groups; and combinations thereof. In the hydrocarbyl group, some or all hydrogen may be replaced by a moiety containing a heteroatom such as oxygen, sulfur, nitrogen or halogen, and some constituent —CH₂— may be replaced by a group containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety. A plurality of R¹ may be identical or different when m3 is 2 or more.

A plurality of R¹ may bond together to form a ring with the carbon atoms to which they are attached when m3 is 2 or more. Examples of the ring formed herein include cyclopropane, cyclobutane, cyclopentane, cyclohexane, norbornane and adamantane rings. In the ring, some or all hydrogen may be replaced by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

In the formula (a), R² is halogen exclusive of iodine, or an electron withdrawing group. a plurality of R² may be identical or different and a plurality of R² may bond together to form a ring with the carbon atoms to which they are attached, when m5 is 2 or more, excluding that all R² are fluorine, When the aryl hydrogen of the benzenesulfonic acid is replaced by fluorine and another electron withdrawing group, or by only an electron withdrawing group, sensitivity is improved and acid diffusion can be reduced as compared to a case where the aryl hydrogen is replaced by only fluorine.

The electron withdrawing group R² is preferably an electron withdrawing group in which the m-position substituent constant σₘ in the Hammett equation, which is described in Chem. Rev. 1991, 91, 165-169, is more than 0, more preferably more than 0.35, with nitro, cyano, acetyl group, ester and carbonyl groups being still more preferred. Halogen and electron withdrawing groups can increase the acid strength of the benzenesulfonic acid through an inductive effect by bonding to a carbon atom on the aromatic ring of the sulfonic acid. Fluorine is an atom having the highest electronegativity, but has a higher ability to activate a benzene ring through a resonance effect than other electron withdrawing groups, and therefore does not give as high acid strength as other halogen substituents such as chlorine and bromine. The foregoing electron withdrawing groups have a larger atomic weight over fluorine, make the benzenesulfonic acid have a rigid structure, and are useful for suppressing acid diffusion. The values of Hammett substituent constant am of the groups herein are as described in Chem. Rev. 1991, 91, 165-169, and am values that are not covered are determined by calculating a difference between pKa of benzoic acid and pKa of a benzoic acid derivative with a substituent at the m-position using the software ACD/ChemSketch (ACD/Labs 8.00 Release Product Version: 8.08).

A plurality of R² may bond together to form a ring with the carbon atoms to which they are attached when m5 is 2 or more. The ring is preferably a 5 to 8-membered ring.

L^{A}, L^{B}, L^{C} and L^{D} are each independently a single bond, ester bond, ether bond, amide bond, sulfonate ester bond, sulfonamide bond, carbonate bond or carbamate bond,

In the formula (a), X¹ and X² are each independently a single bond, or a C₁-C₄₀ hydrocarbylene group which may contain a heteroatom. Examples thereof include alkanediyl, cyclic saturated hydrocarbylene, and arylene groups. Examples of the heteroatom include oxygen, nitrogen and sulfur atoms.

Examples of the C₁-C₄₀ hydrocarbylene groups X¹ and X² which may contain a heteroatom are shown below, but not limited thereto. In the following formulae, * is a point of attachment to L^{A} and L^{B} or L^{C} and L^{D}.

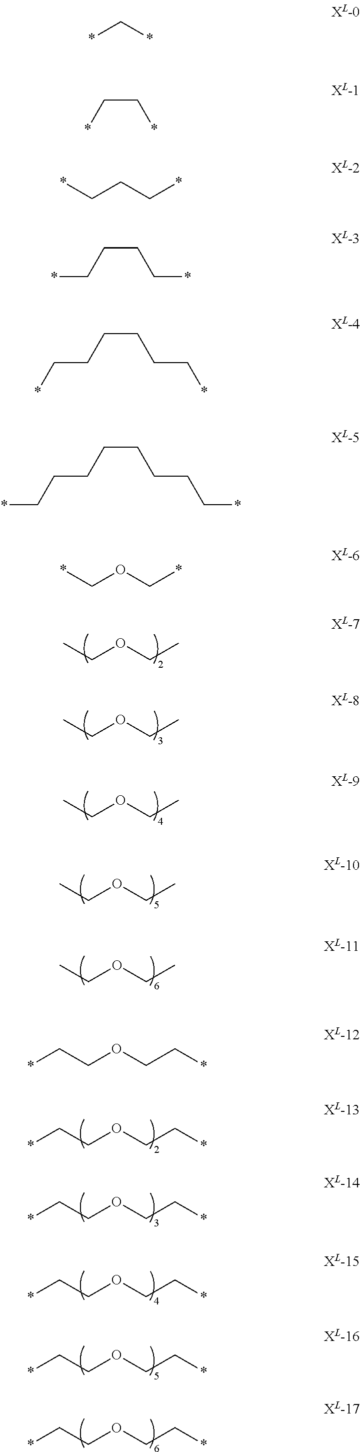

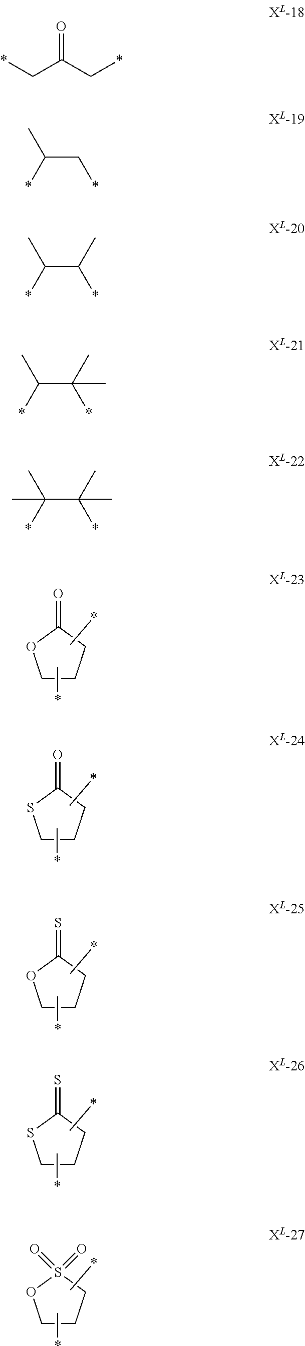

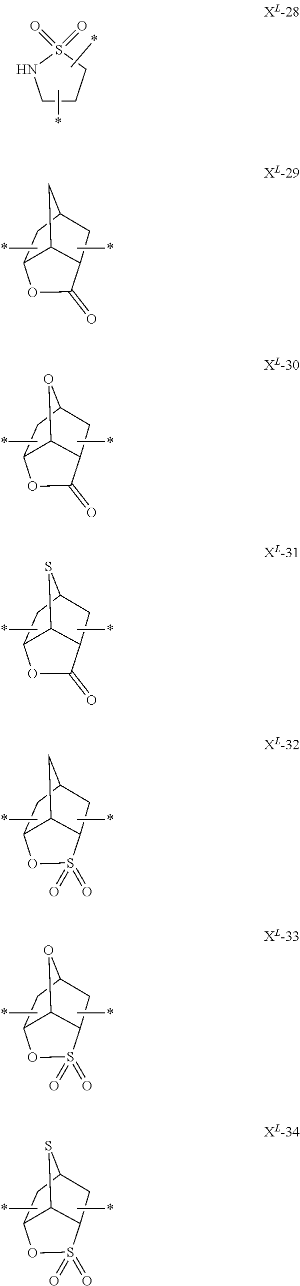

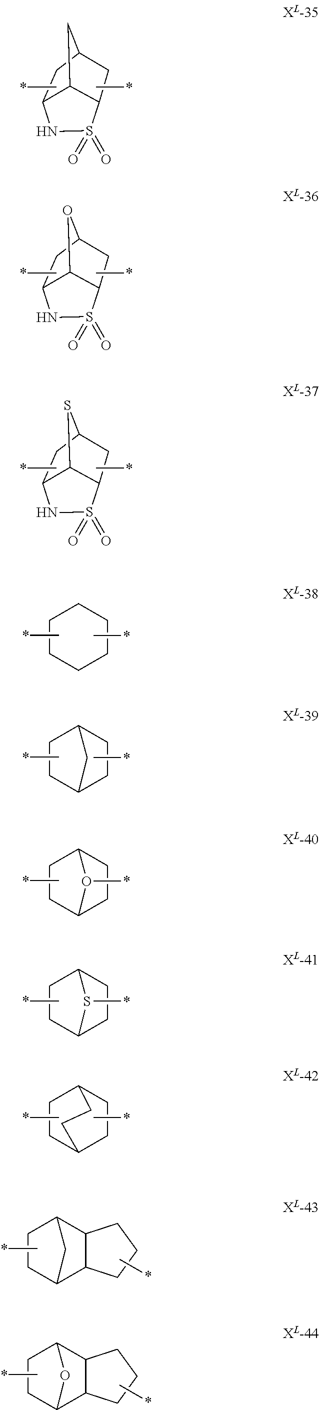

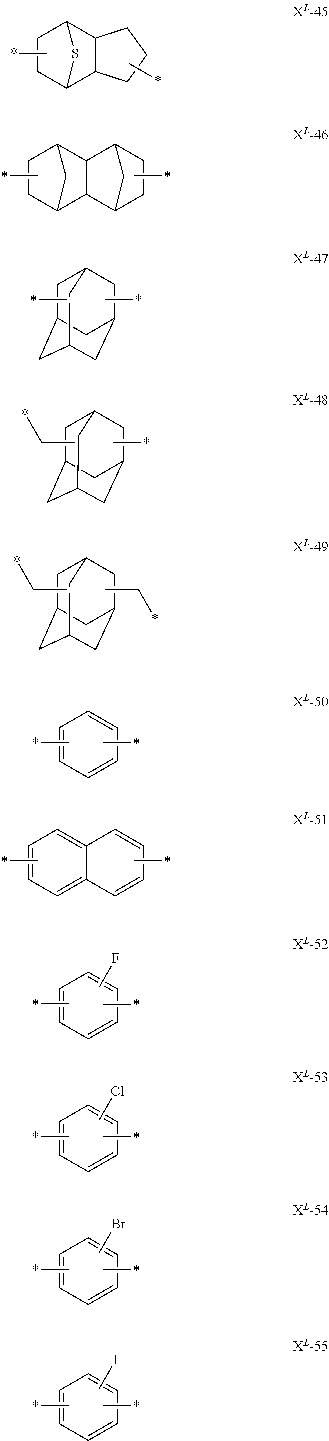

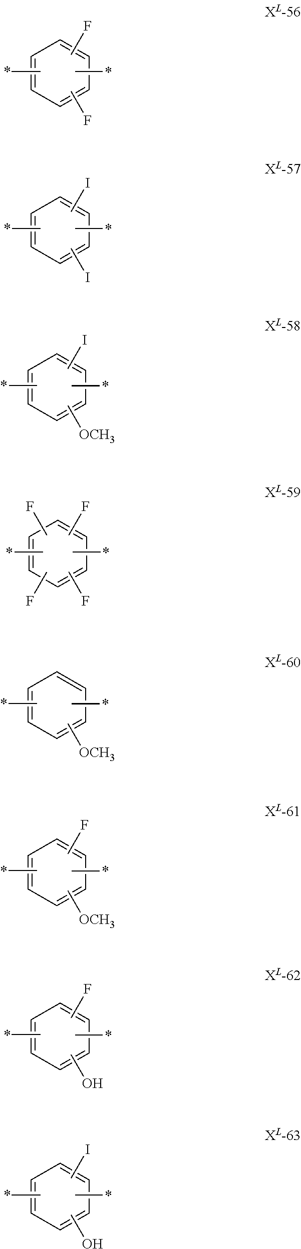

Of these, X^{L}-0 to X^{L}-22, X^{L}-29 to X^{L}-34, and X^{L}-47 to X^{L}-63 are preferred.

In the formula (a), L^{A} is preferably a single bond or ester bond, more preferably a single bond. Of these, a single bond, ester bond or ether bond is preferred as L^{B}. In particular, when -L^{A}-X¹-L^{B}- is a single bond (i.e., each of L^{A}, X¹ and L^{B} is a single bond), ease with which the photoacid generator incorporated in the polymer backbone moves around the backbone is reduced, so that acid diffusion can be further suppressed. On the other hand, for improving solvent solubility, a linear alkyl group including an ether group or an ester group is also preferred.

In the formula (a), L^{C} is preferably a single bond, ether bond, ester bond or sulfonic ester bond, more preferably a single bond. The distance between the polymer backbone and the sulfonic acid decreases, so that acid diffusion can be further suppressed. L^{D} is preferably a single bond, ether bond, ester bond or sulfonic ester bond, more preferably an ester bond or sulfonic ester bond.

Of the onium salt monomers of formula (a), a structure having formula (a1) is preferred.

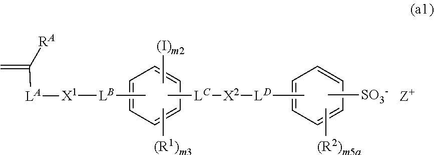

wherein m2, m3, R^{A}, R¹, R², X¹, X², L^{A} to L^{D} and Z⁺ are as defined above, and m5a is 1, 2, 3 or 4.

Of the onium salt monomers of formula (a1), a structure having formula (a2) is preferred.

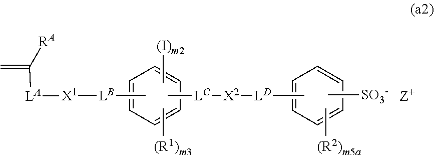

wherein m2, m3, m5a, R^{A}, R¹, X¹, X², L^{A} to L^{D} and Z⁺ are as defined above, and R^{2A} is an electron withdrawing group exclusive of halogen.

Examples of the anion in the onium salt having formula (a) are shown below, but not limited thereto. In the following formulae, R^{A} is as defined above, and Me is methyl. Ac is an acetyl group. The bond positions of the substituents on the aromatic ring are interchangeable.

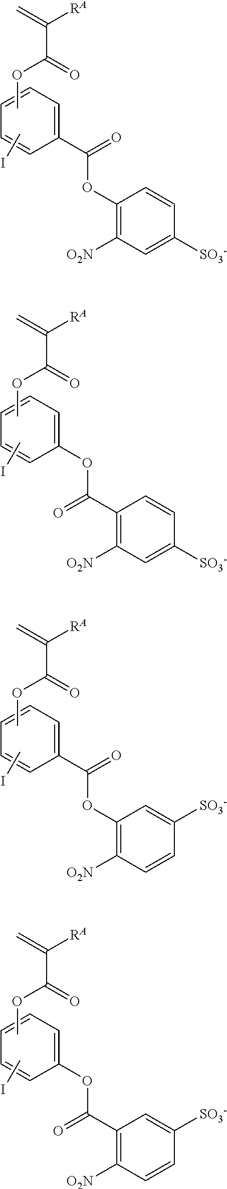

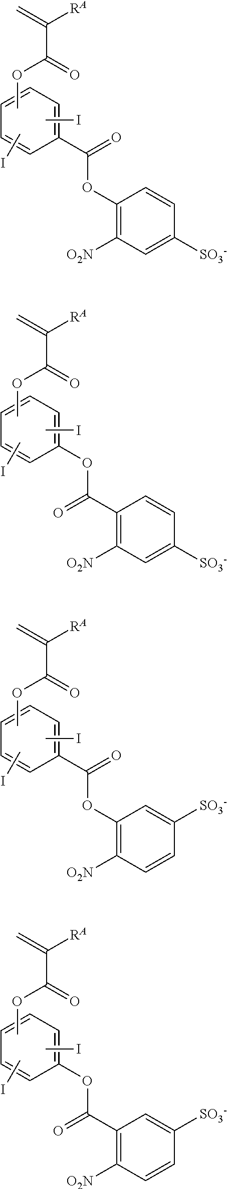

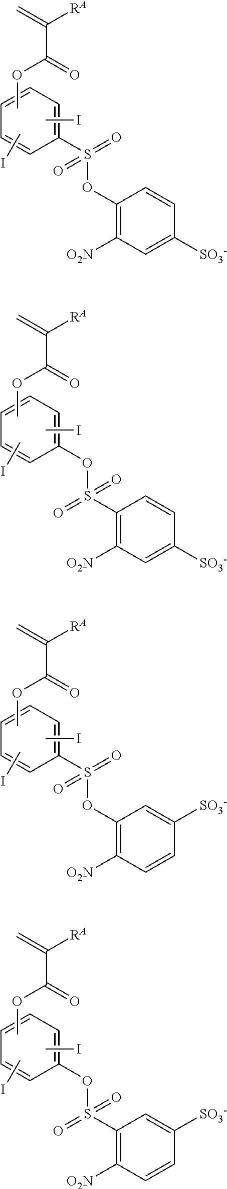

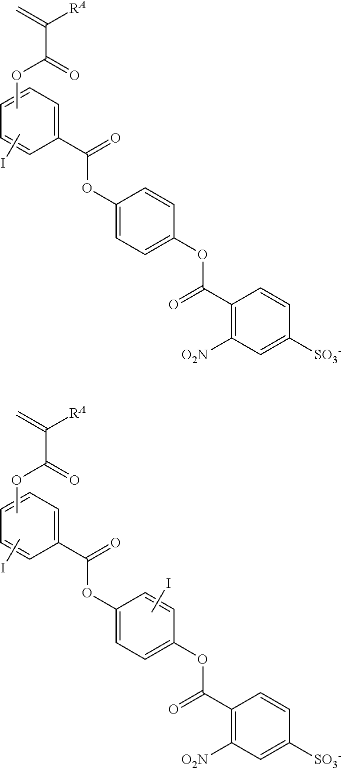

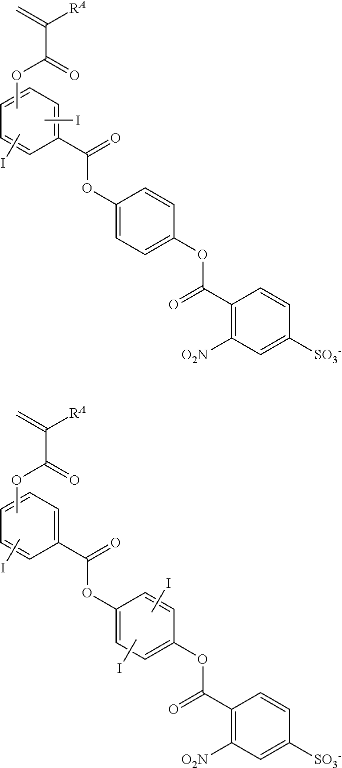

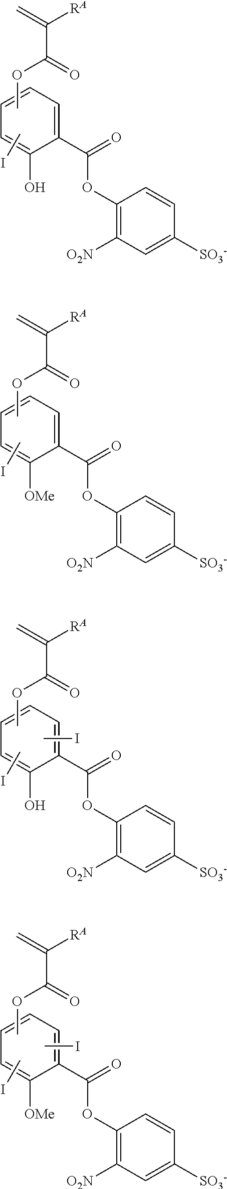

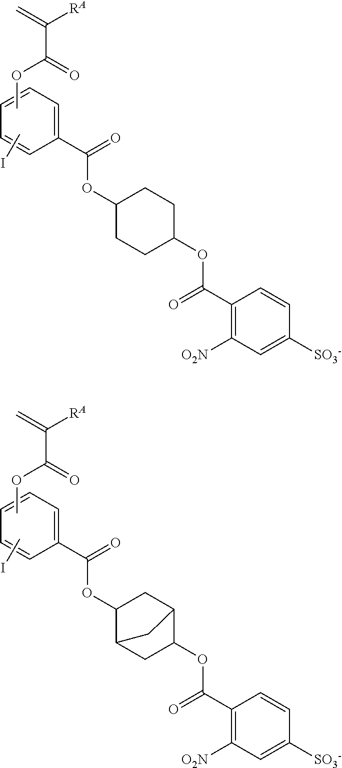

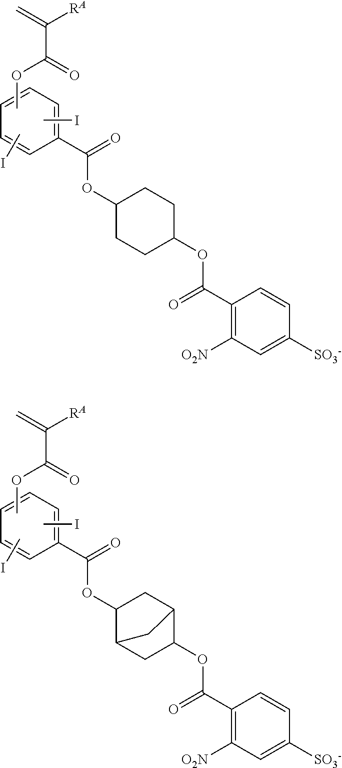

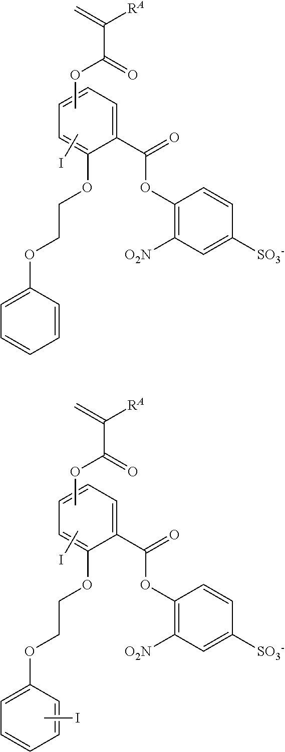

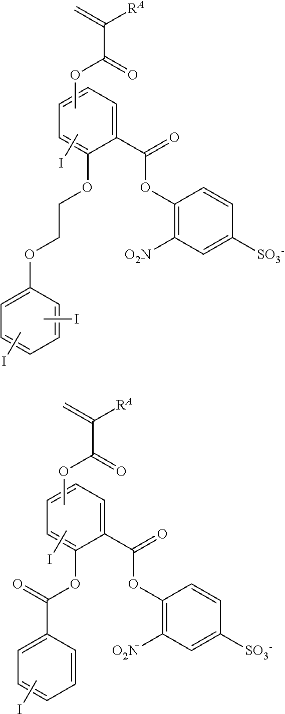

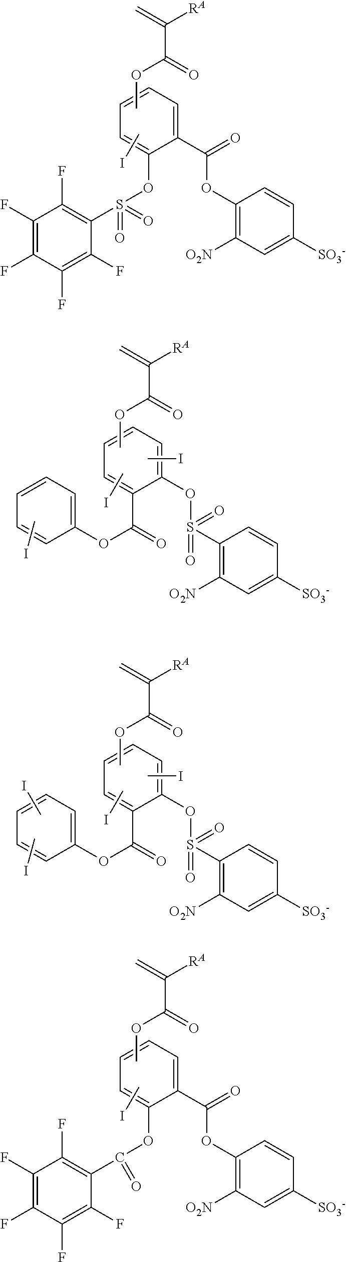

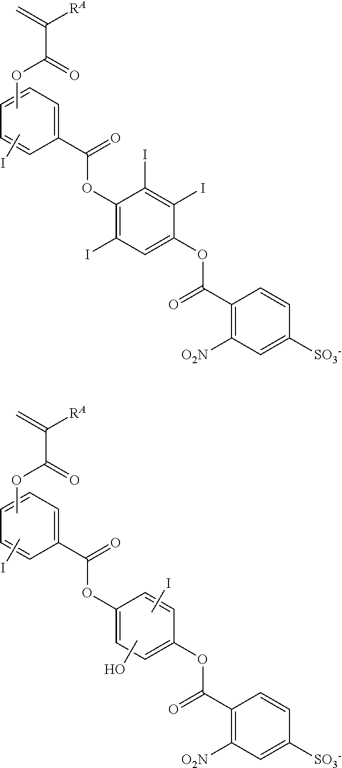

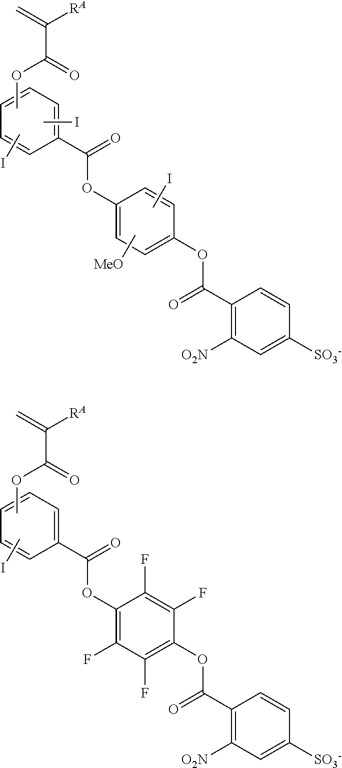

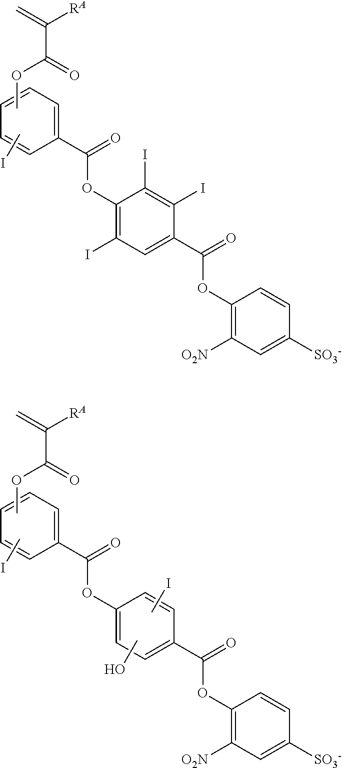

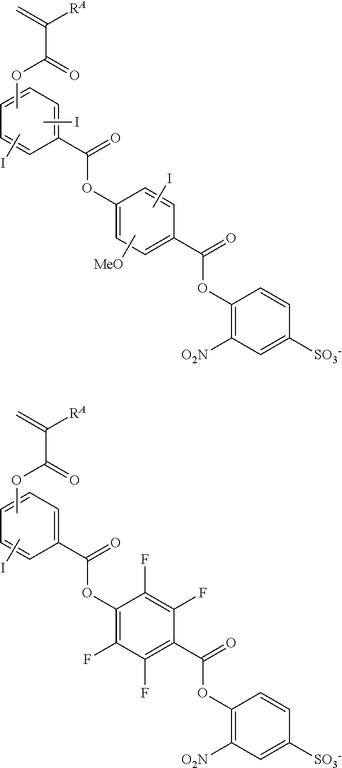

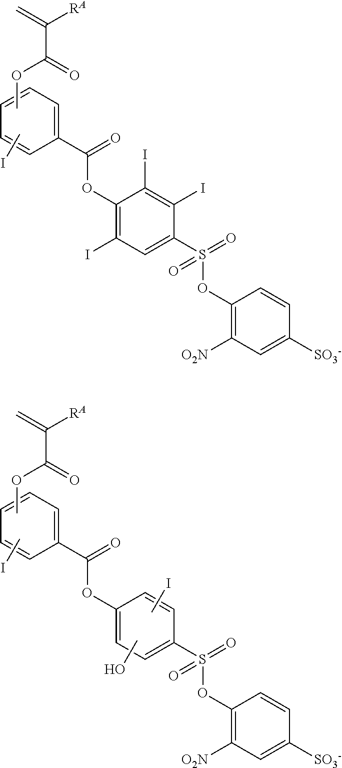

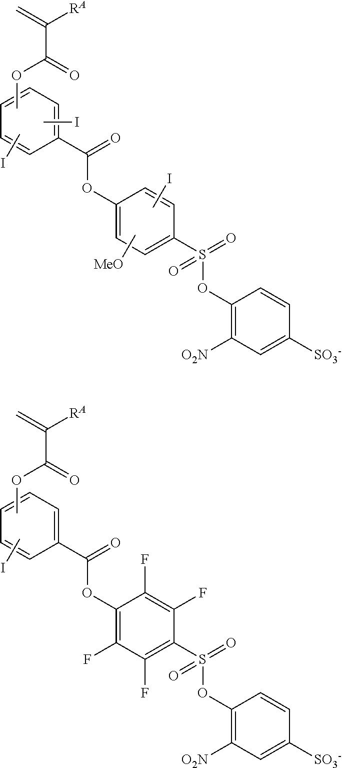

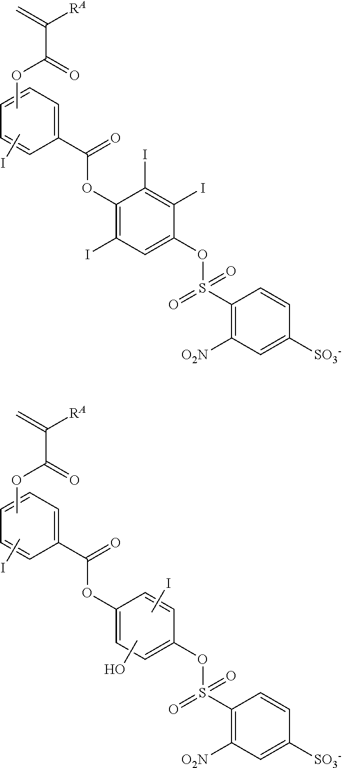

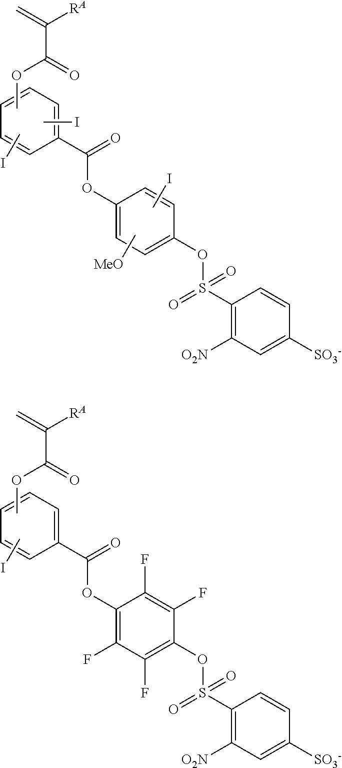

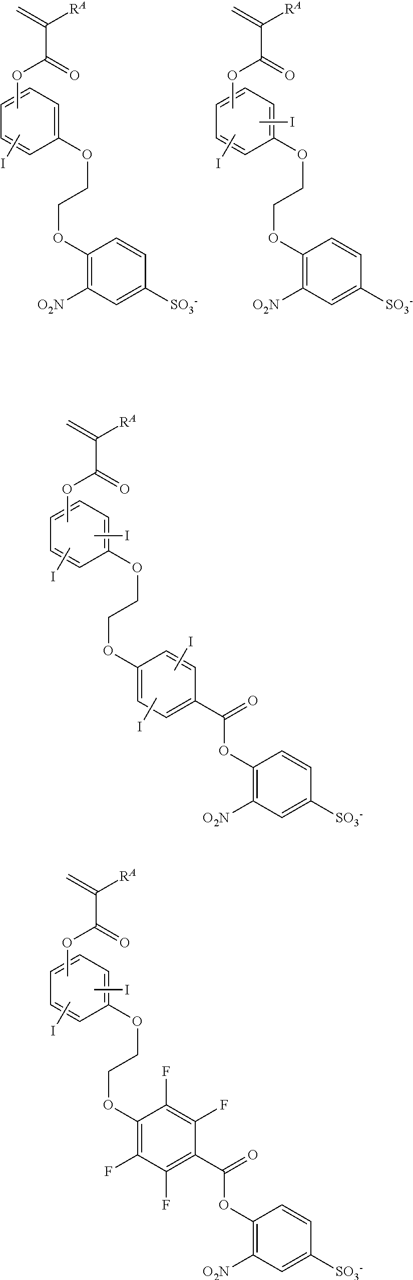

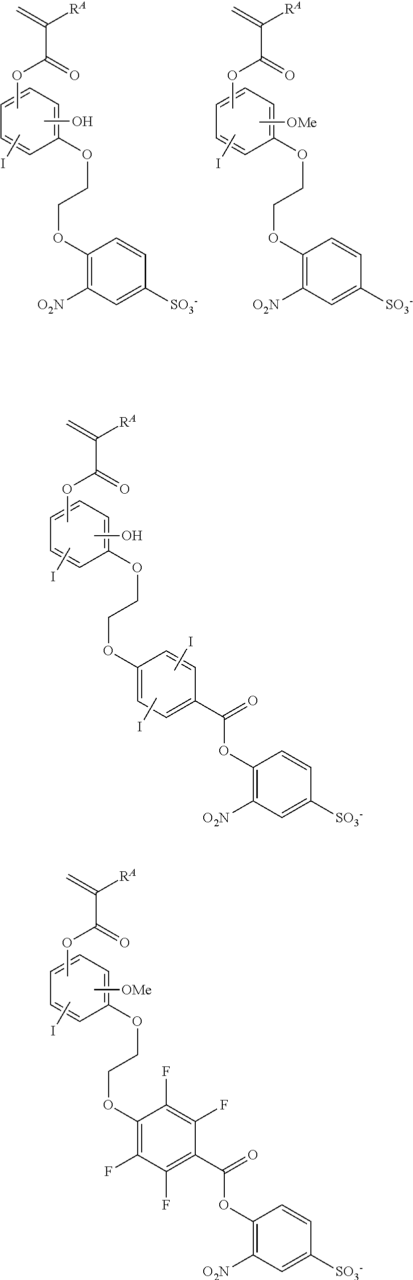

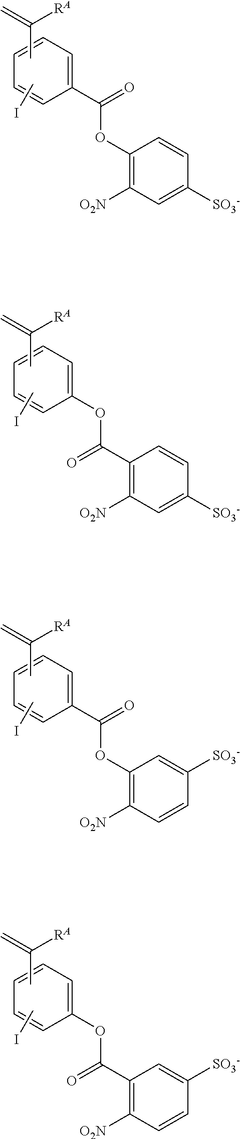

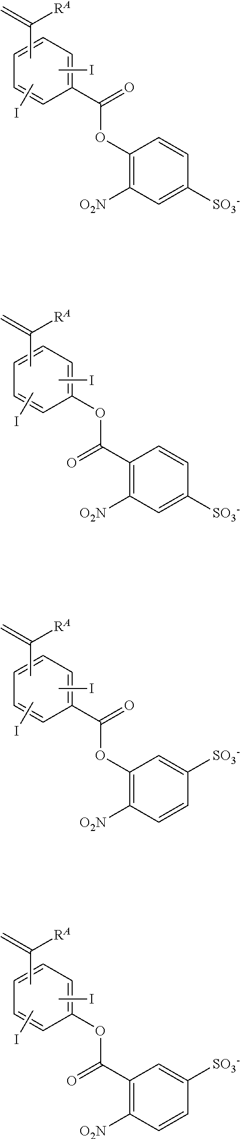

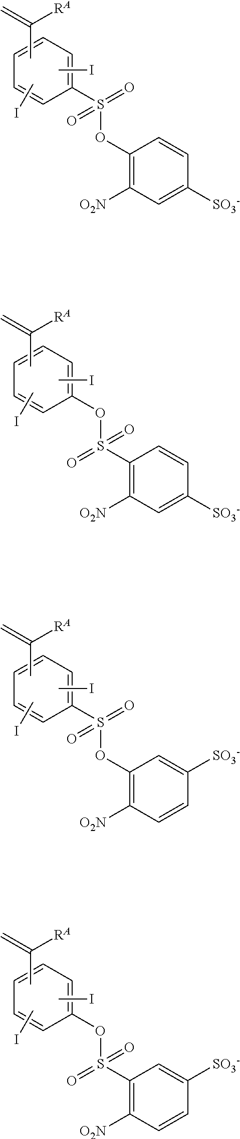

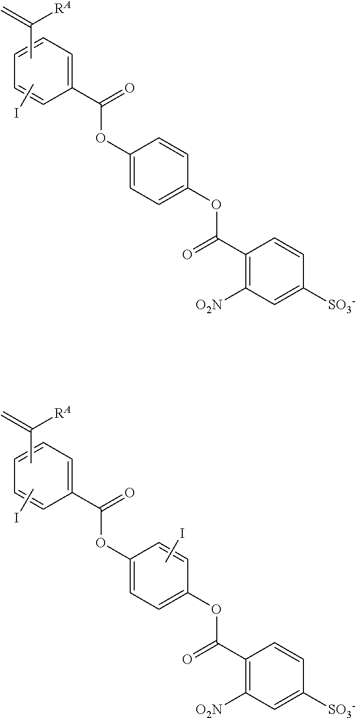

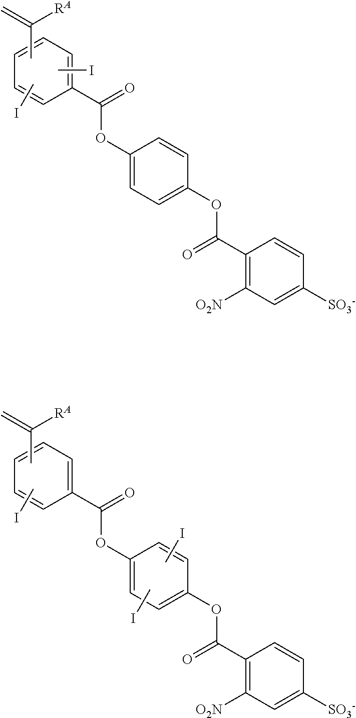

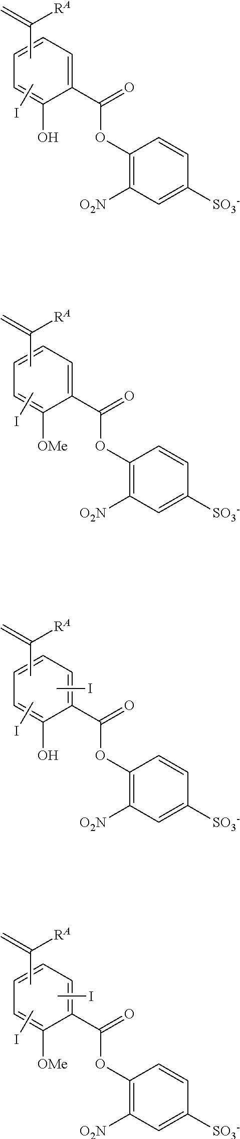

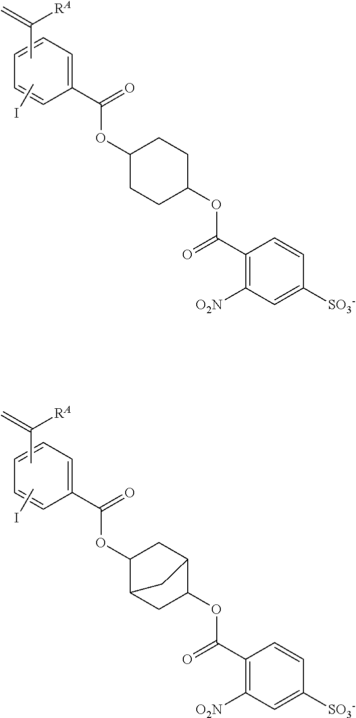

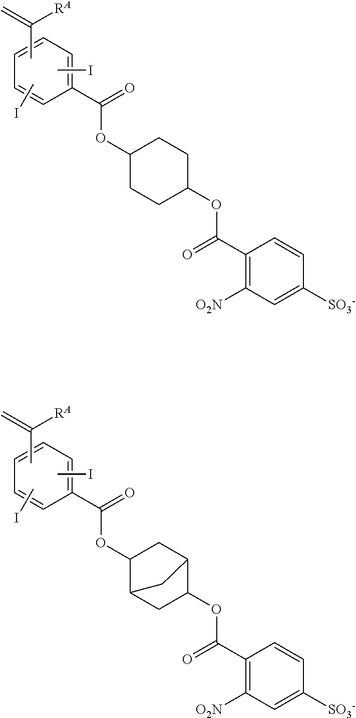

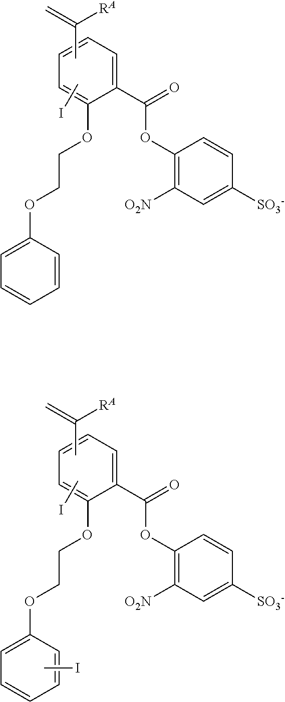

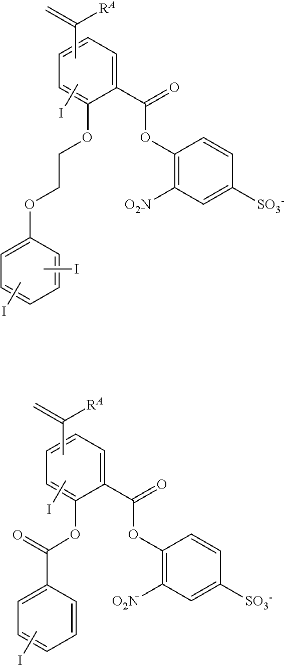

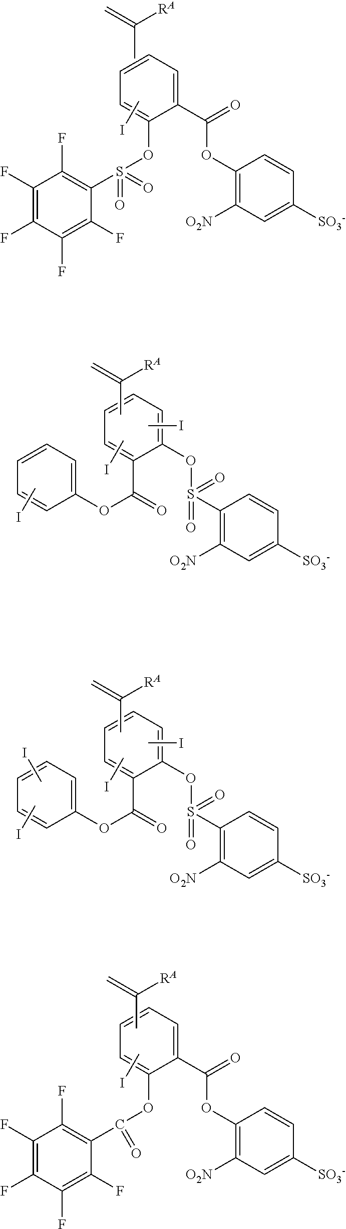

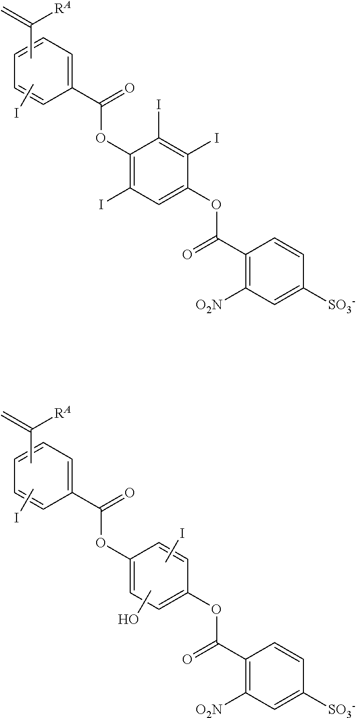

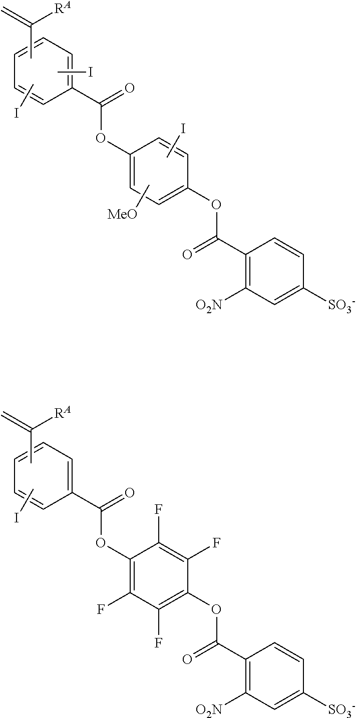

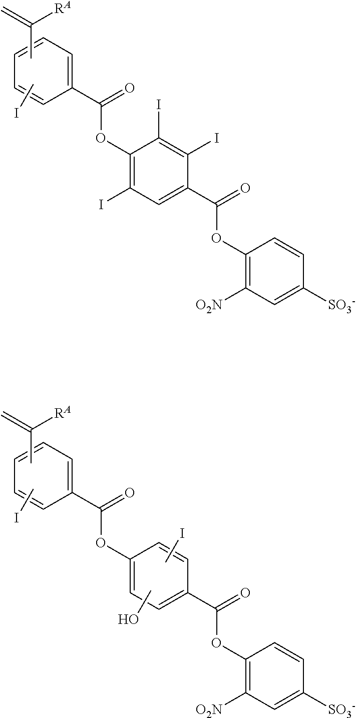

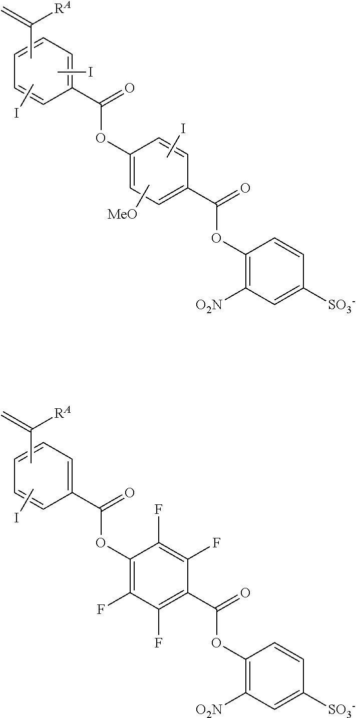

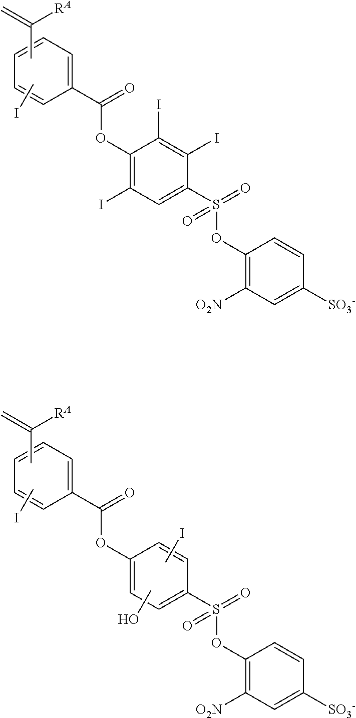

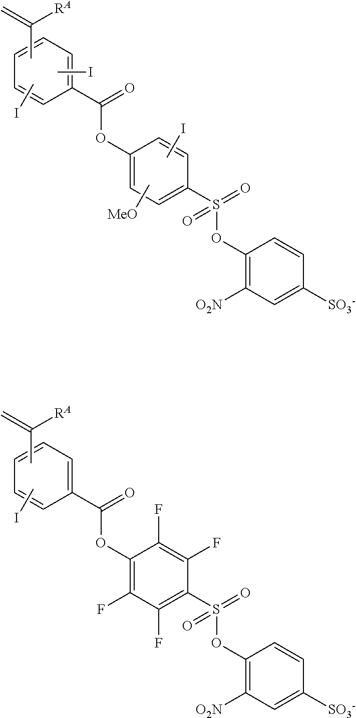

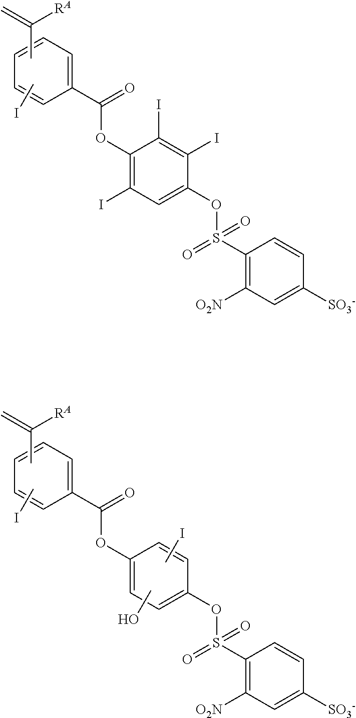

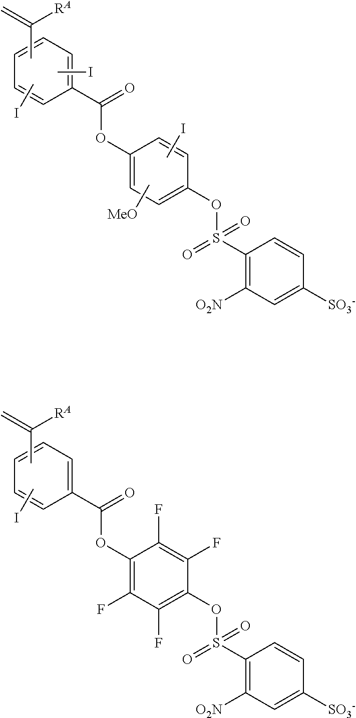

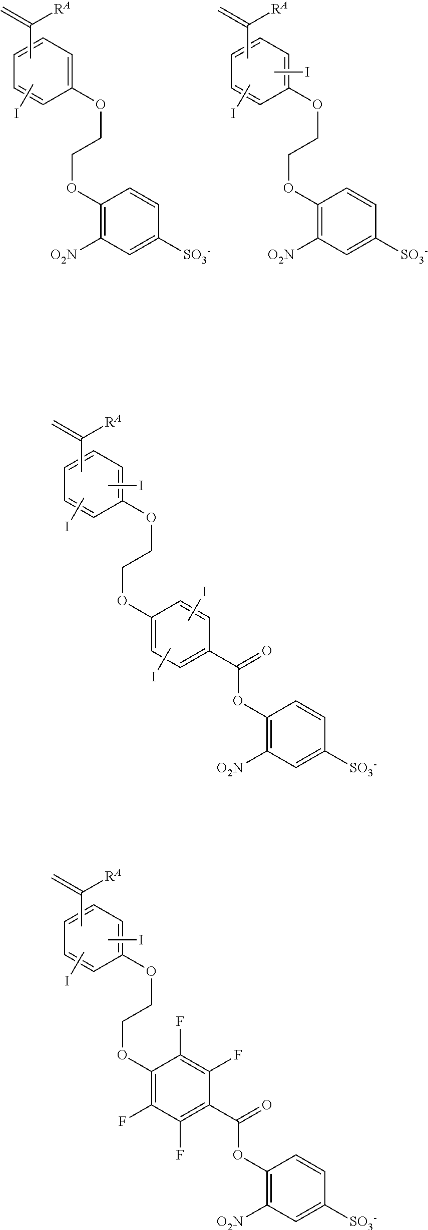

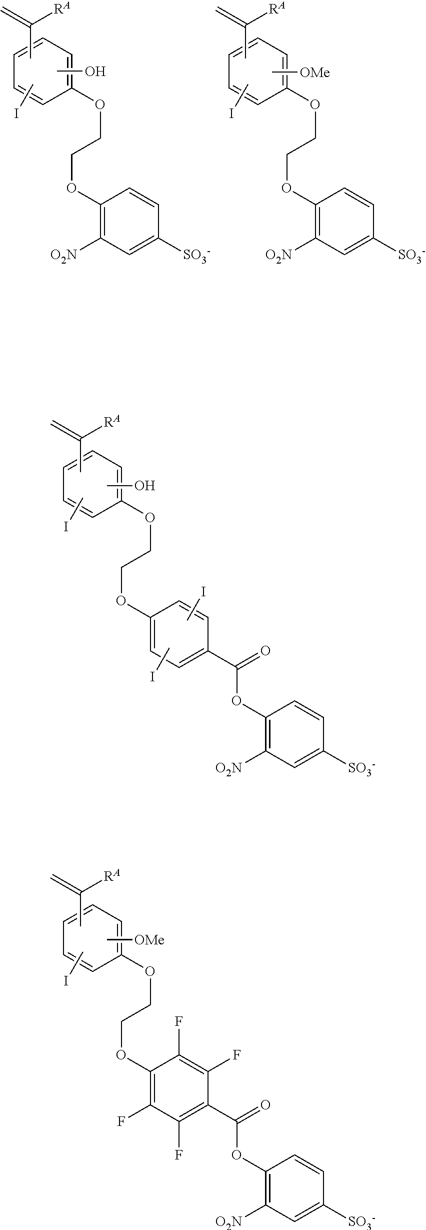

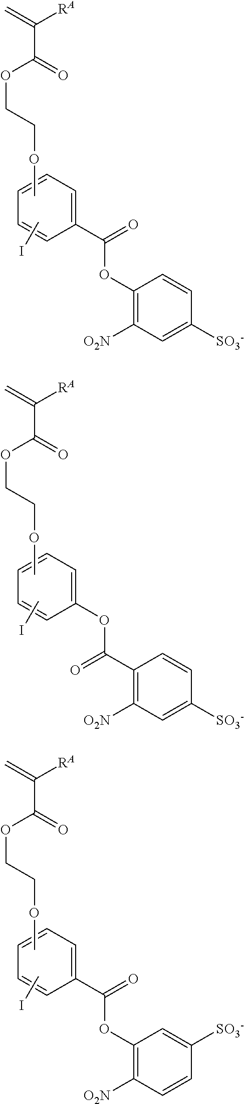

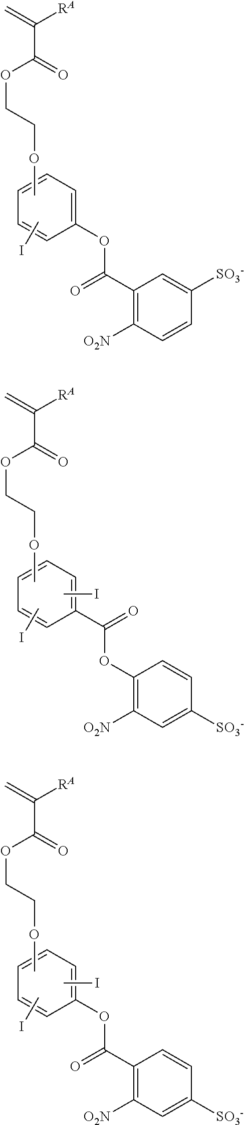

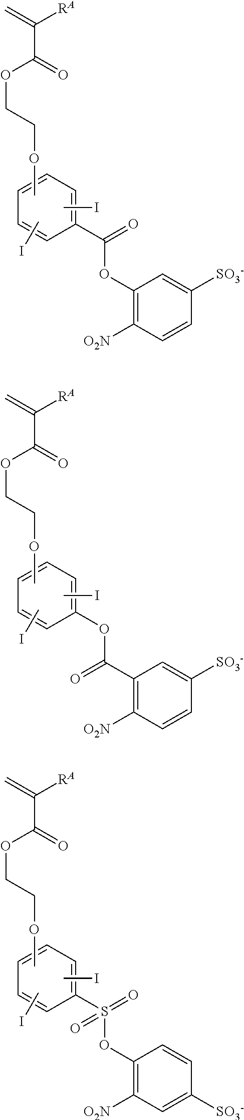

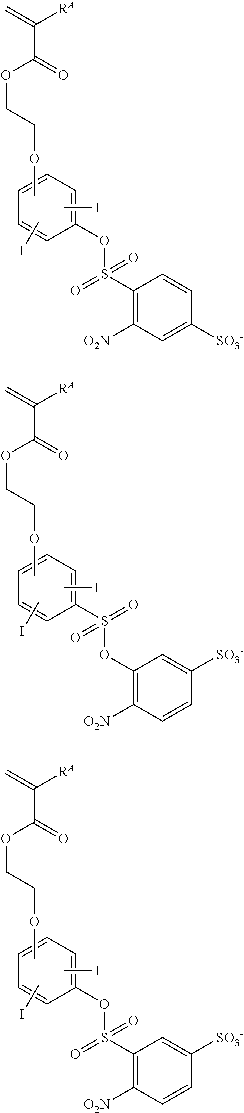

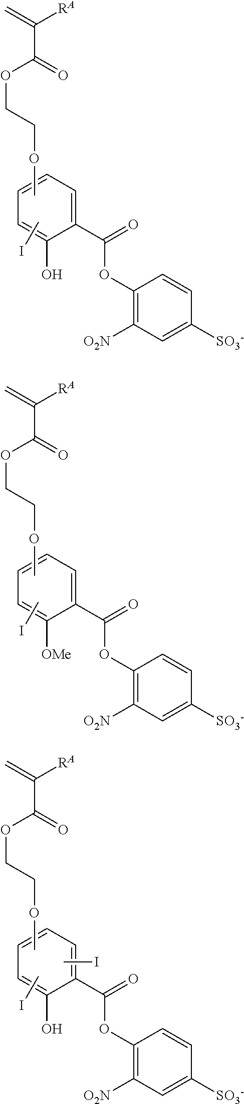

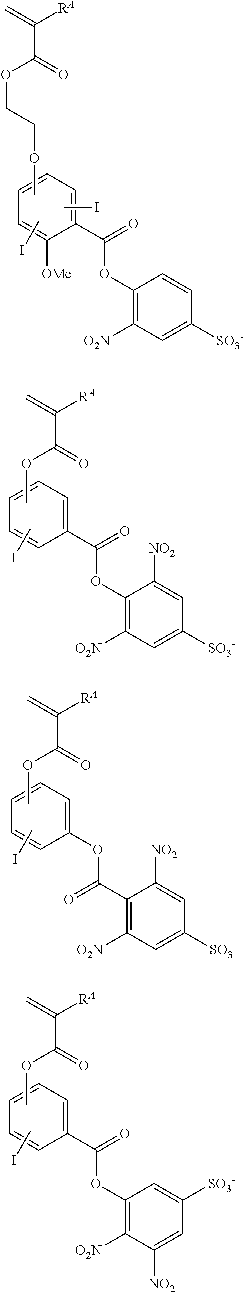

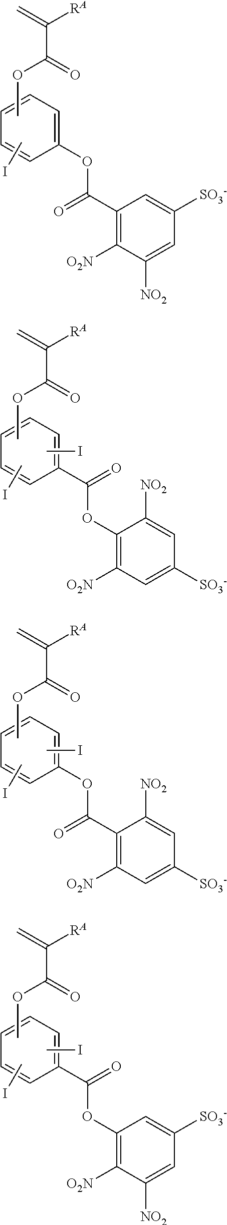

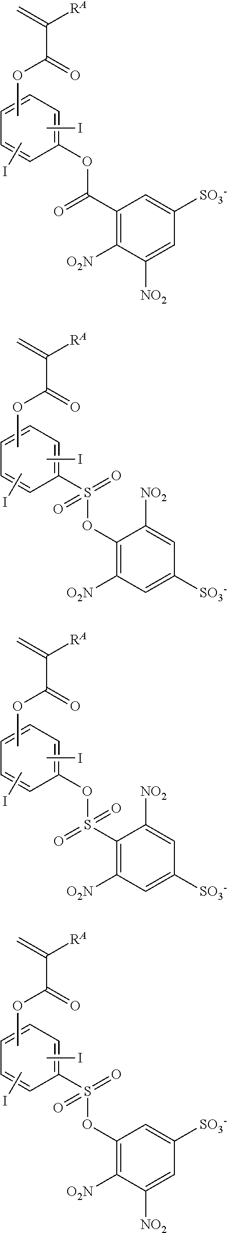

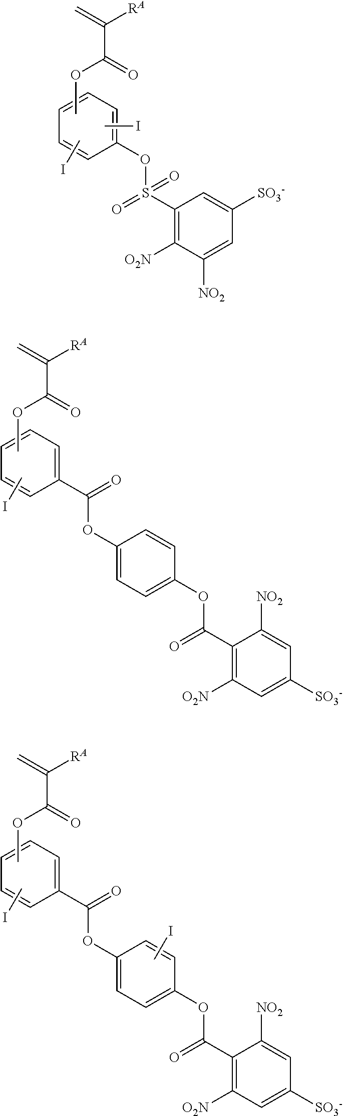

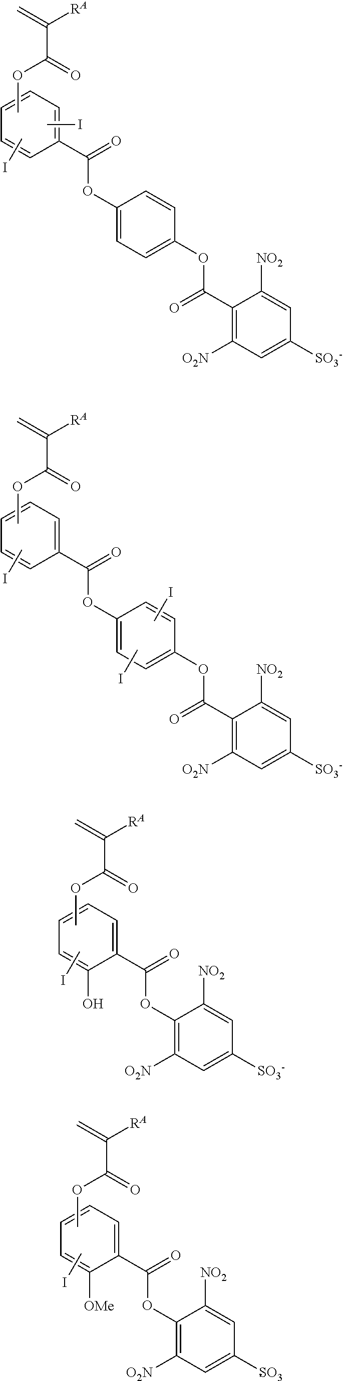

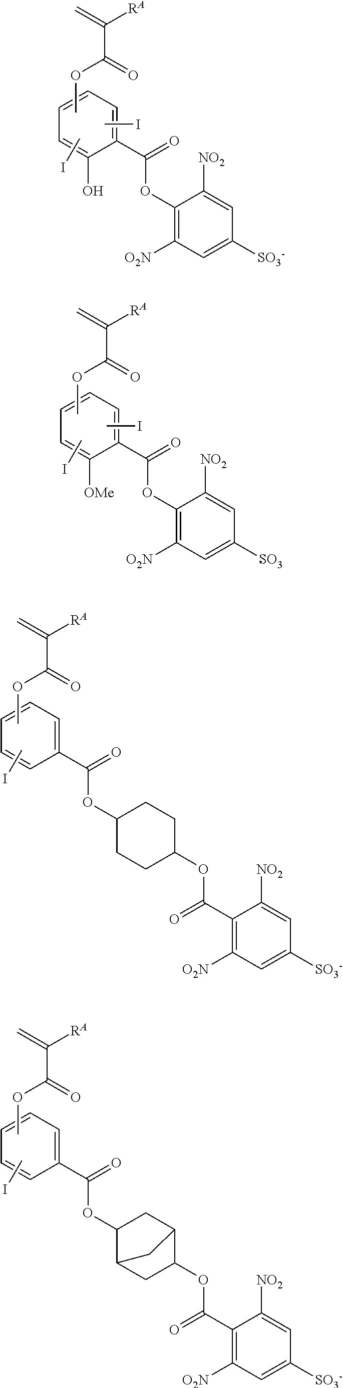

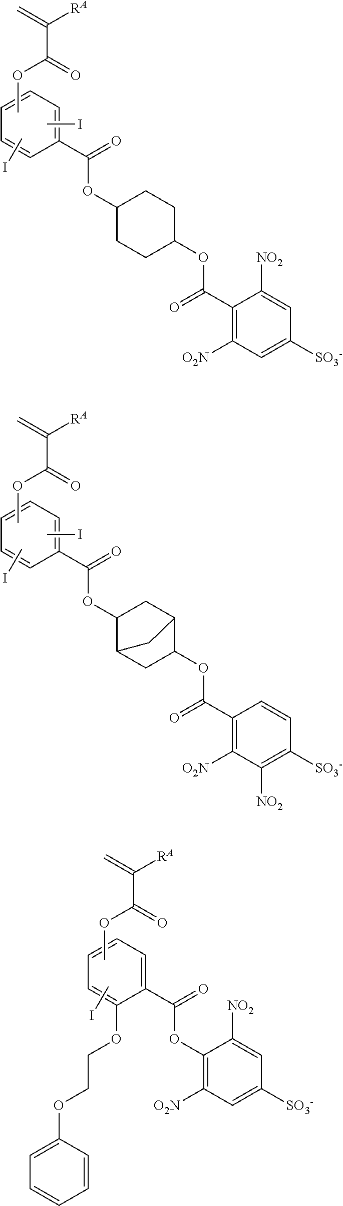

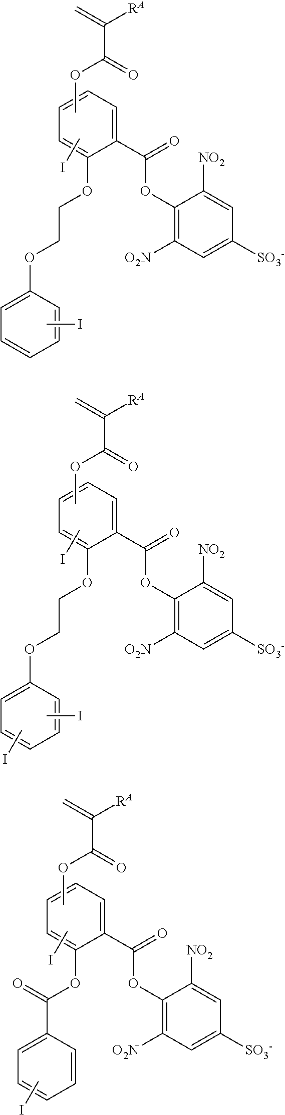

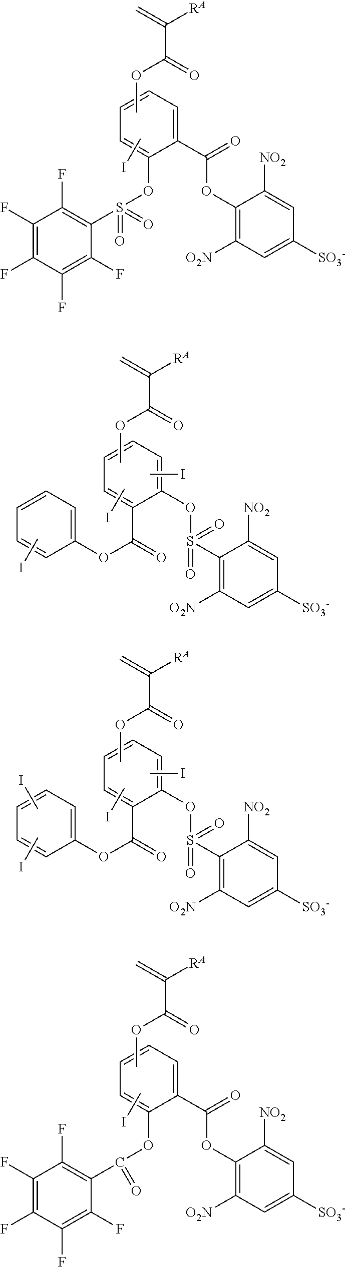

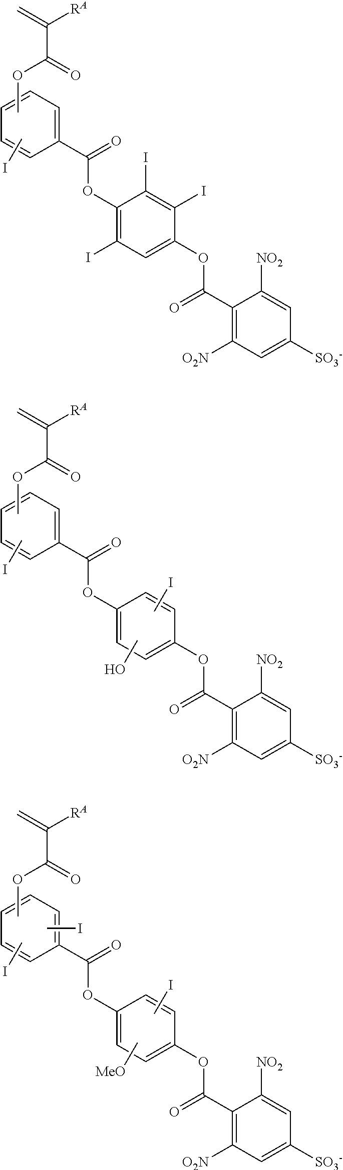

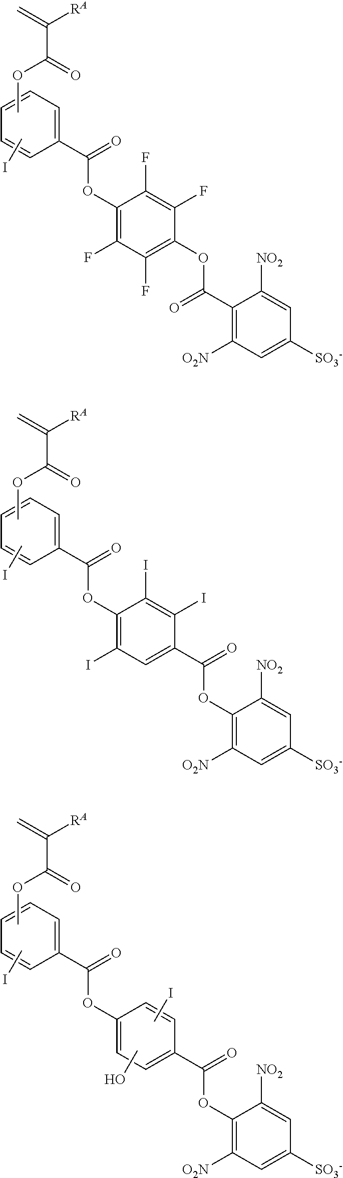

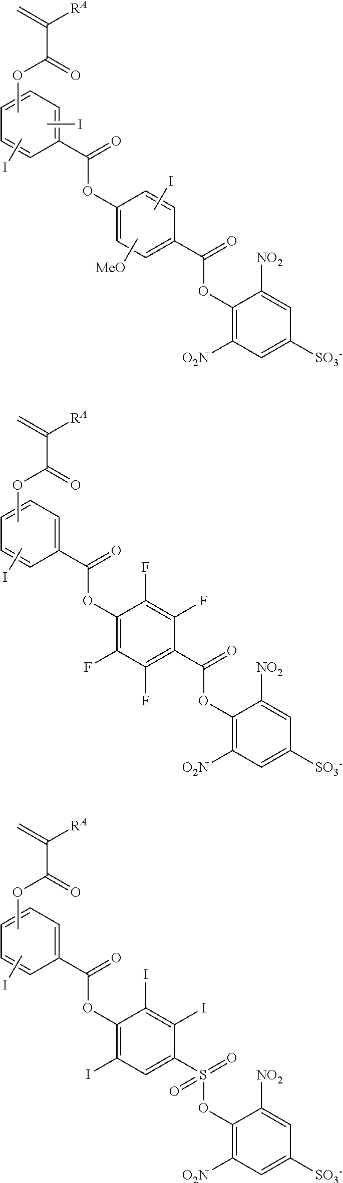

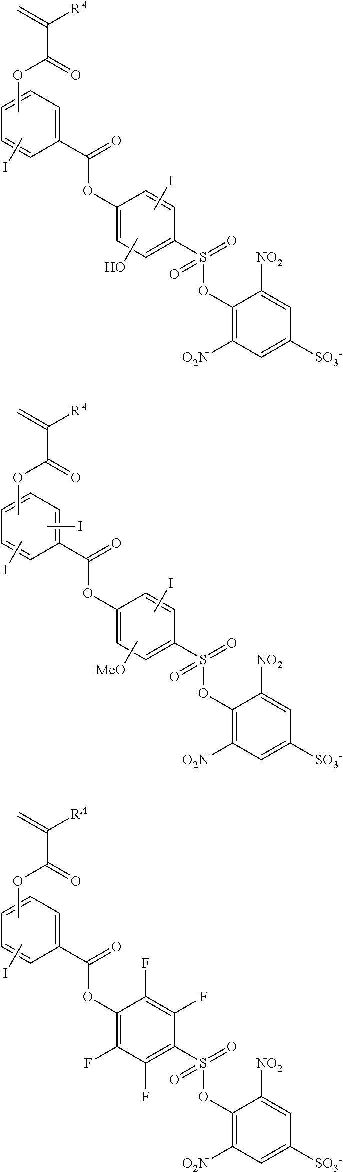

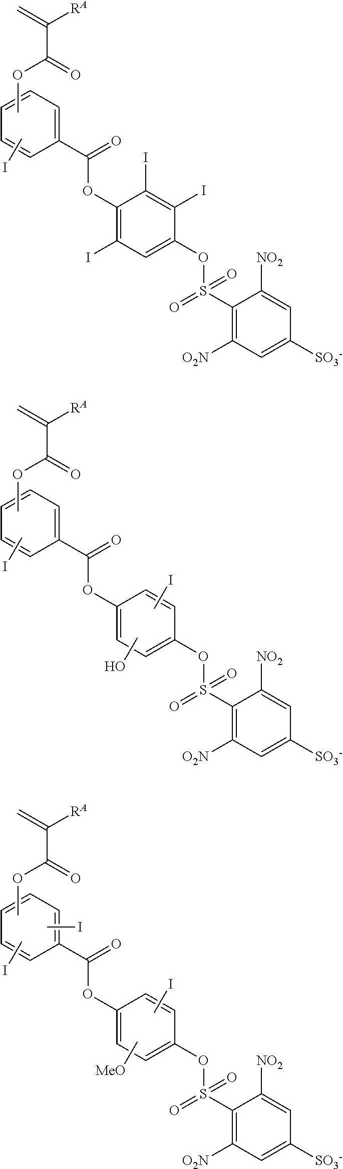

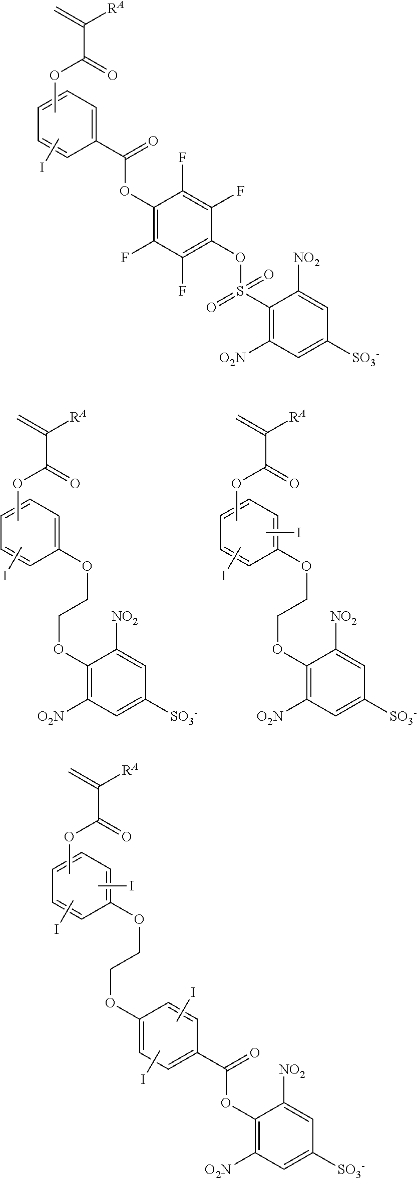

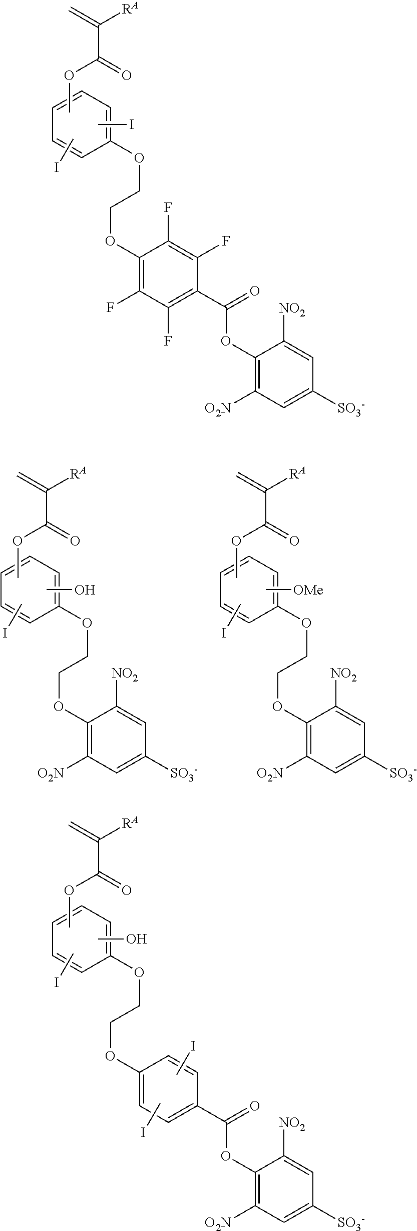

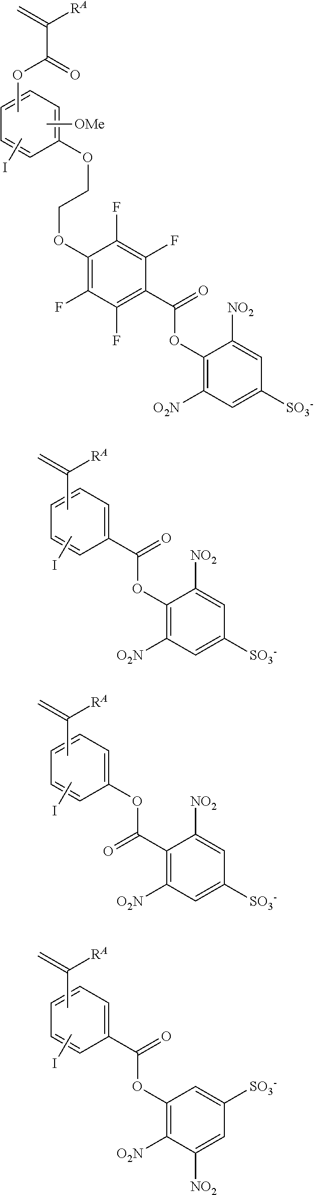

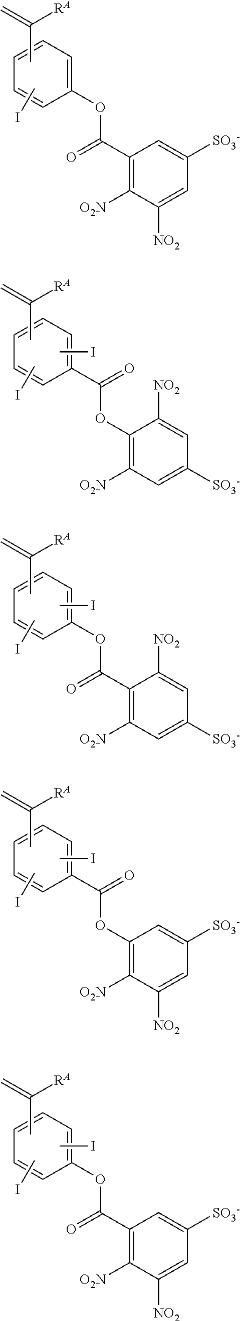

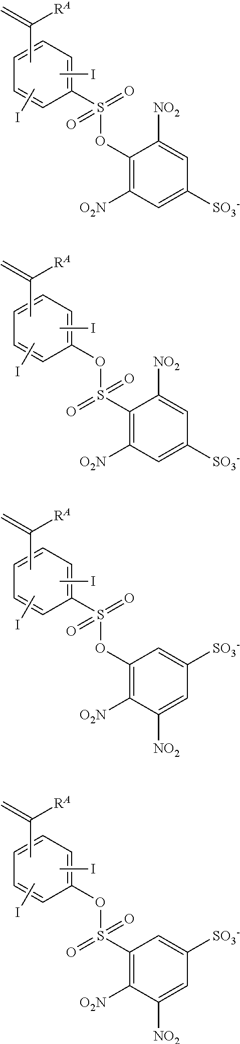

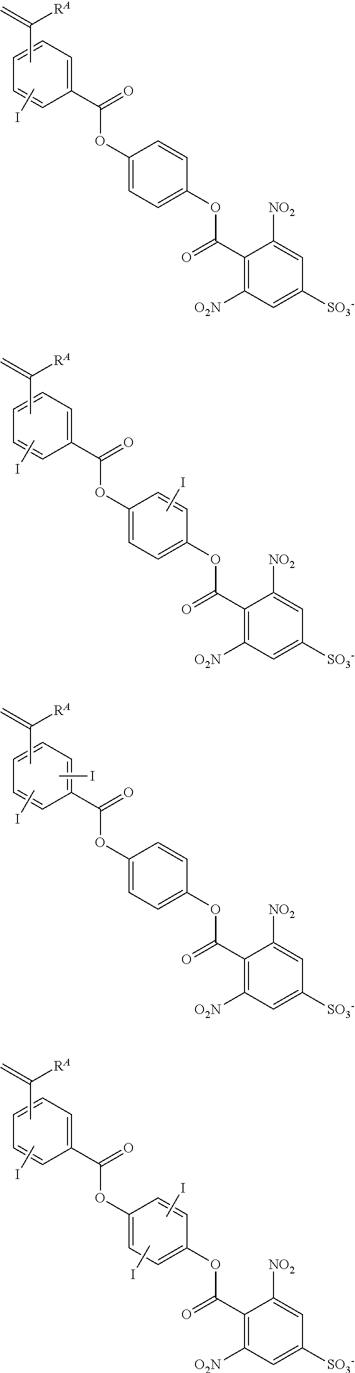

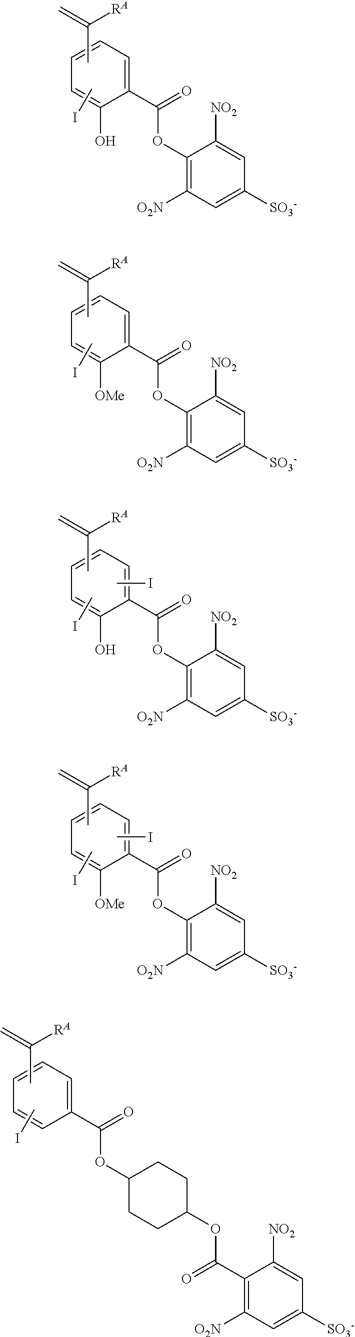

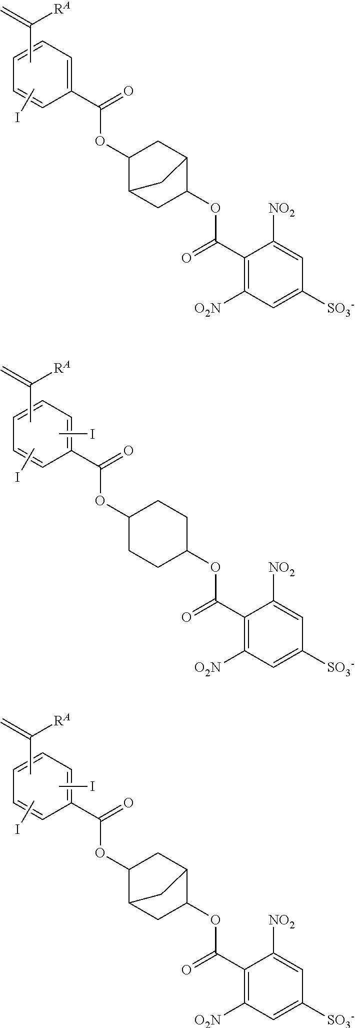

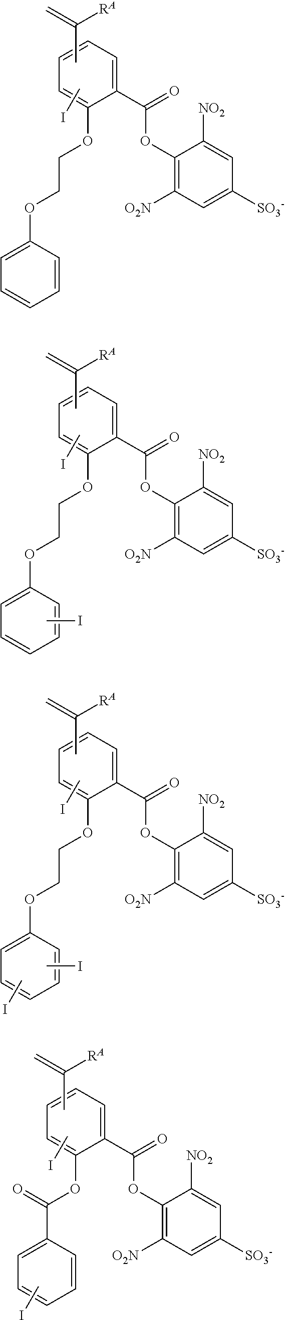

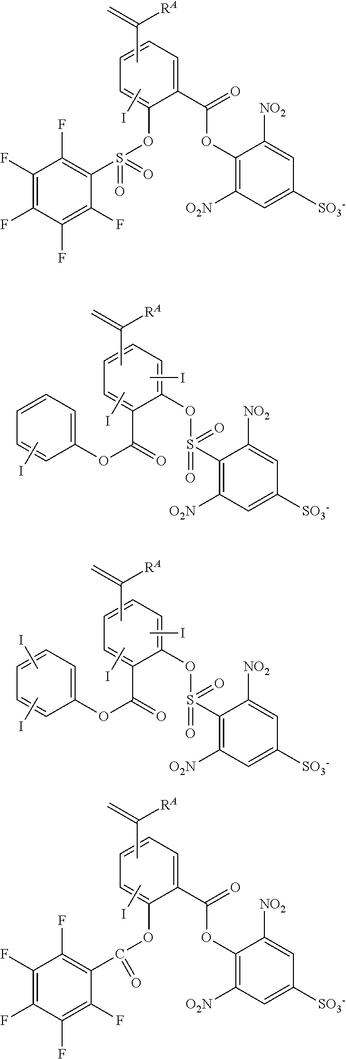

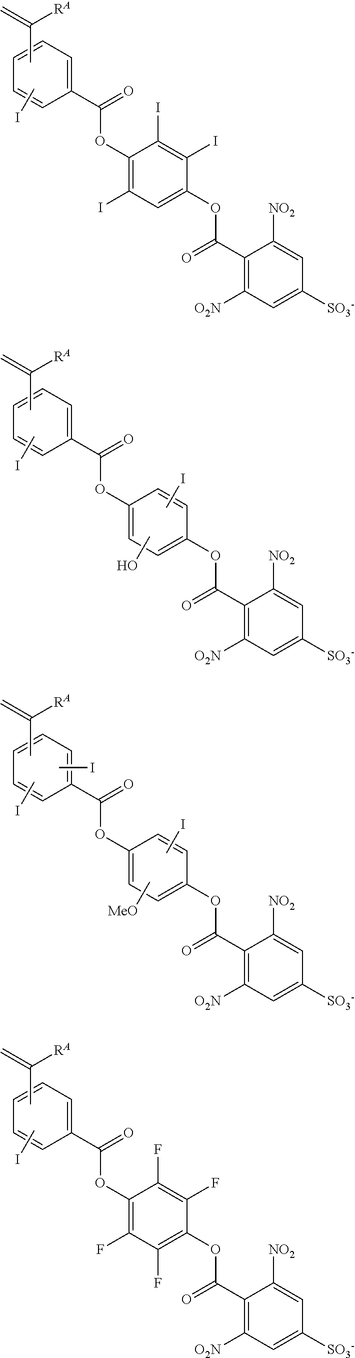

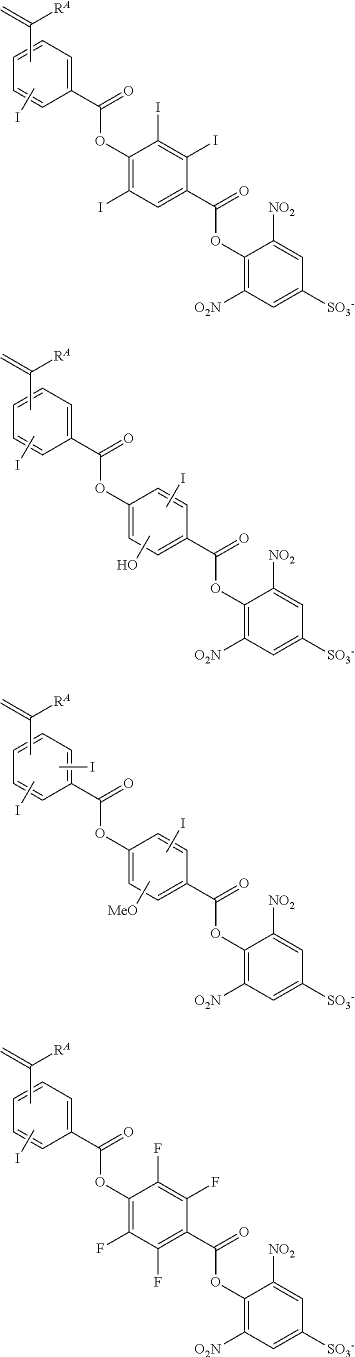

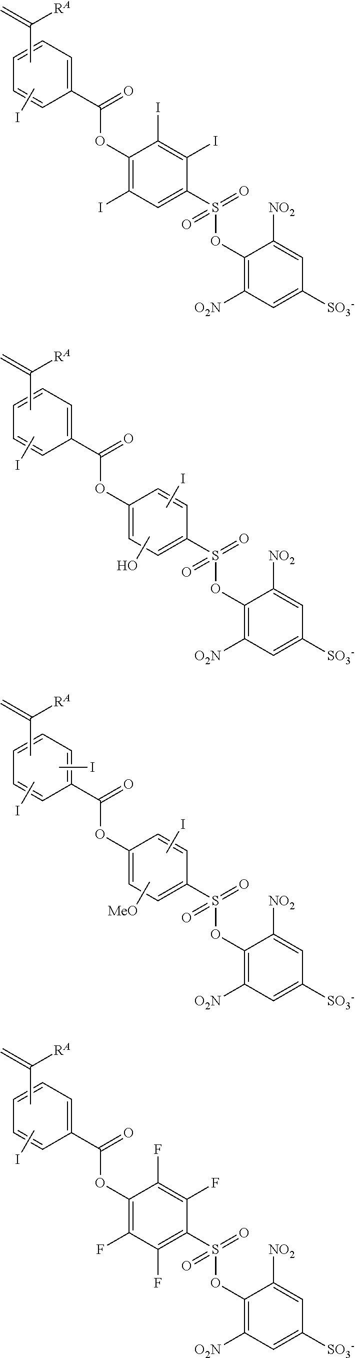

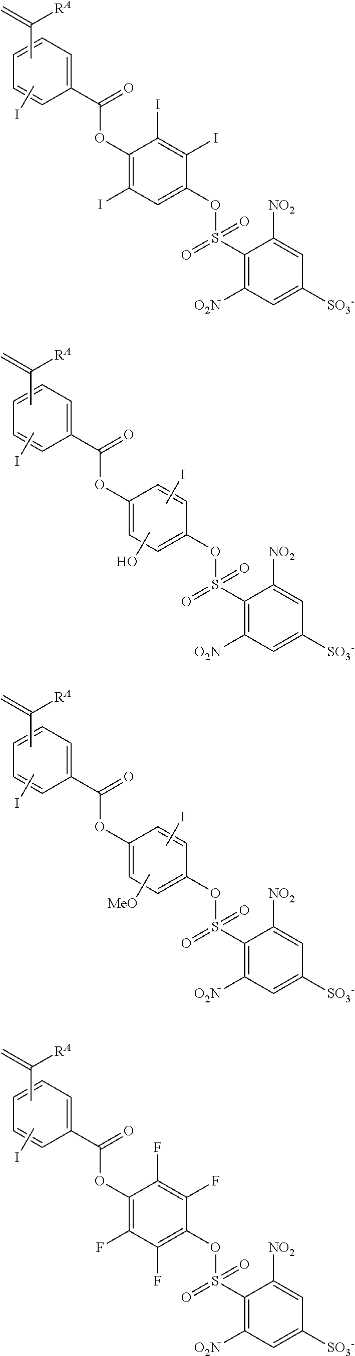

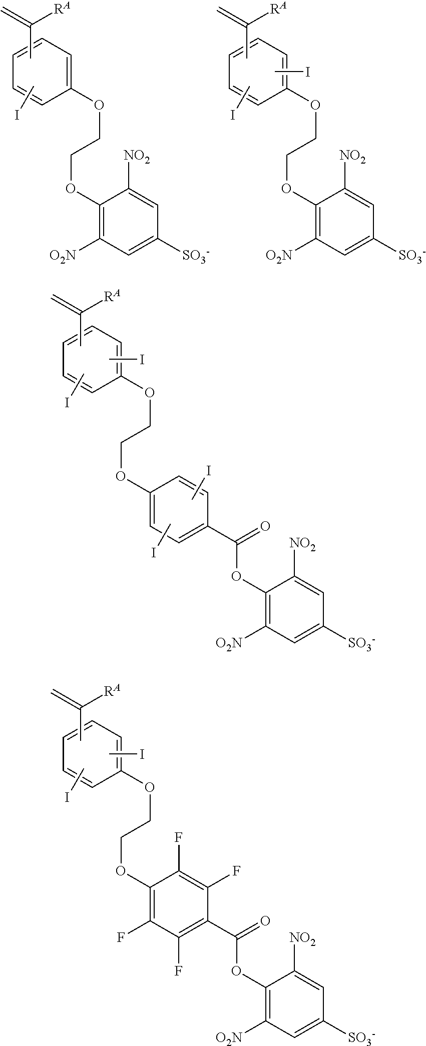

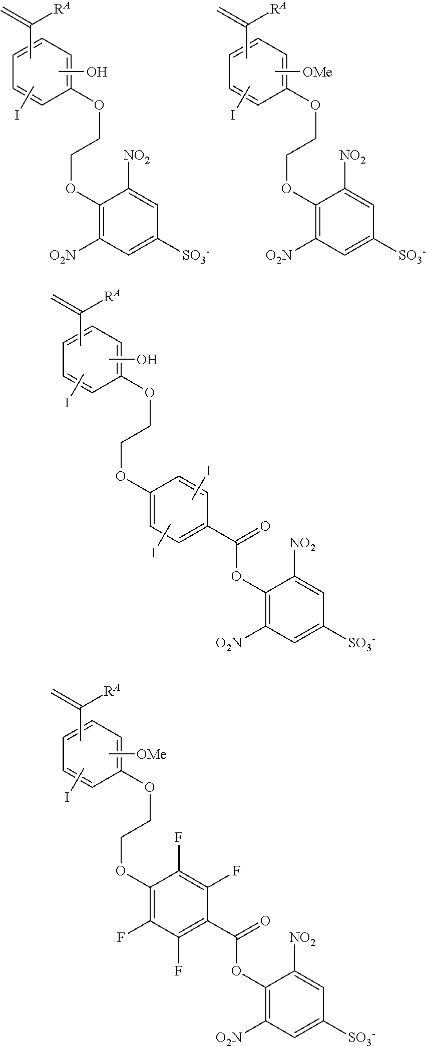

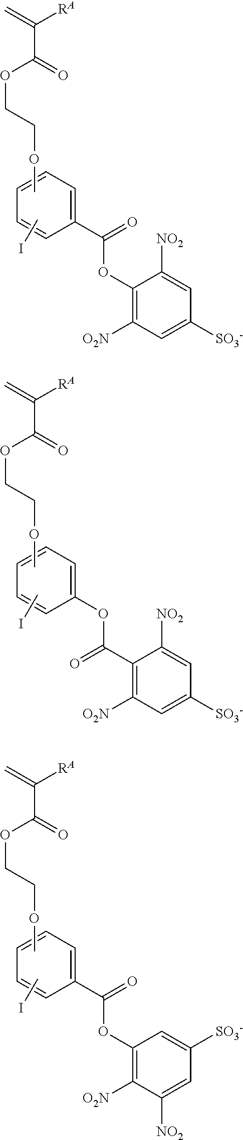

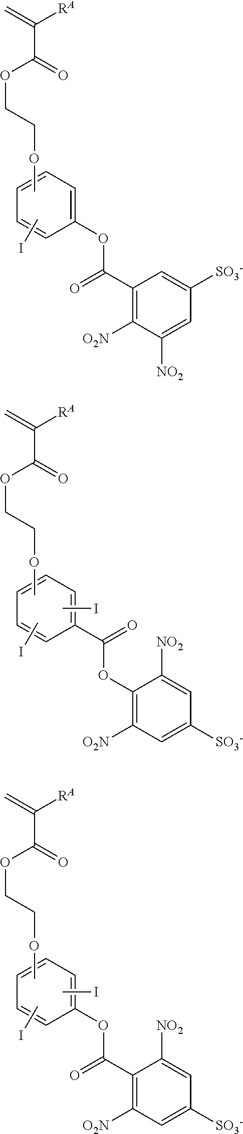

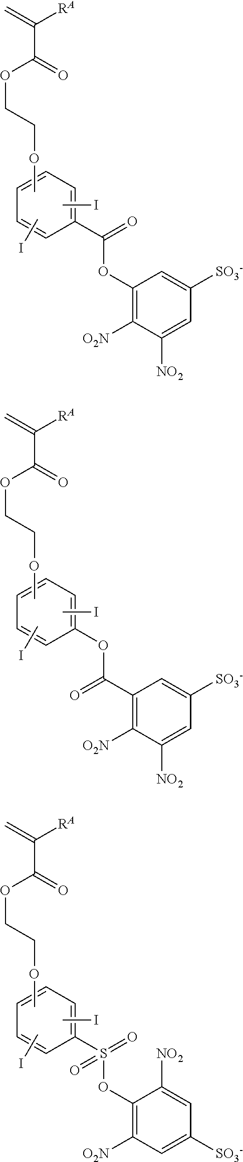

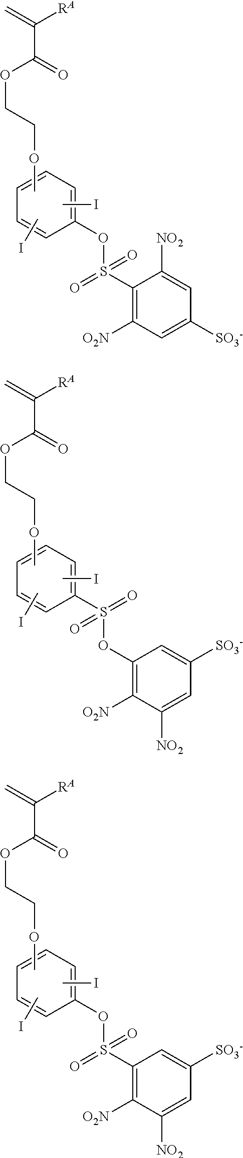

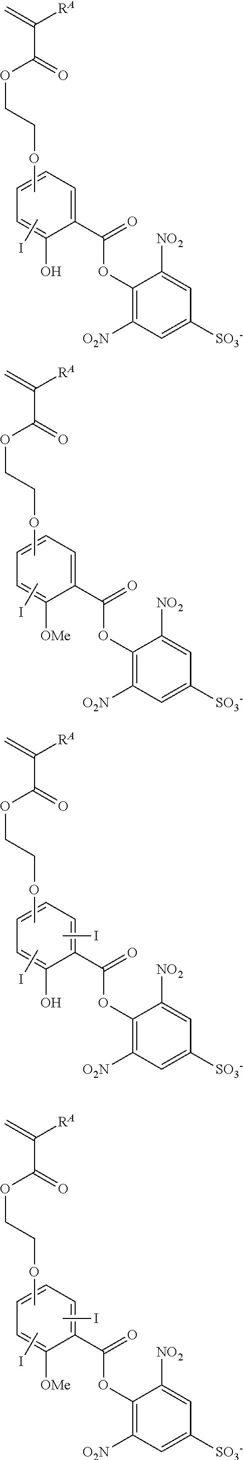

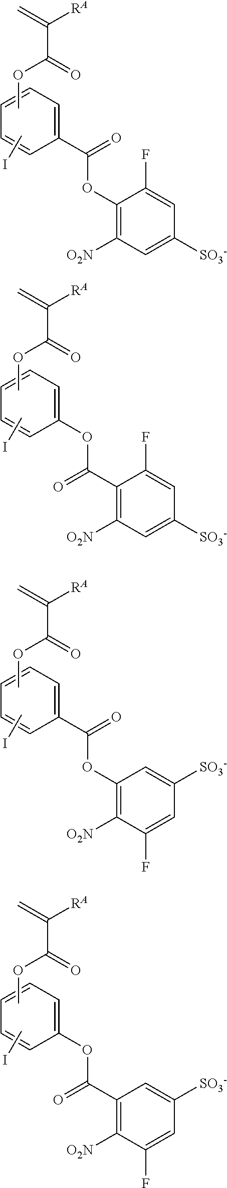

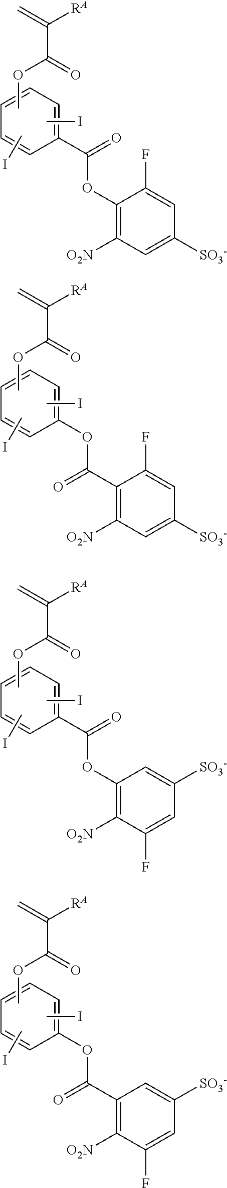

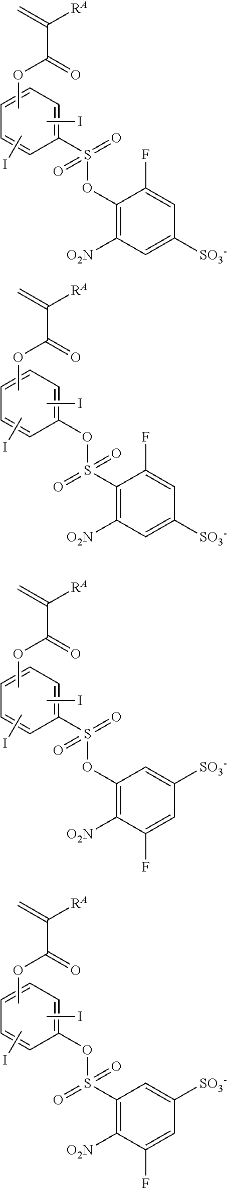

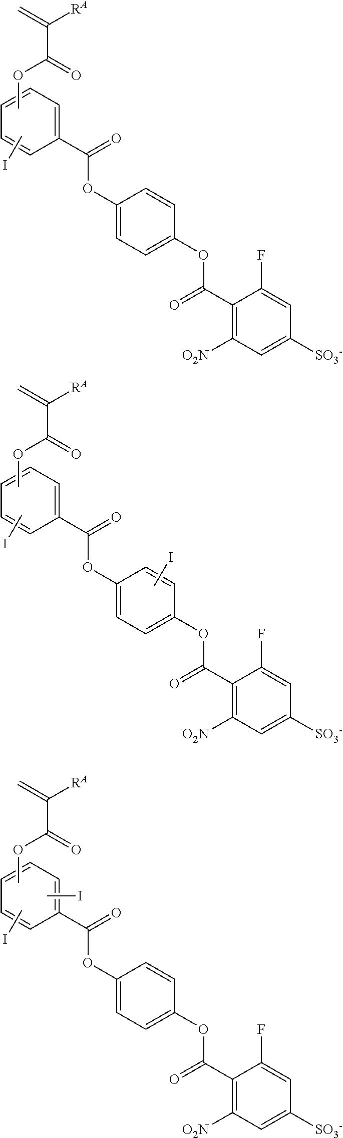

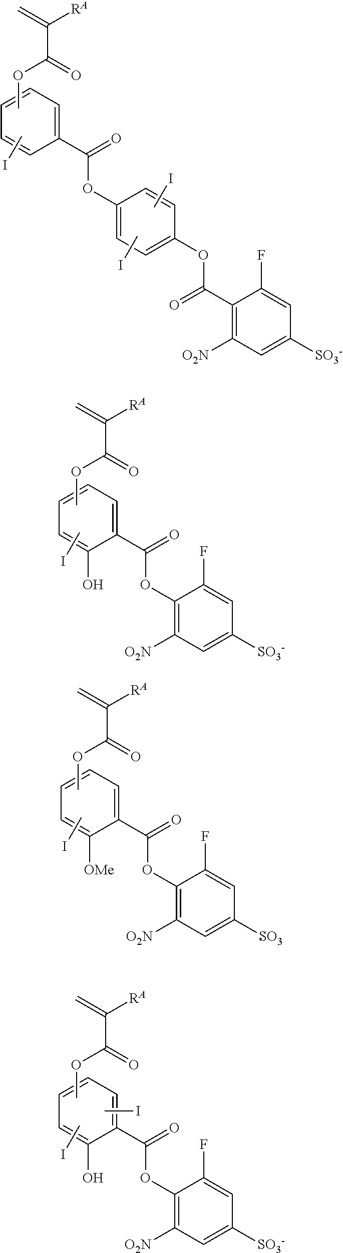

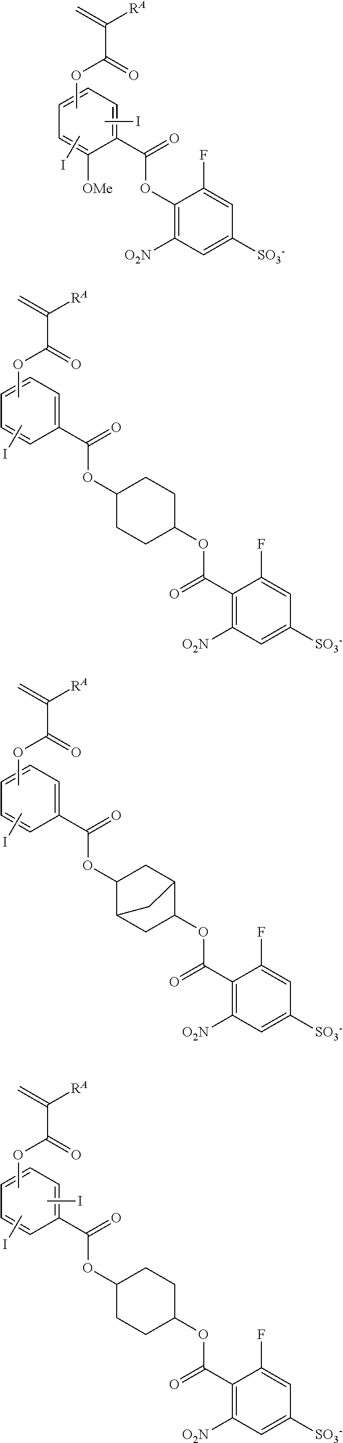

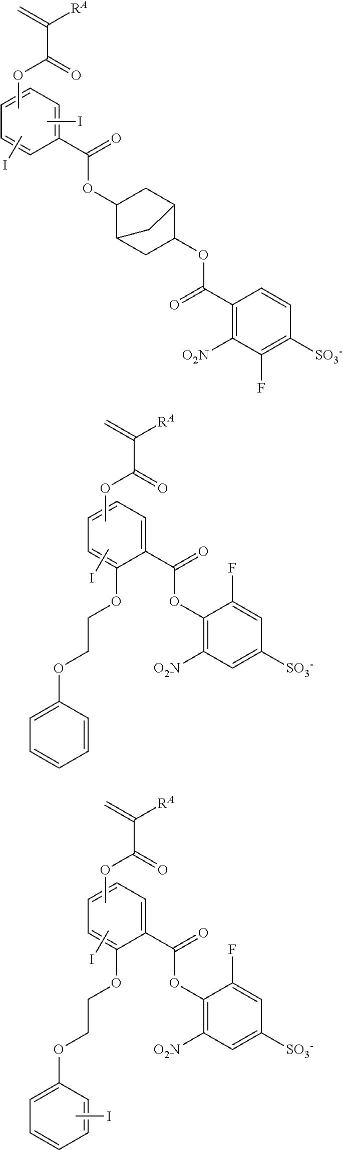

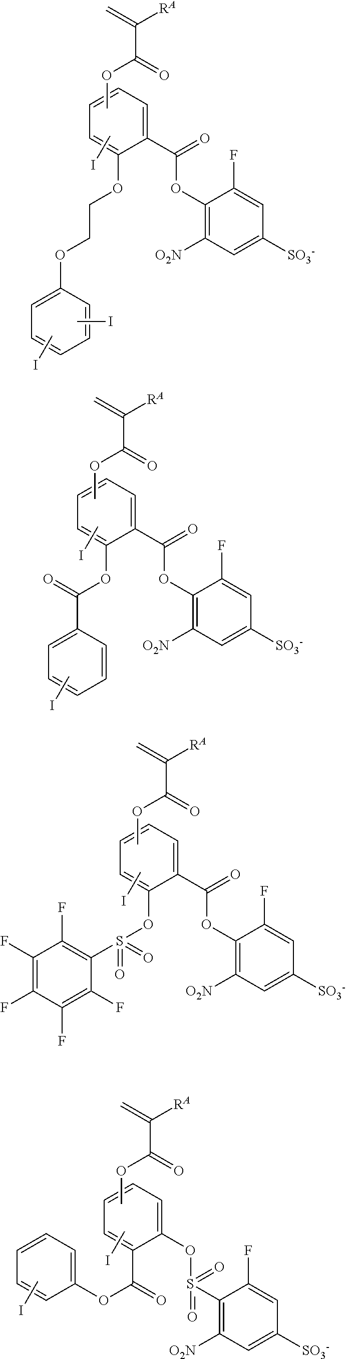

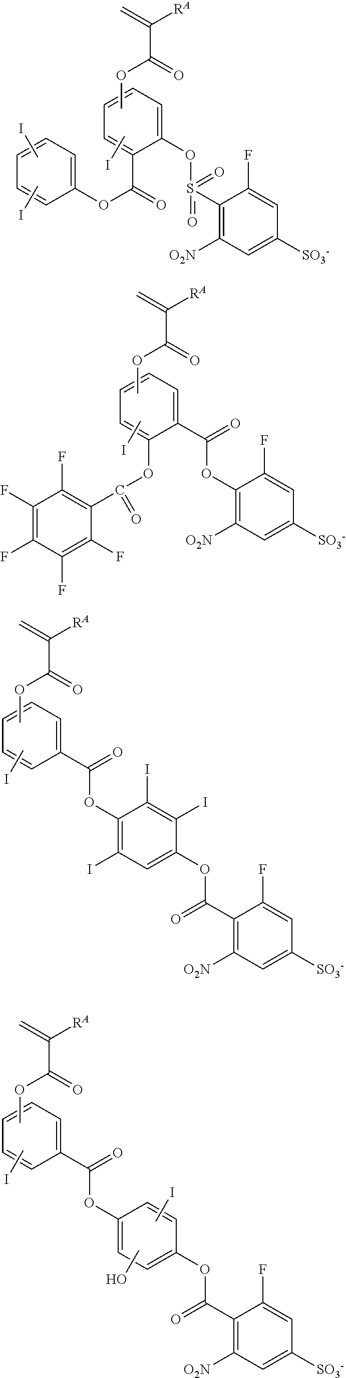

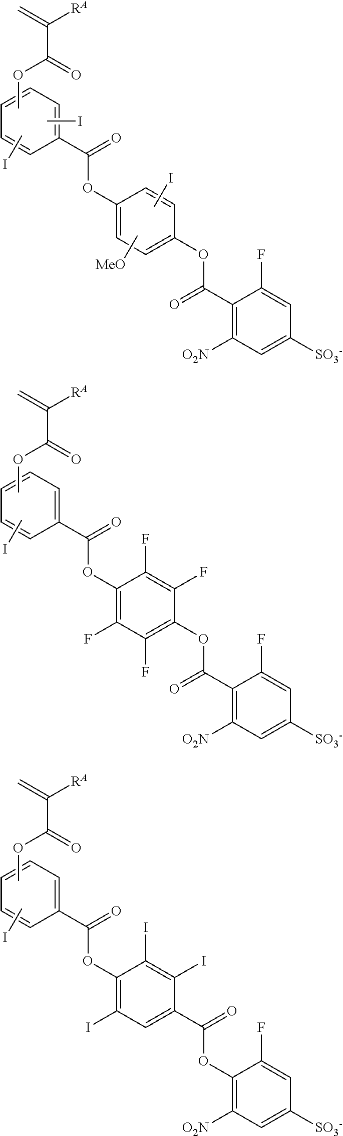

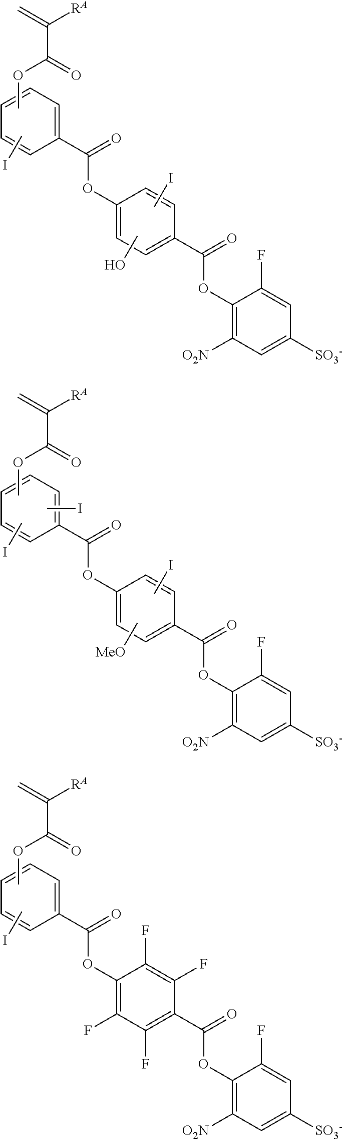

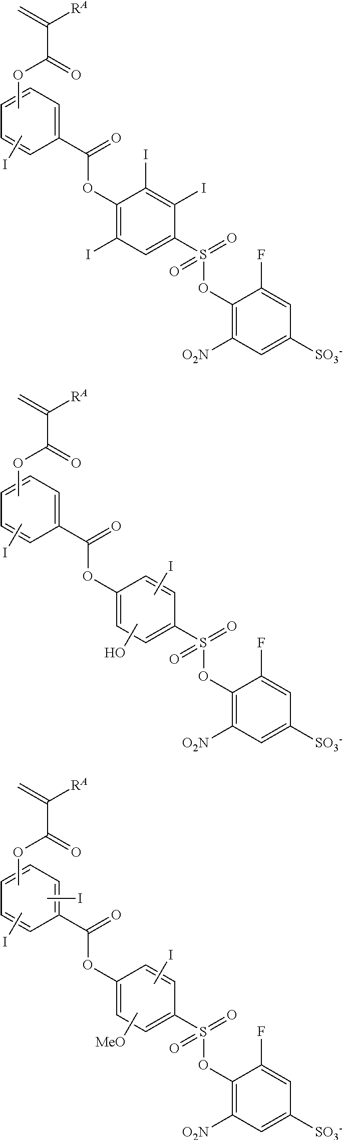

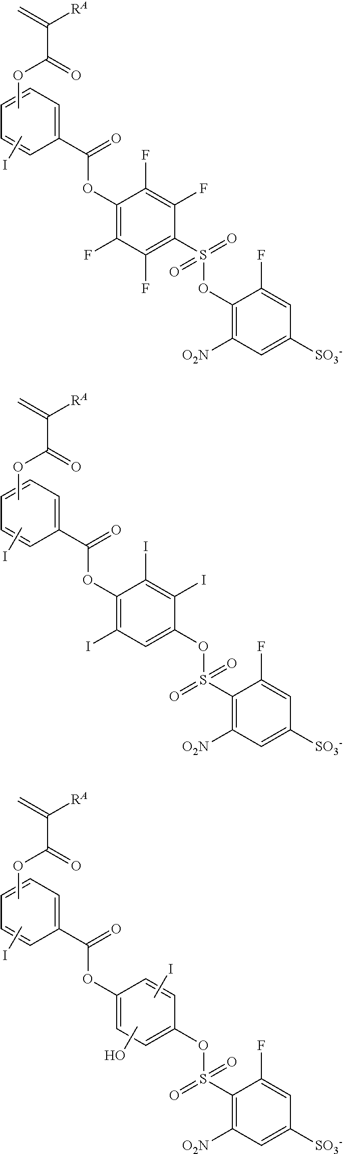

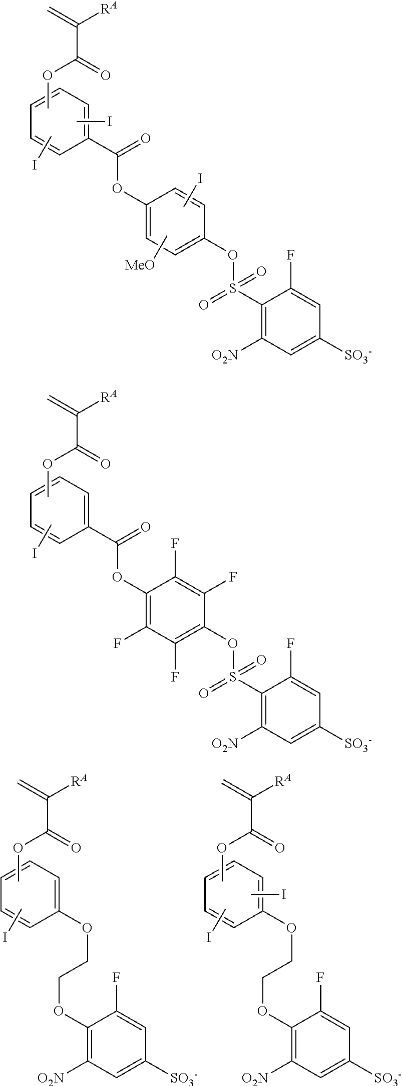

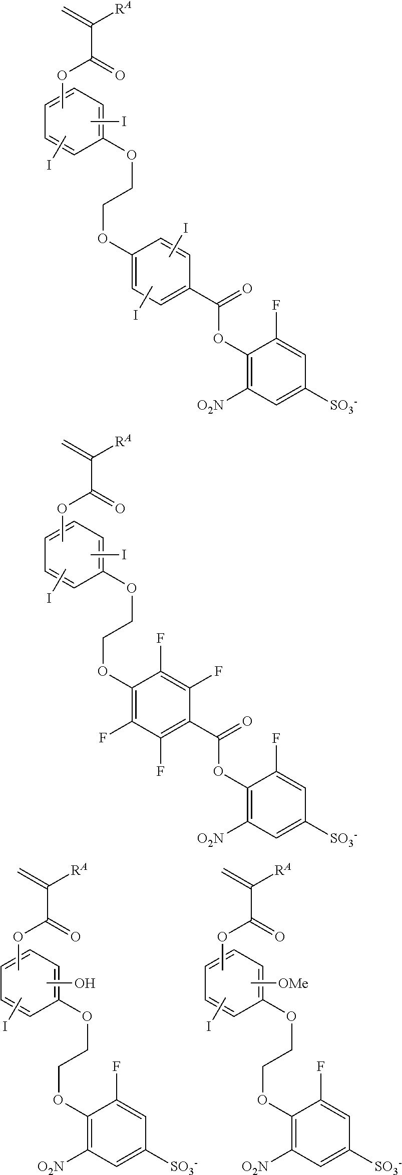

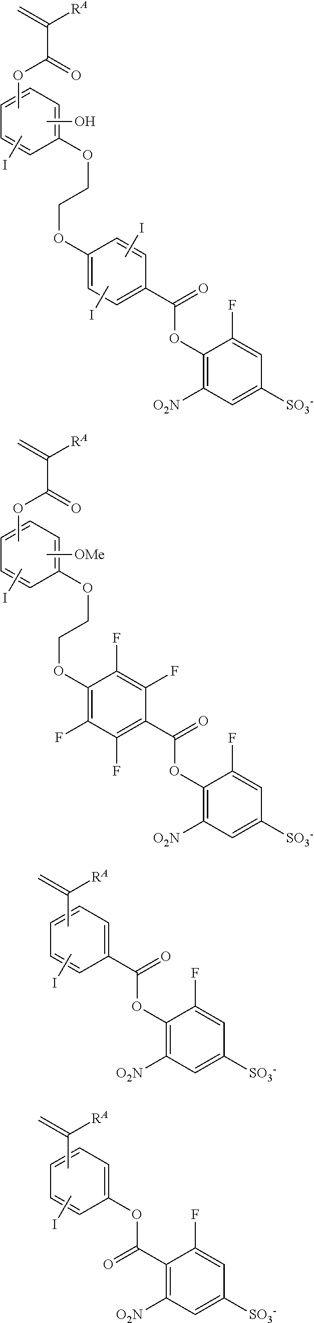

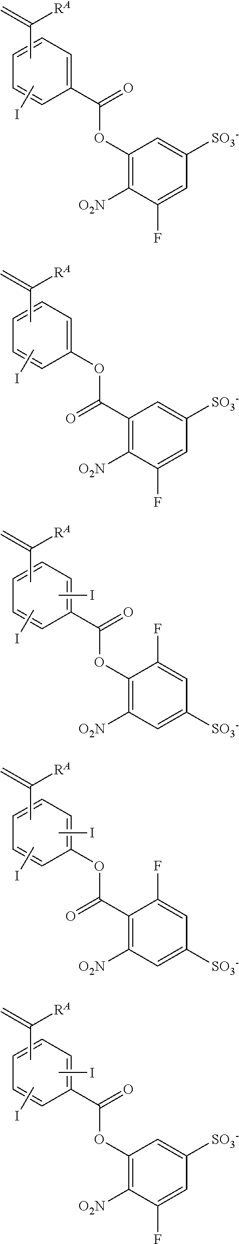

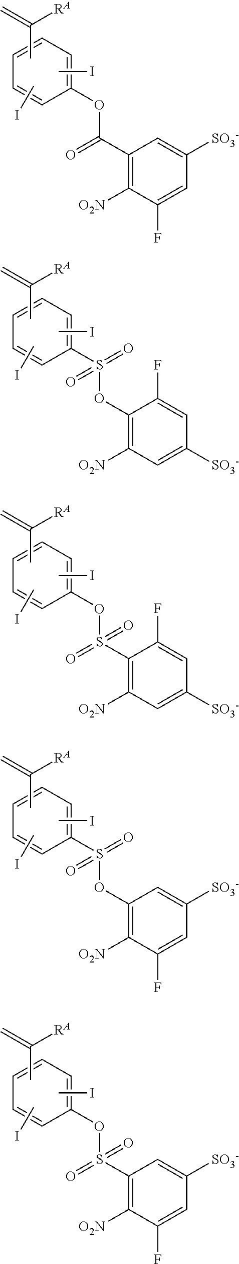

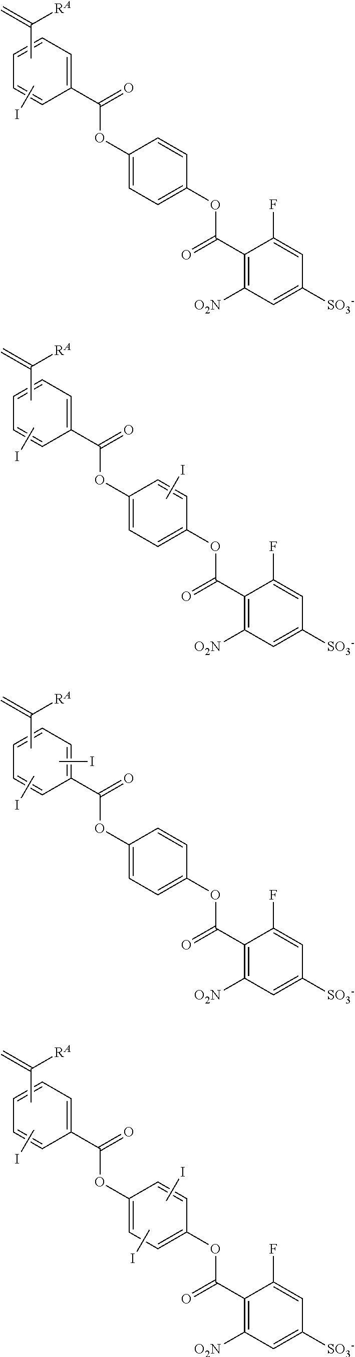

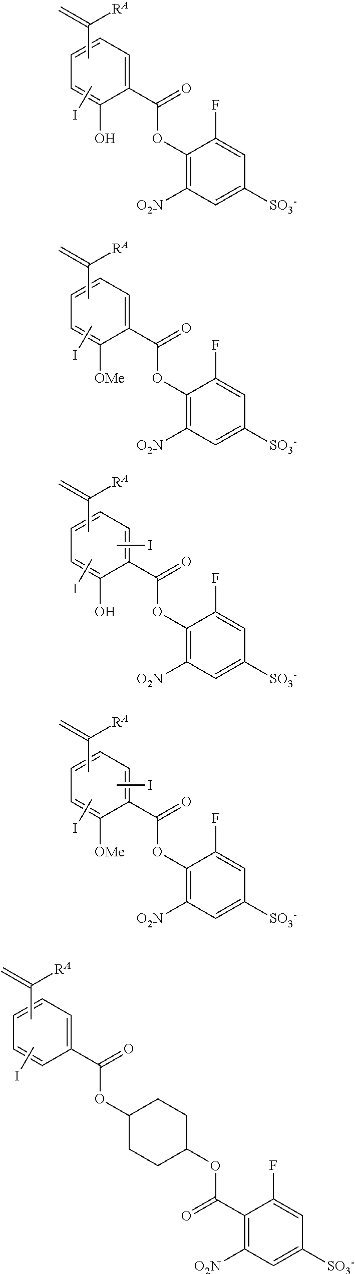

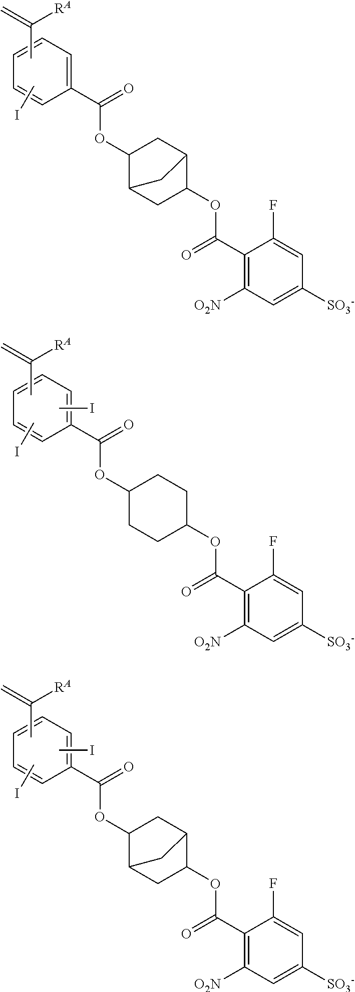

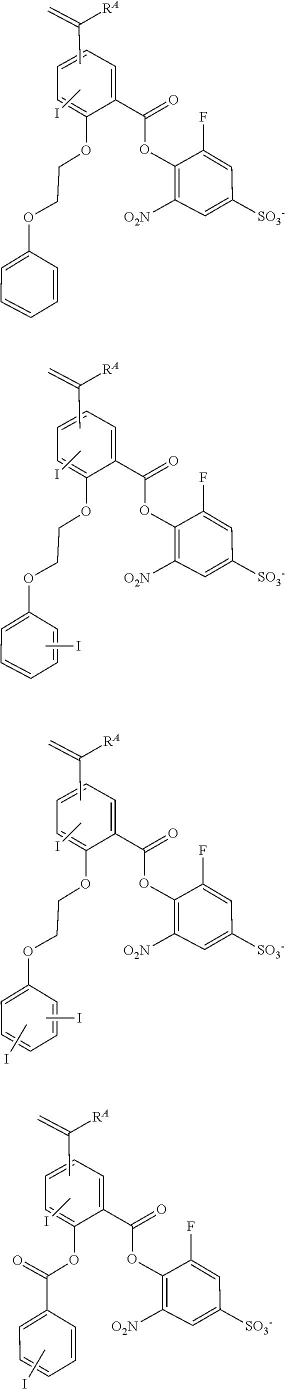

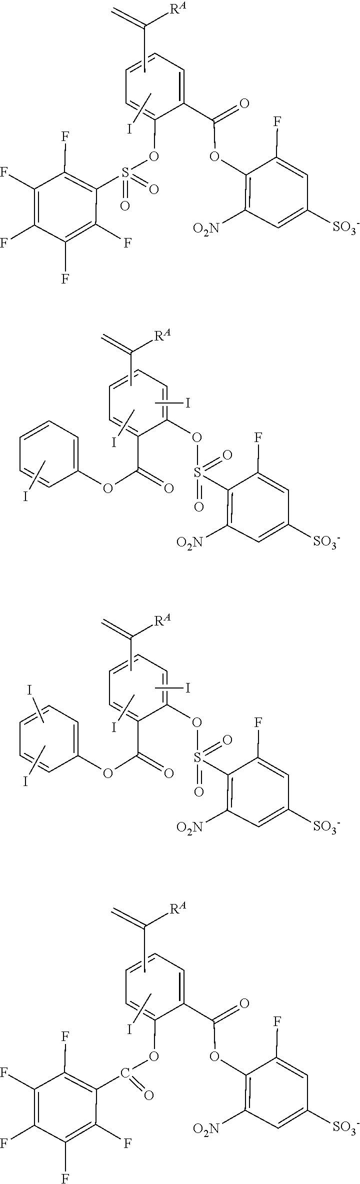

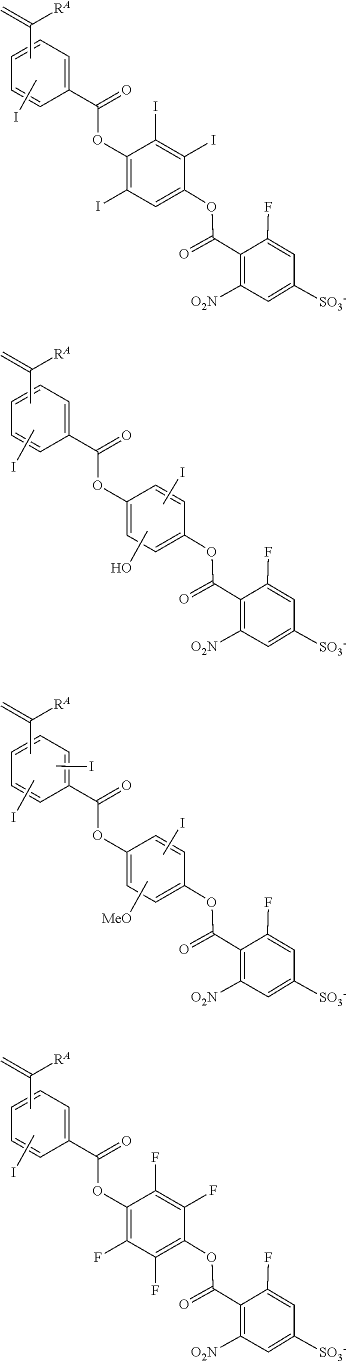

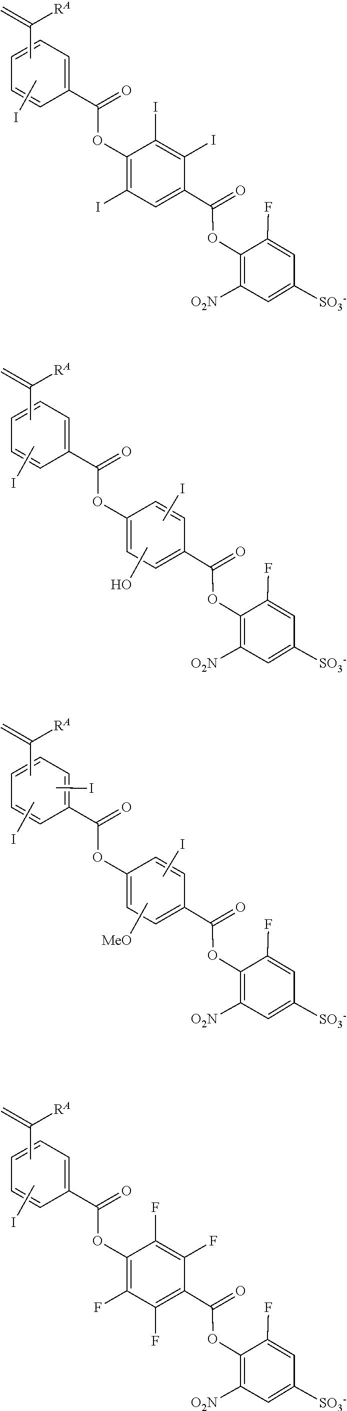

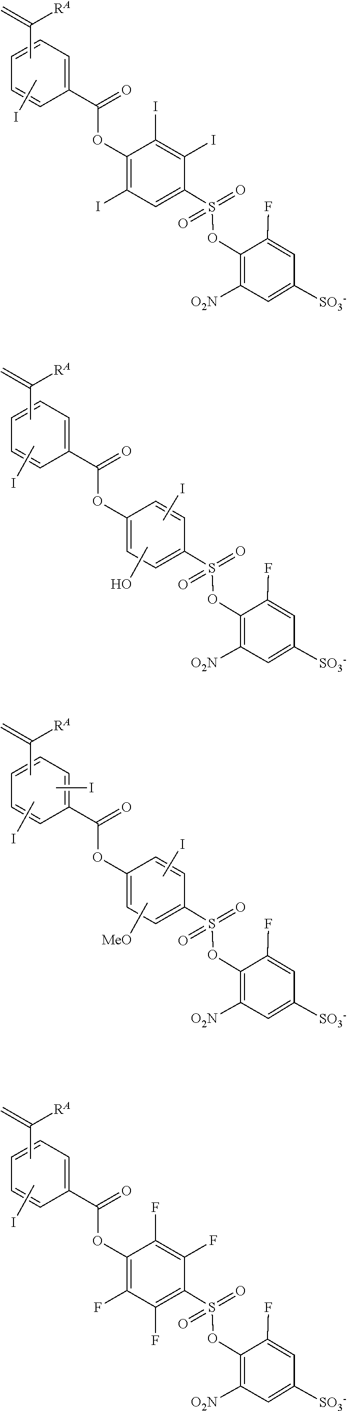

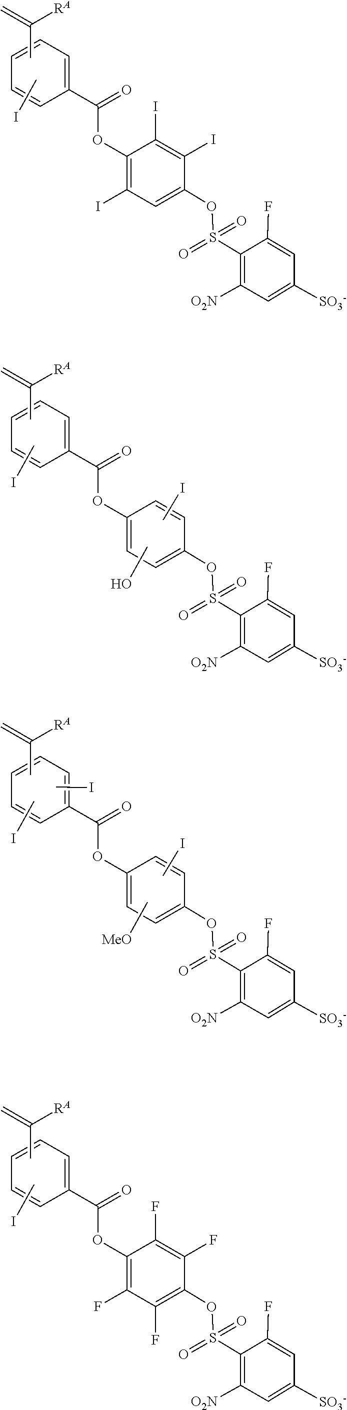

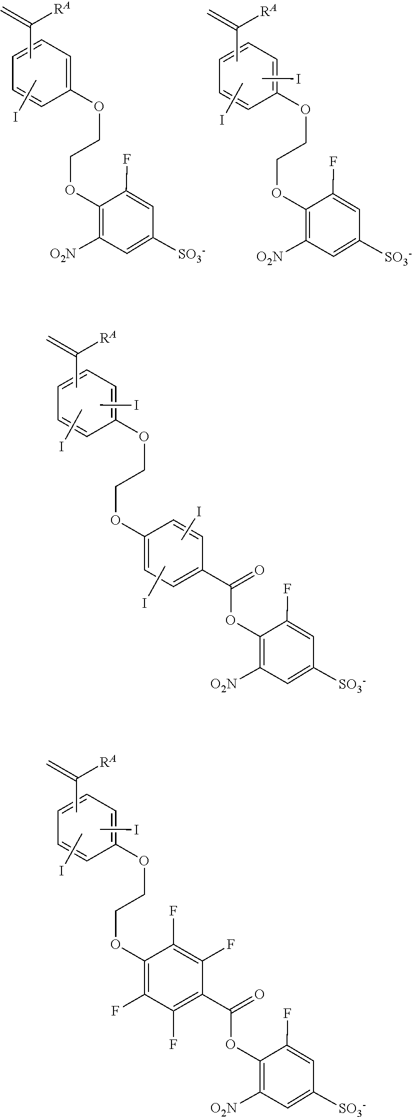

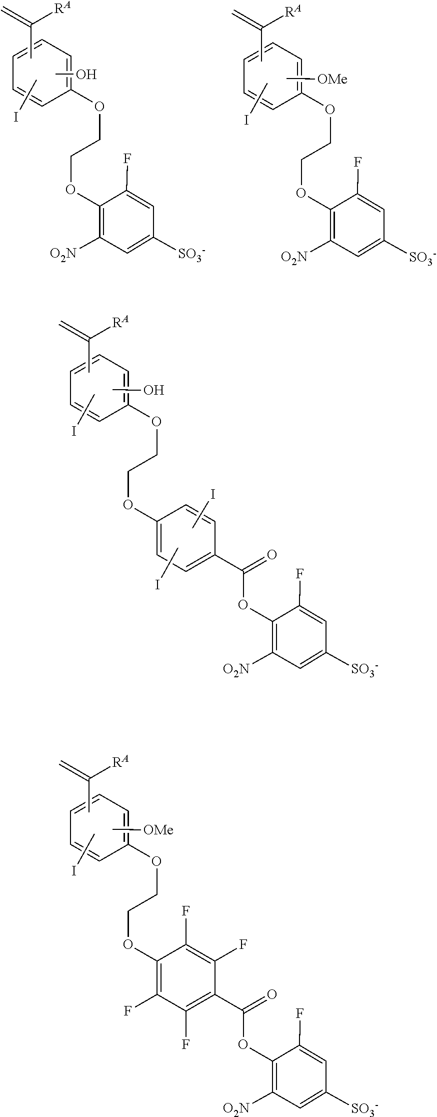

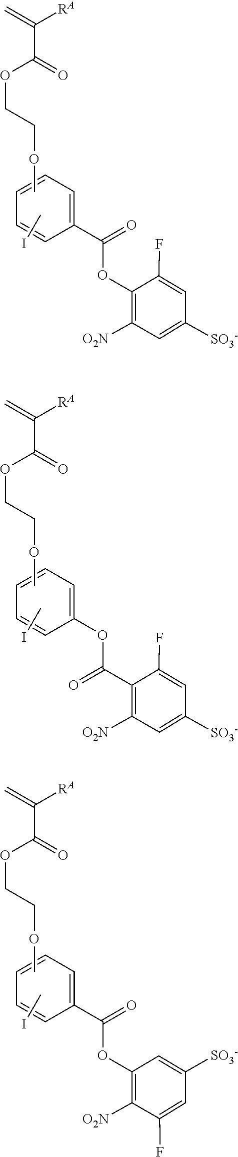

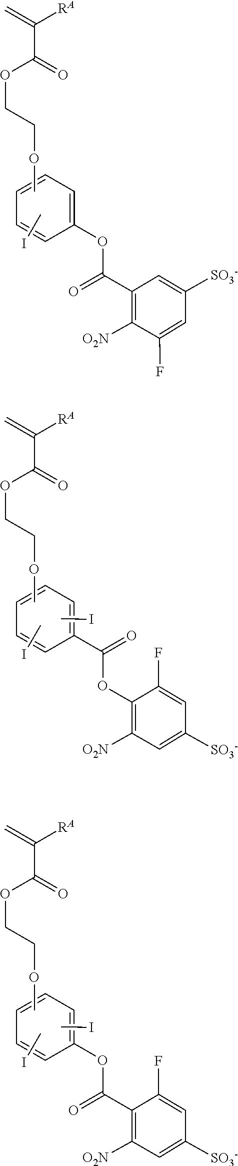

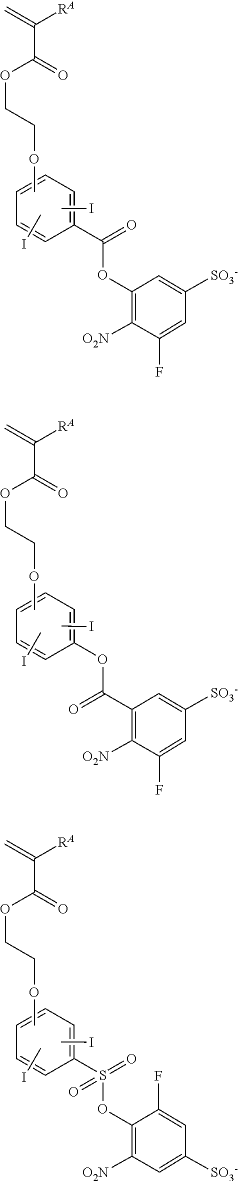

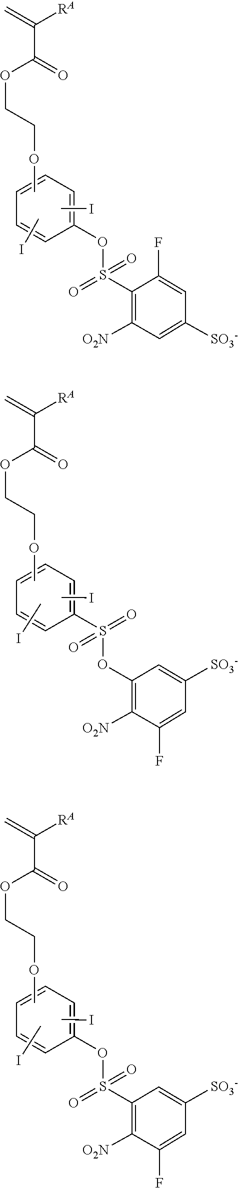

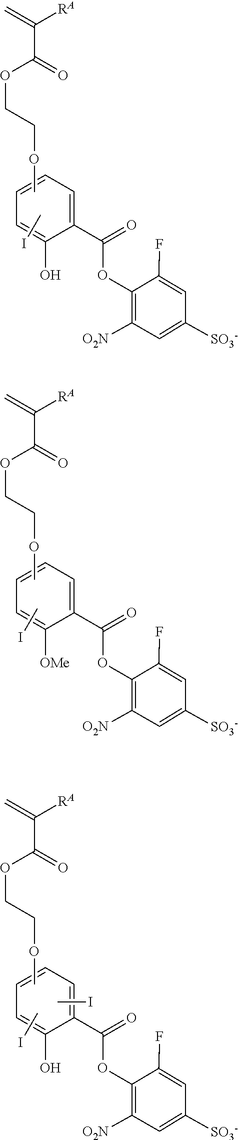

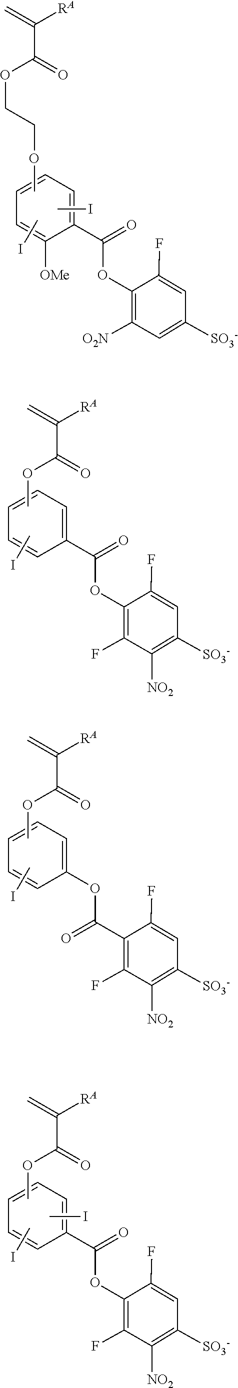

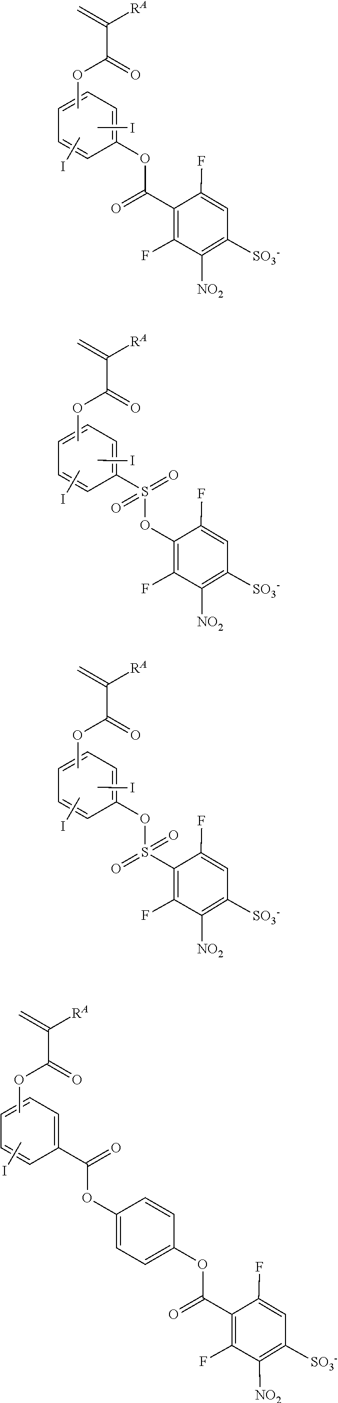

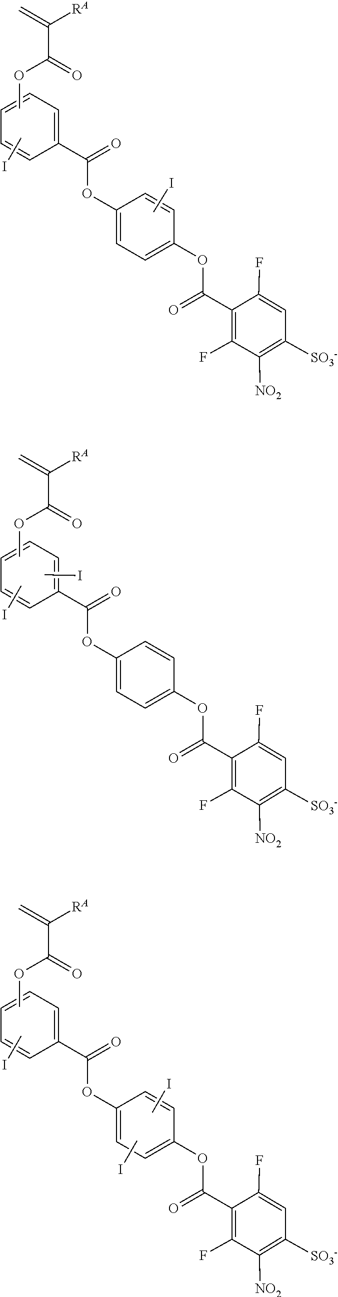

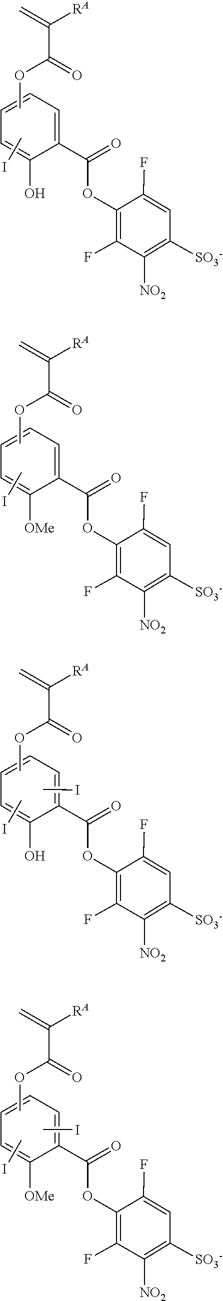

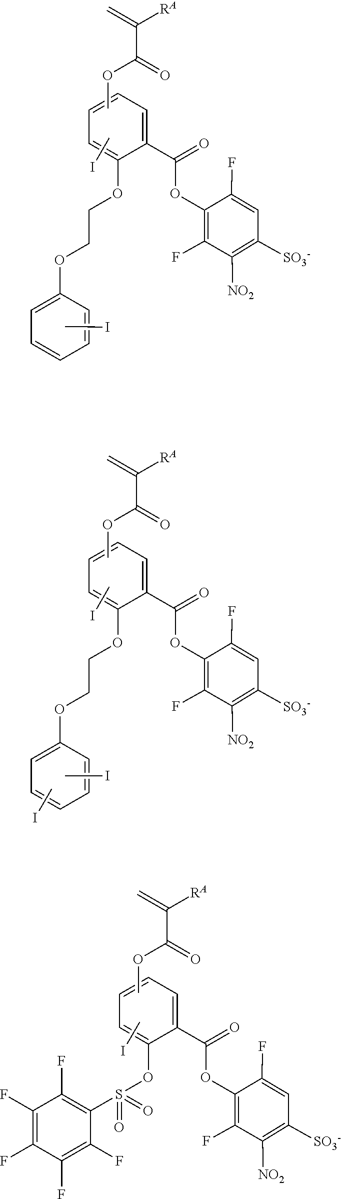

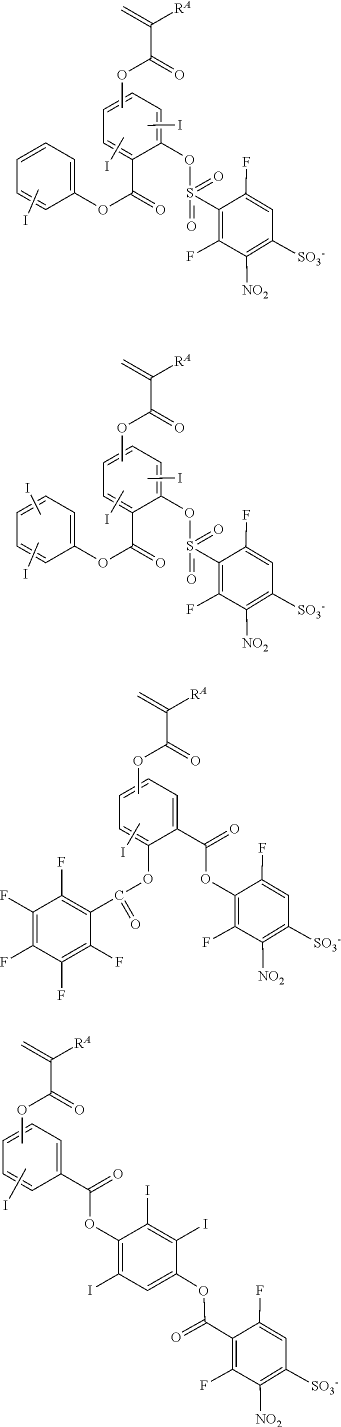

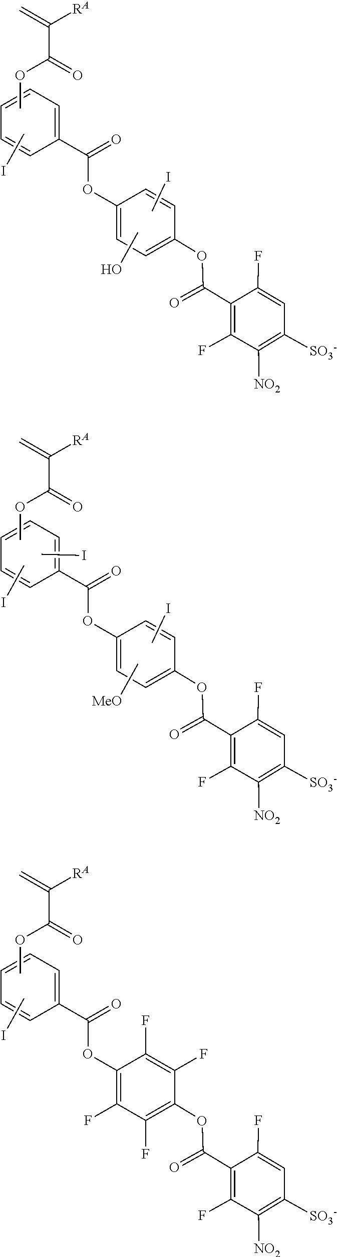

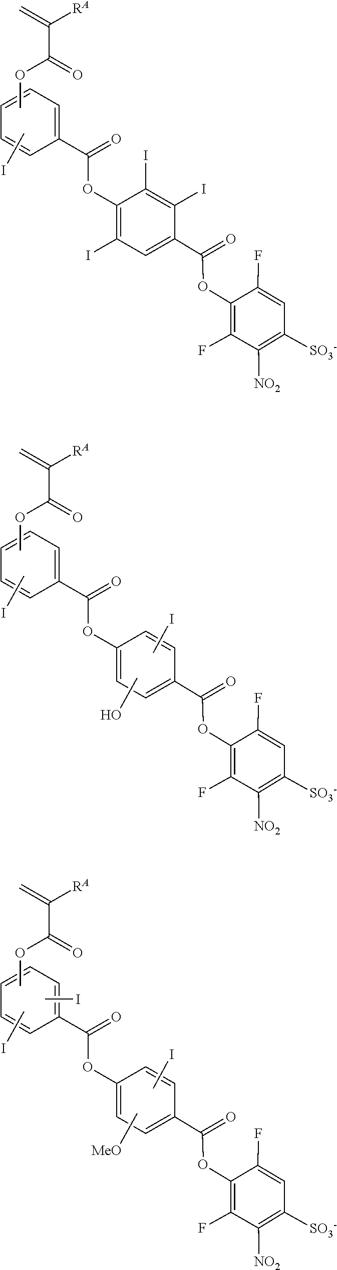

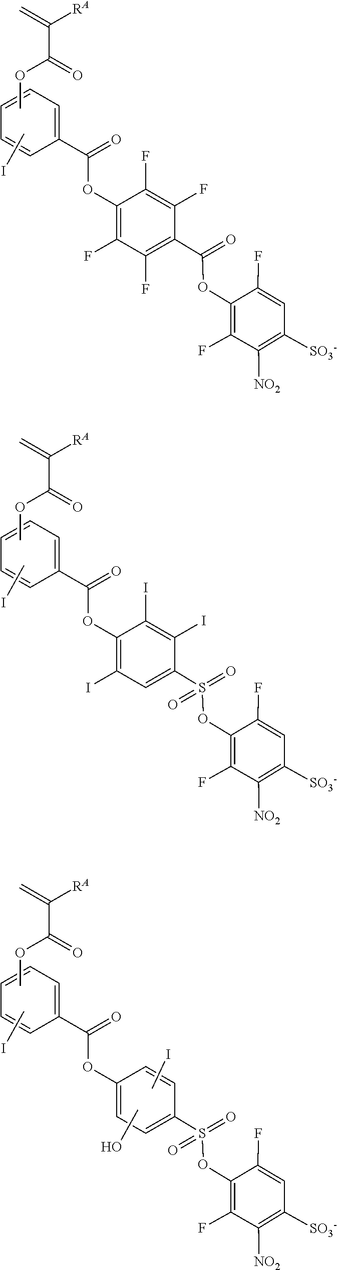

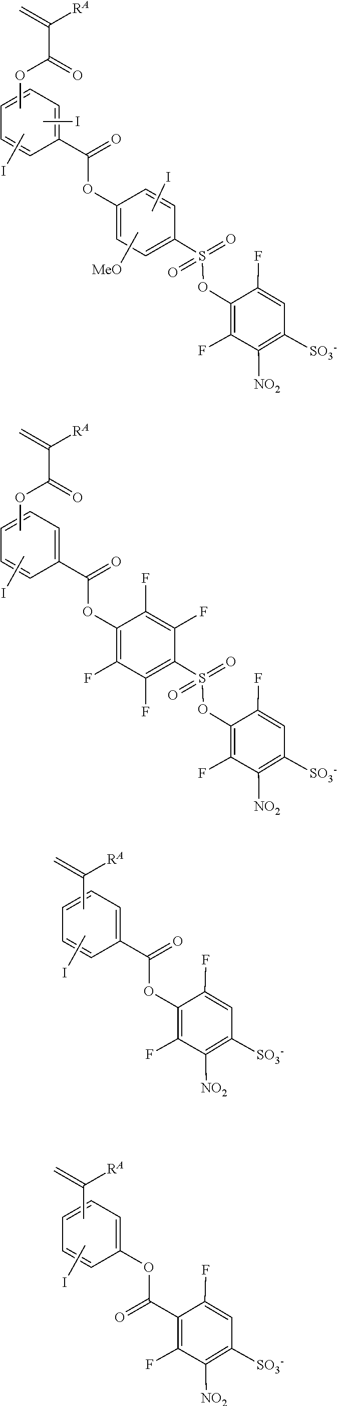

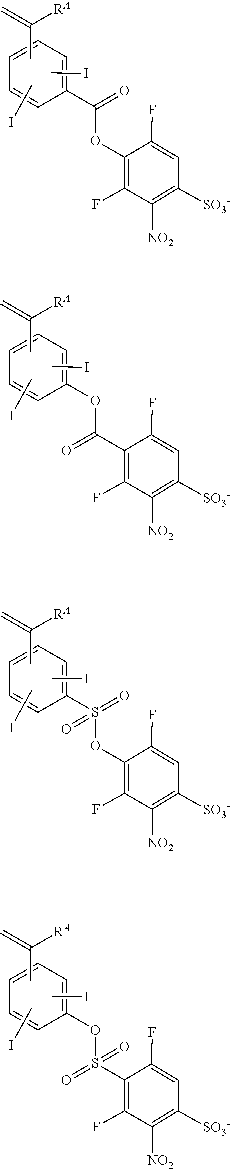

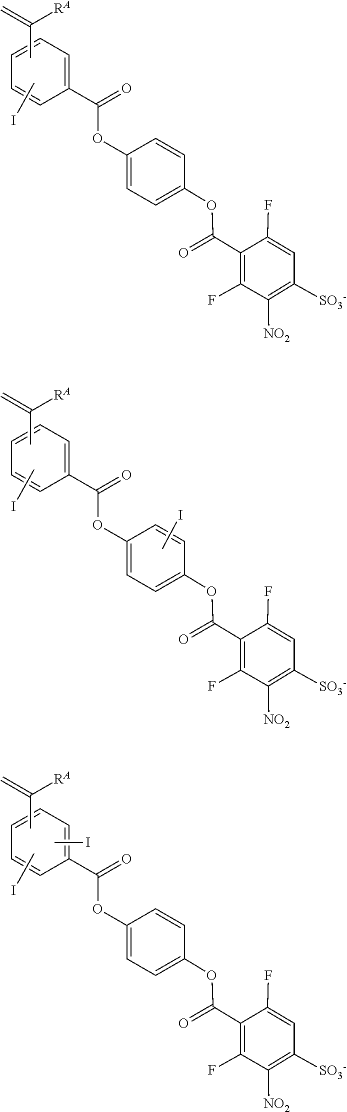

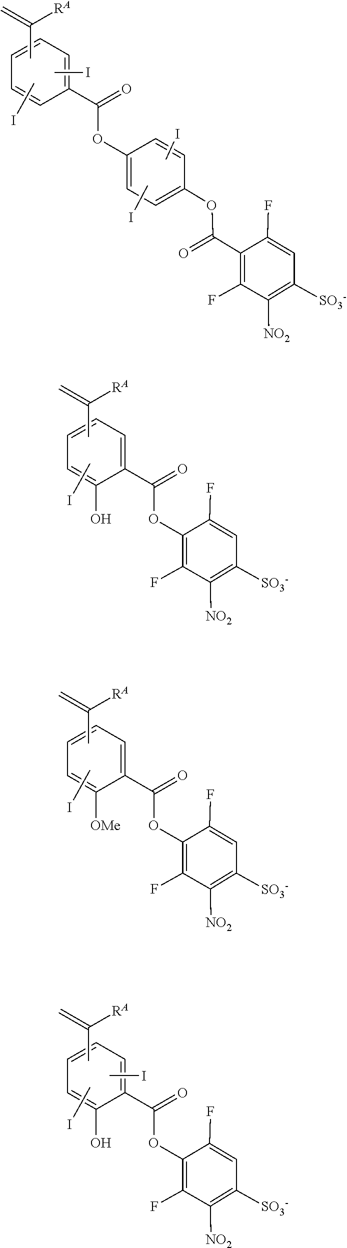

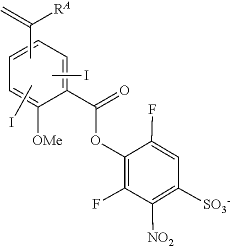

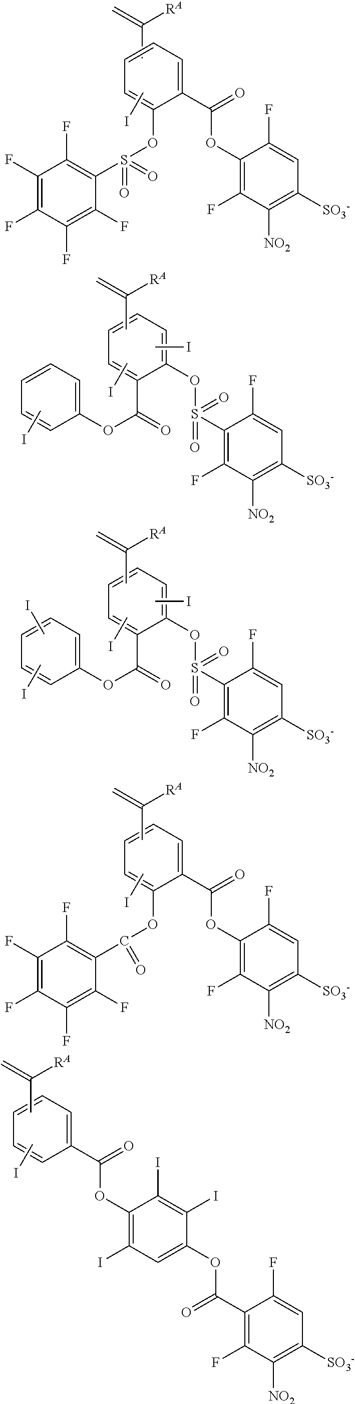

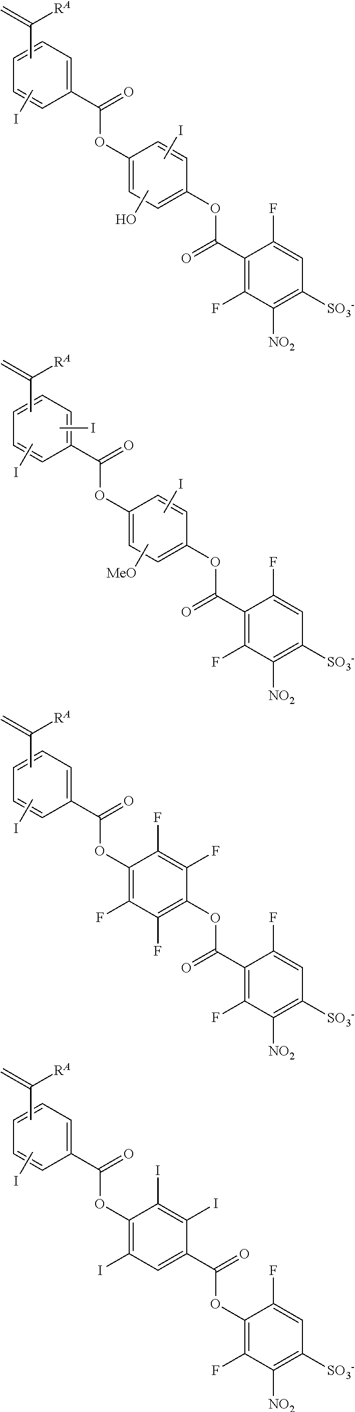

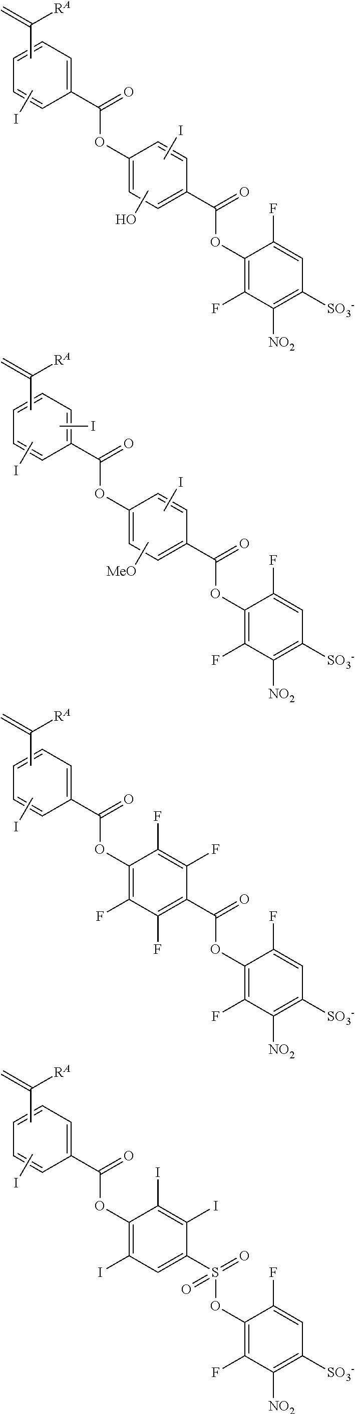

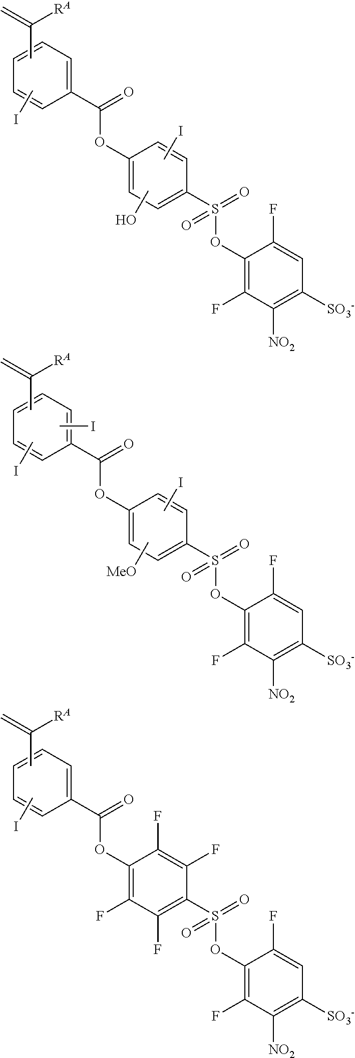

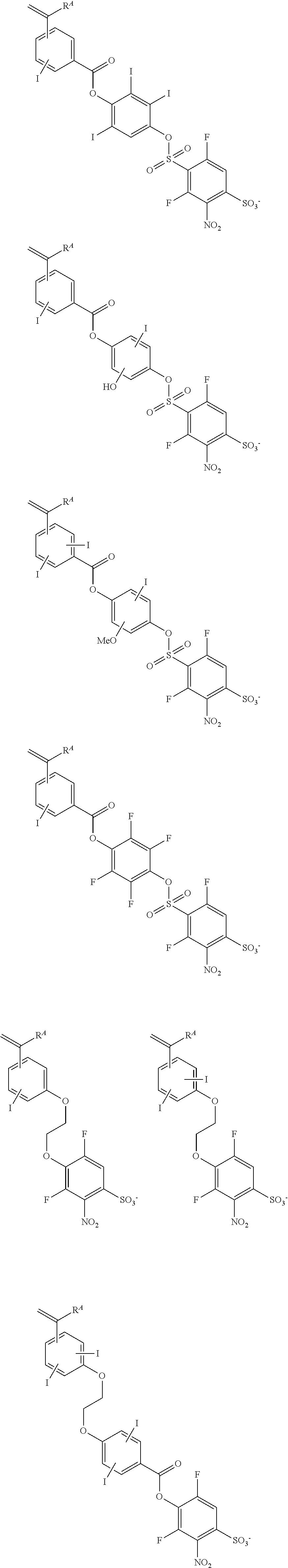

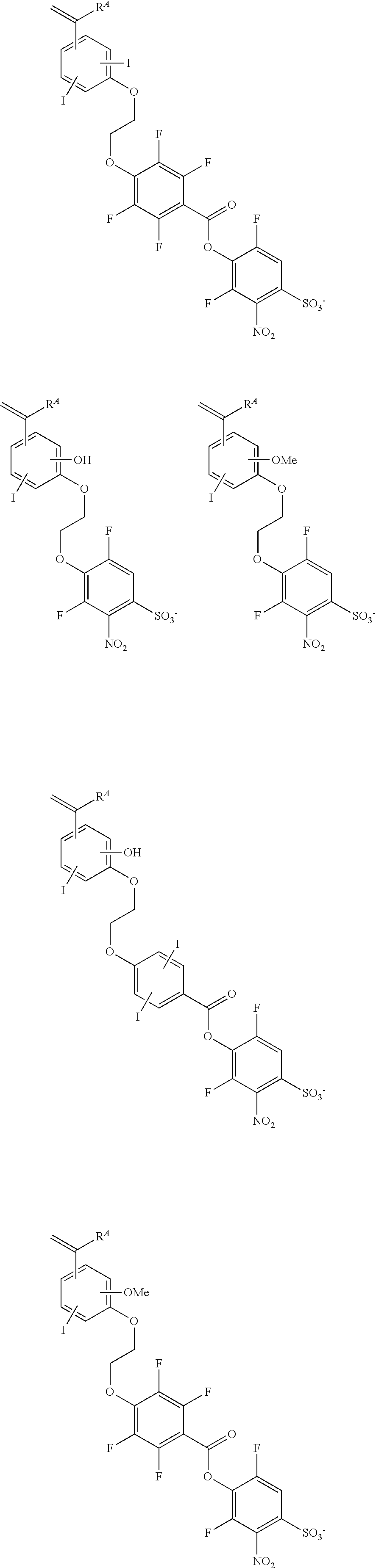

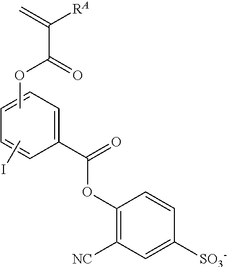

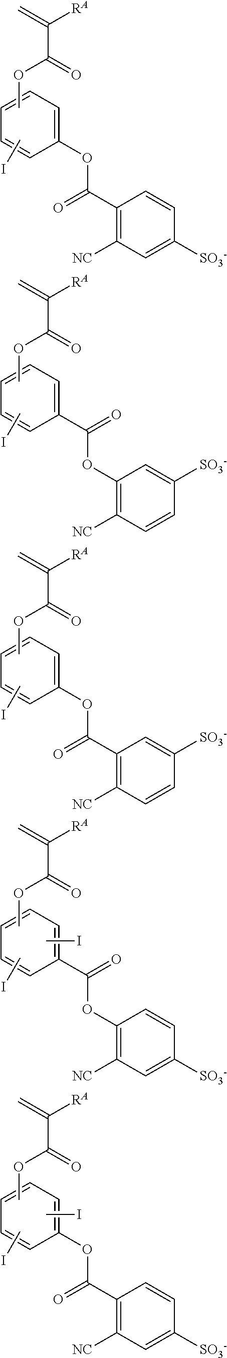

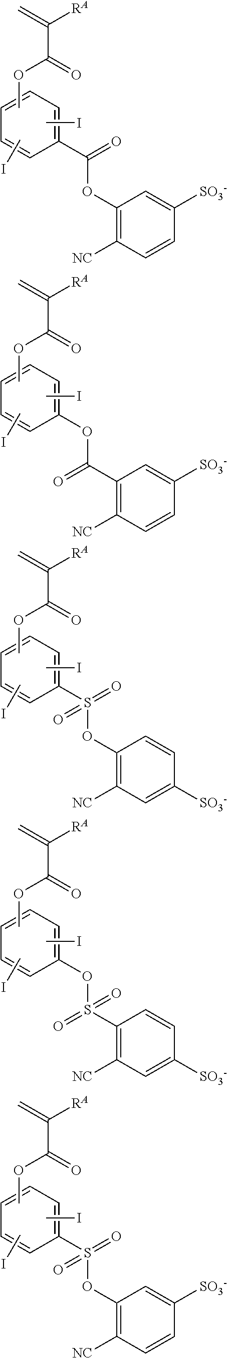

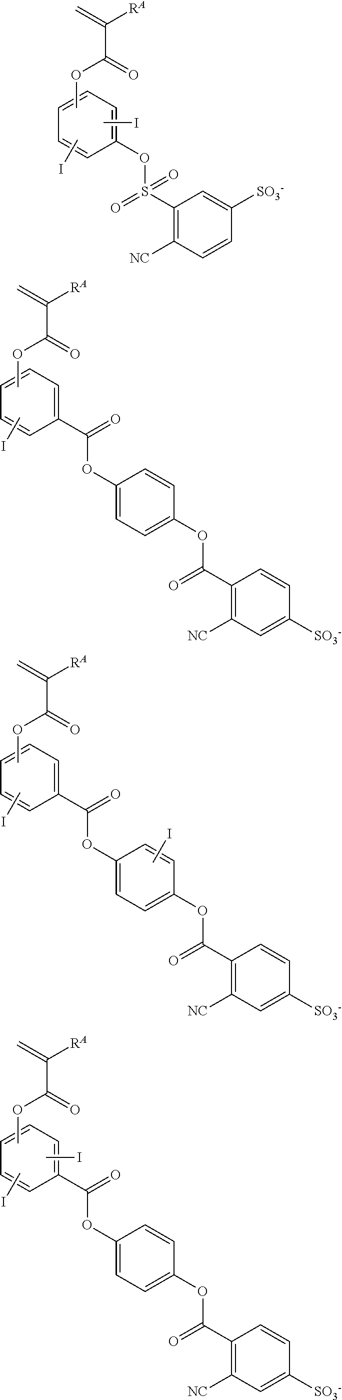

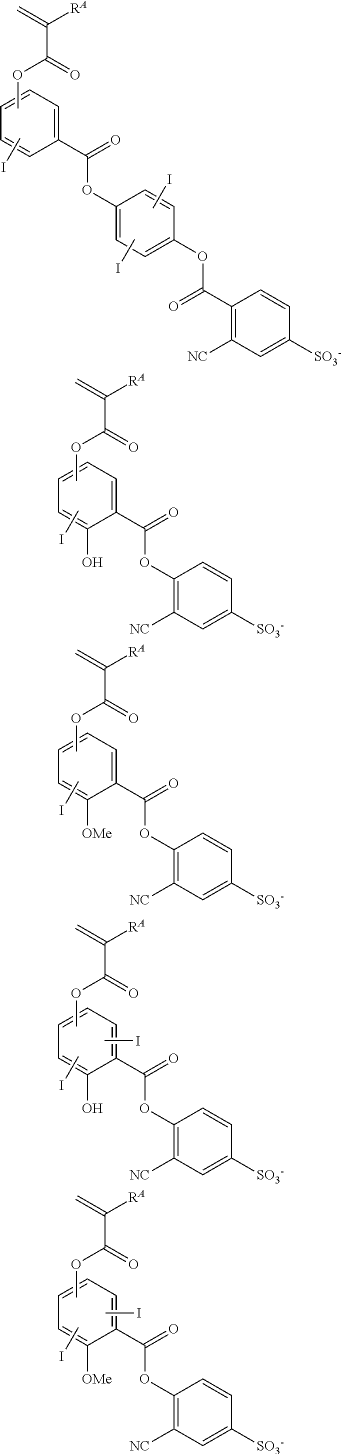

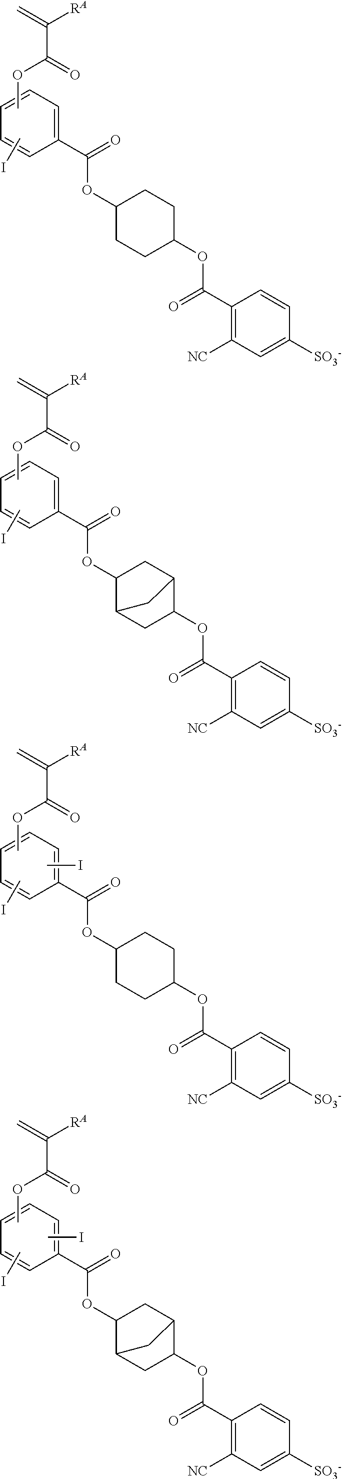

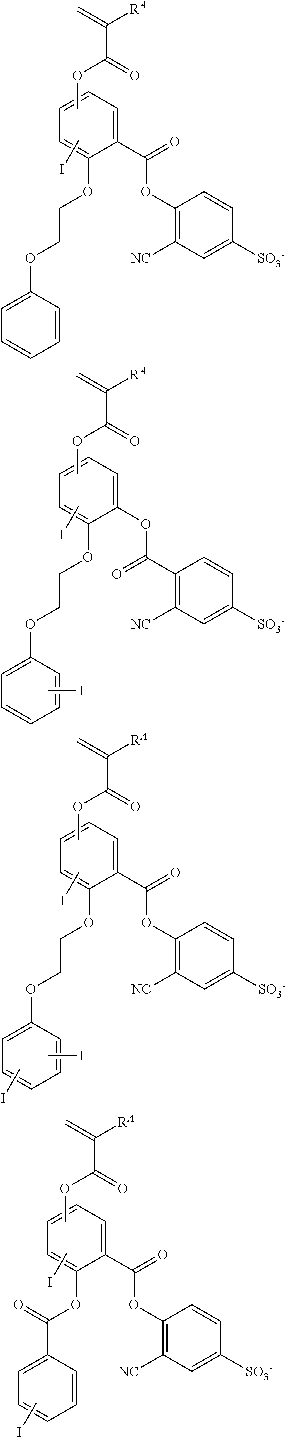

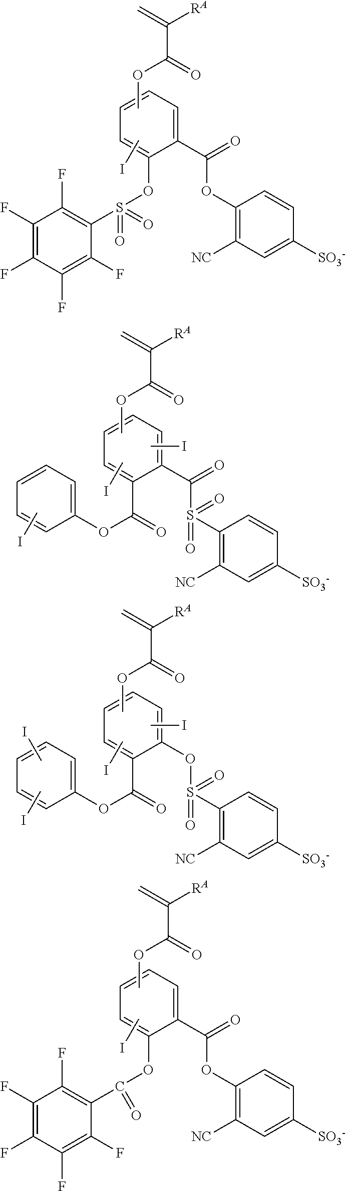

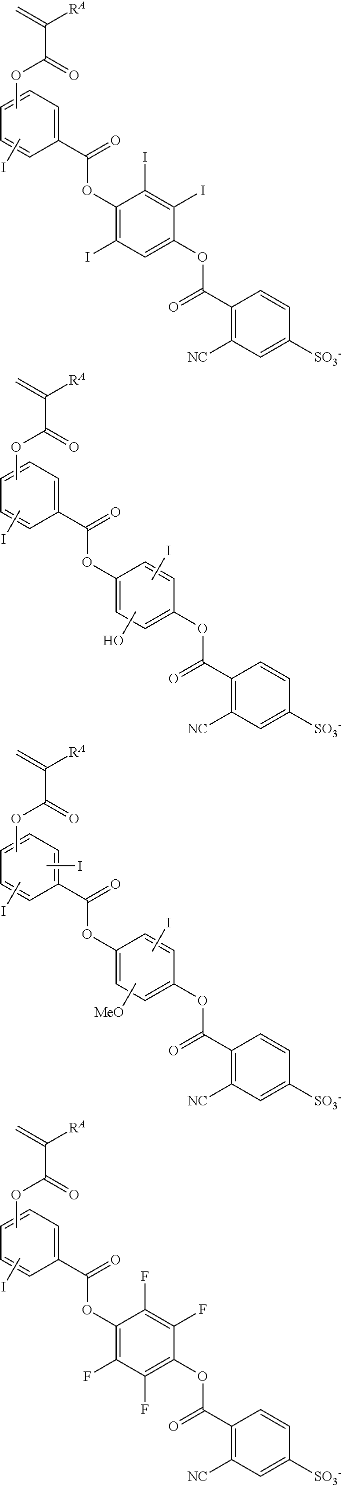

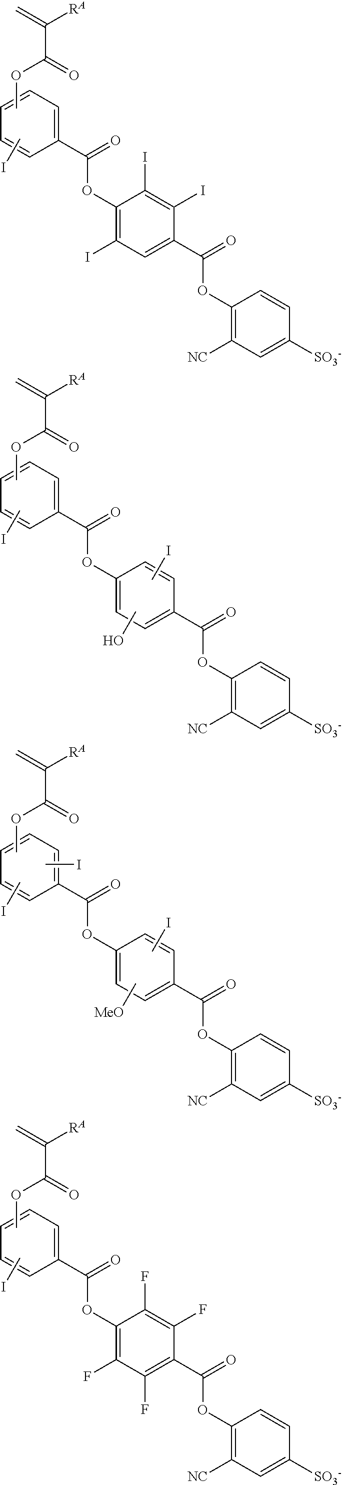

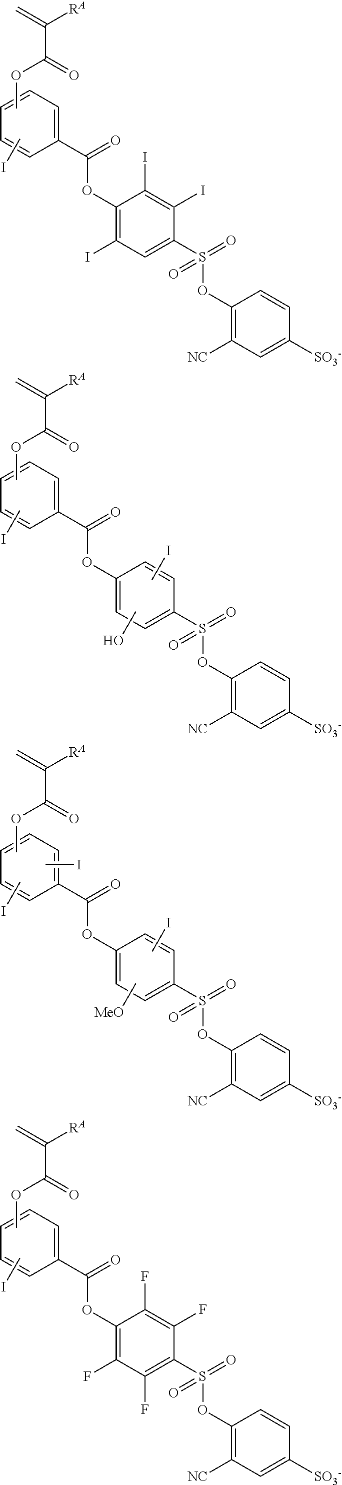

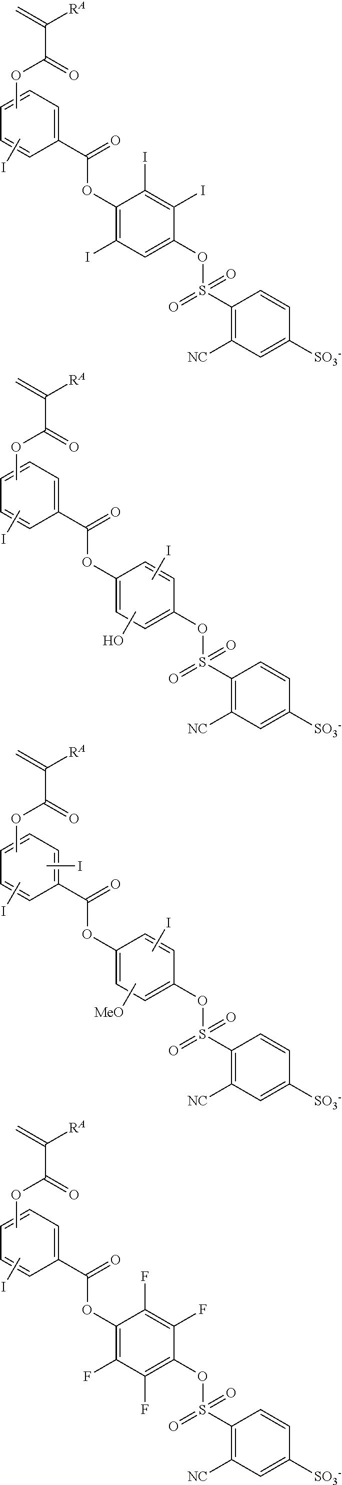

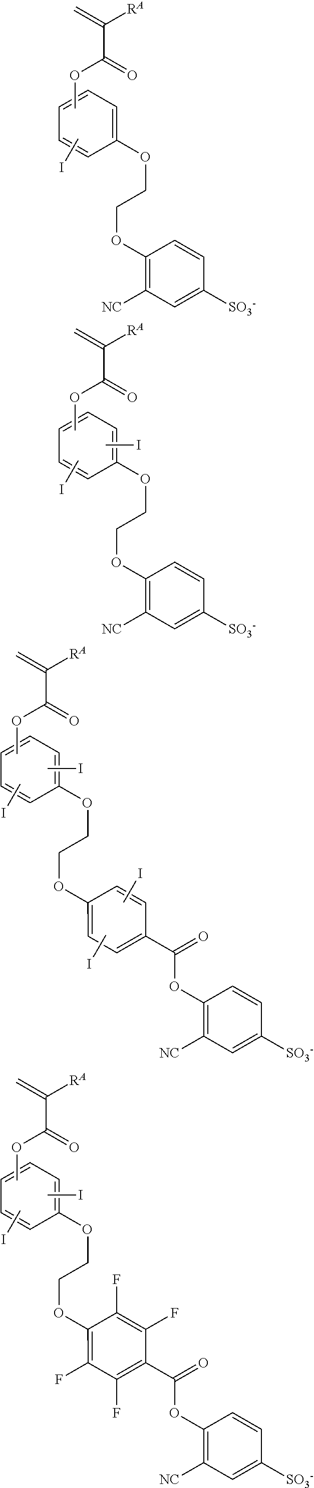

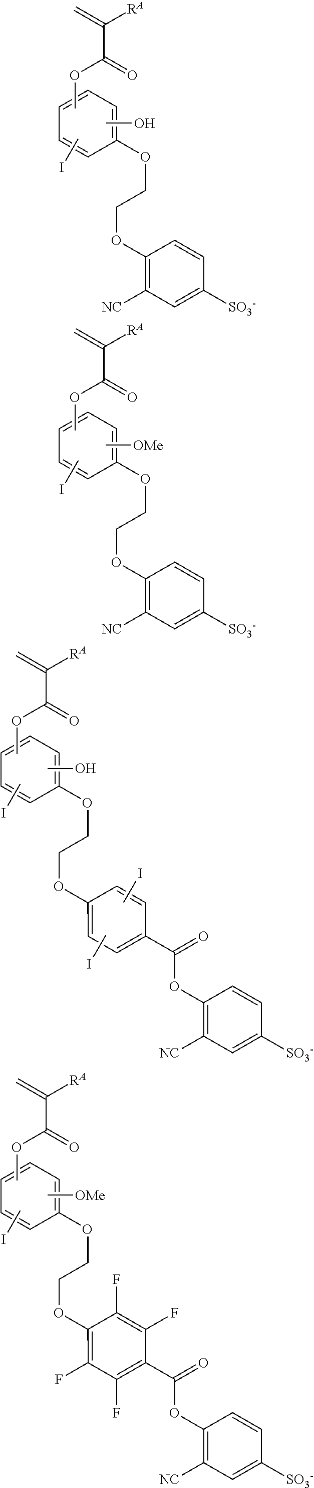

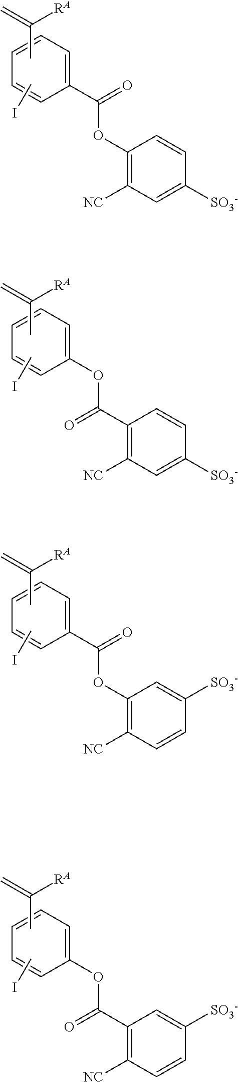

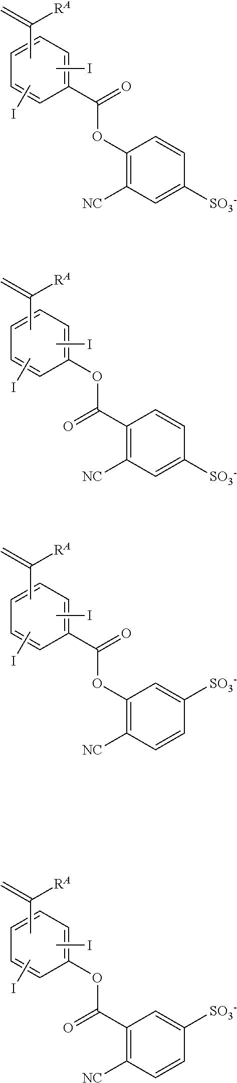

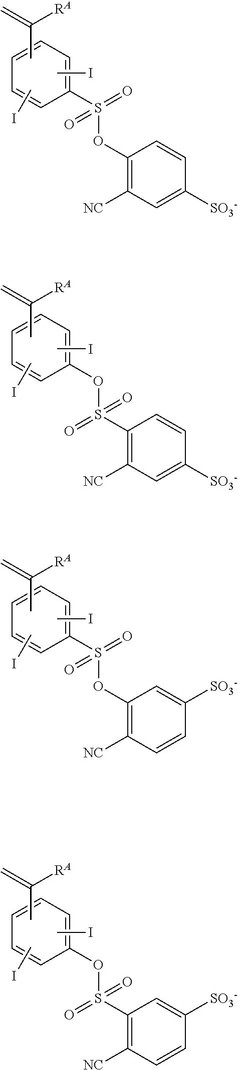

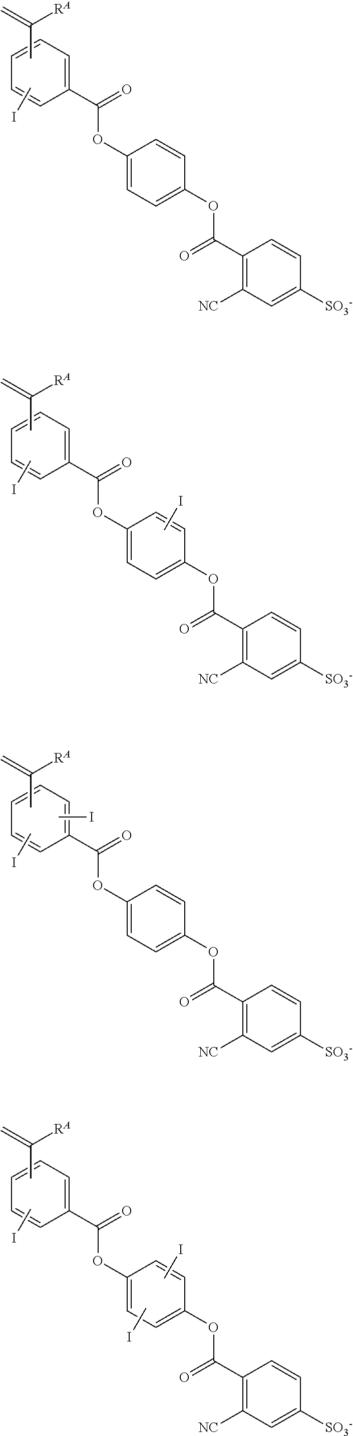

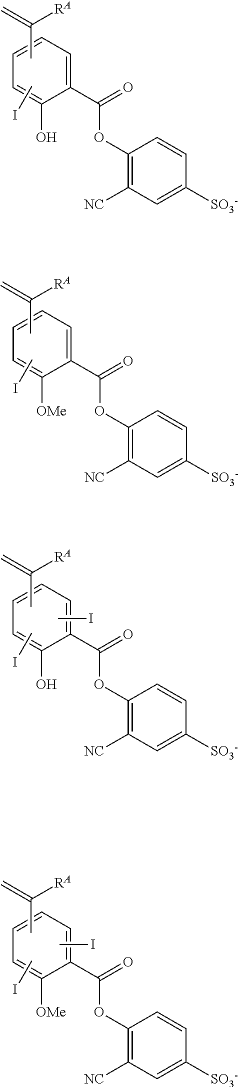

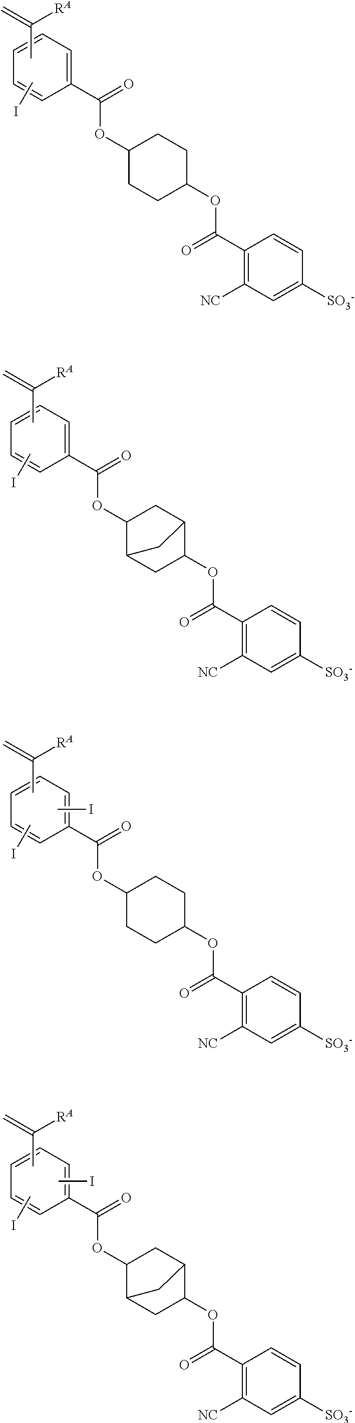

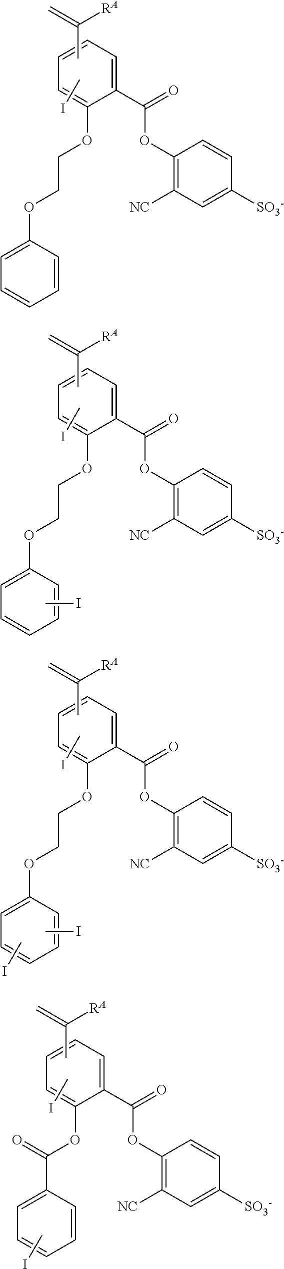

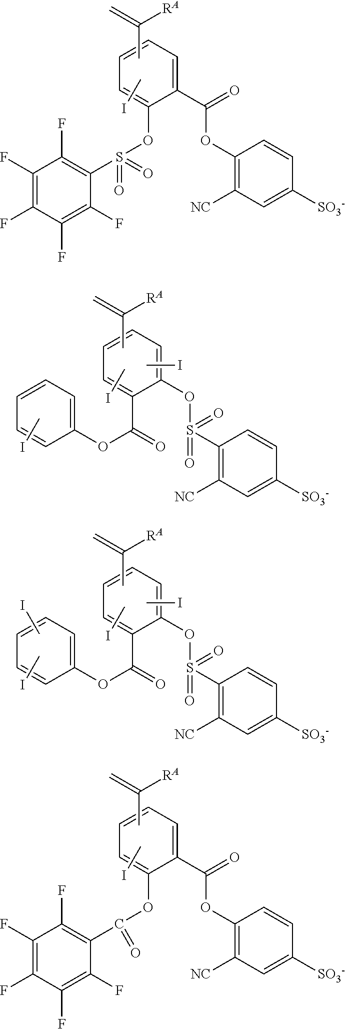

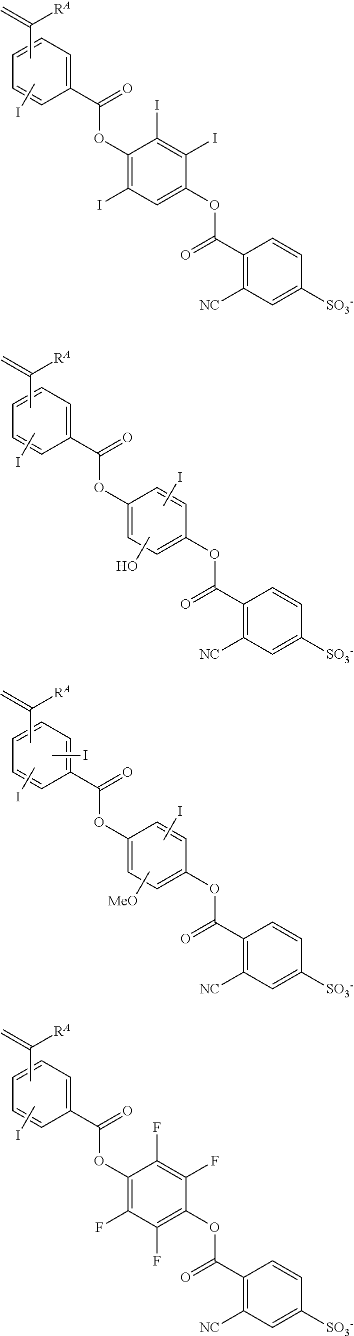

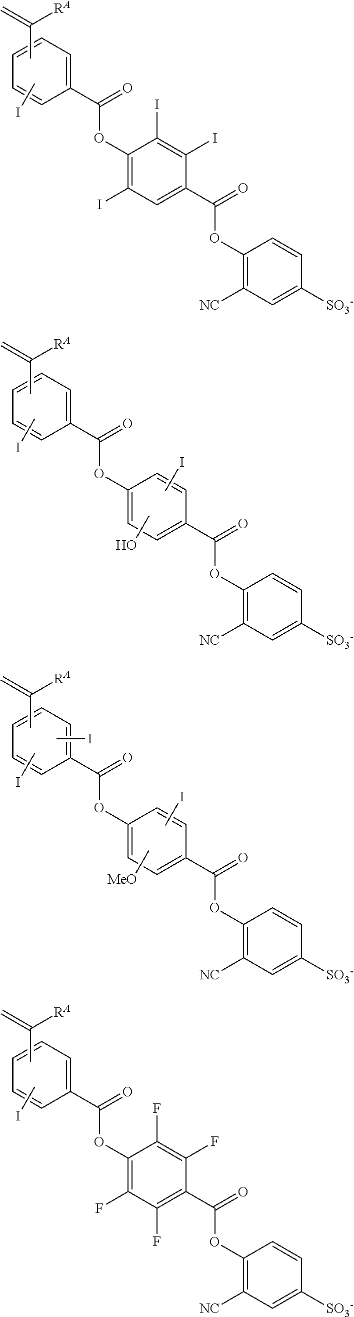

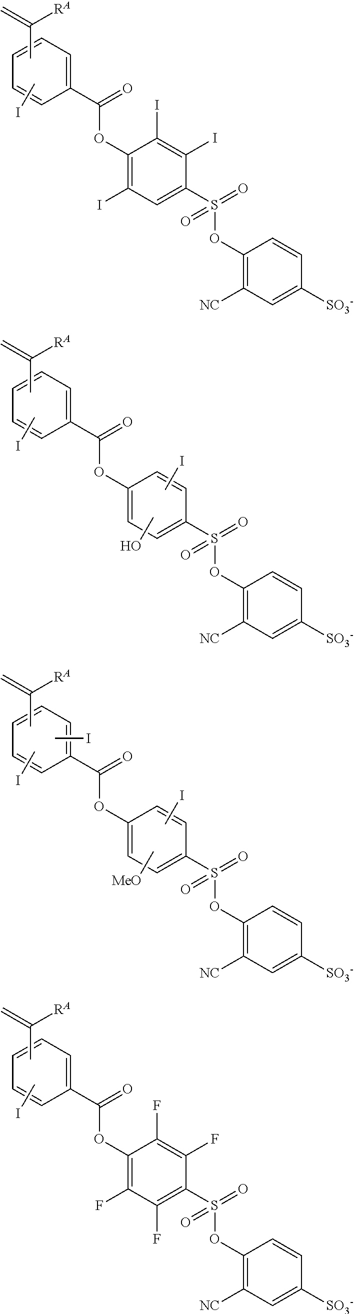

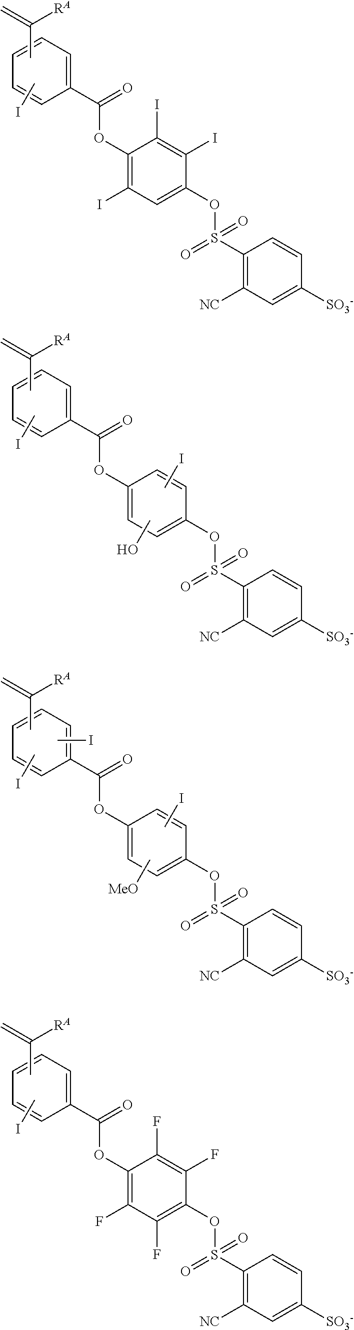

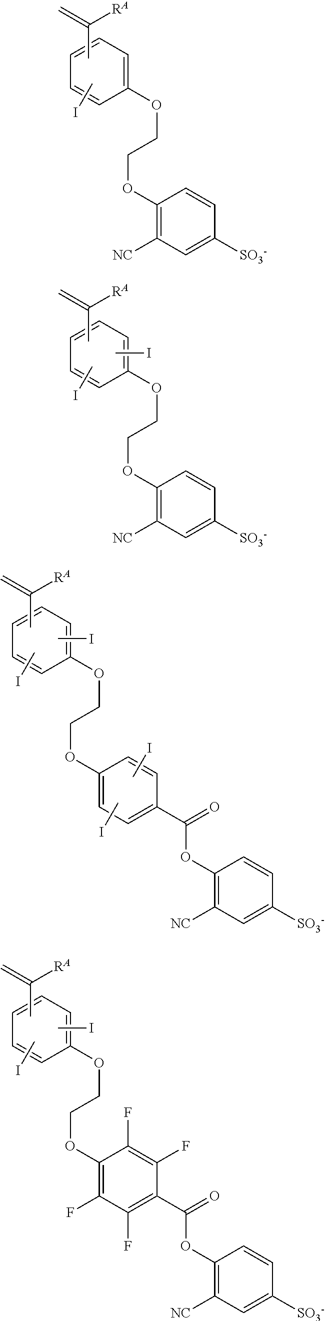

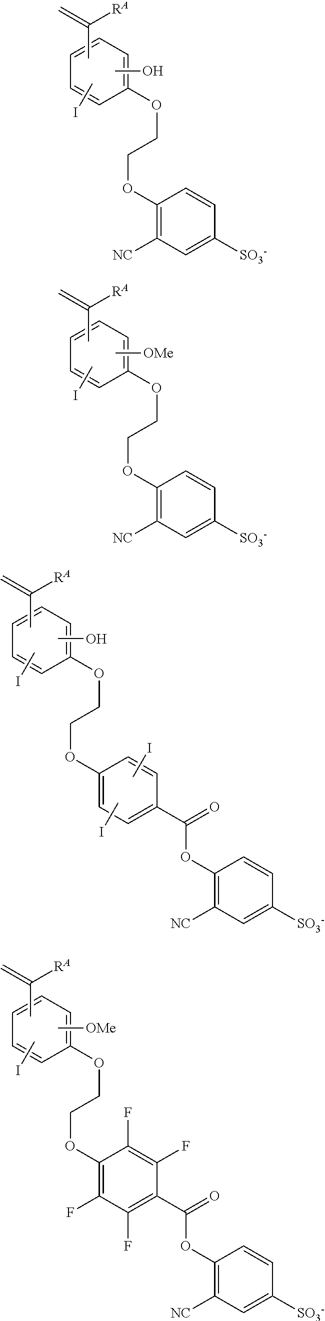

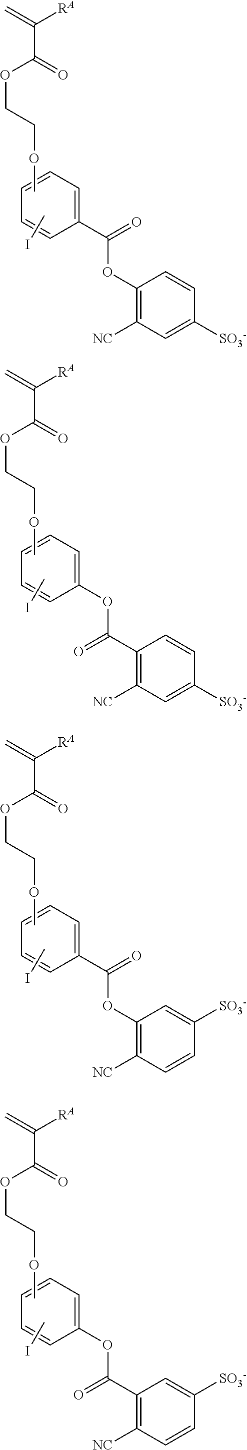

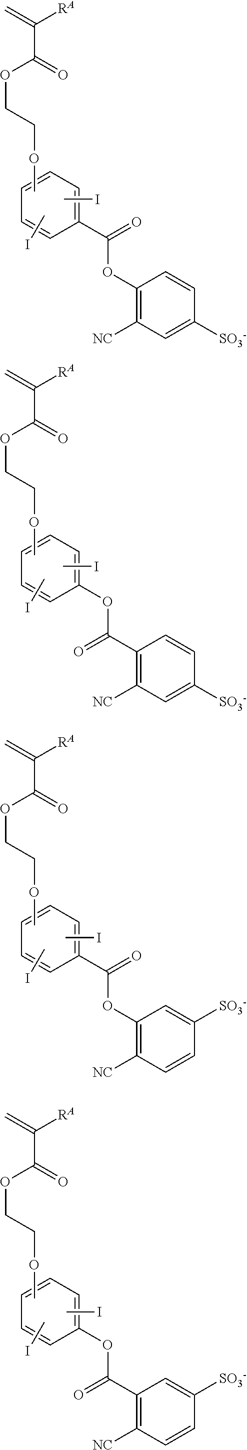

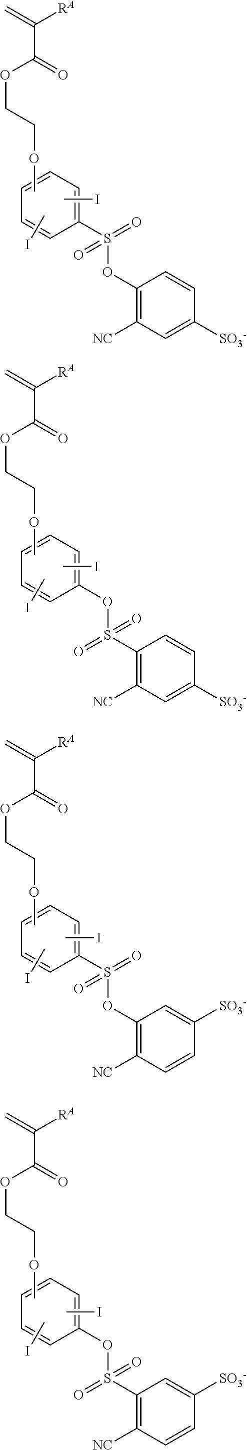

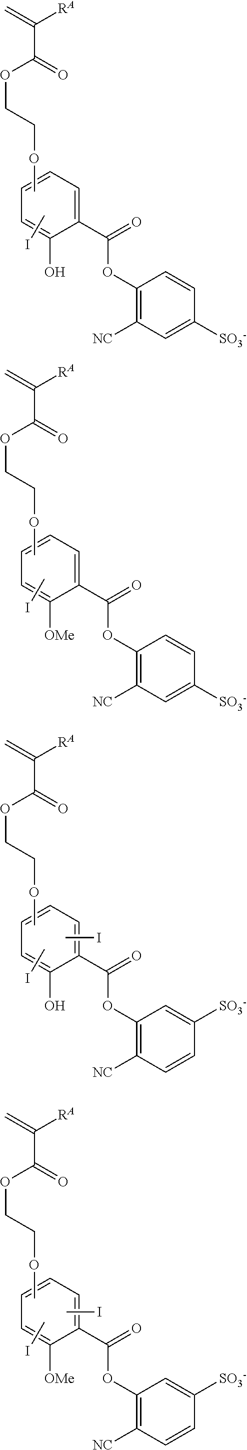

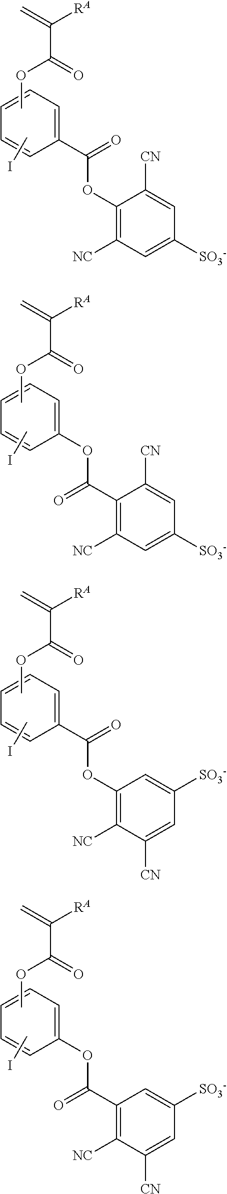

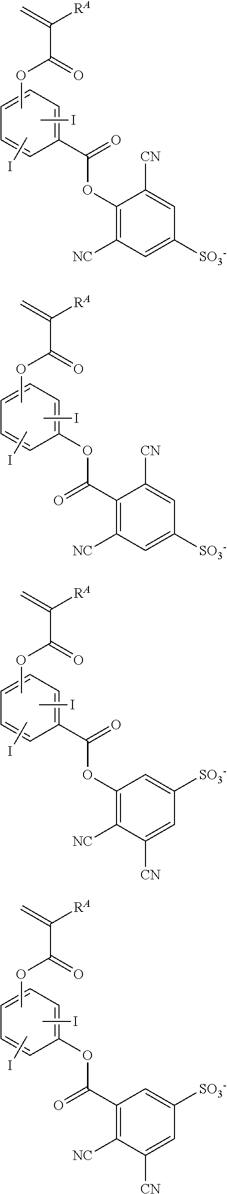

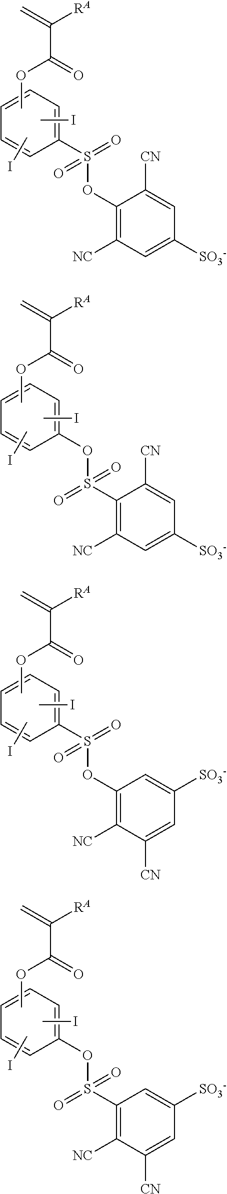

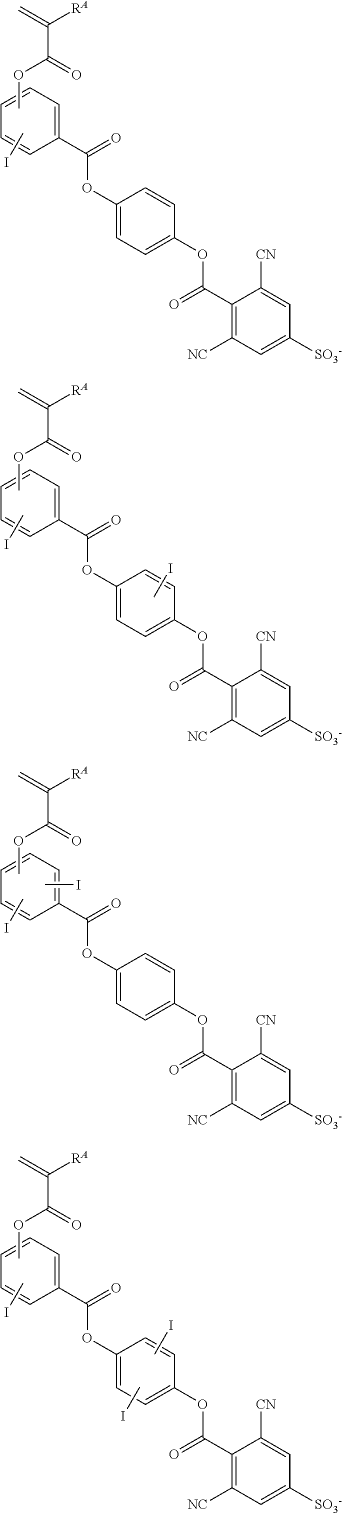

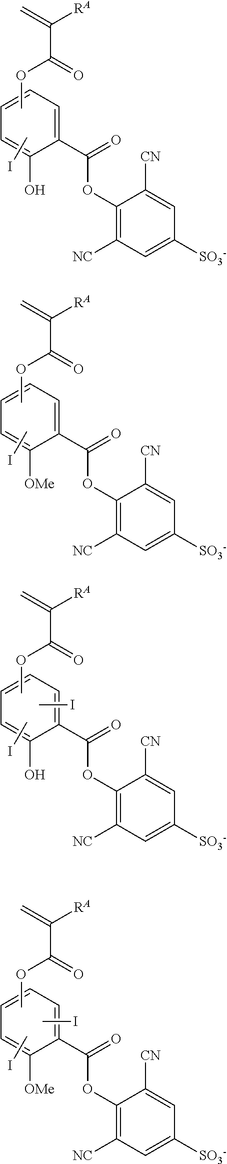

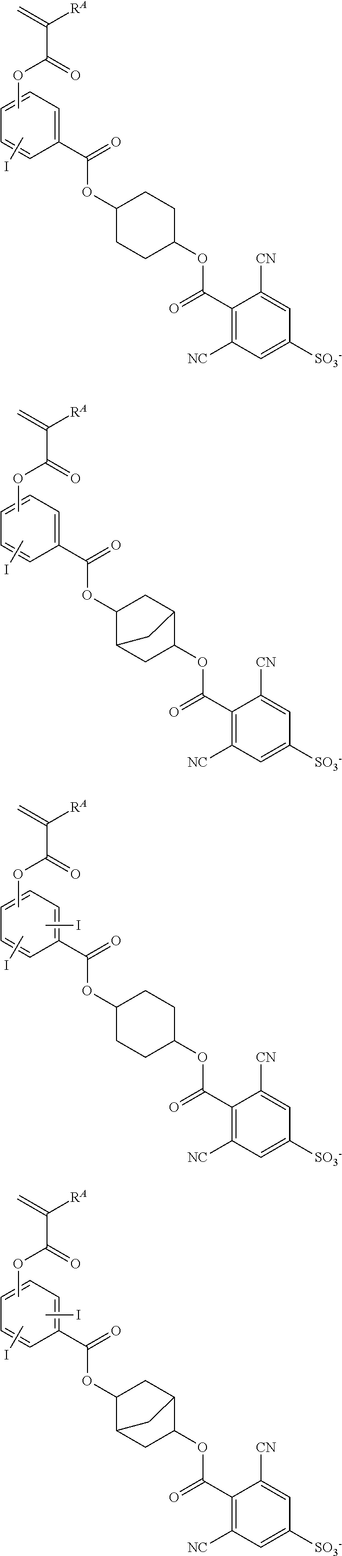

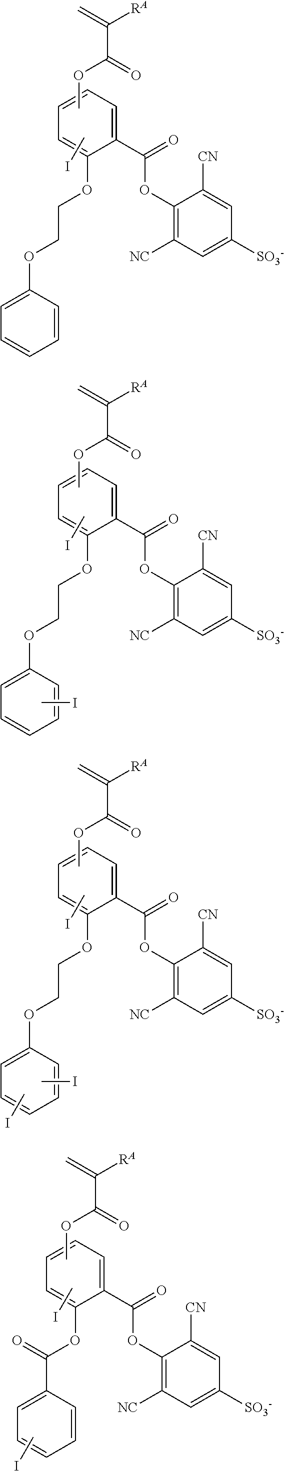

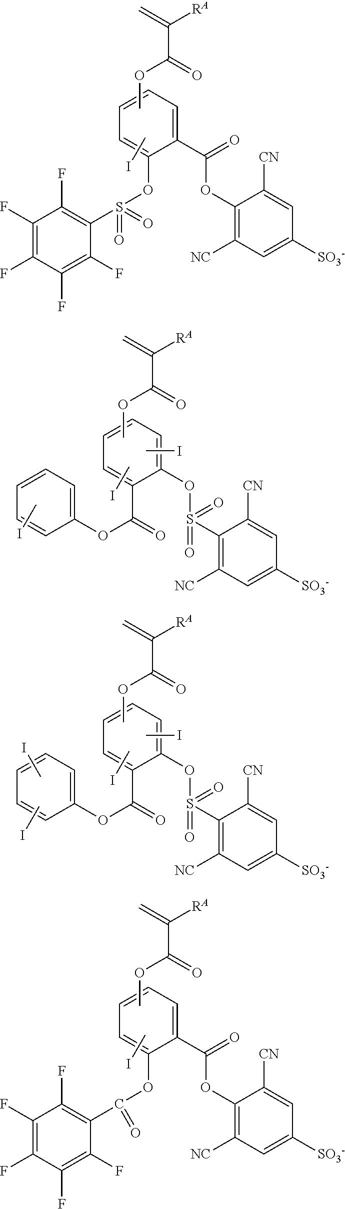

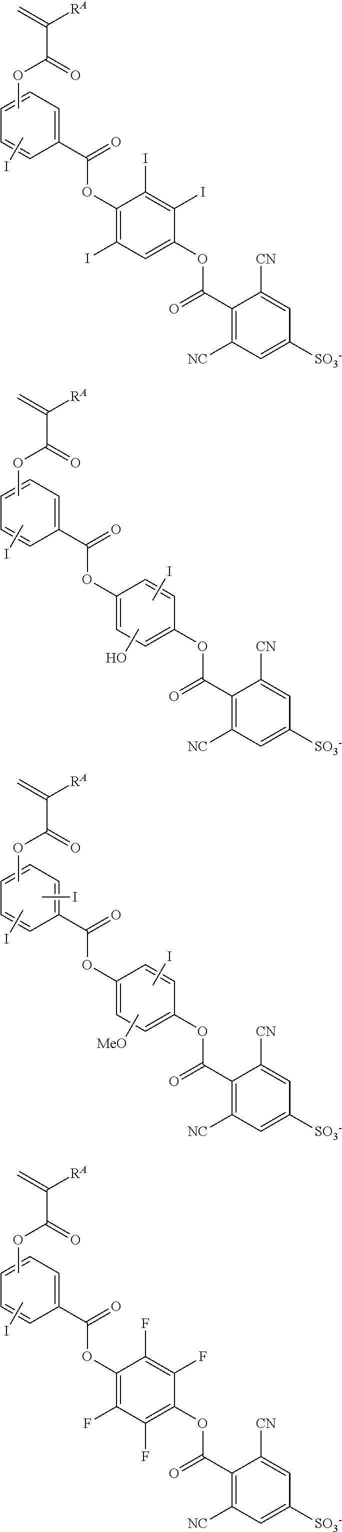

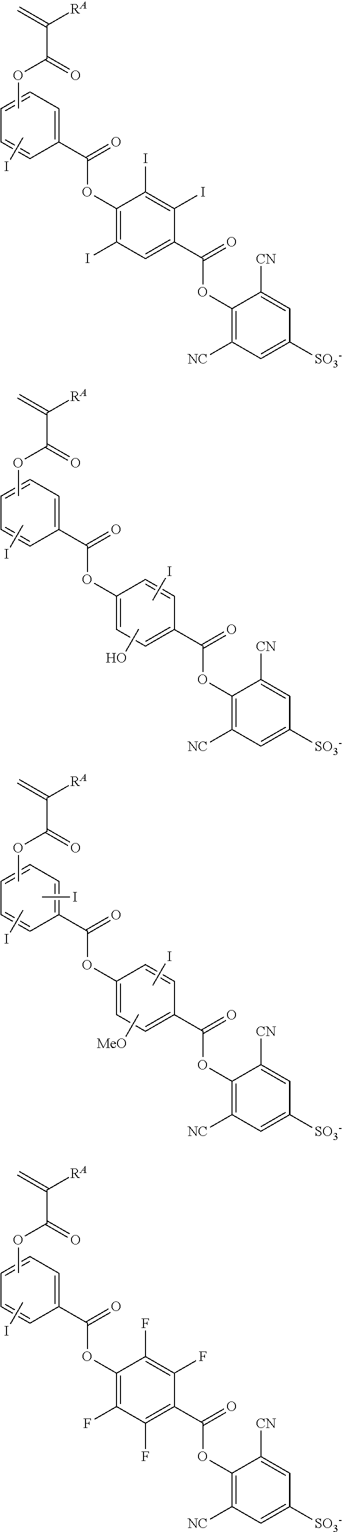

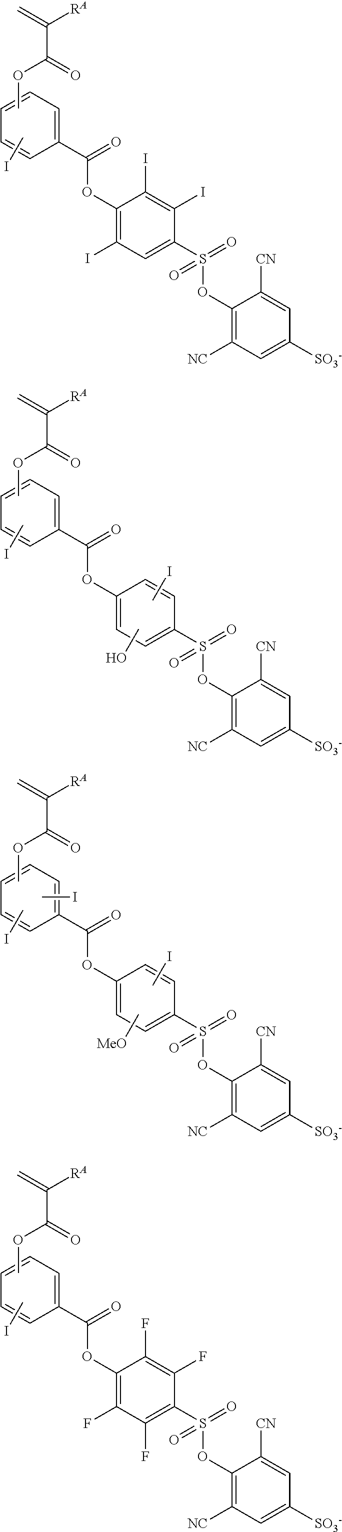

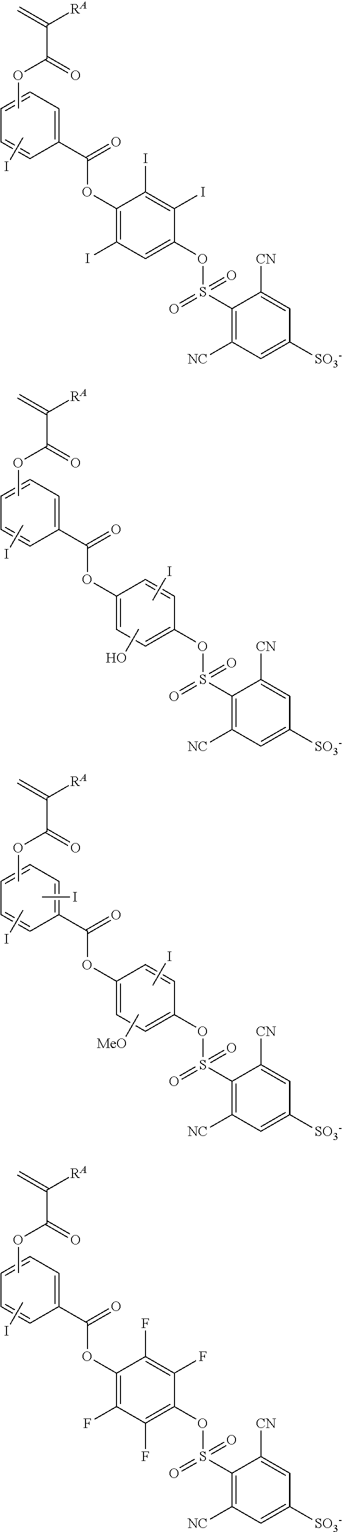

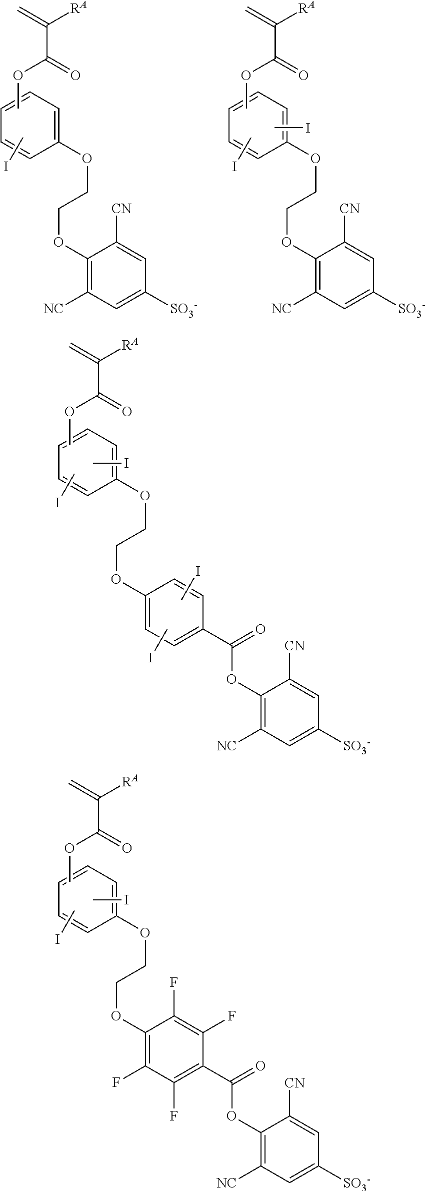

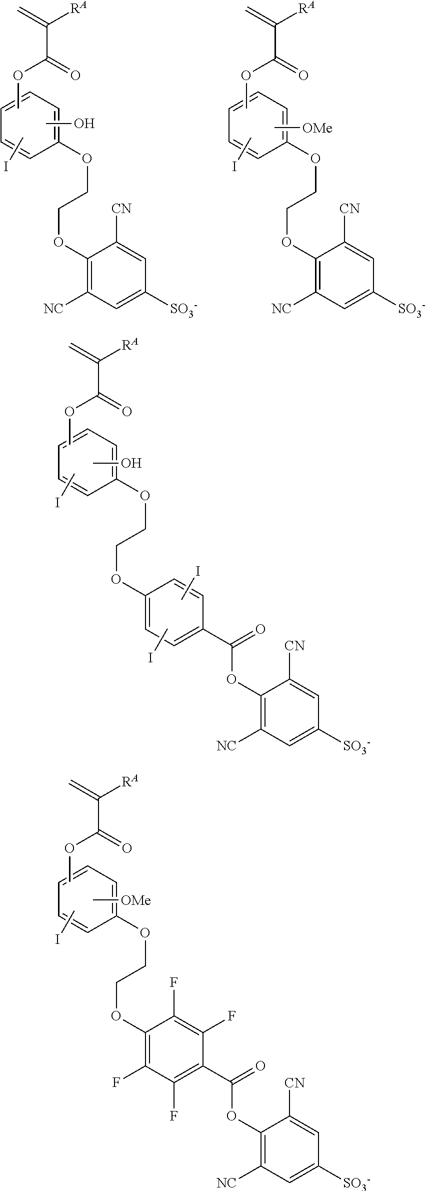

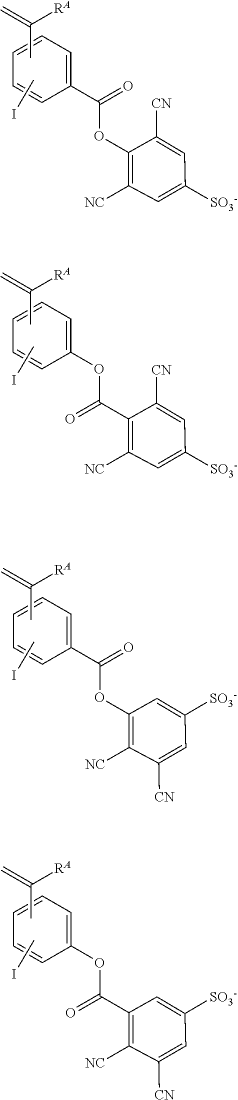

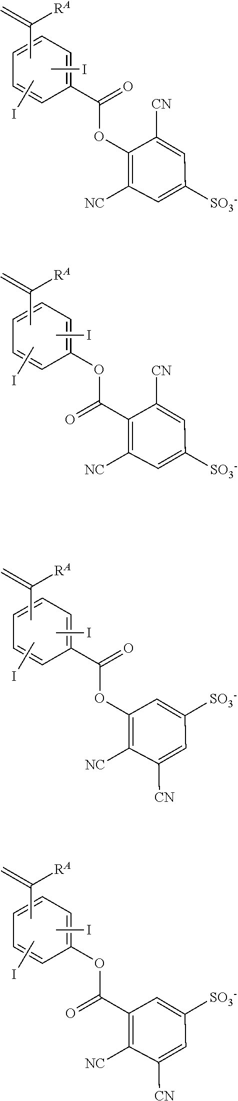

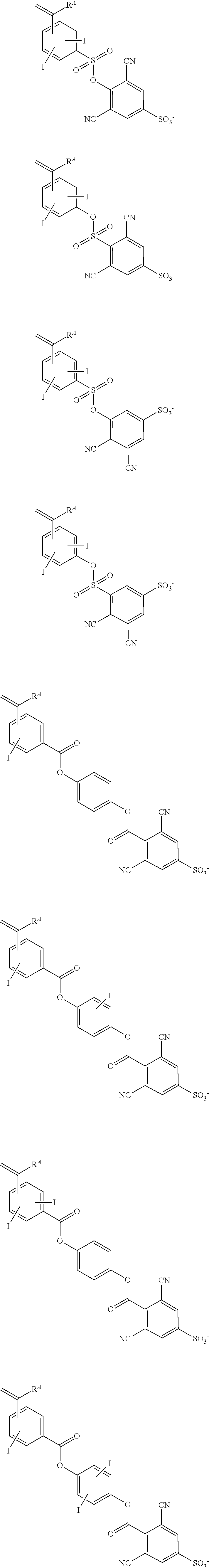

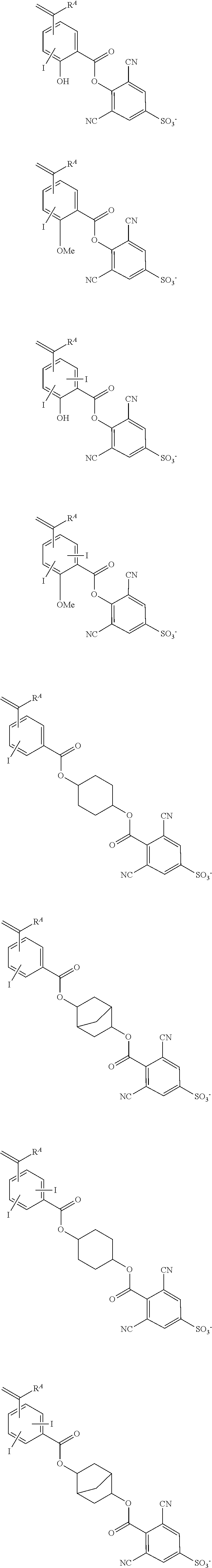

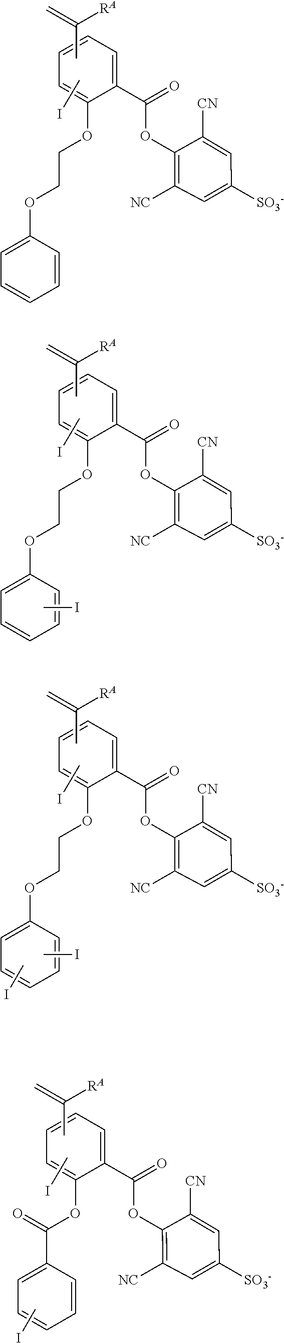

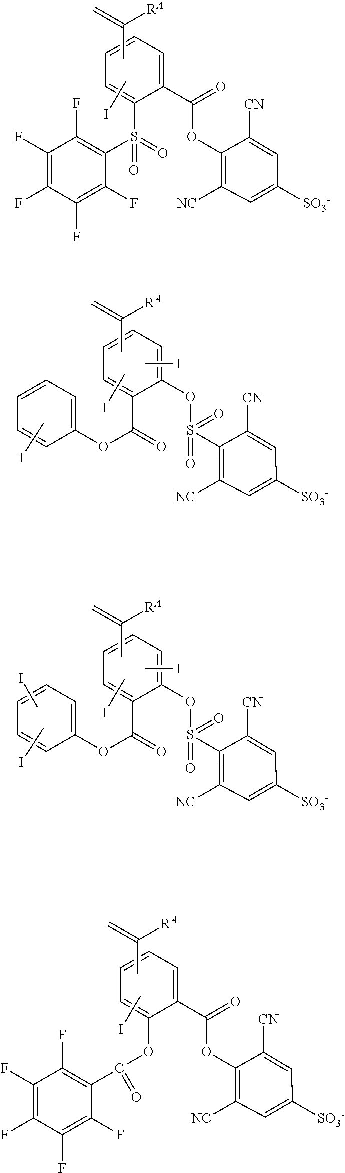

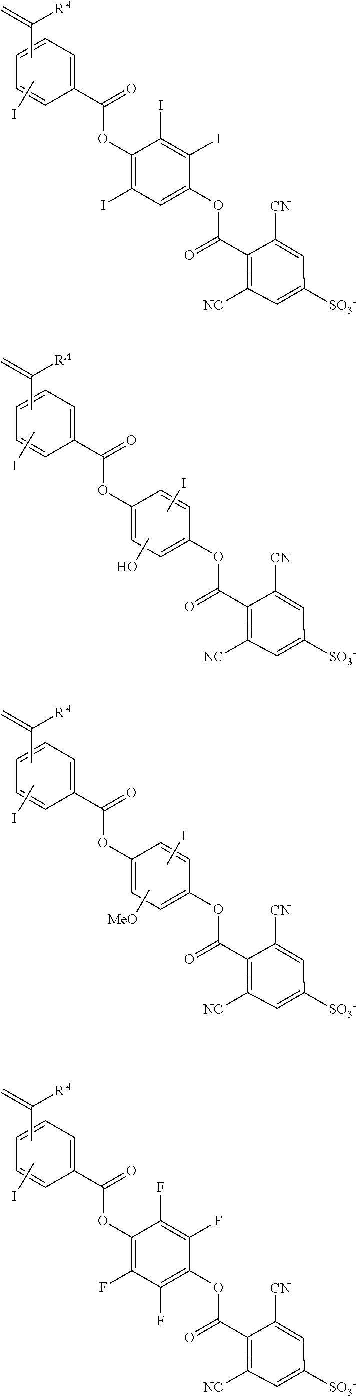

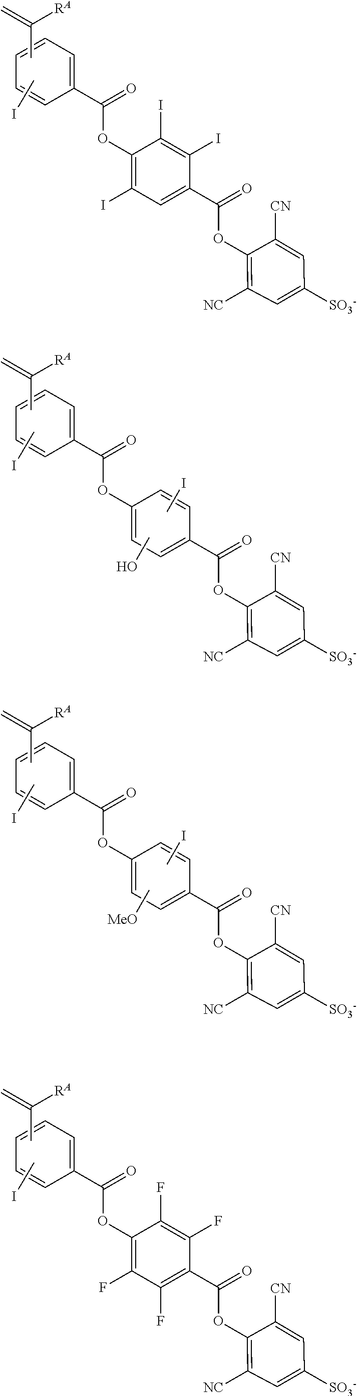

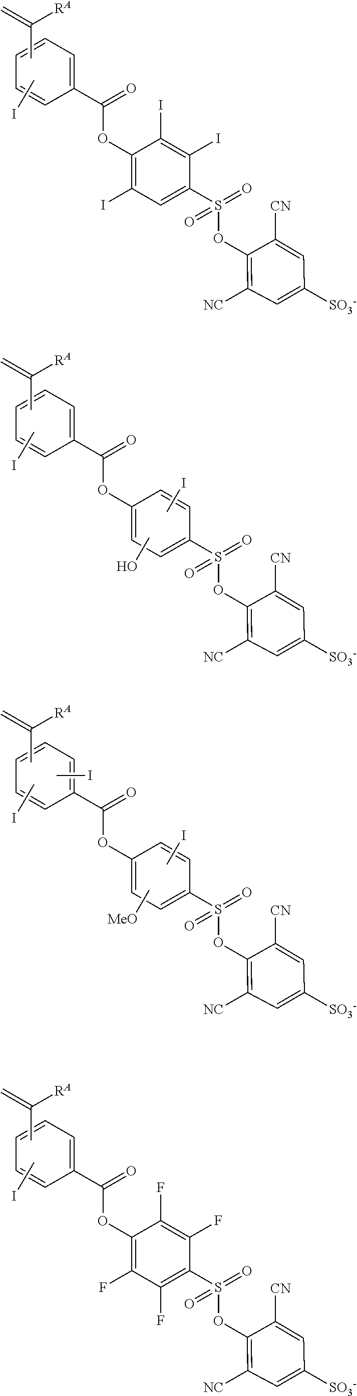

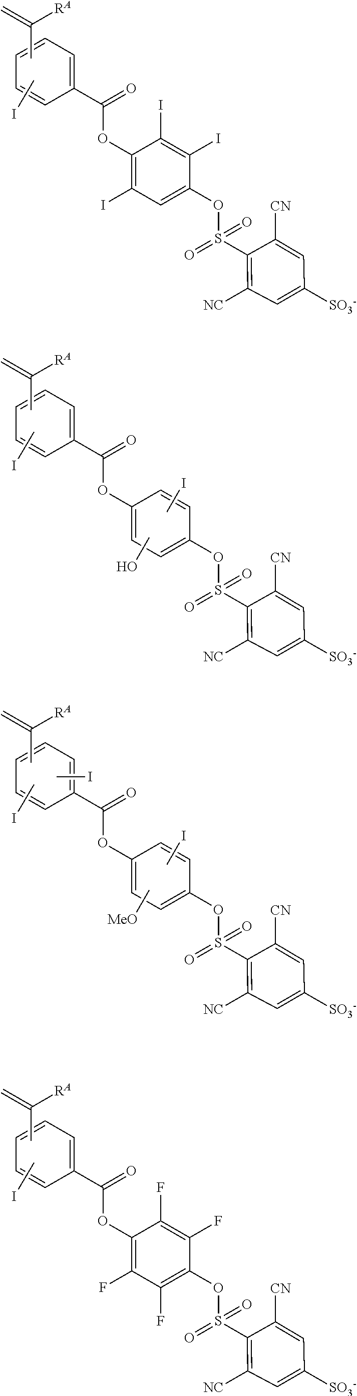

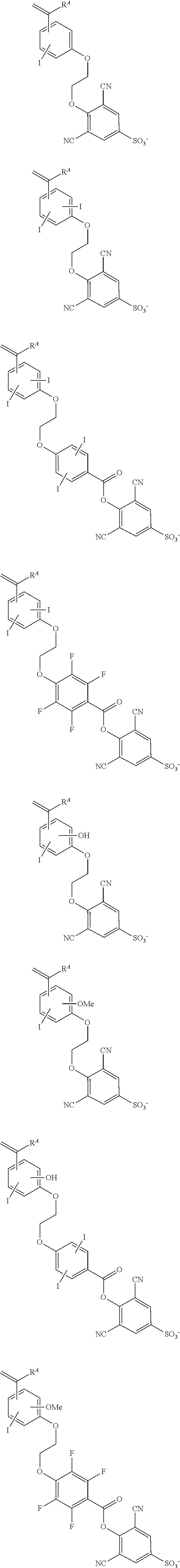

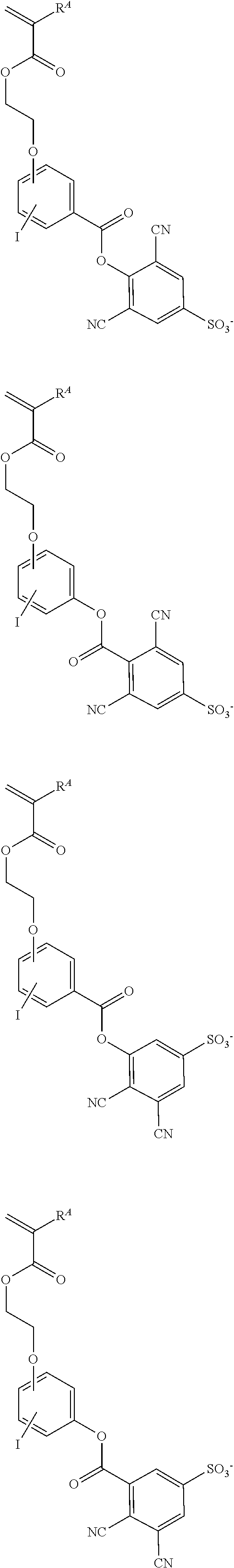

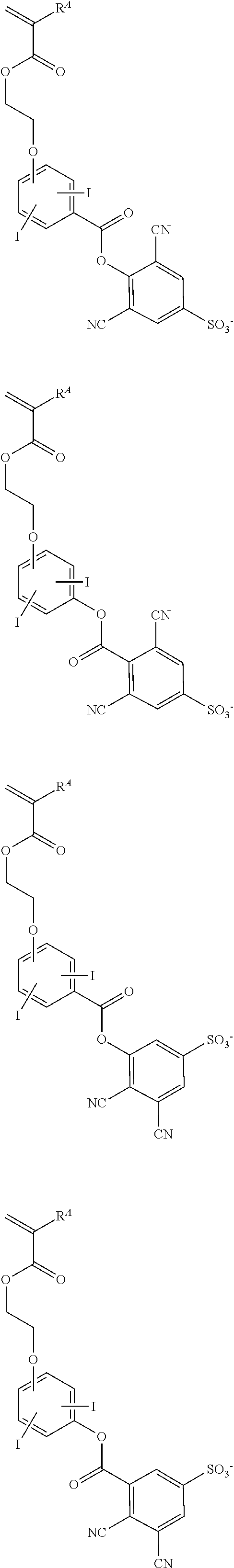

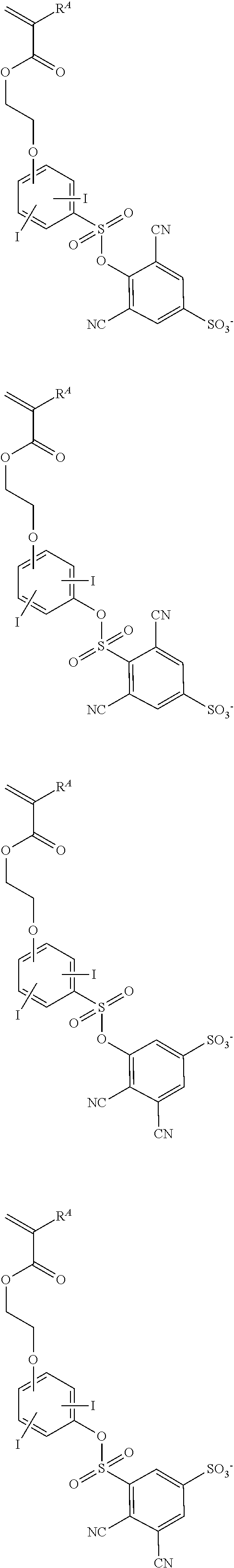

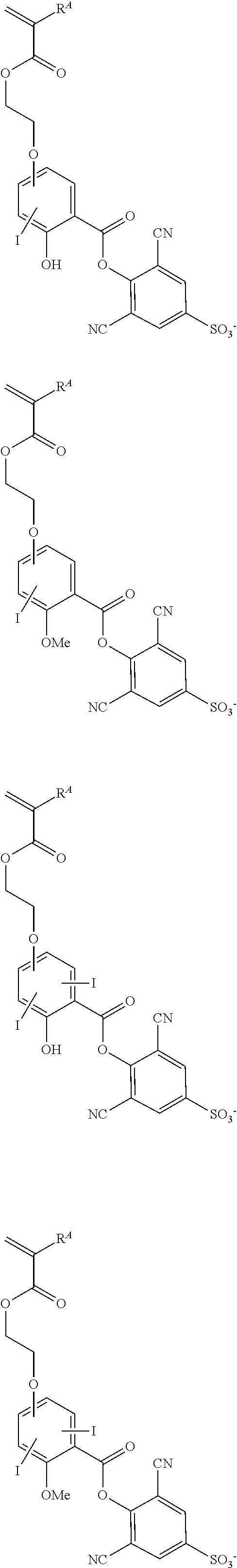

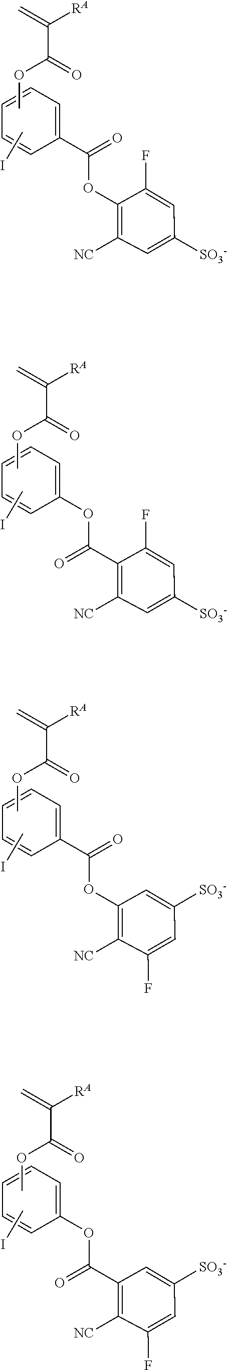

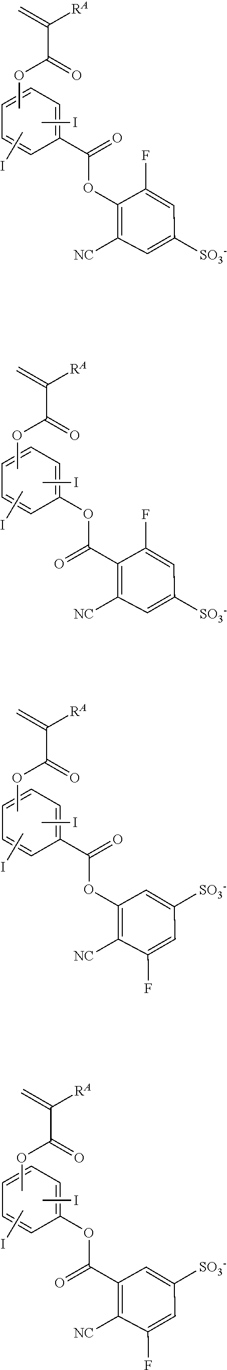

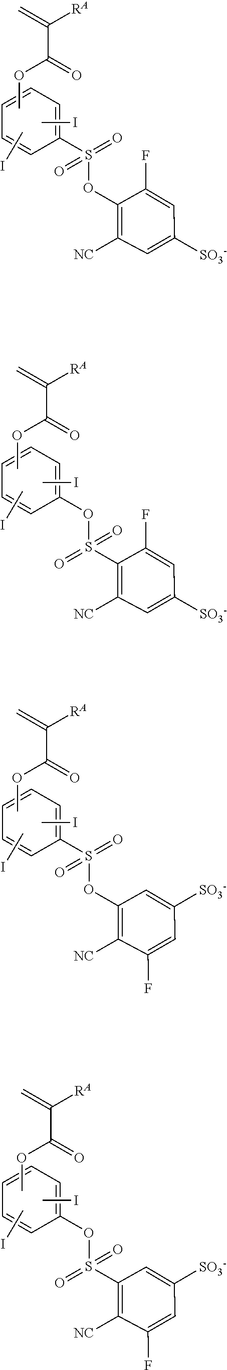

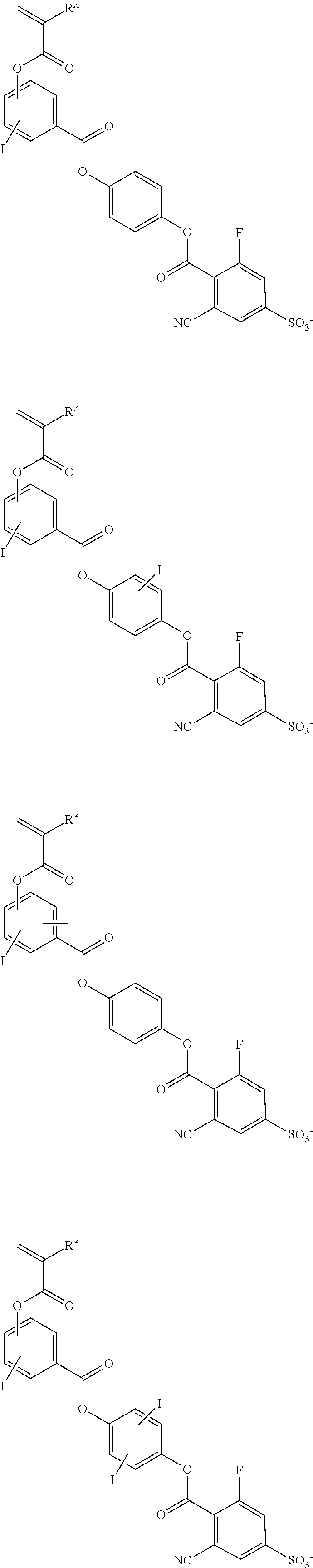

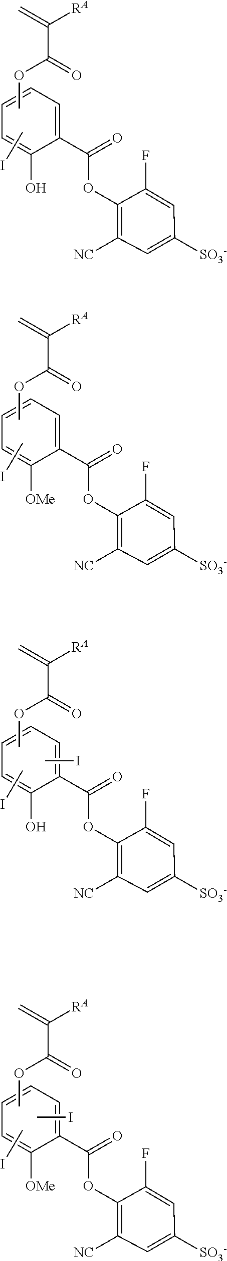

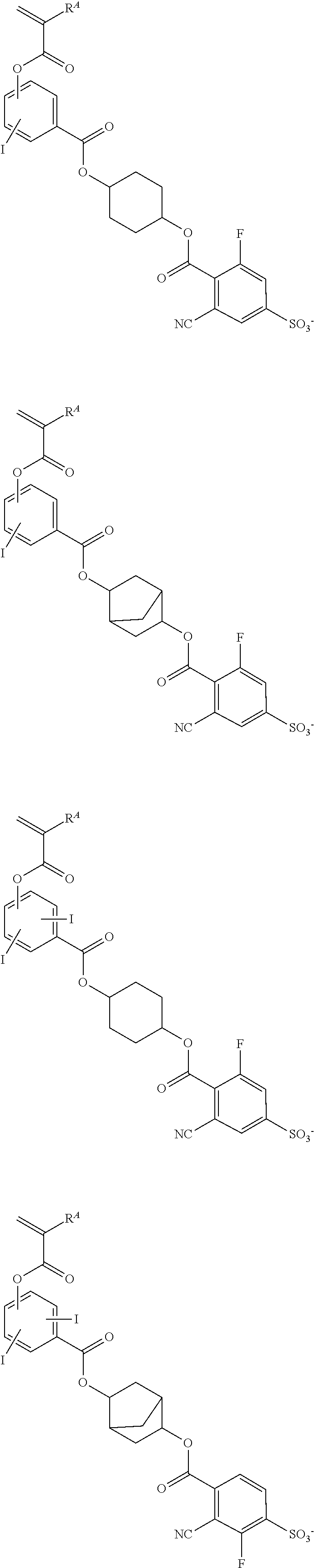

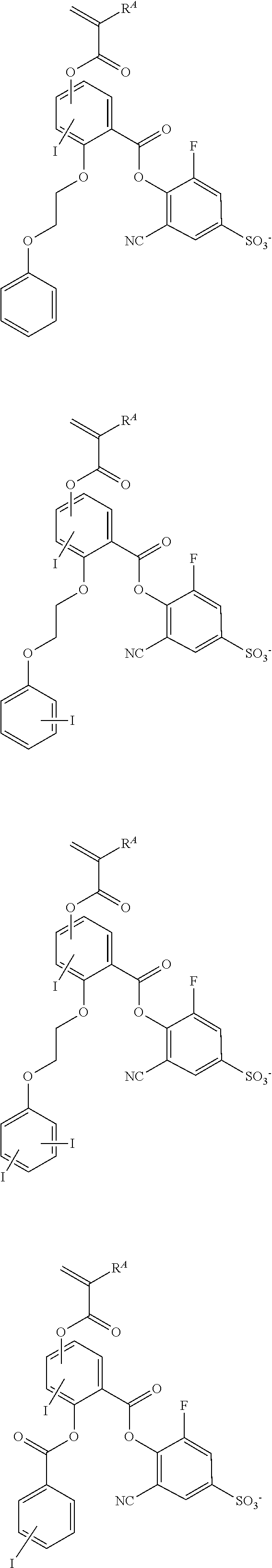

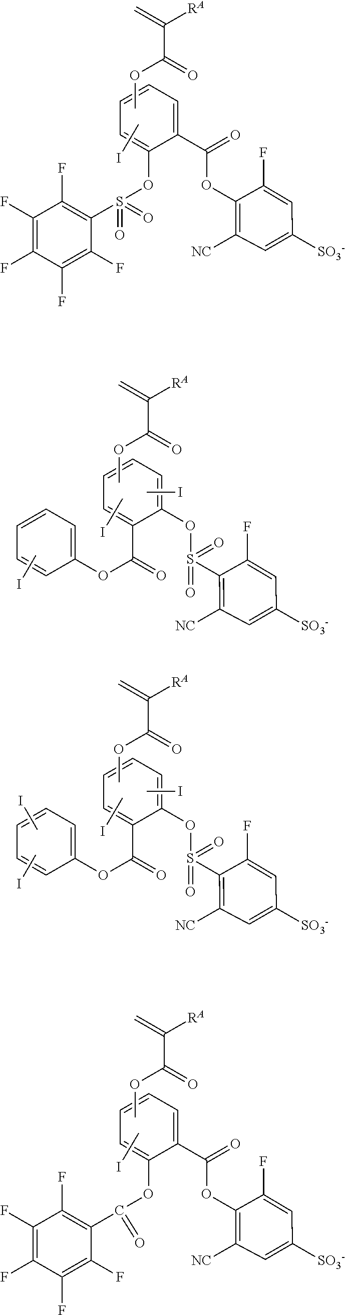

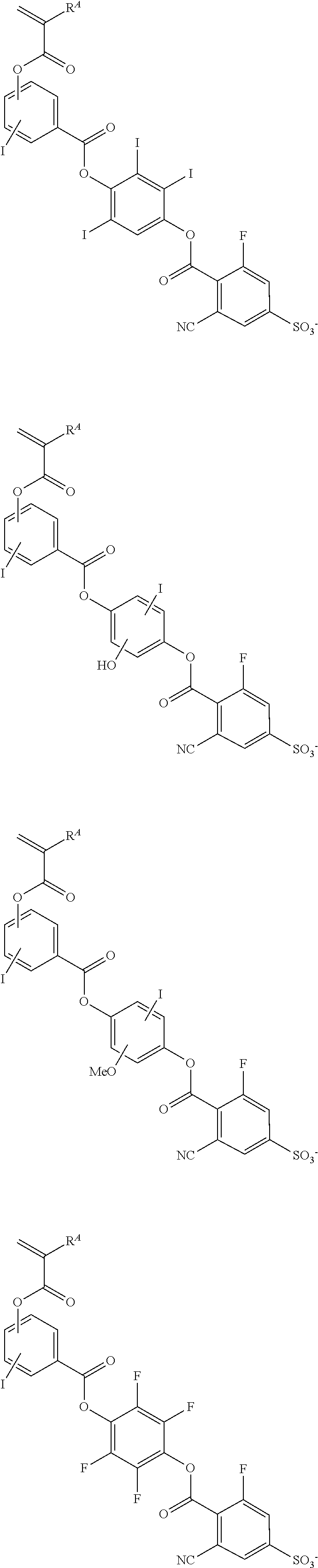

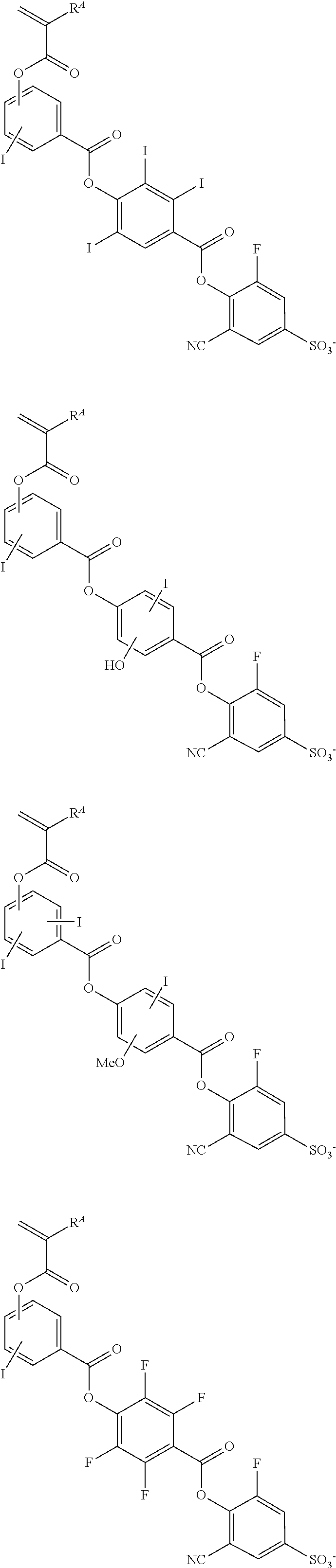

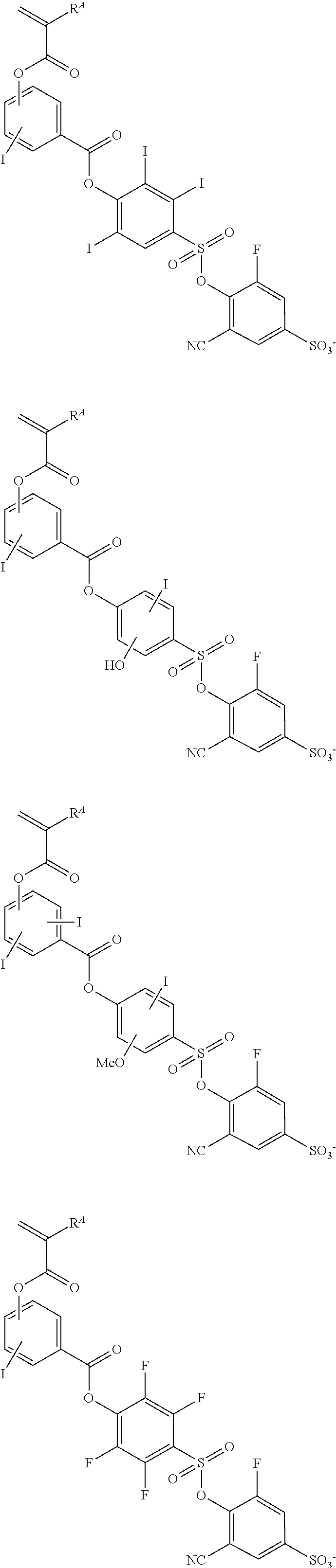

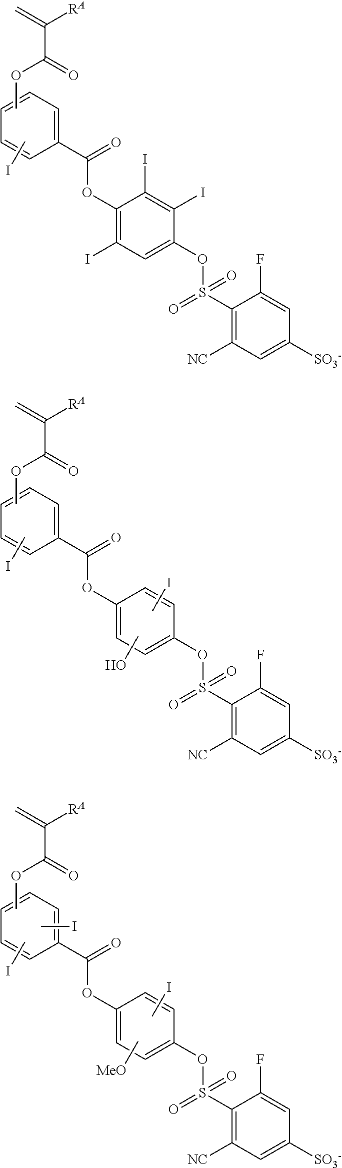

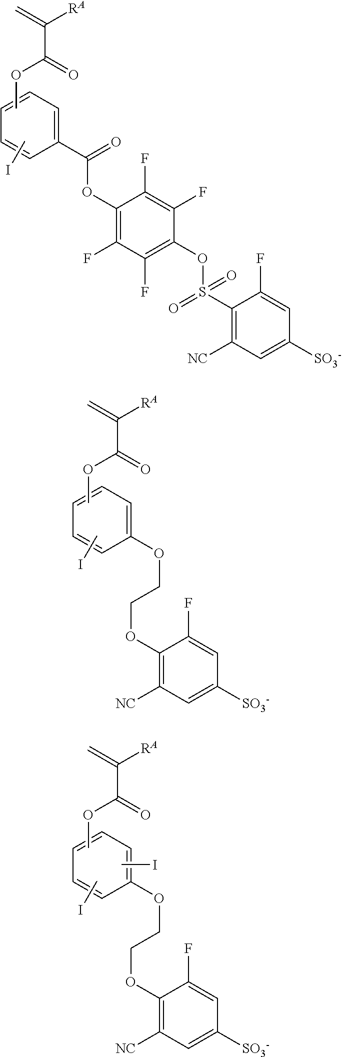

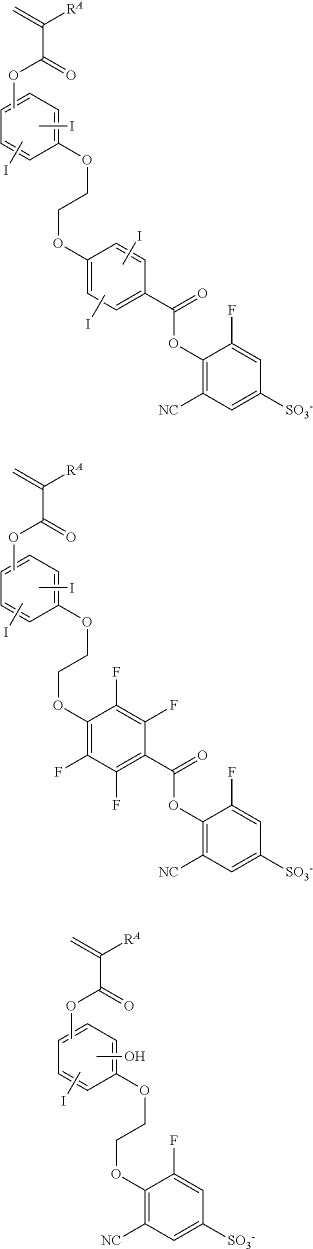

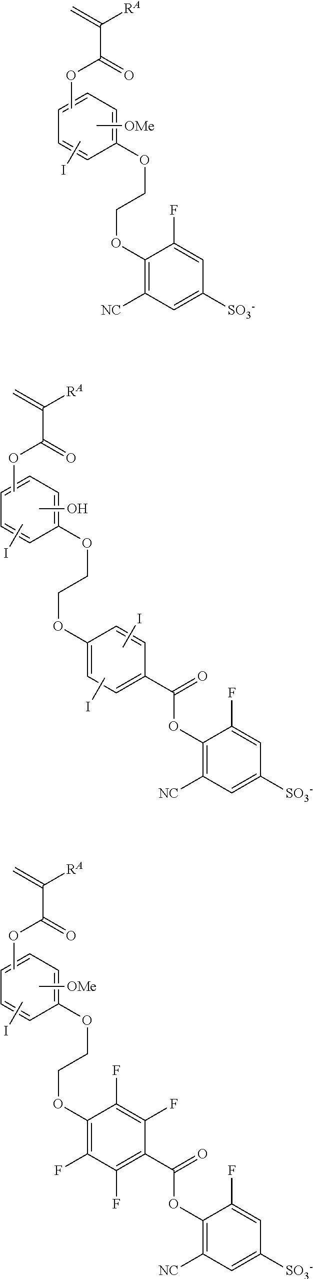

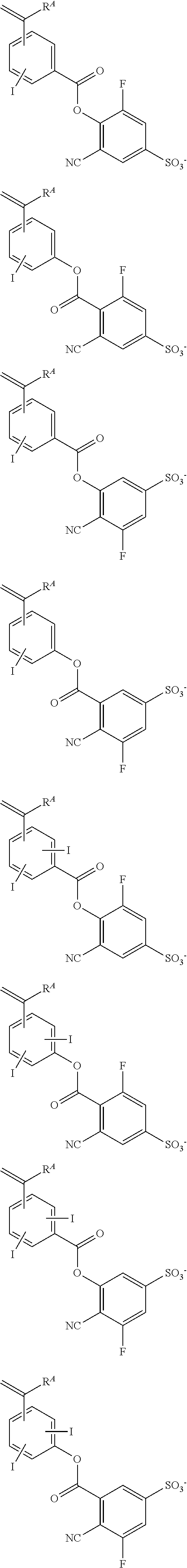

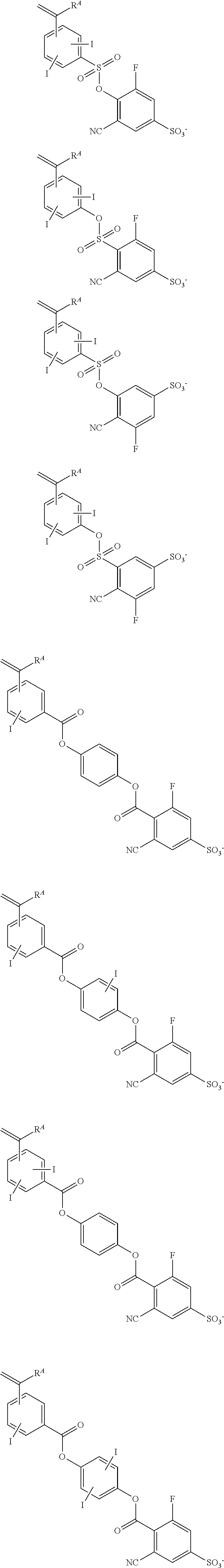

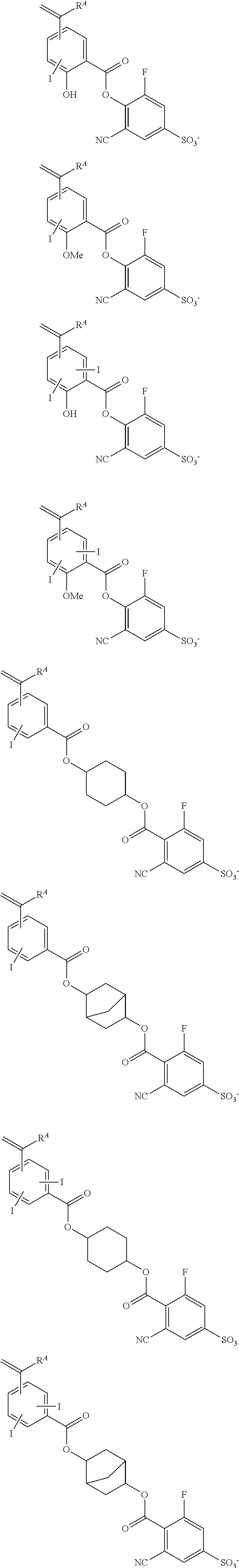

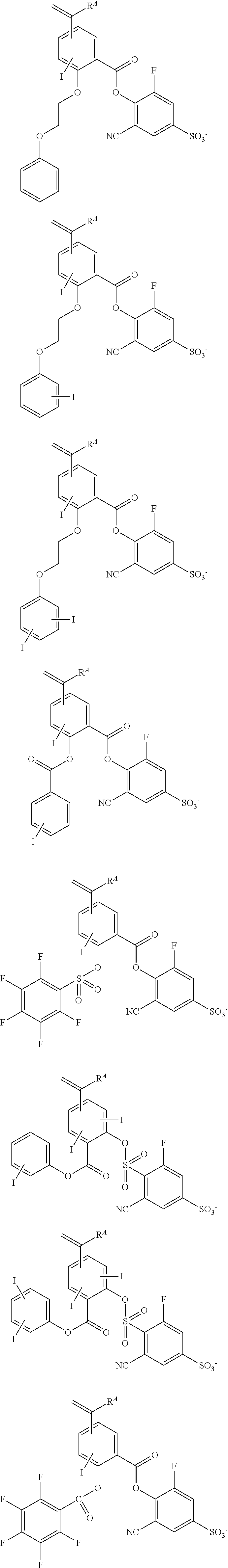

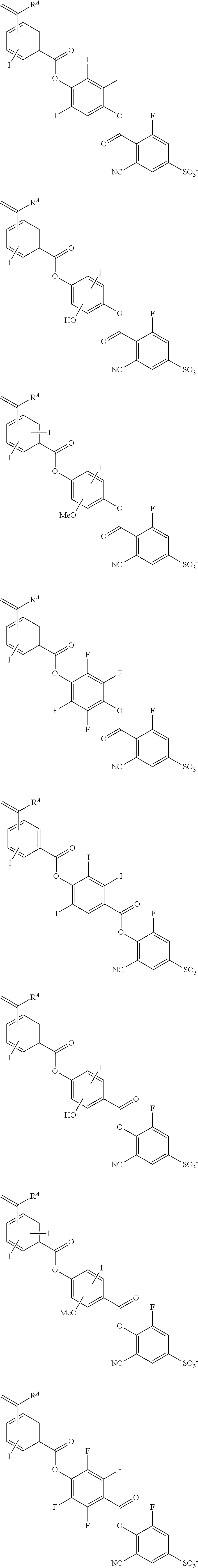

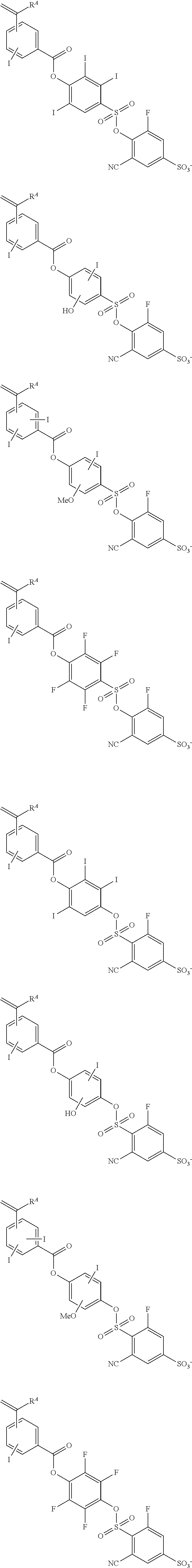

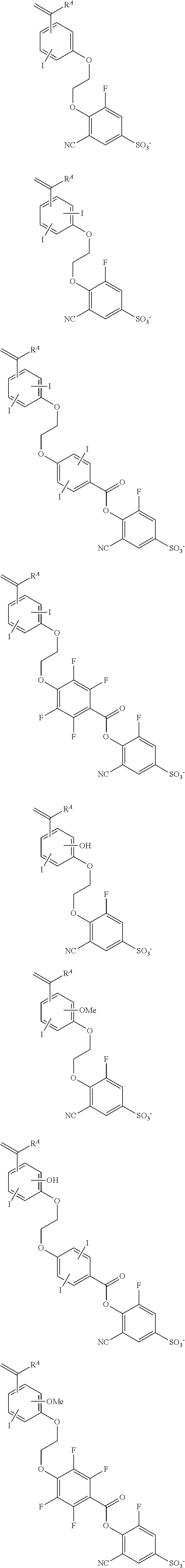

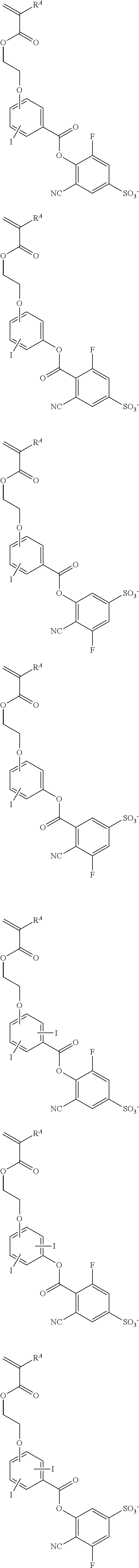

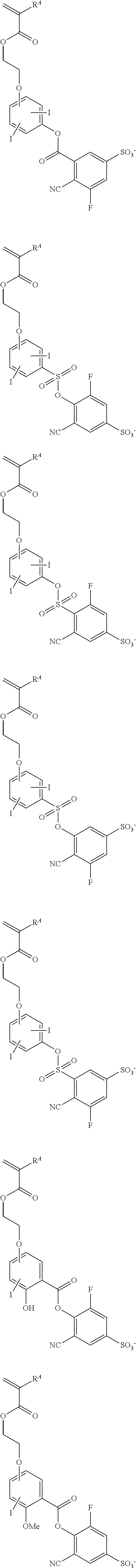

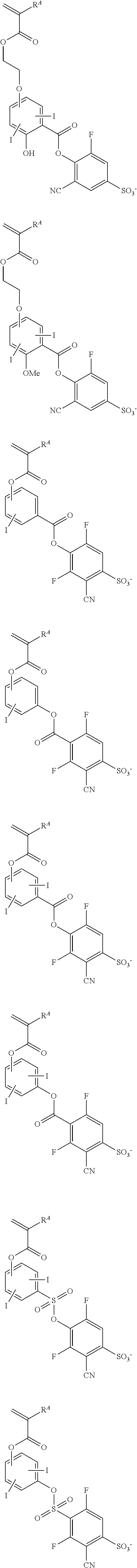

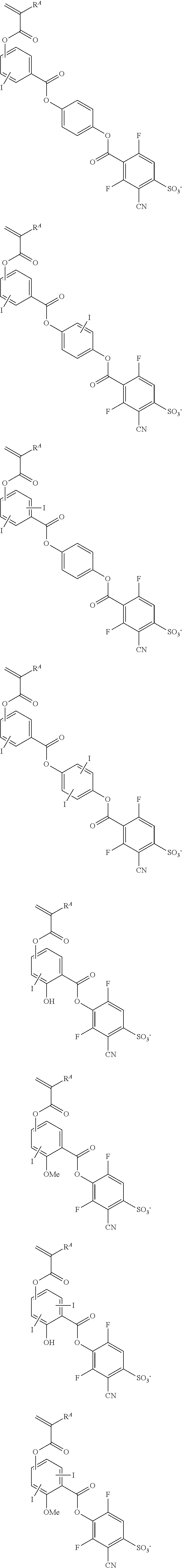

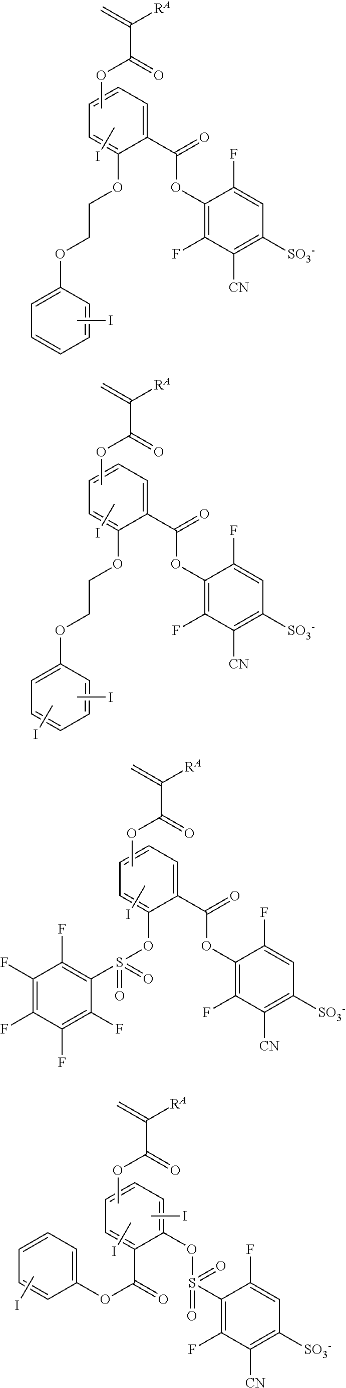

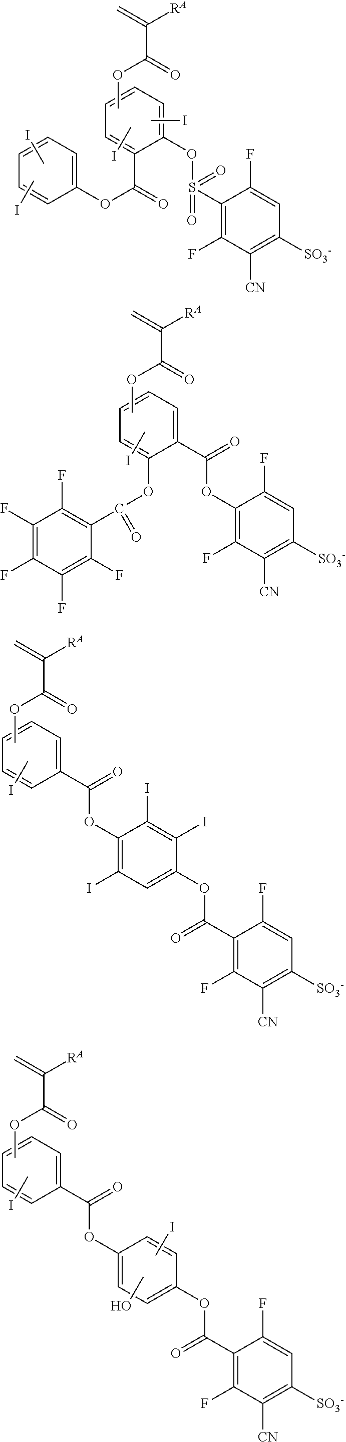

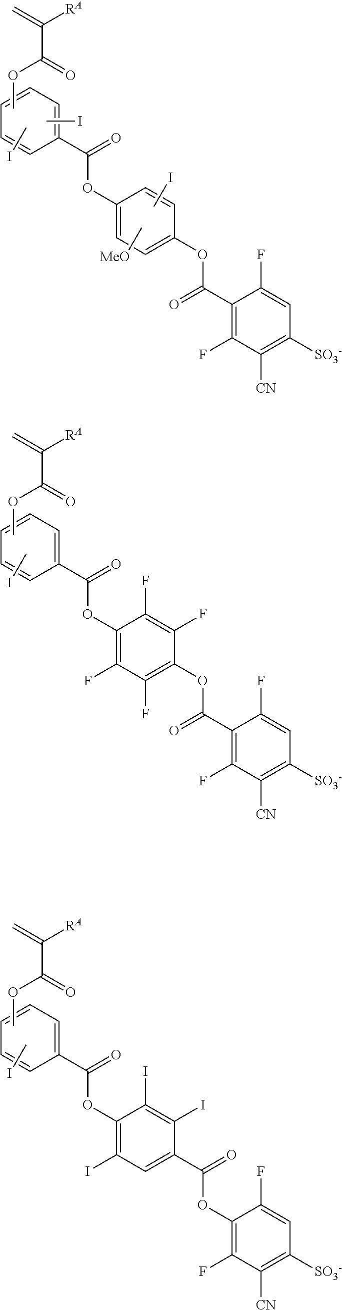

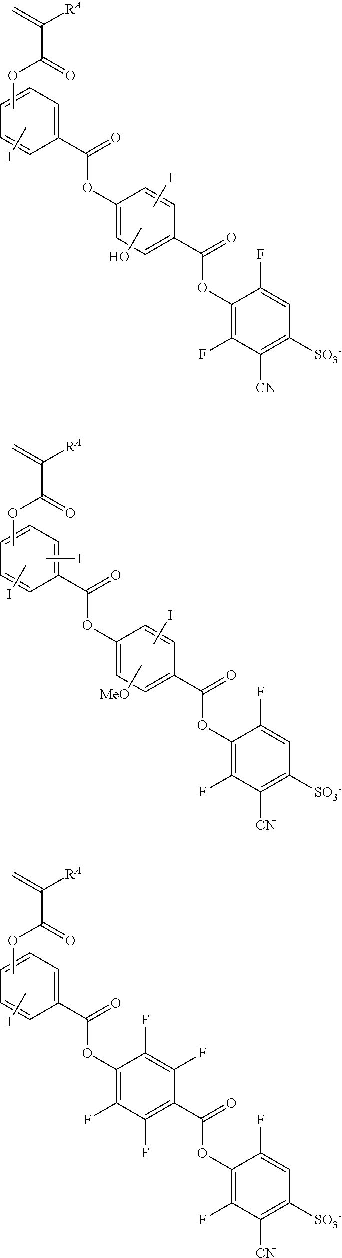

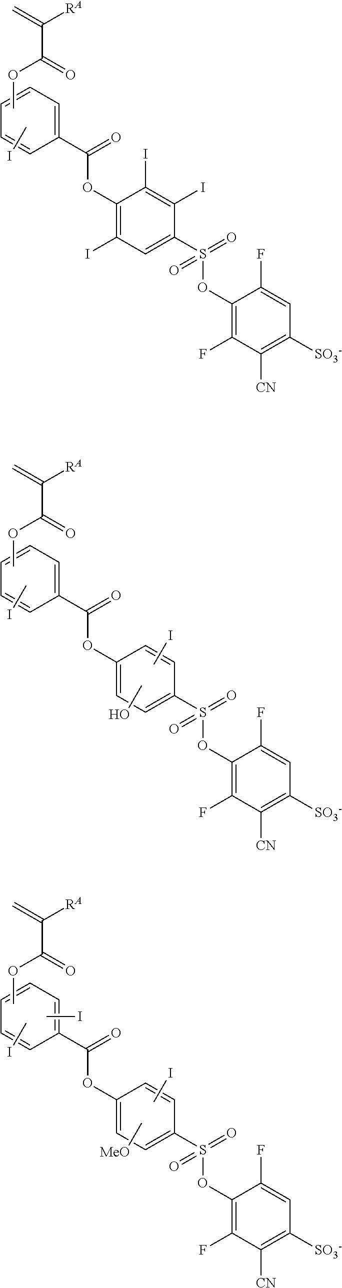

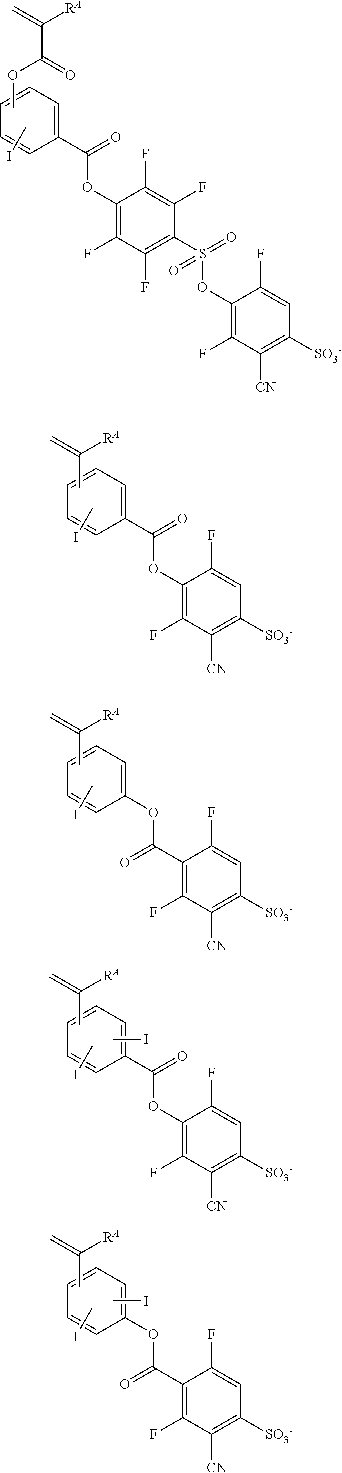

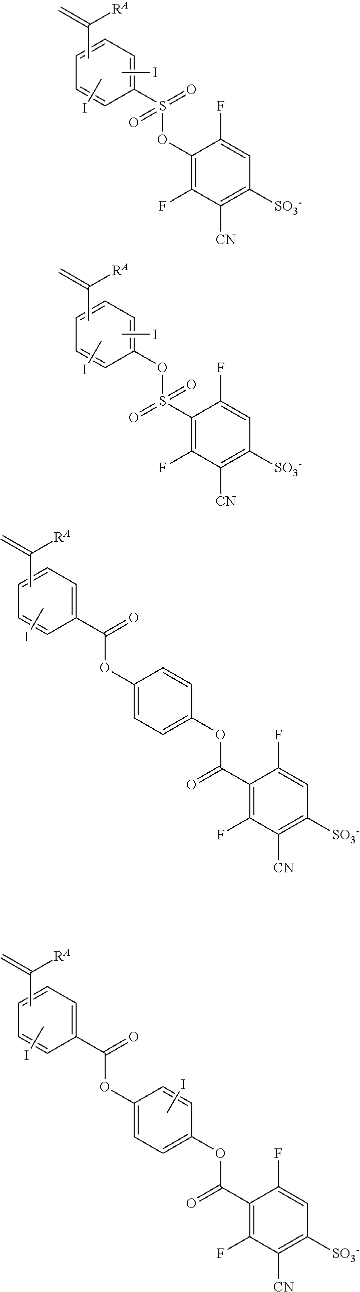

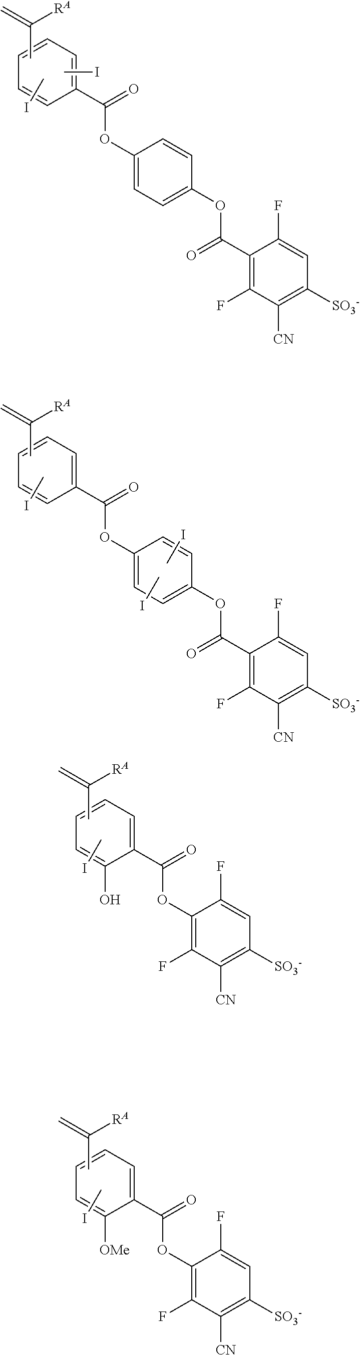

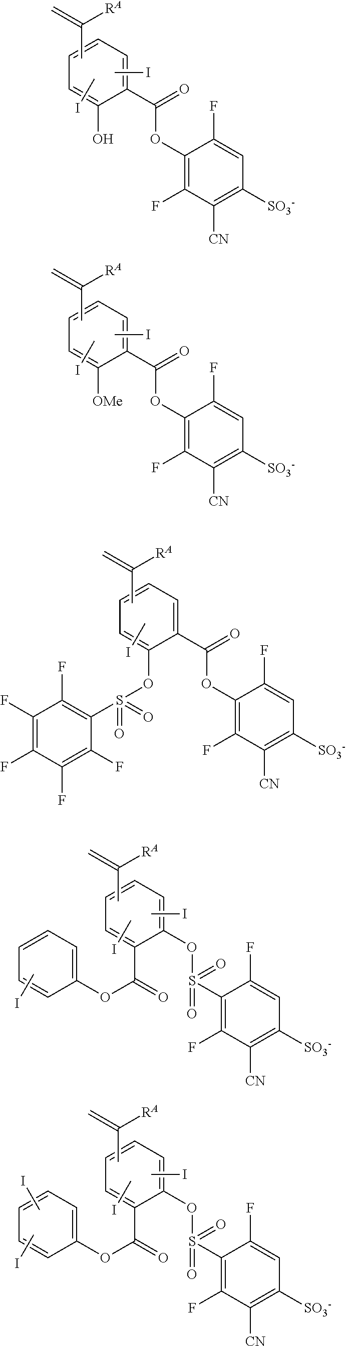

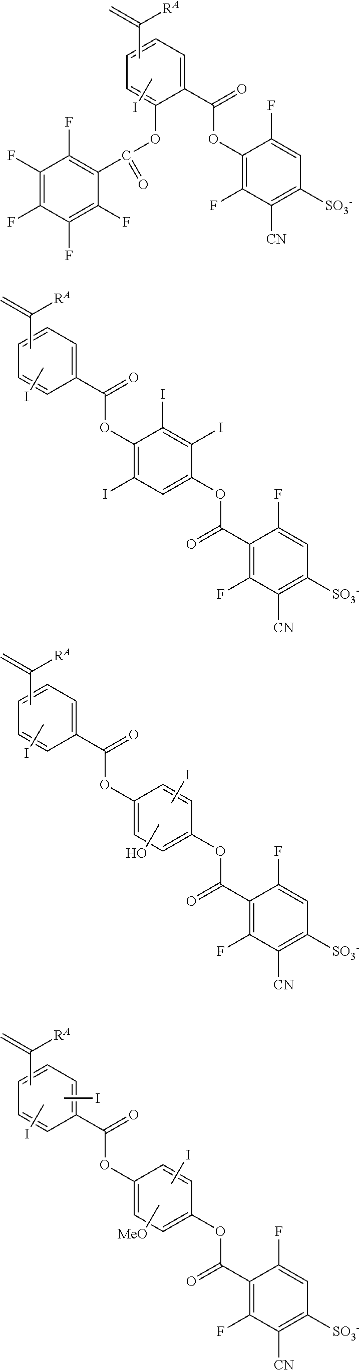

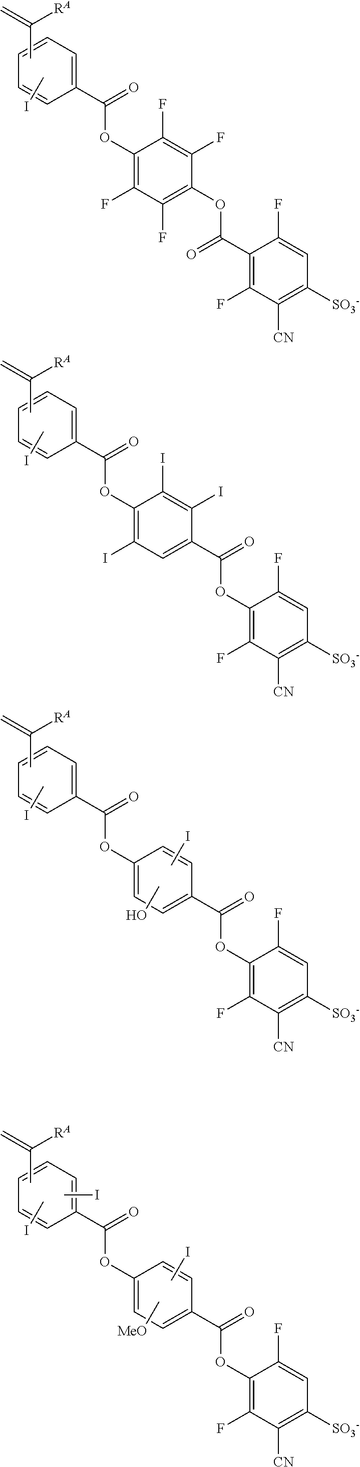

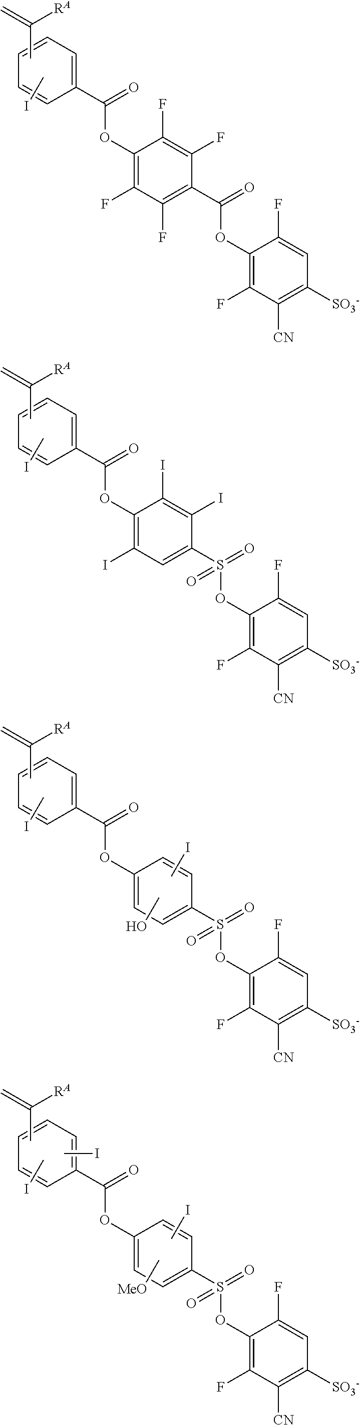

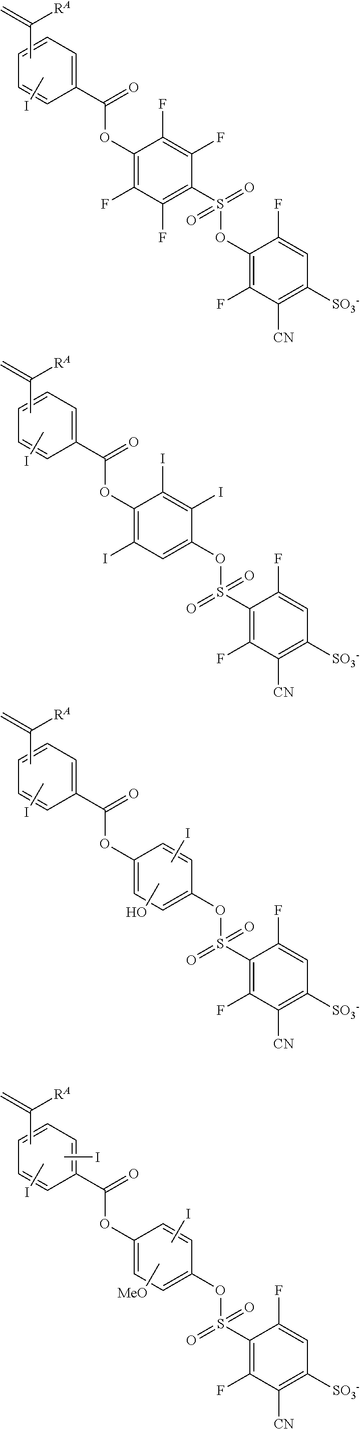

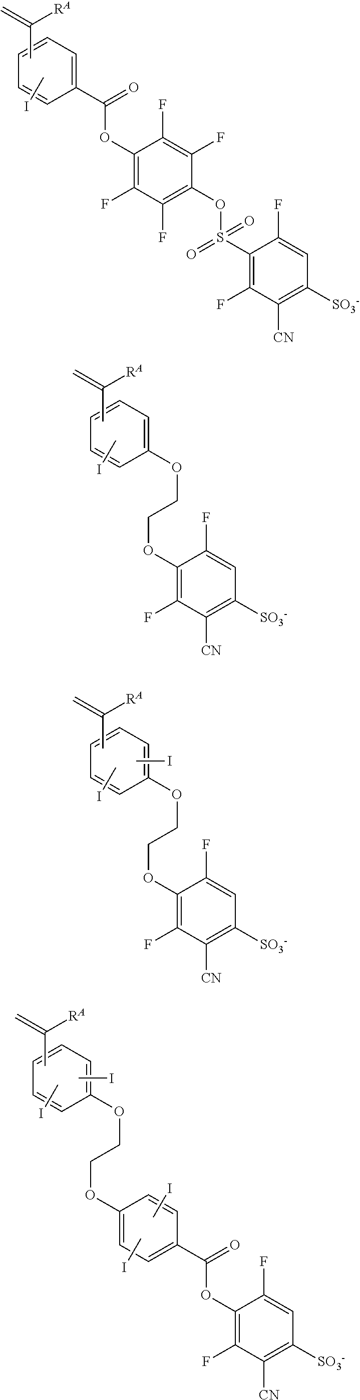

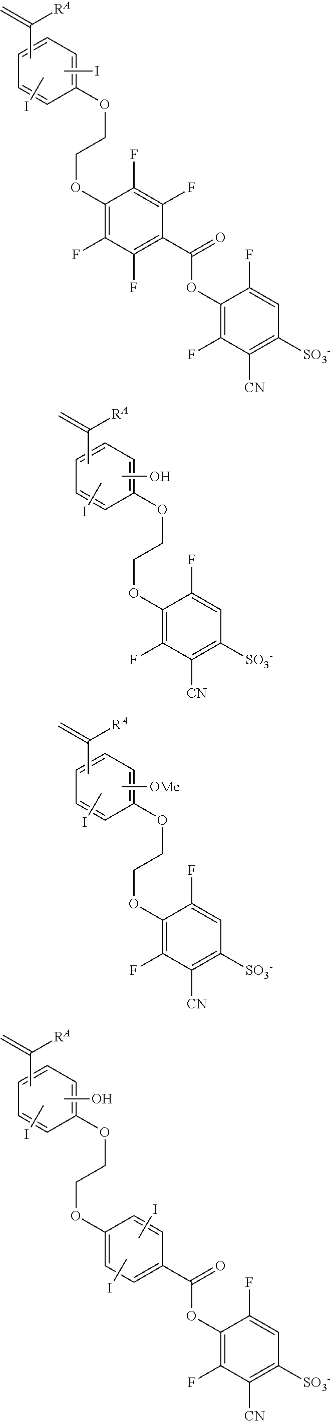

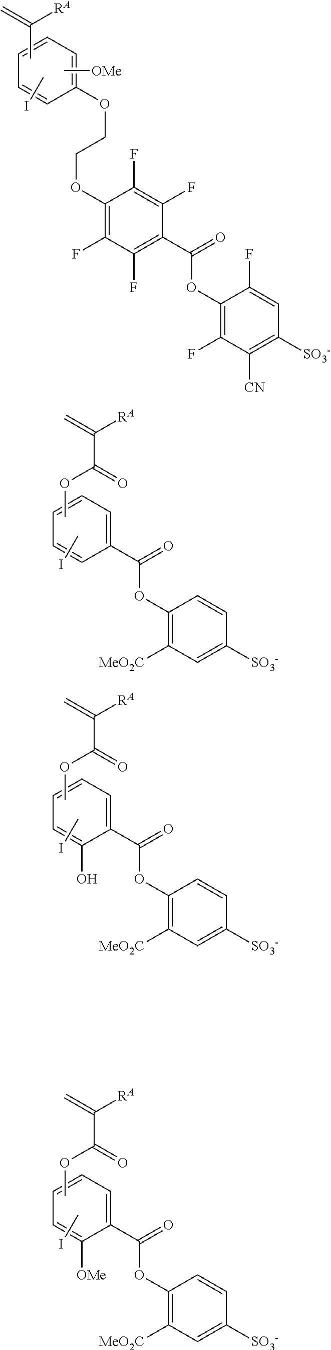

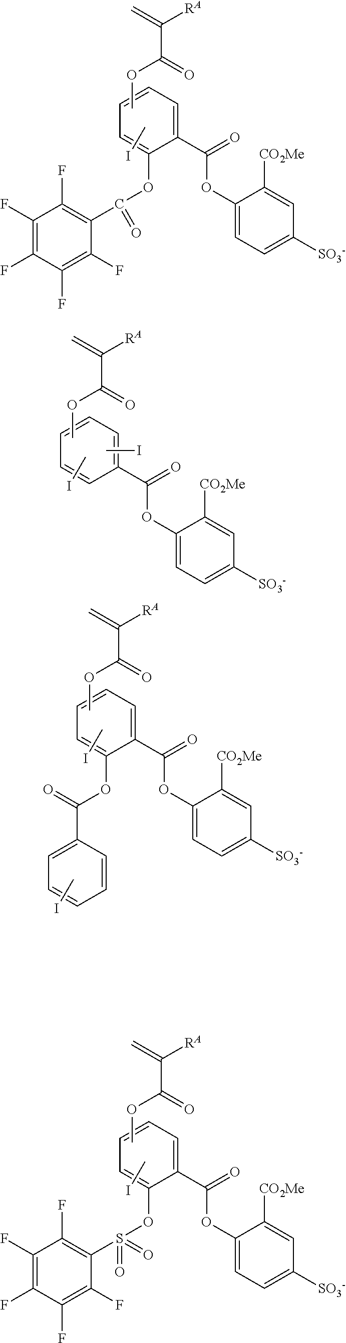

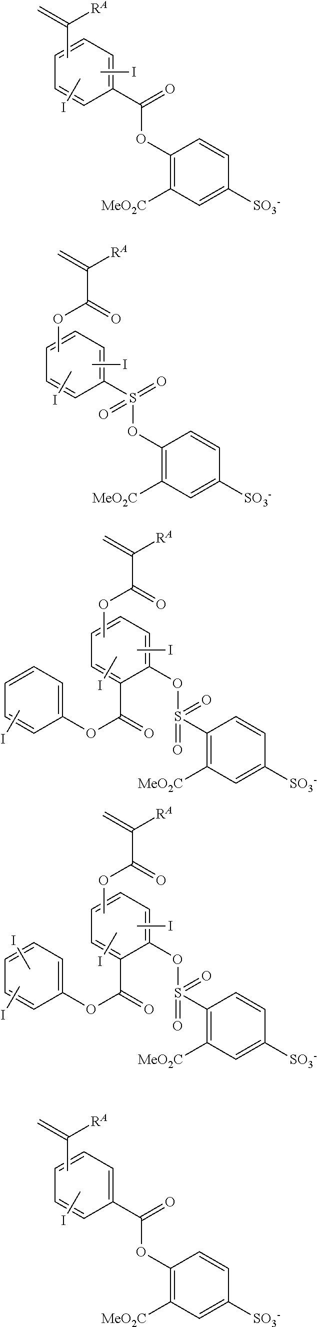

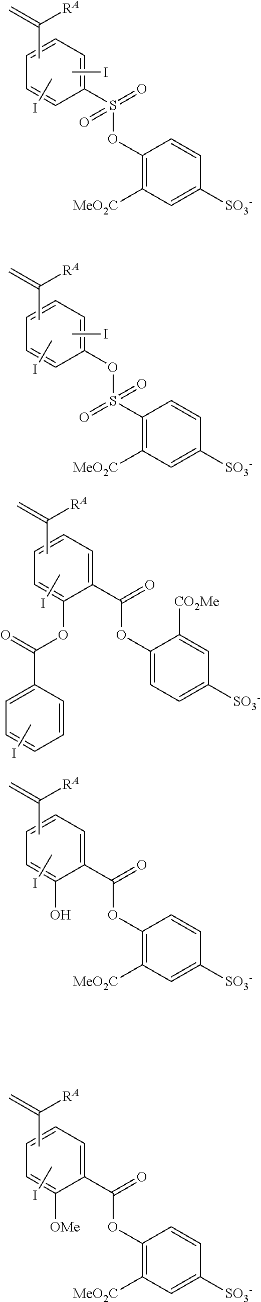

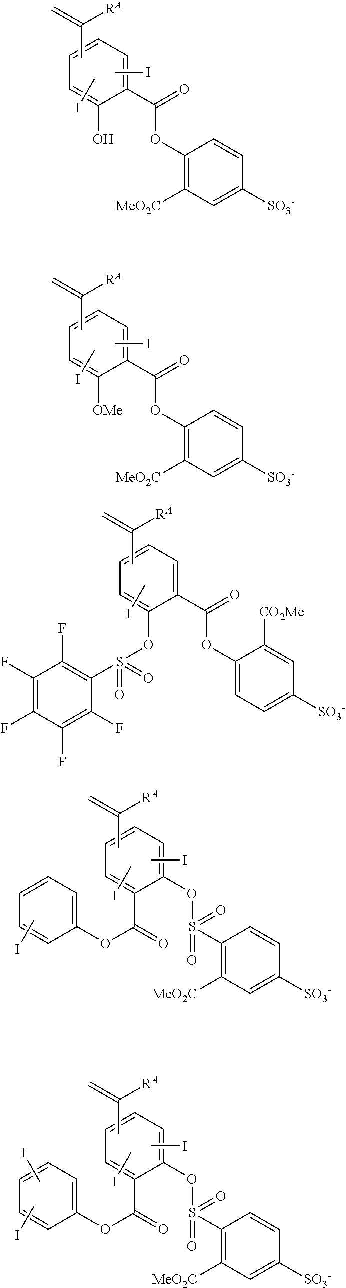

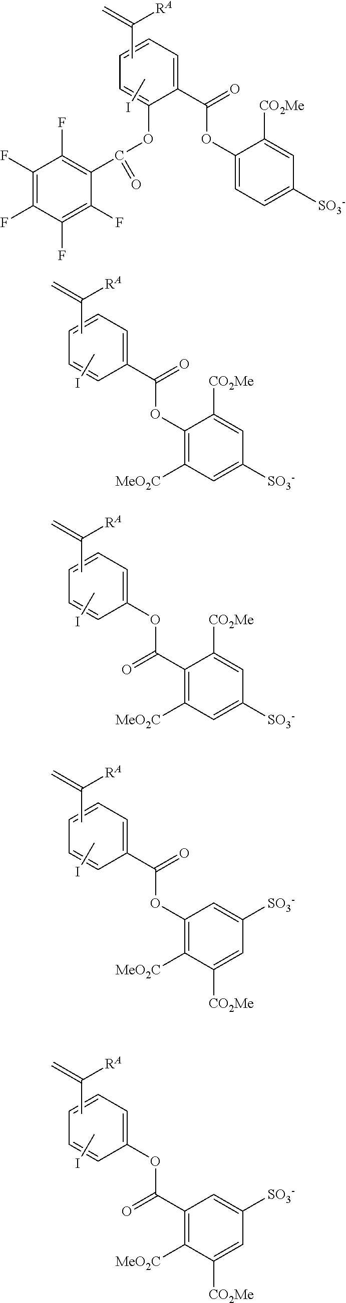

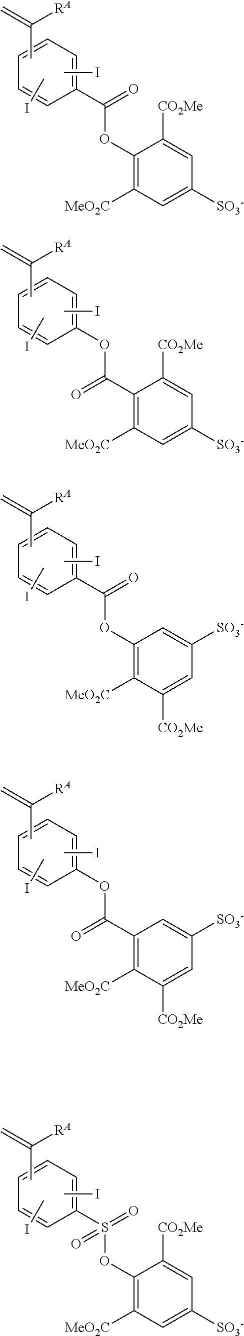

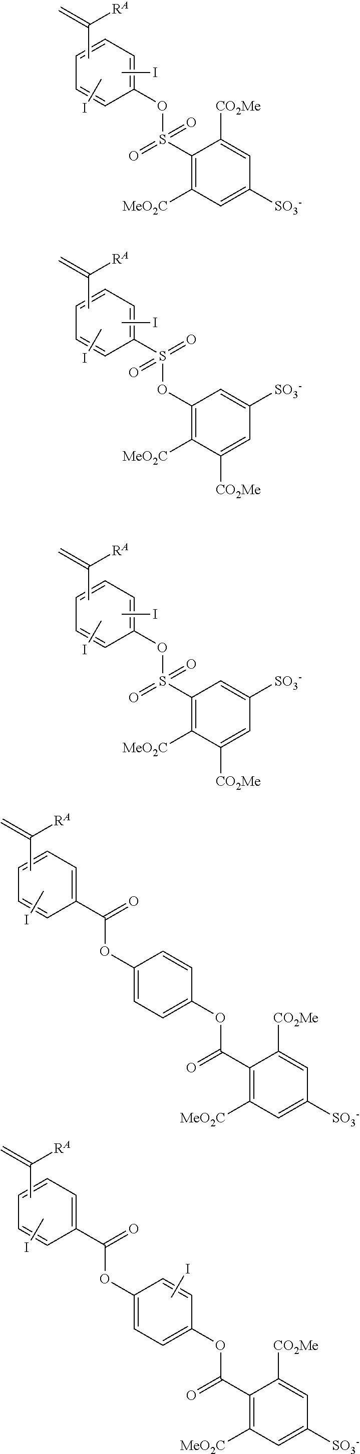

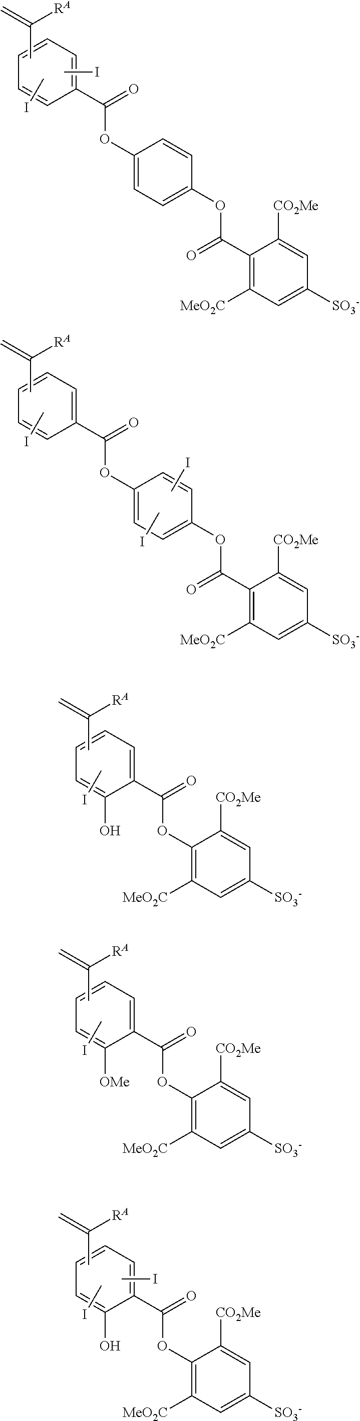

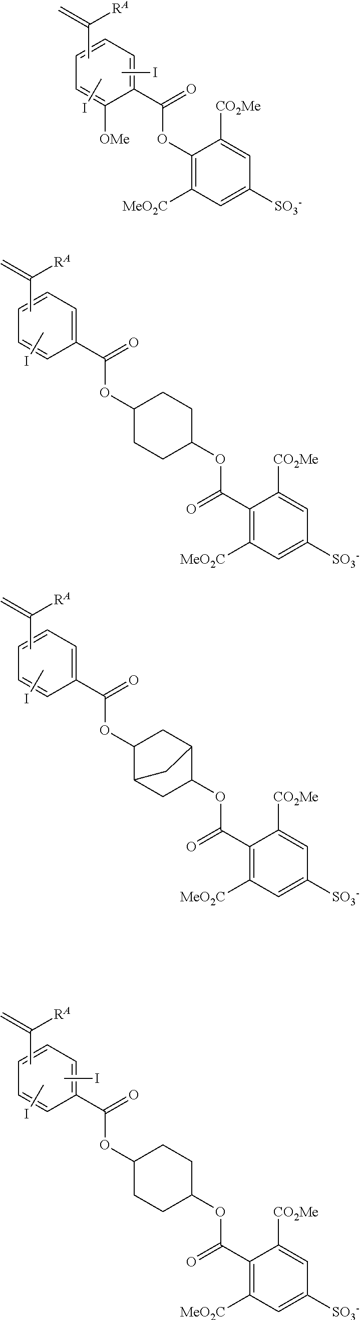

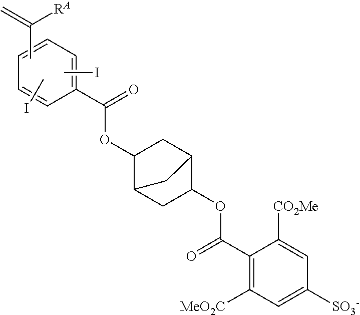

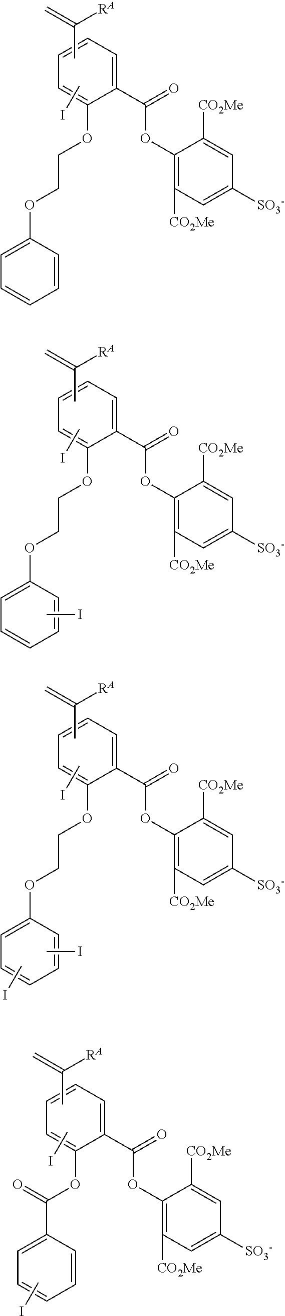

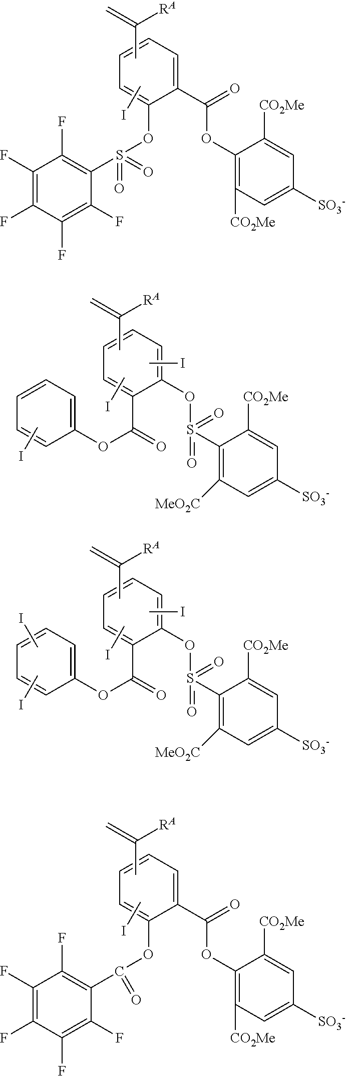

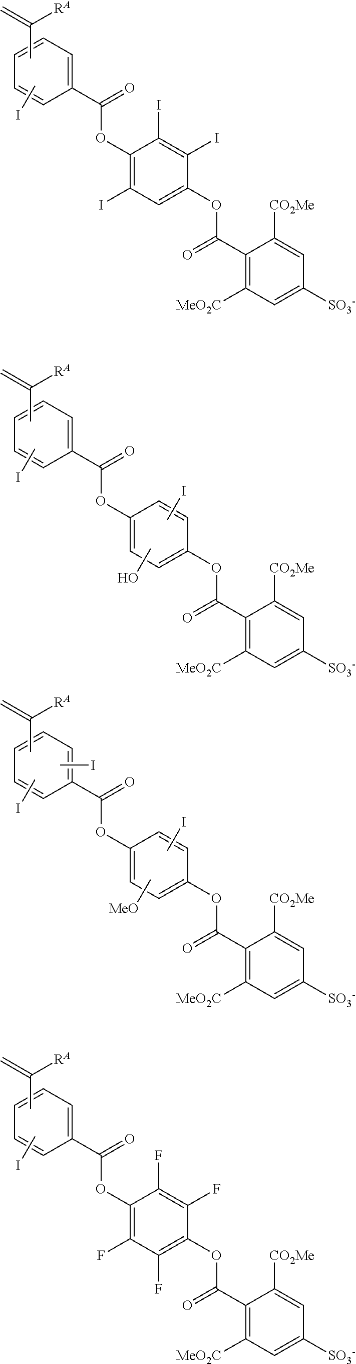

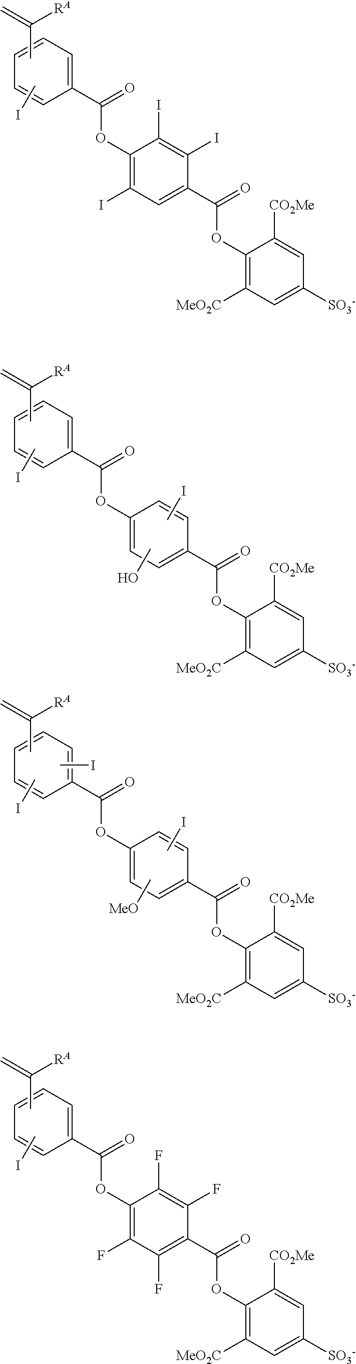

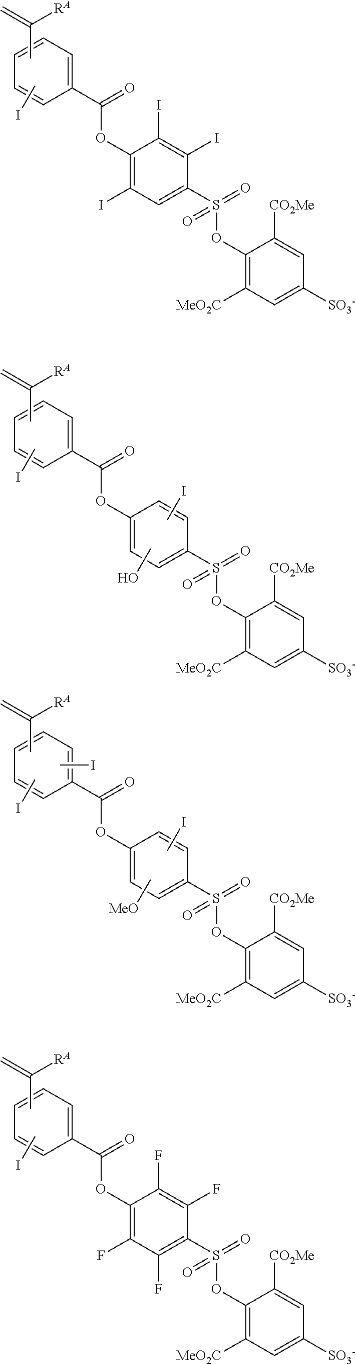

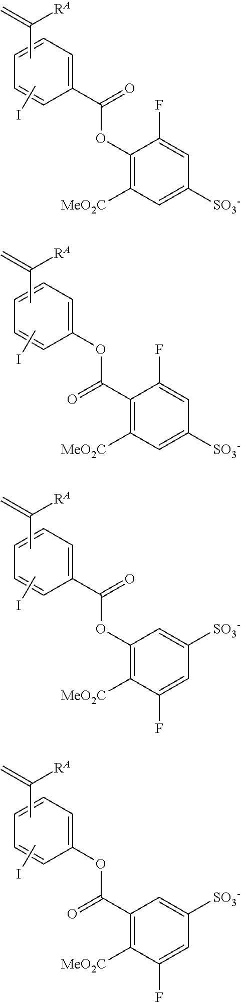

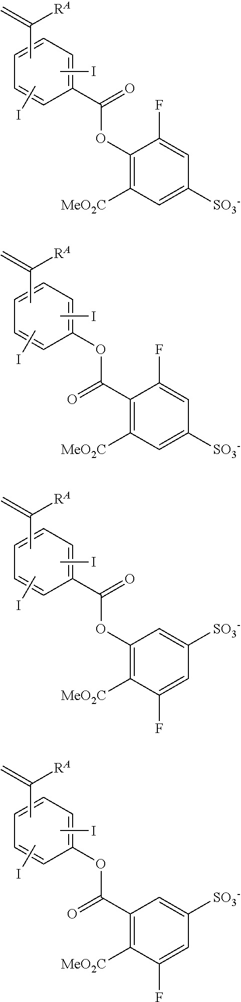

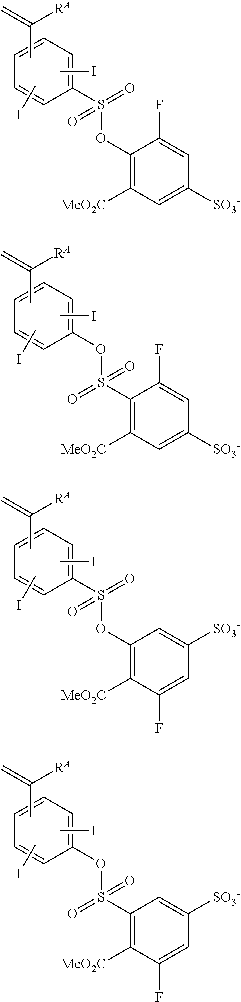

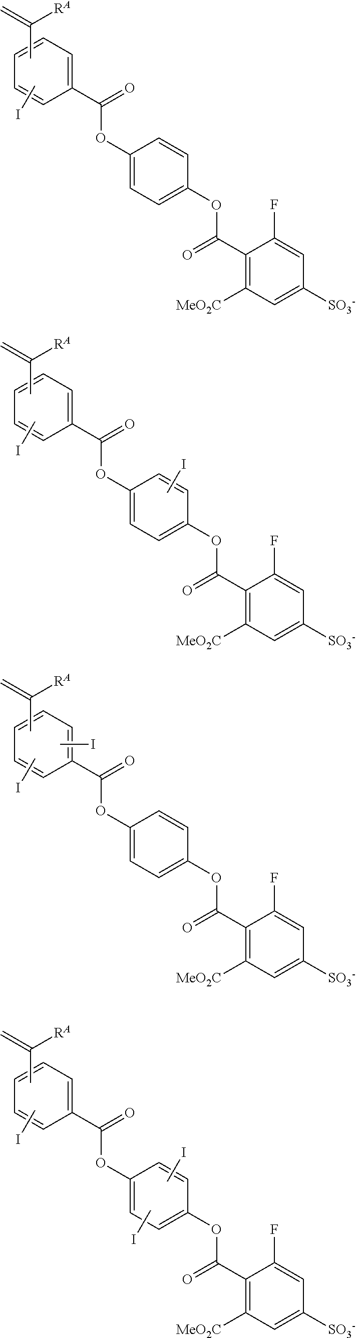

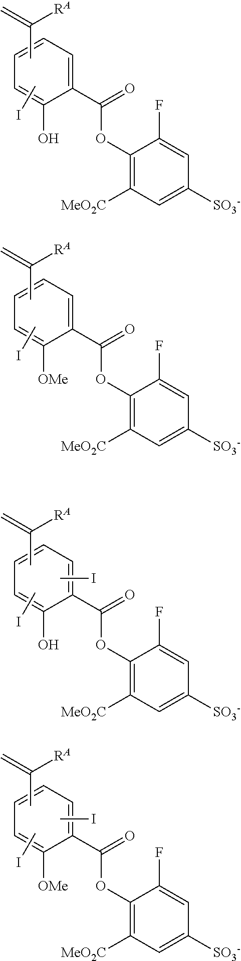

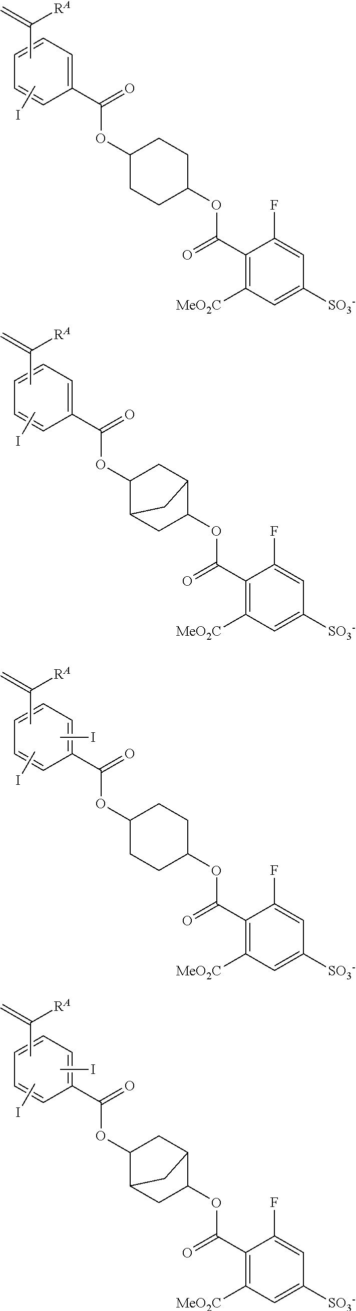

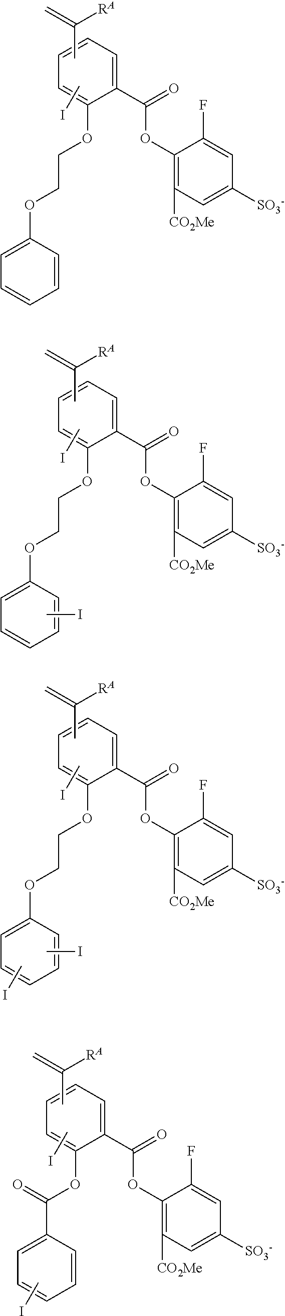

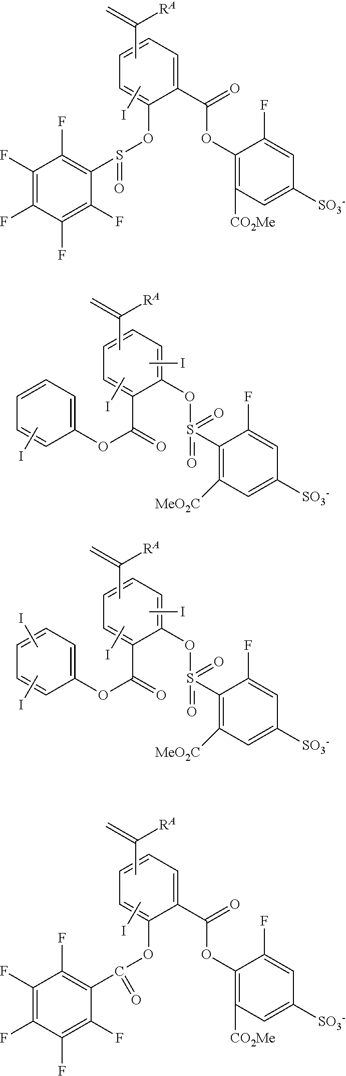

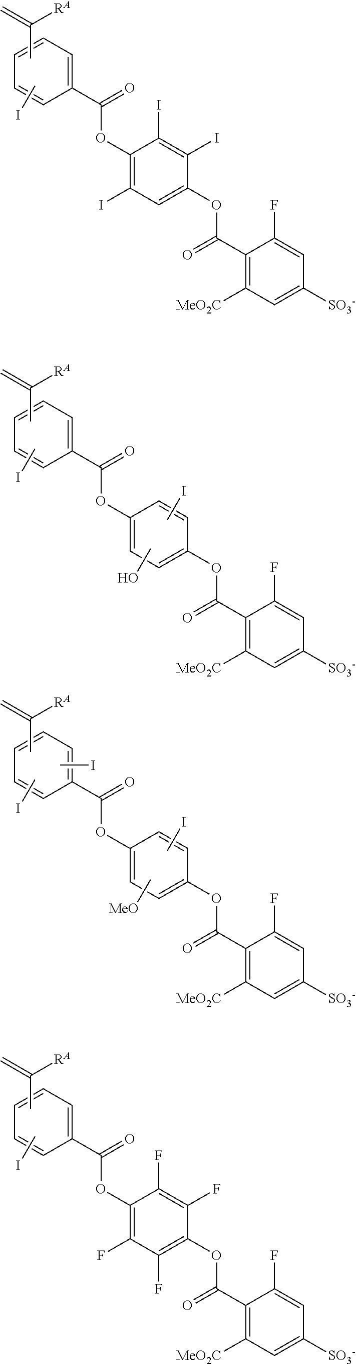

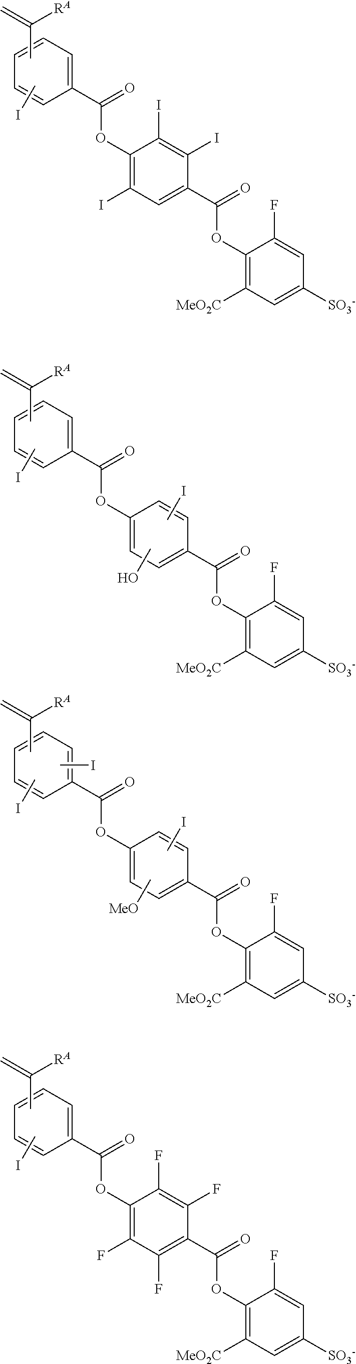

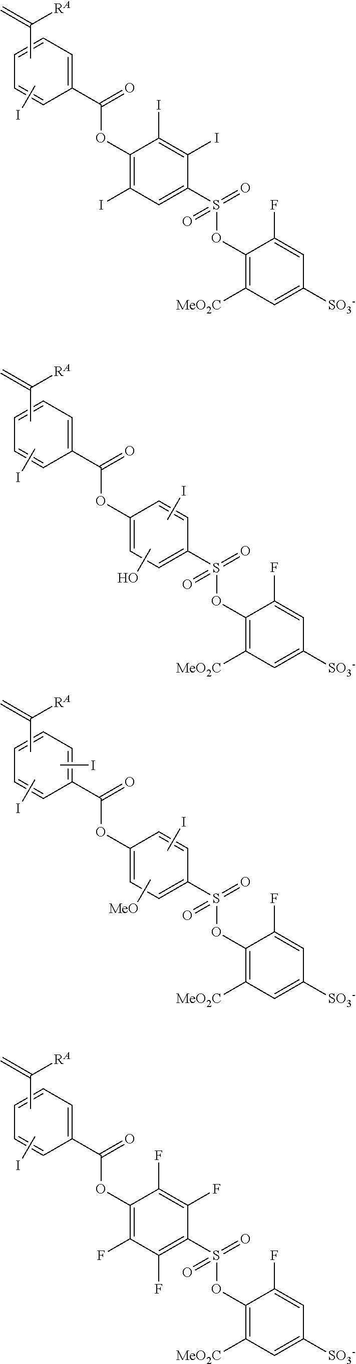

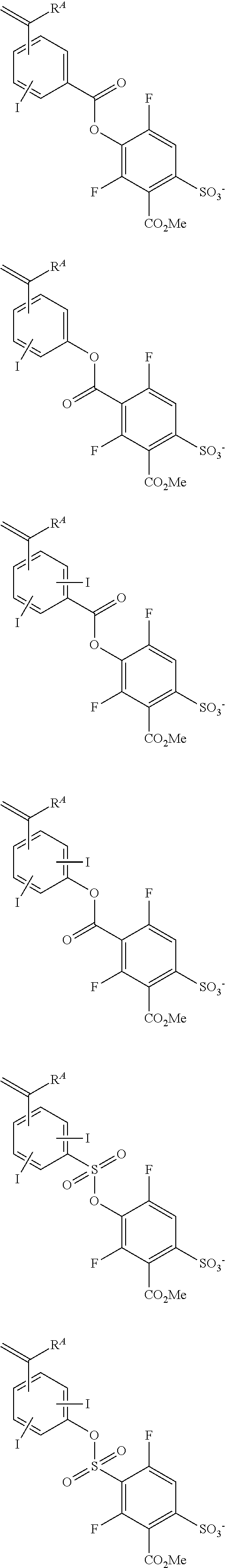

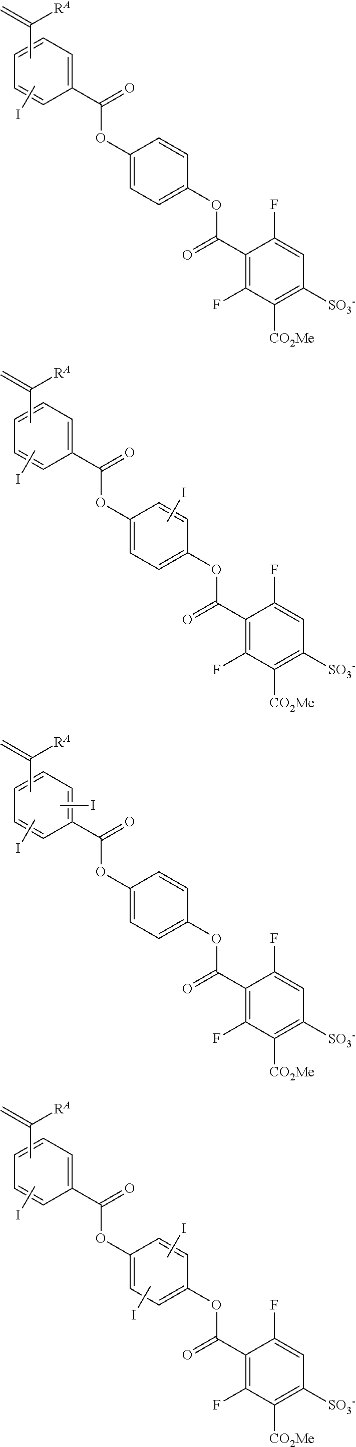

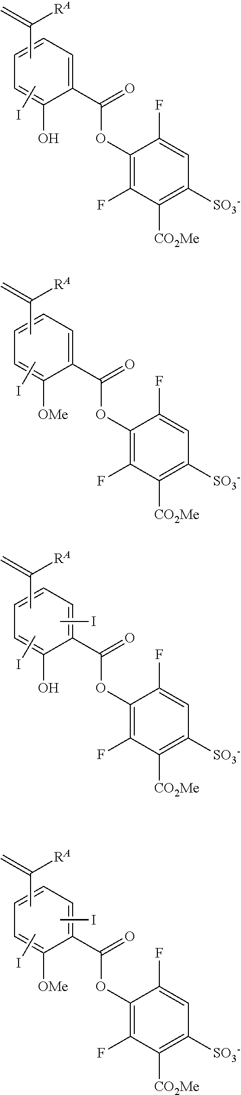

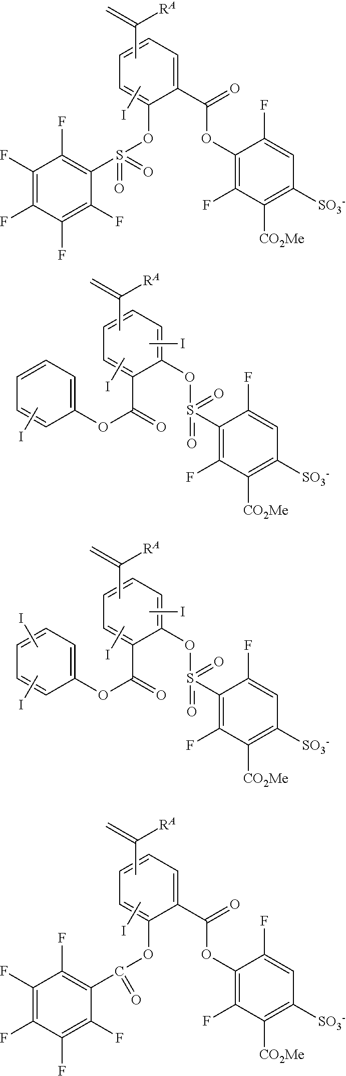

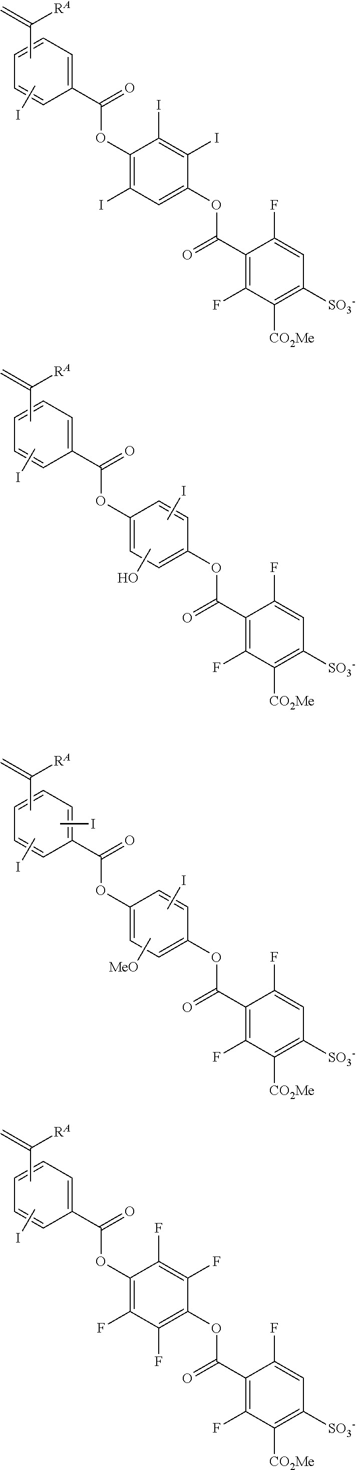

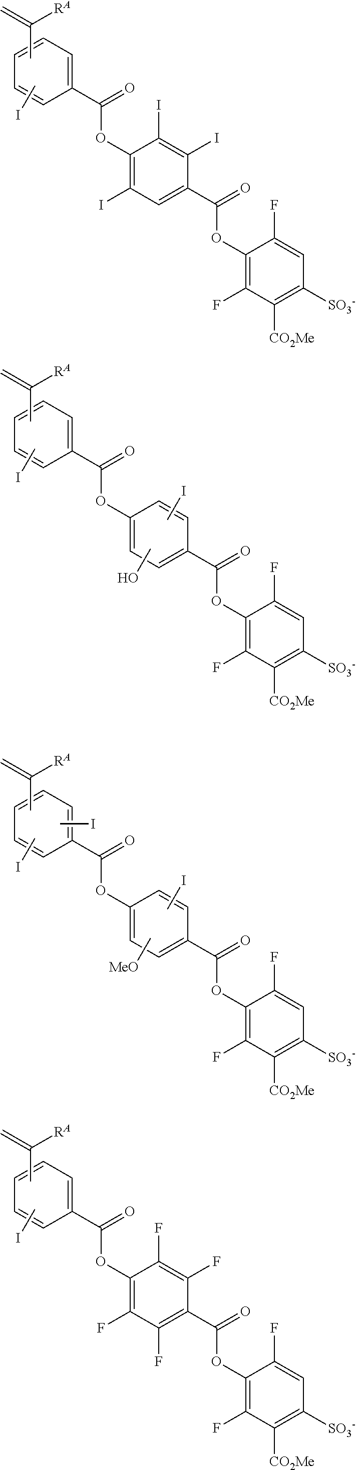

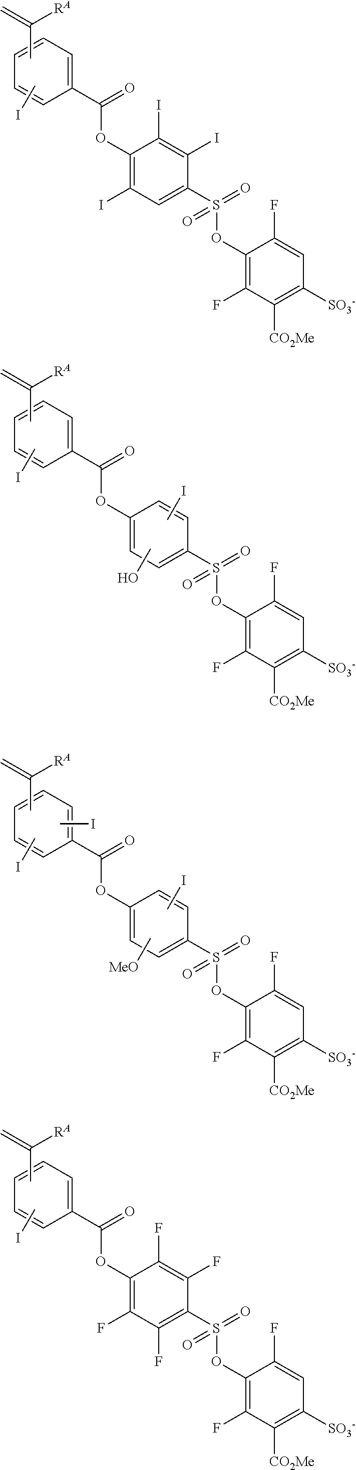

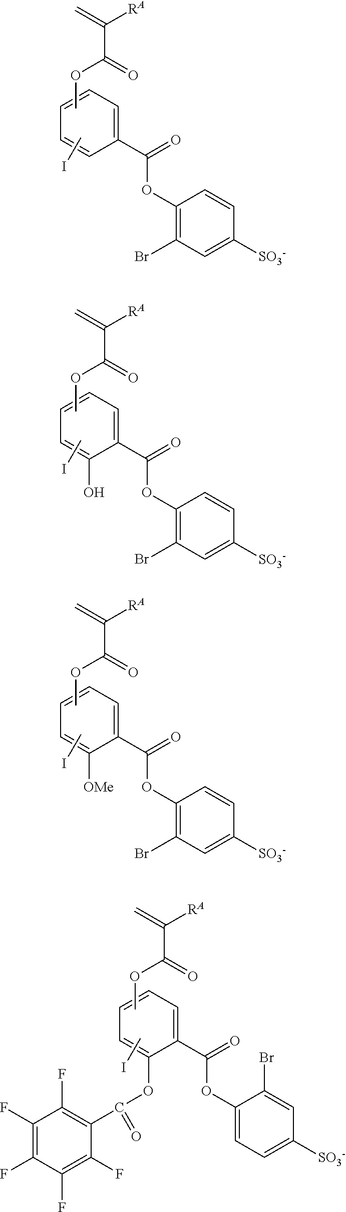

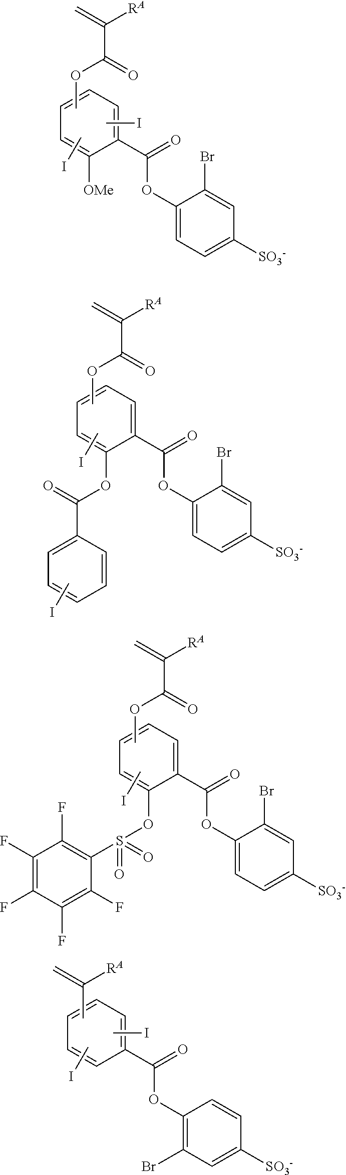

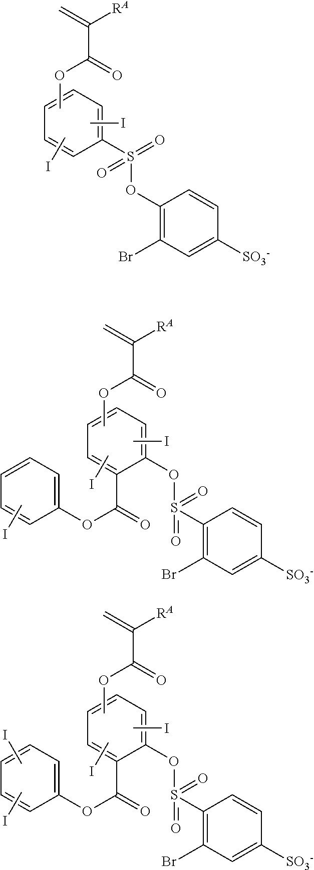

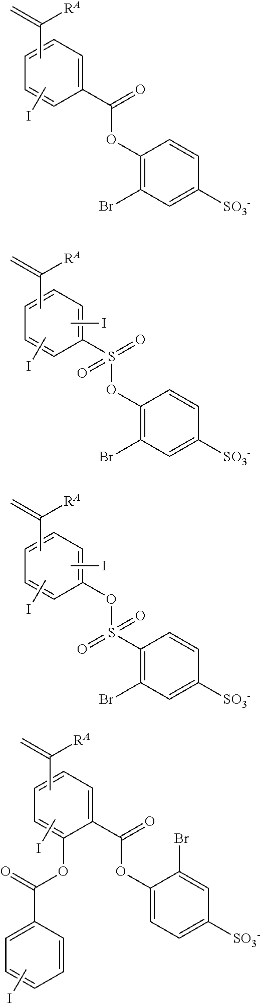

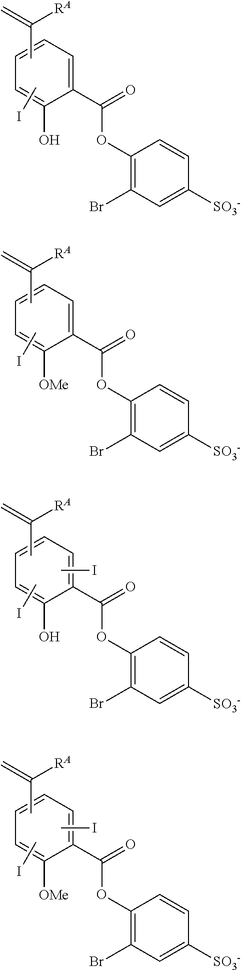

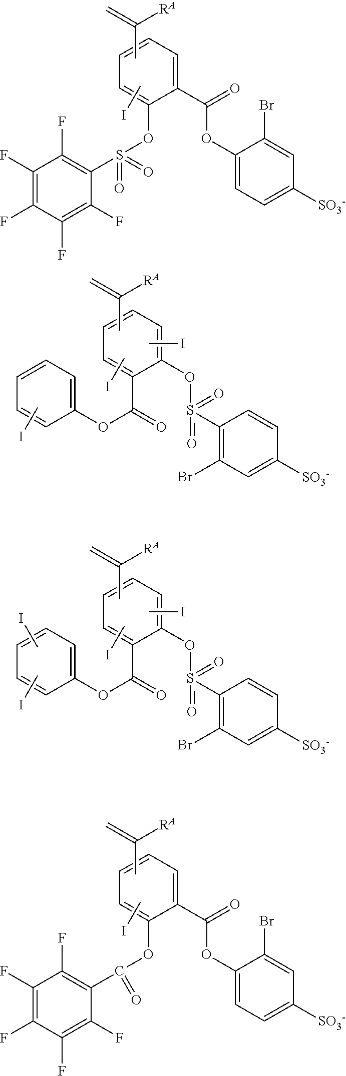

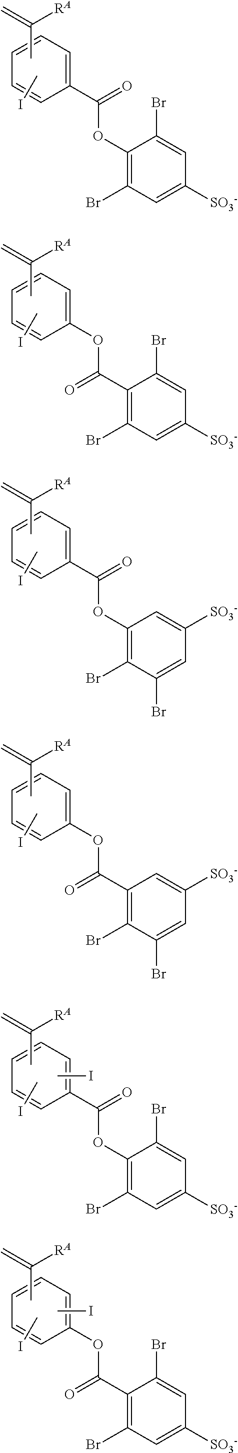

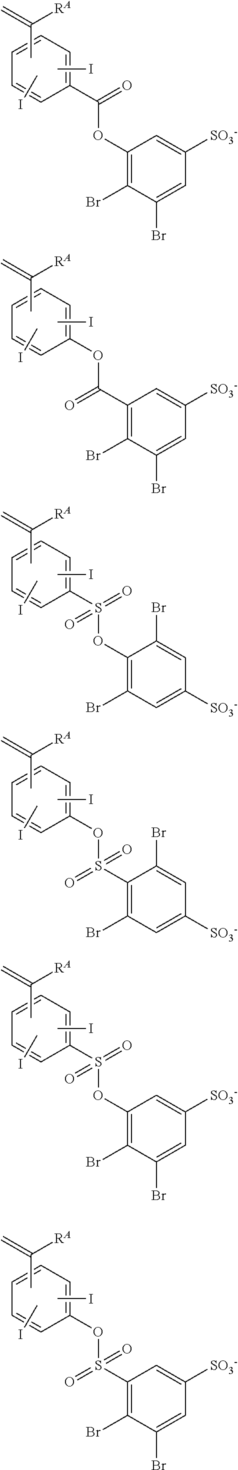

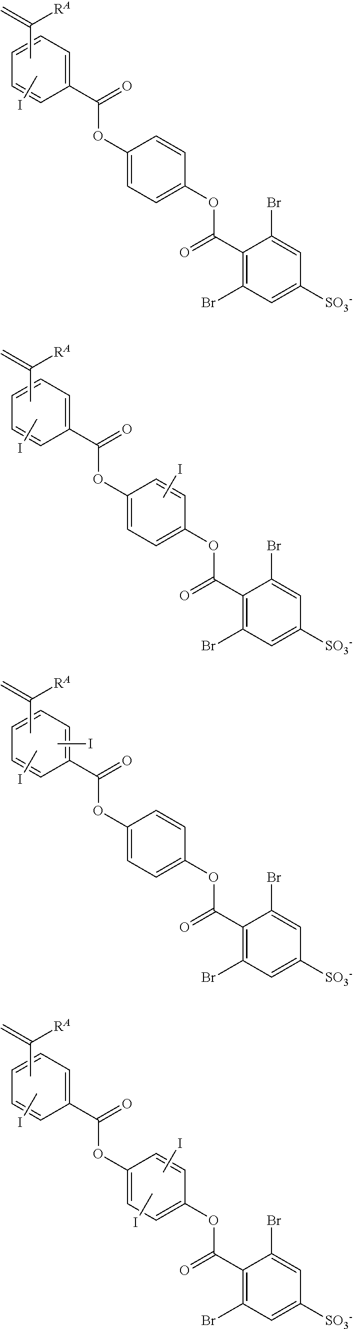

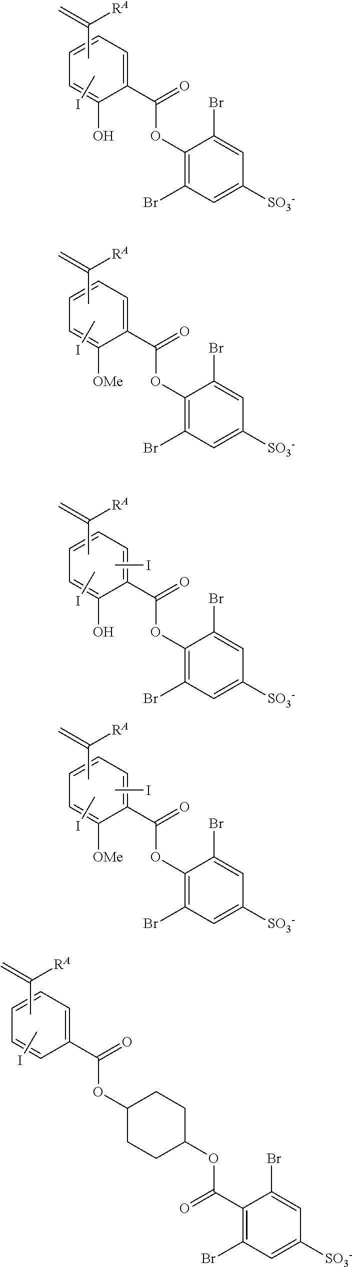

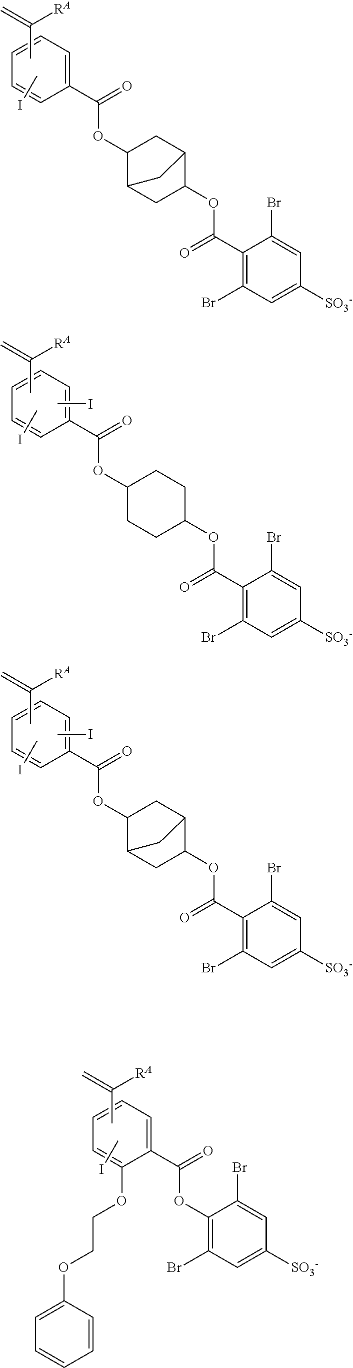

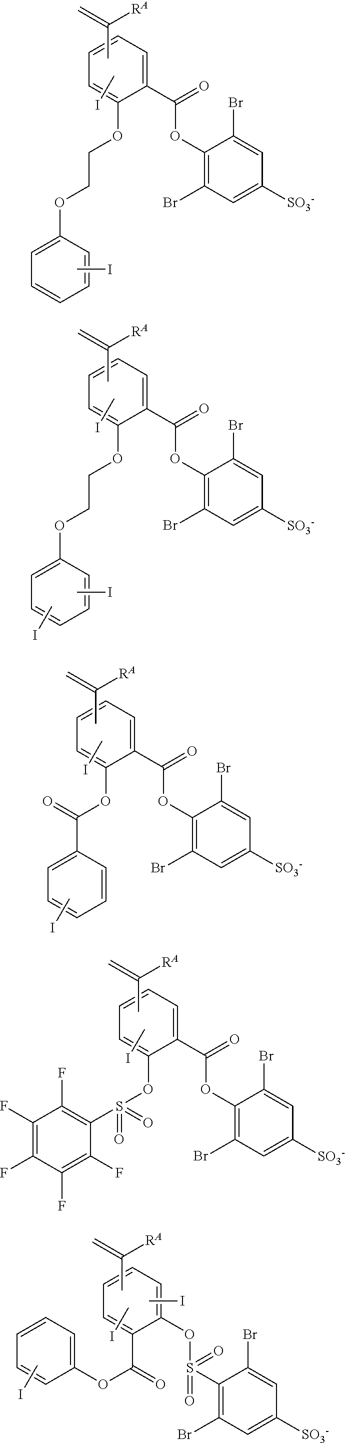

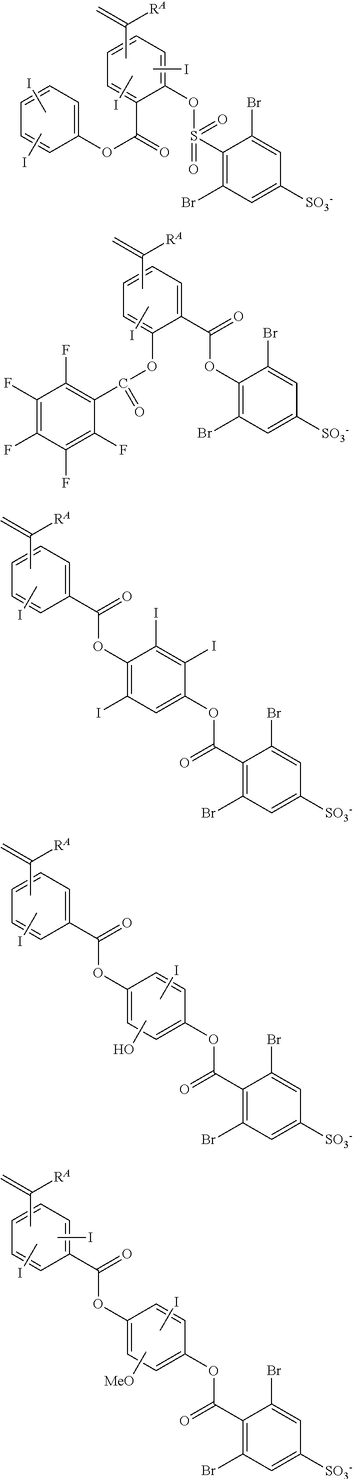

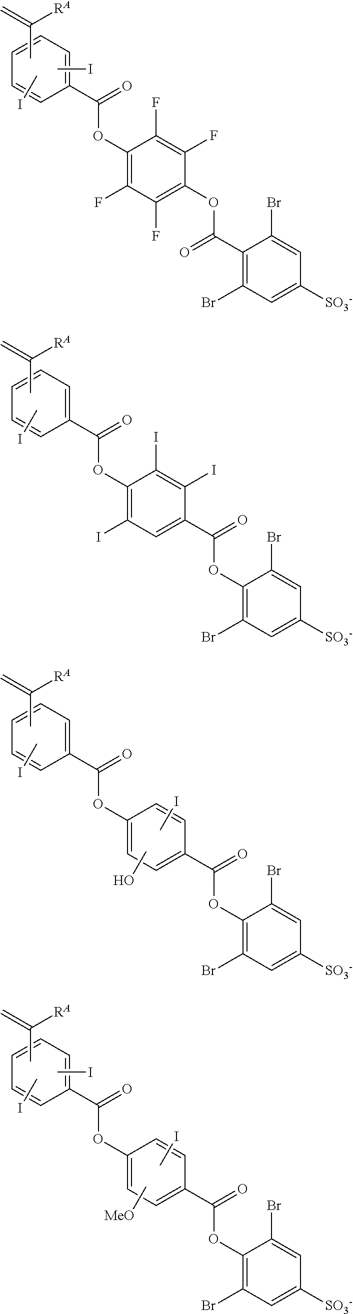

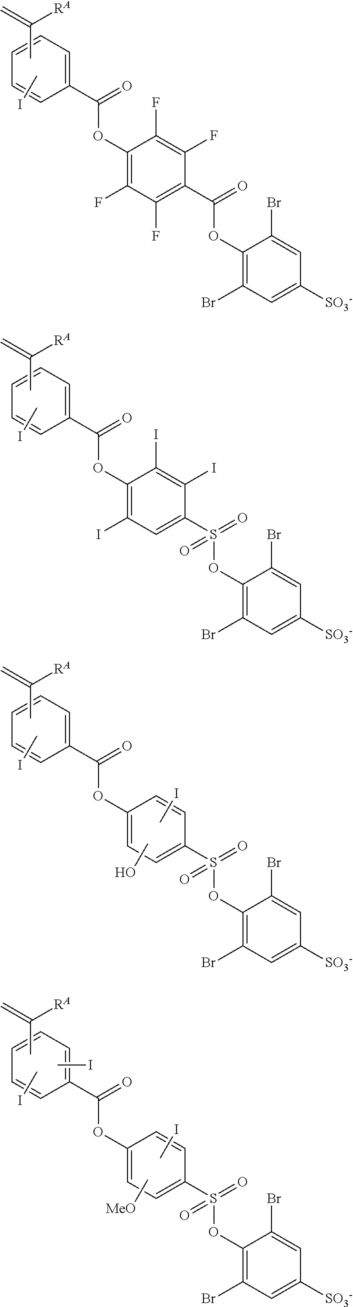

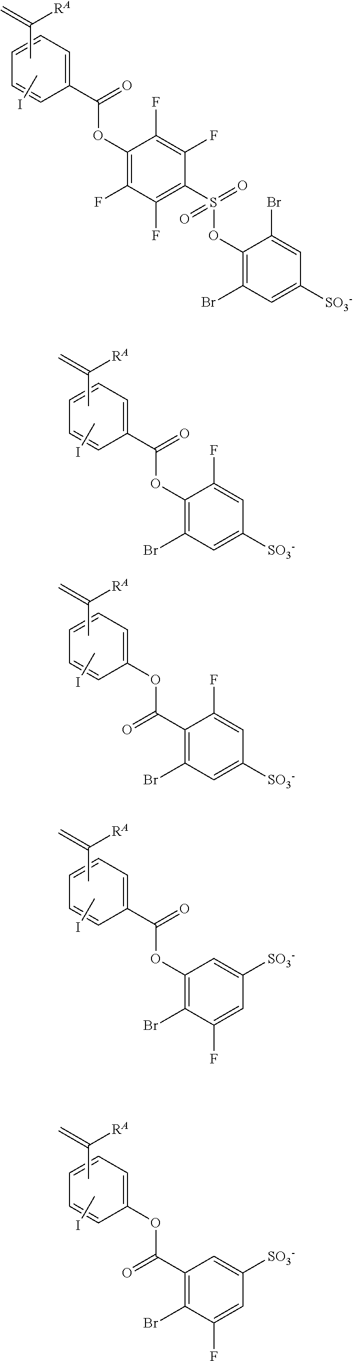

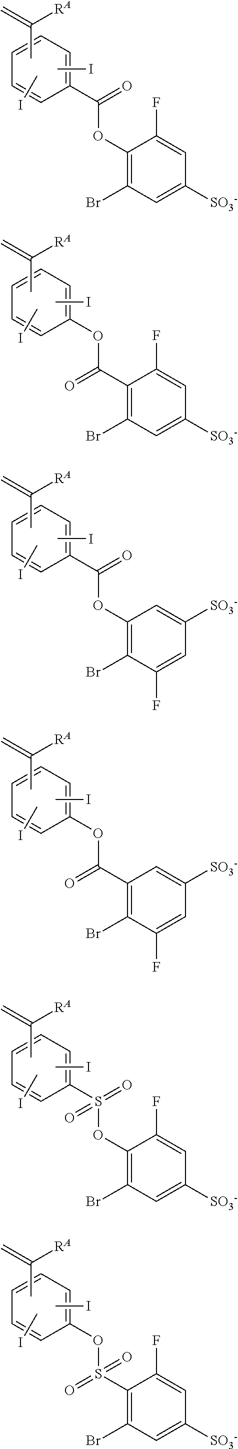

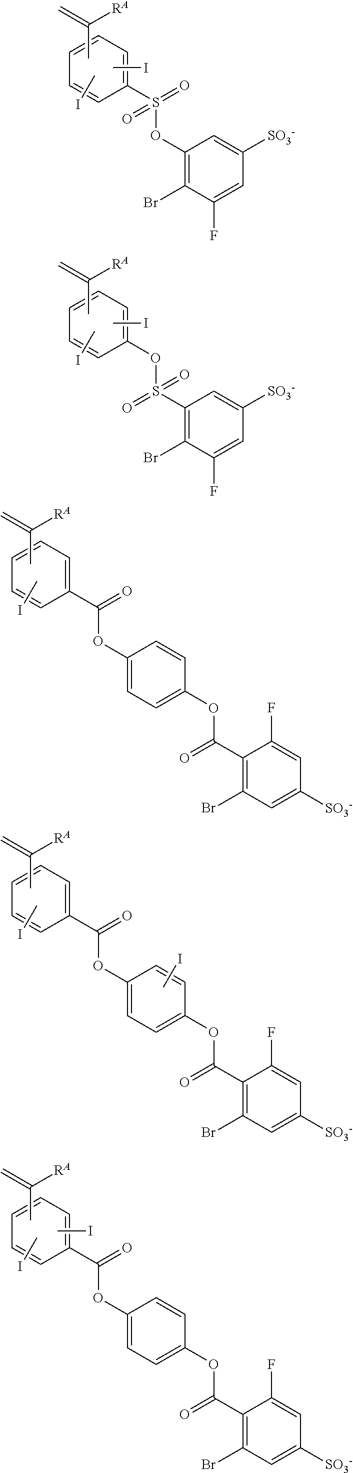

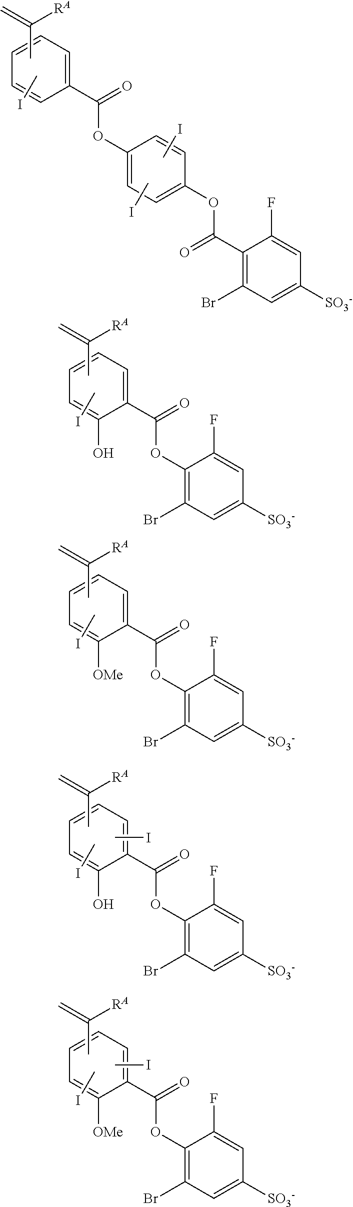

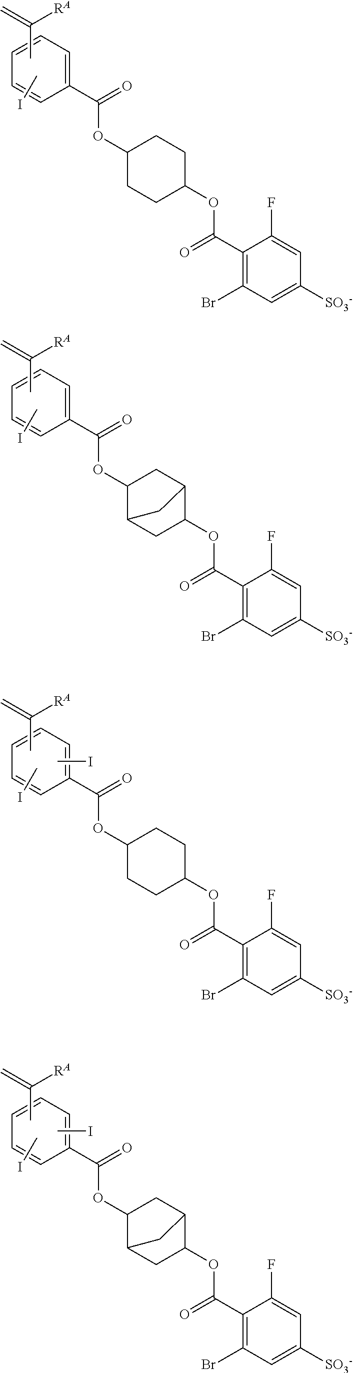

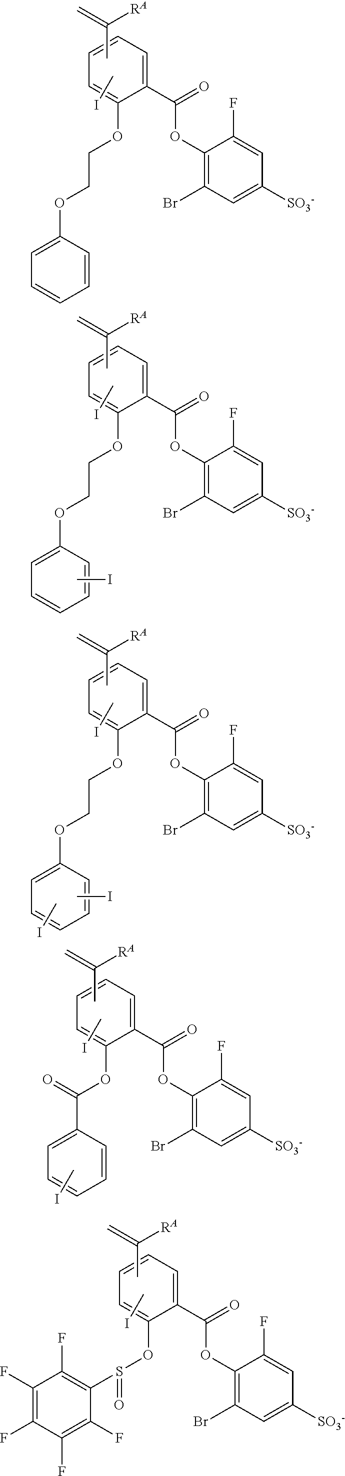

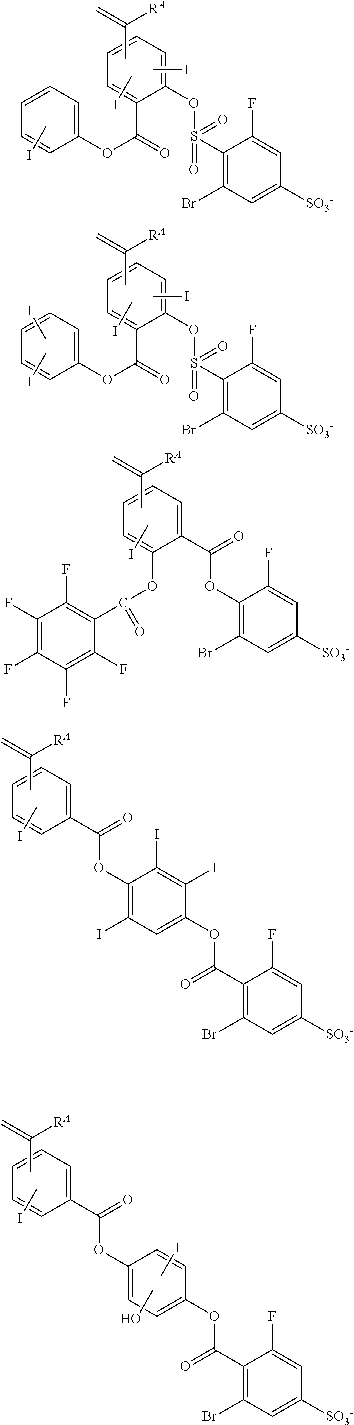

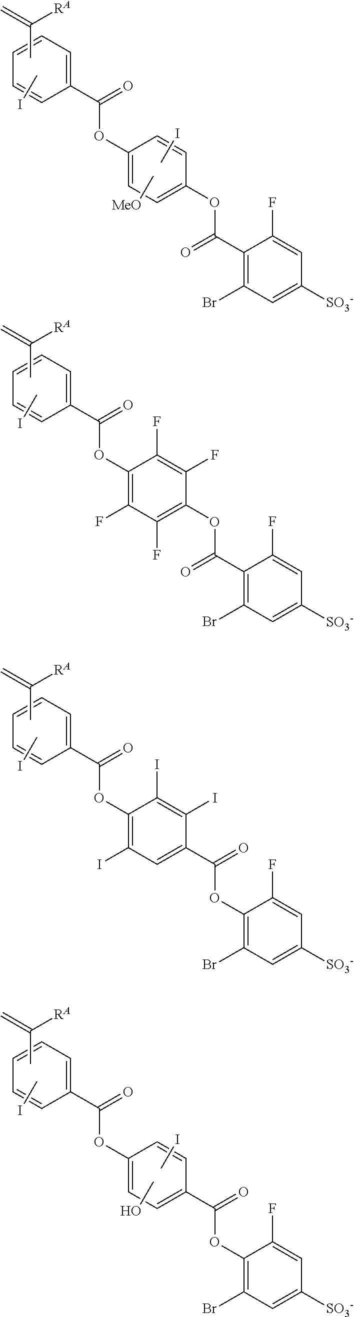

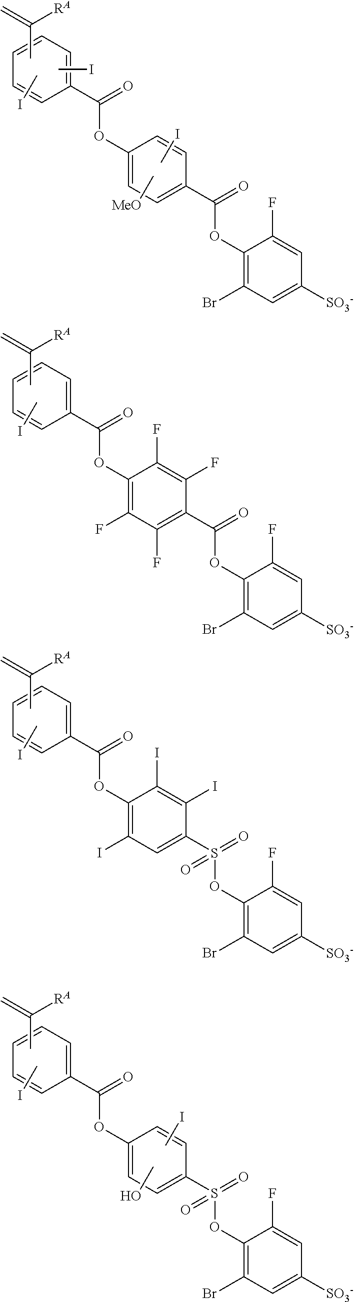

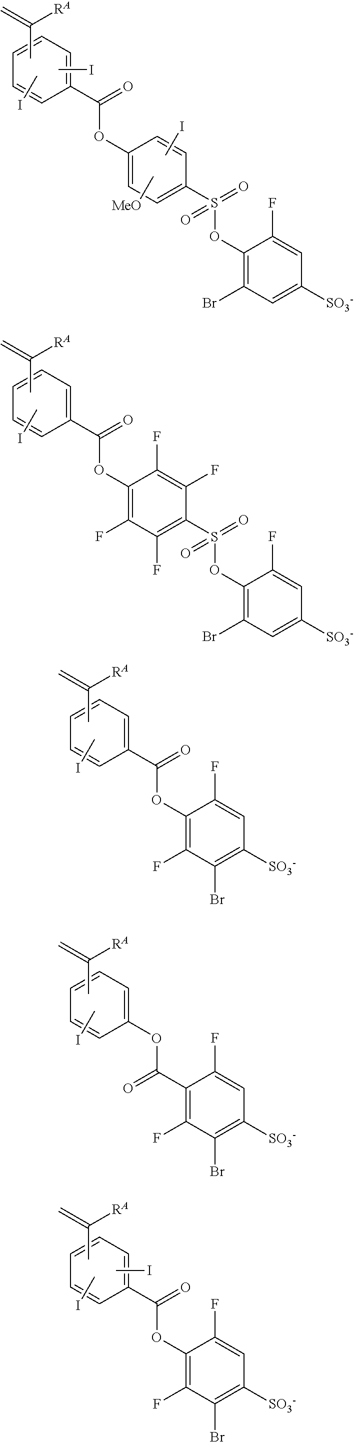

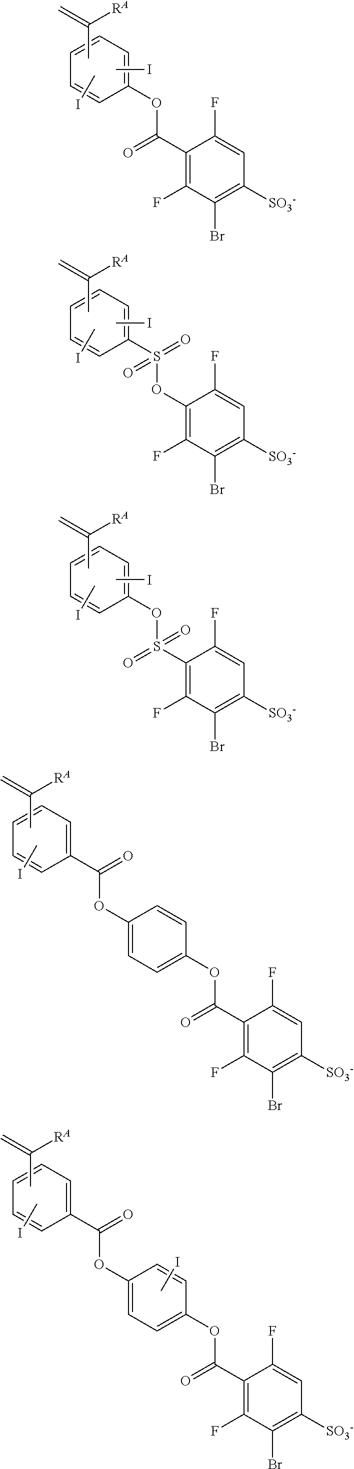

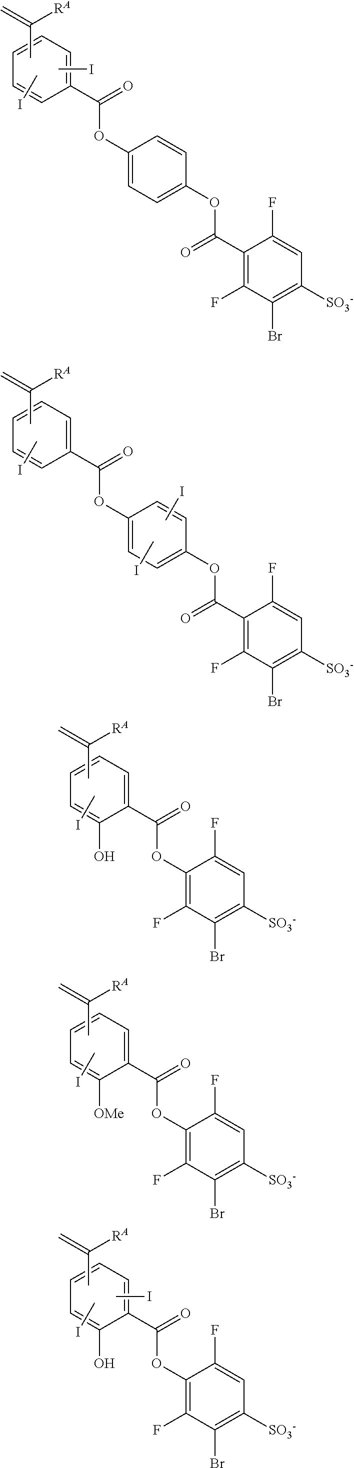

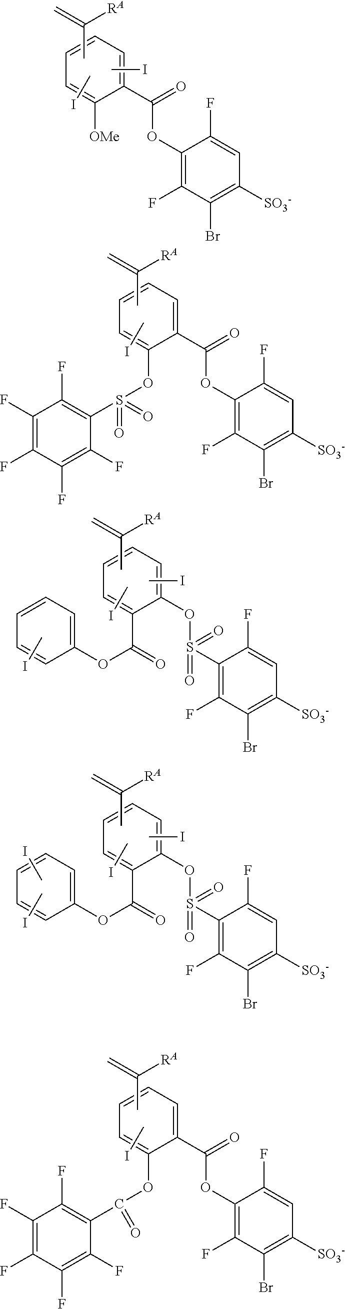

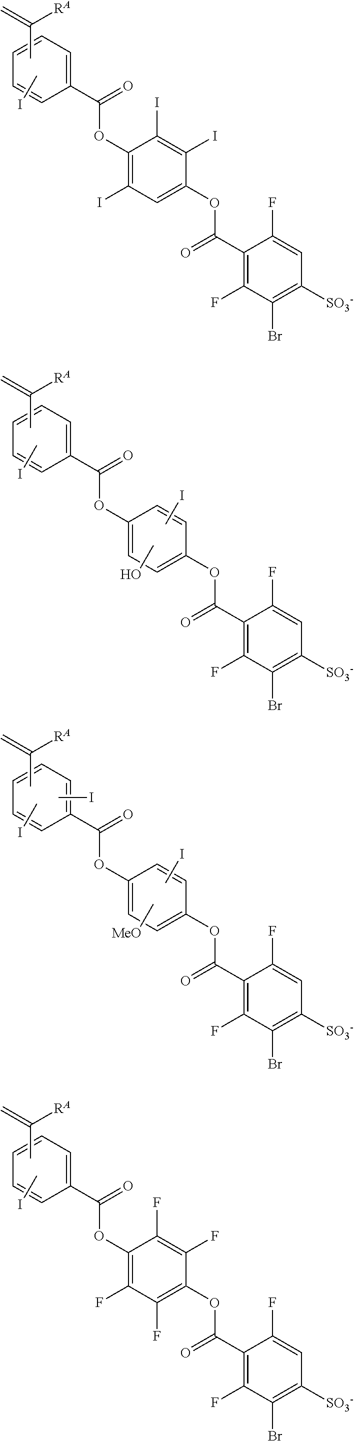

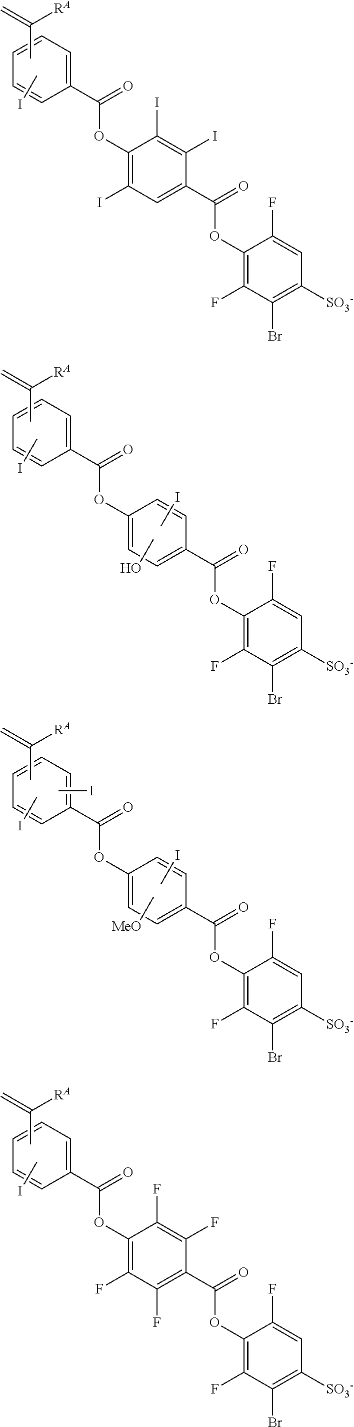

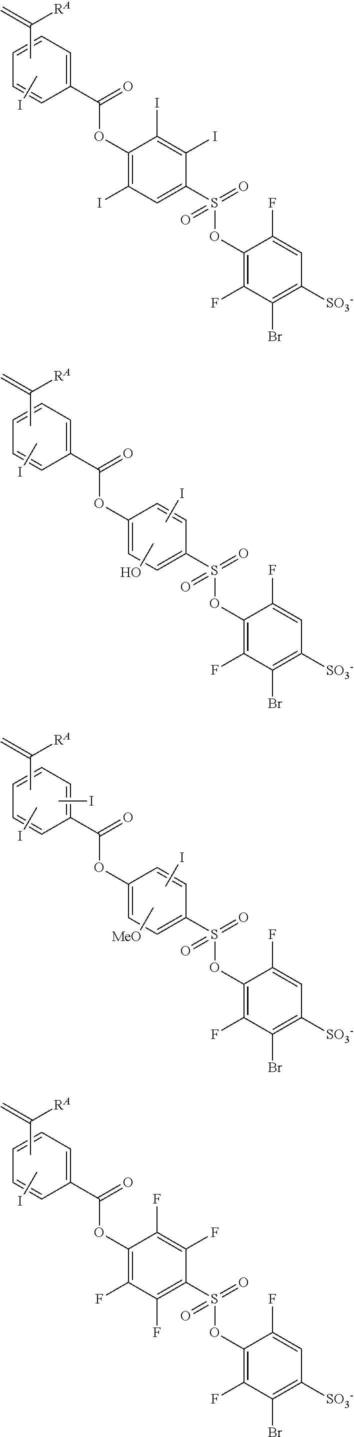

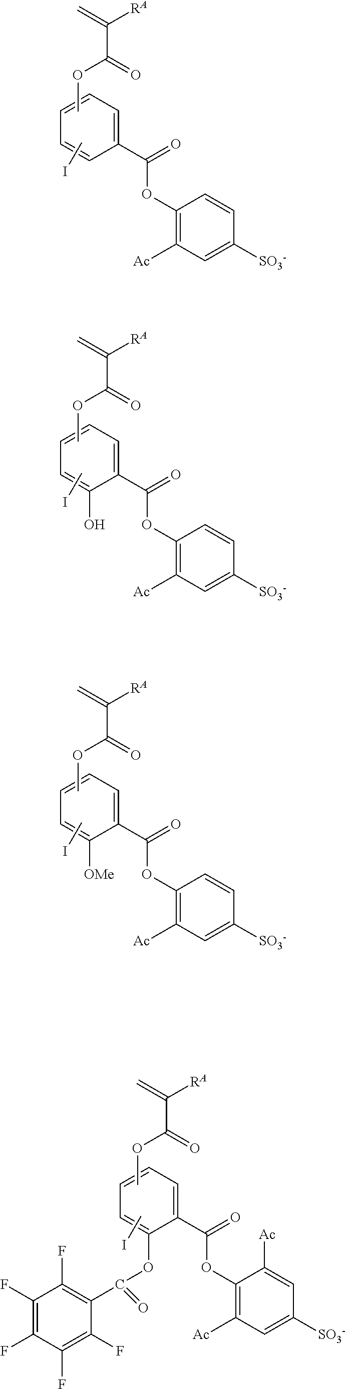

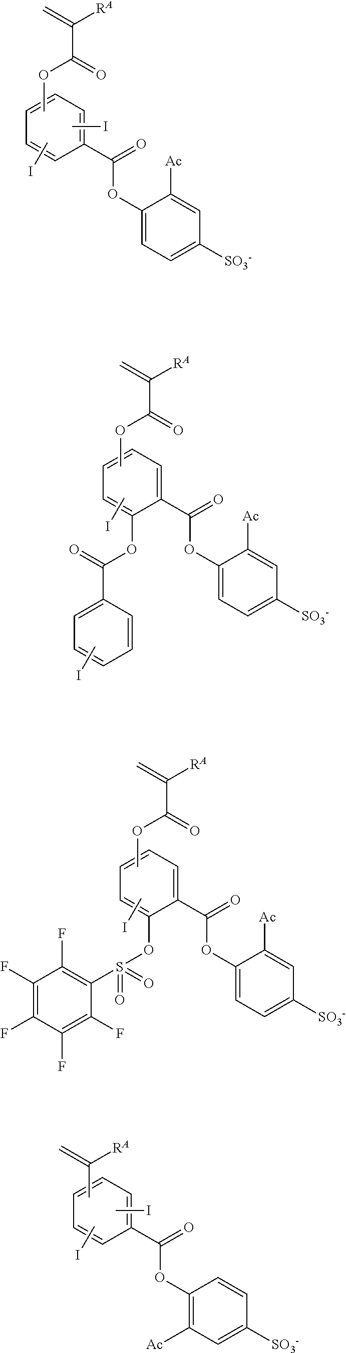

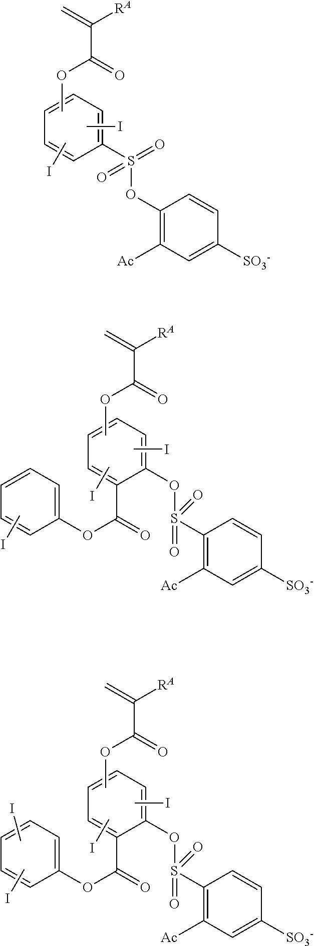

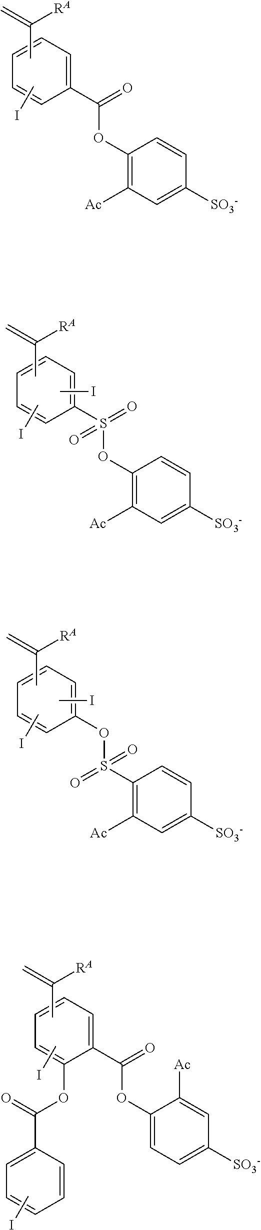

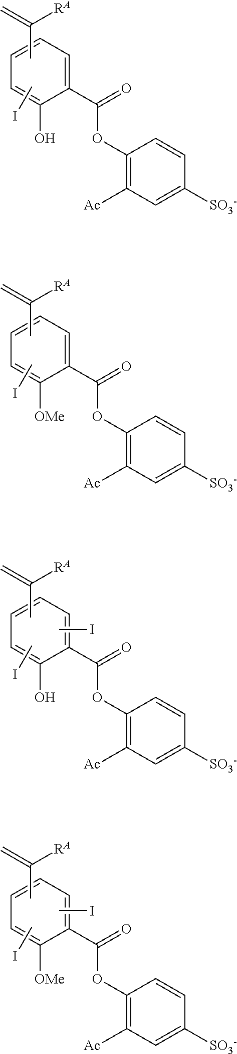

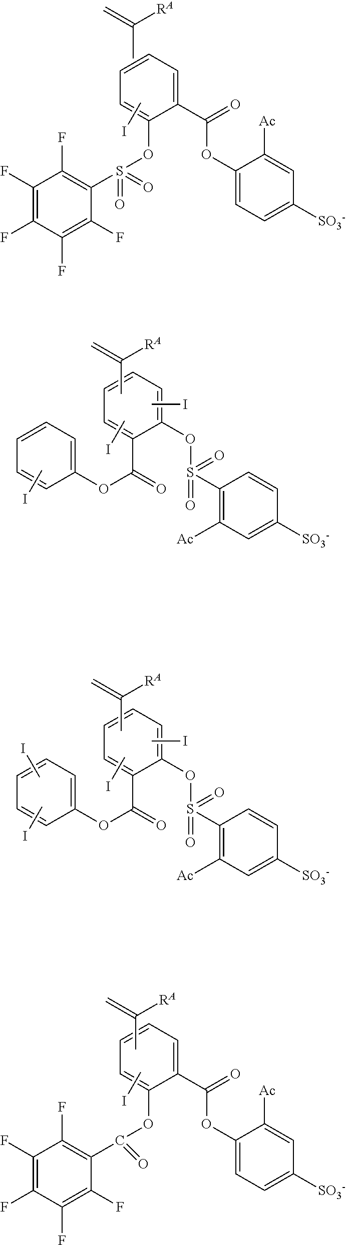

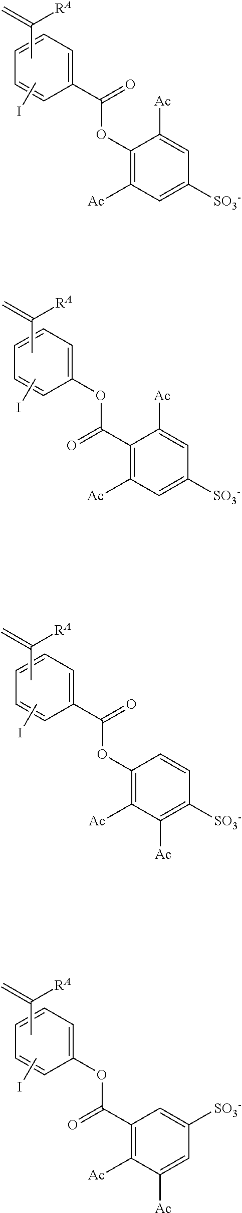

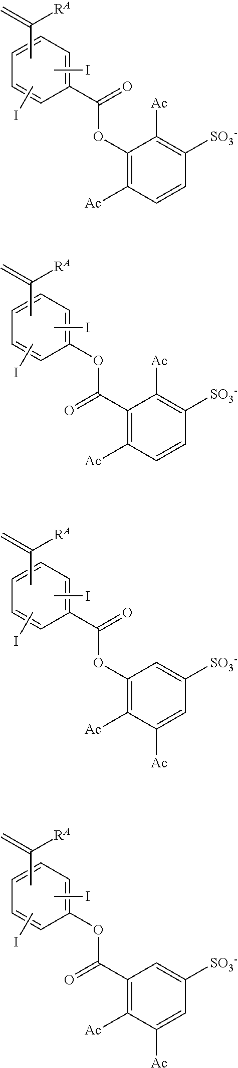

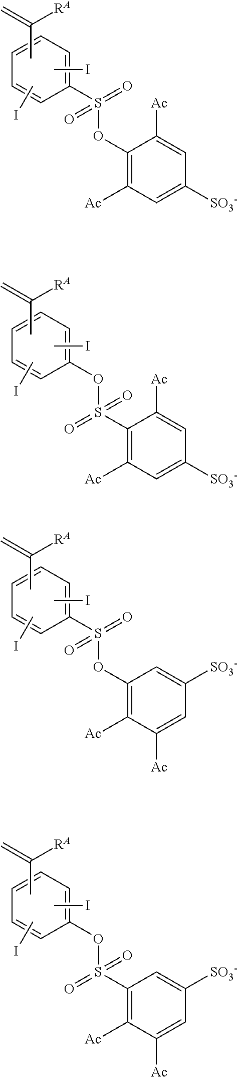

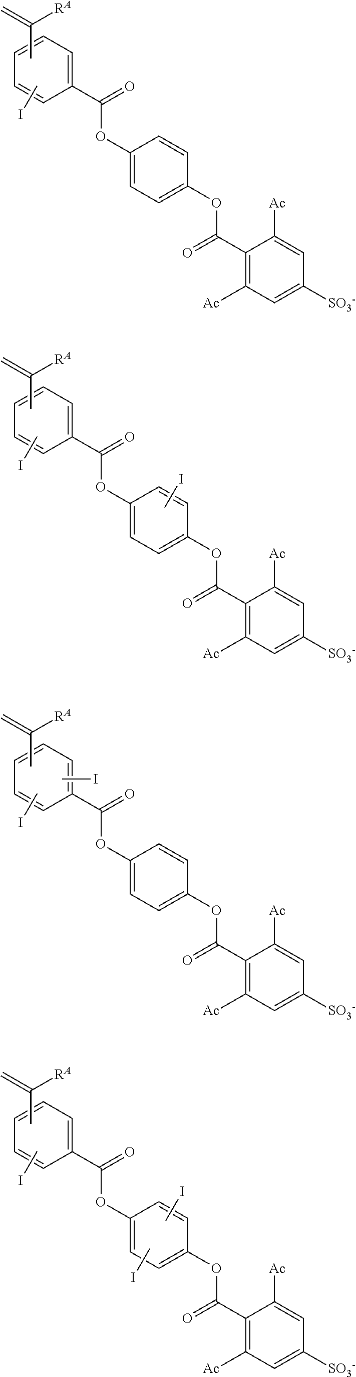

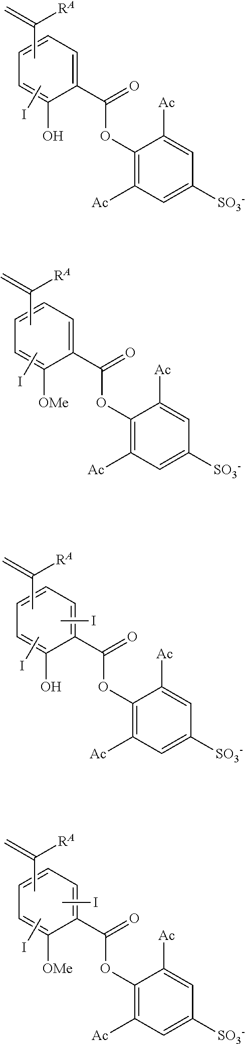

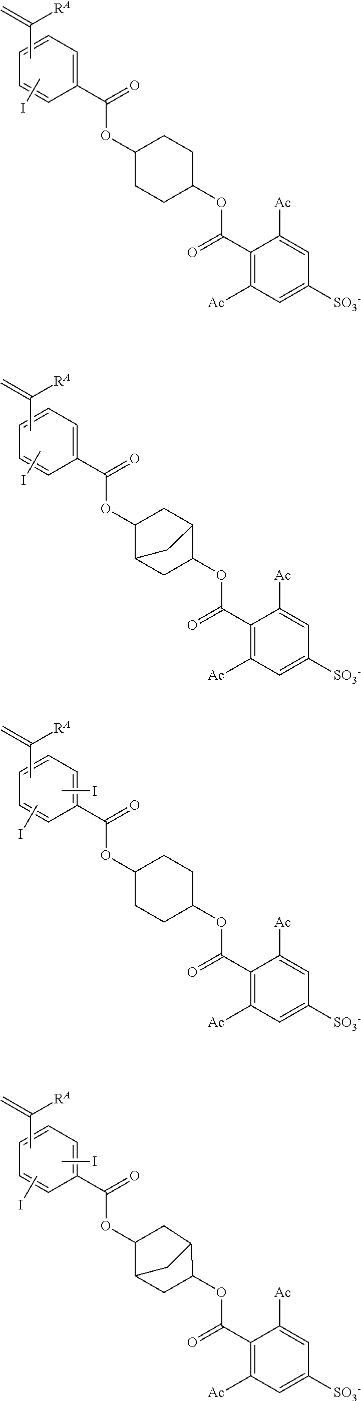

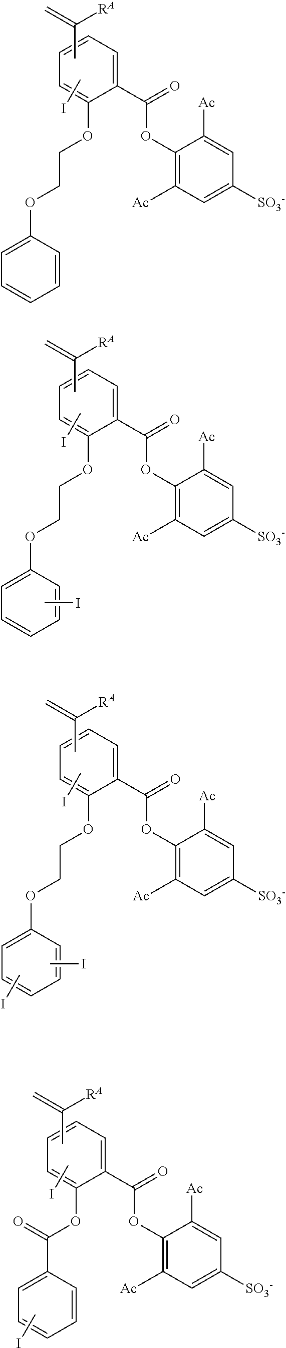

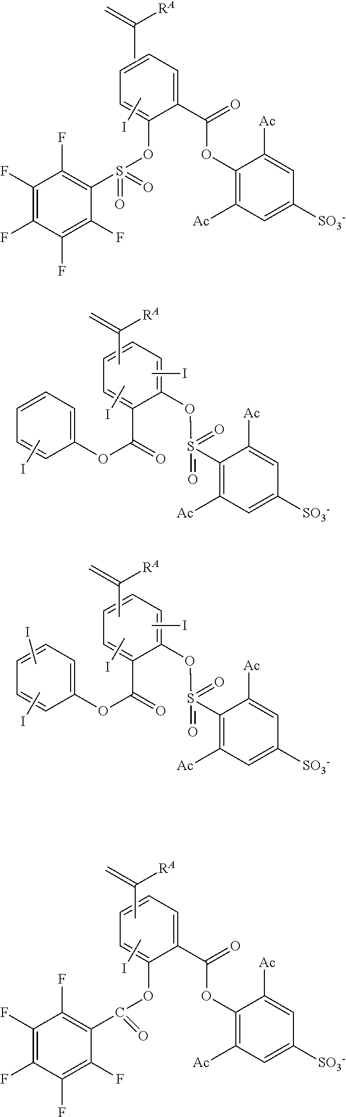

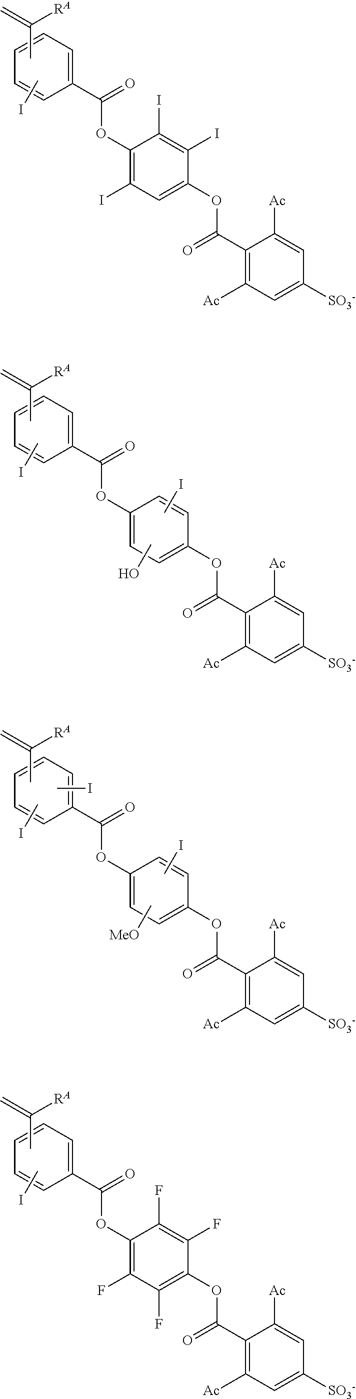

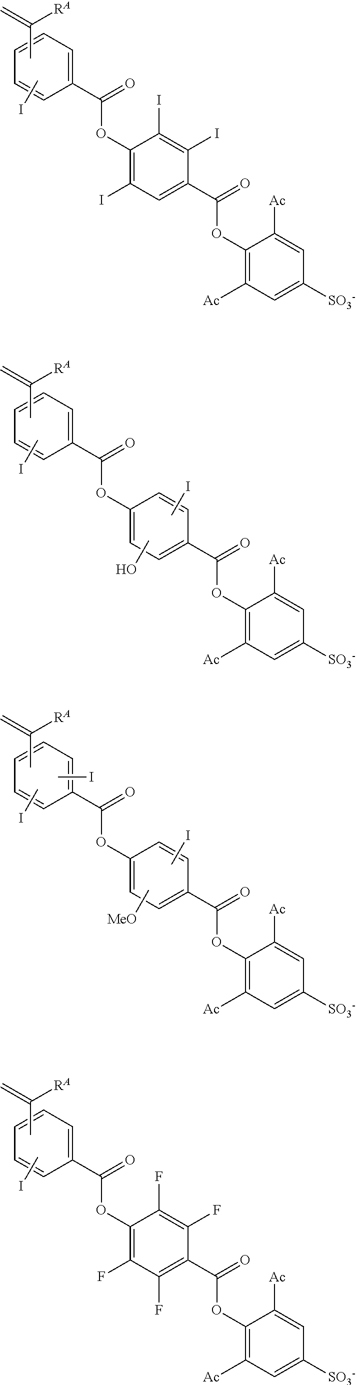

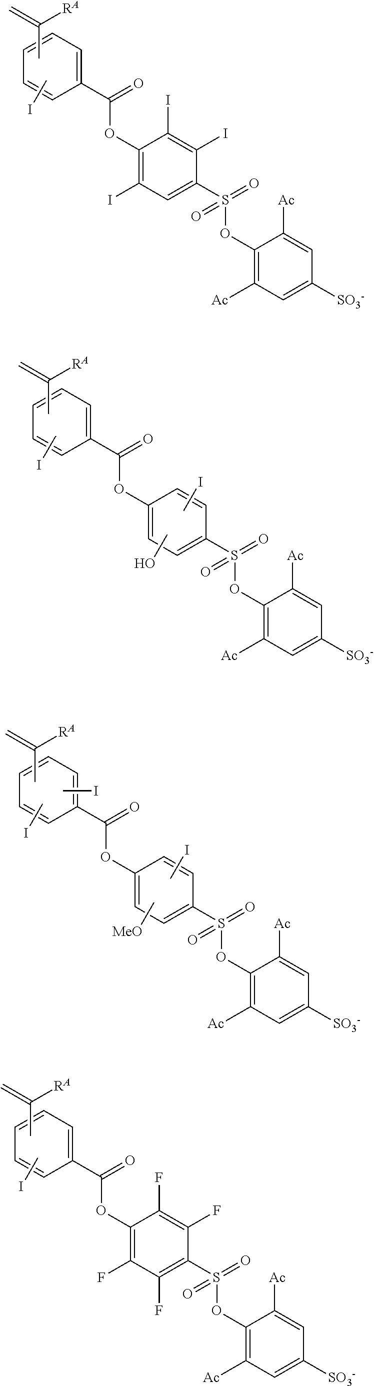

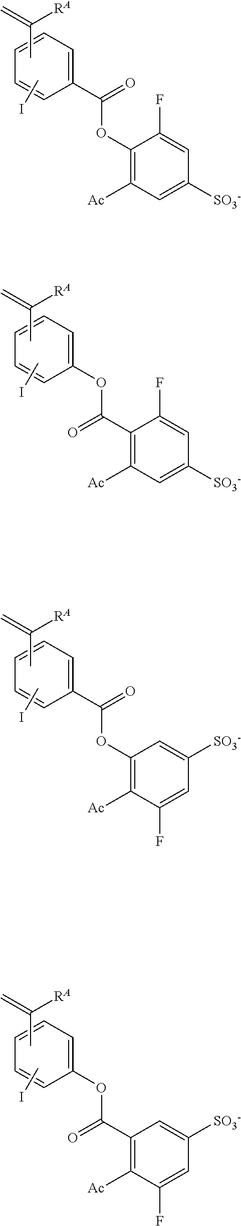

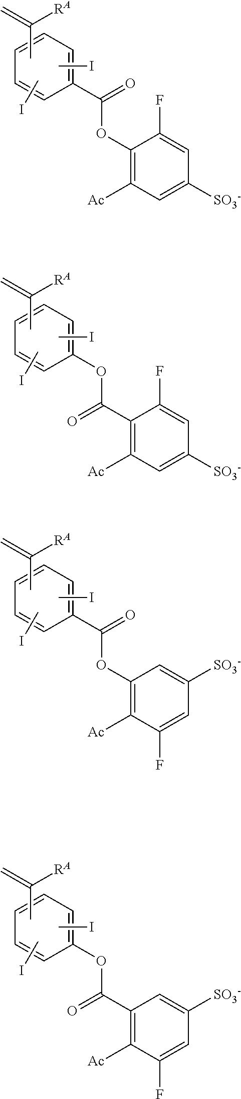

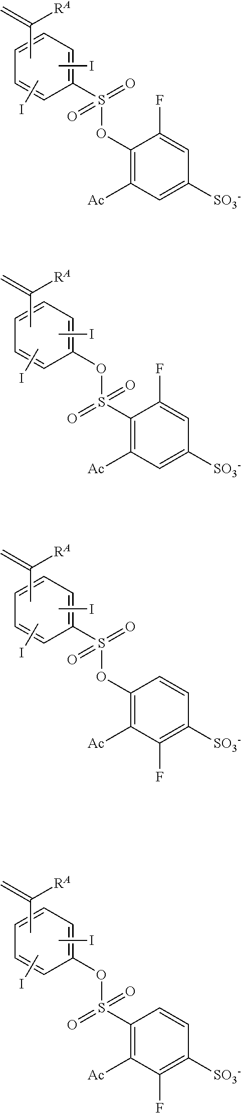

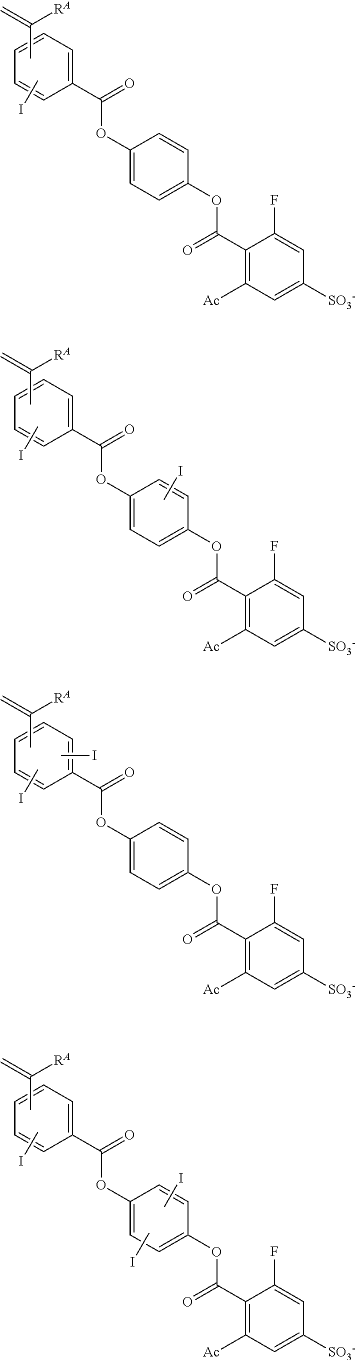

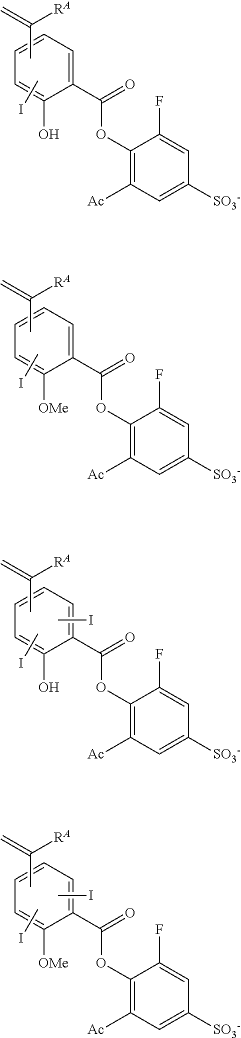

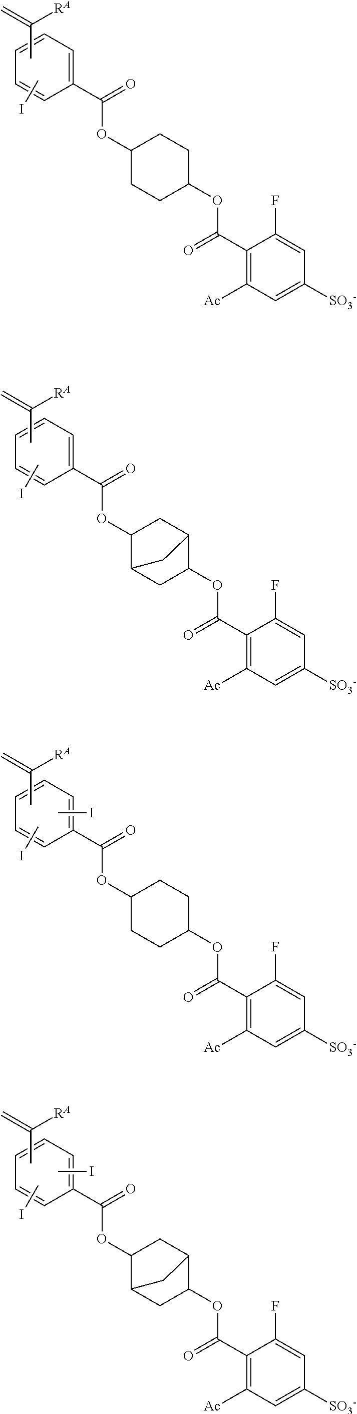

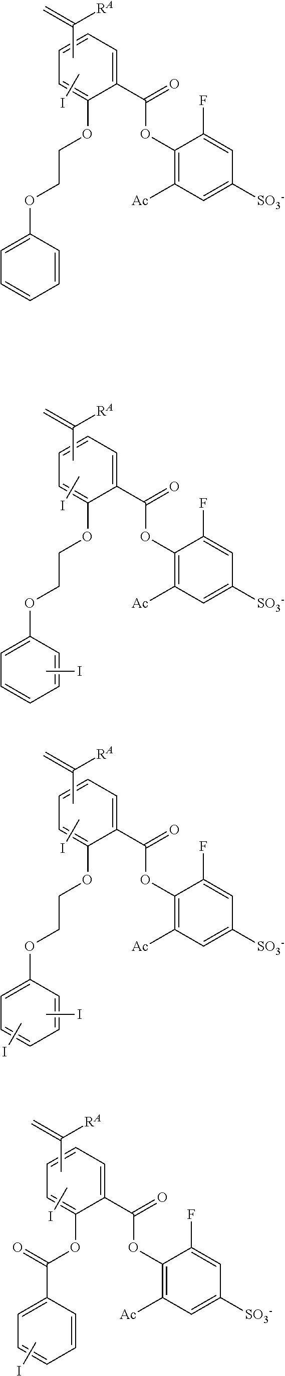

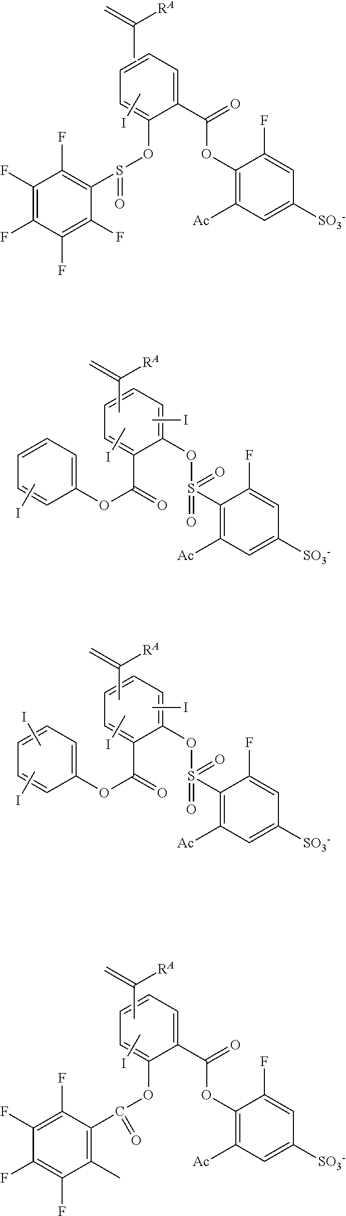

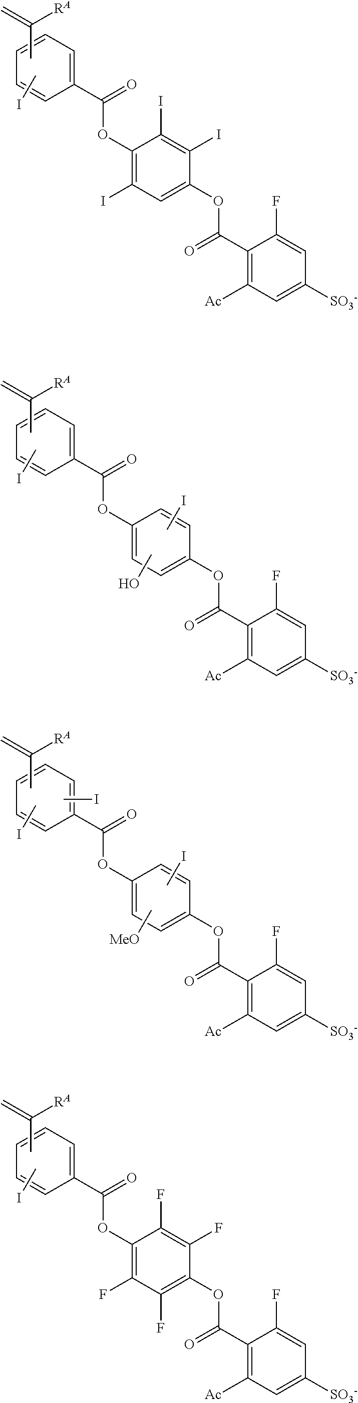

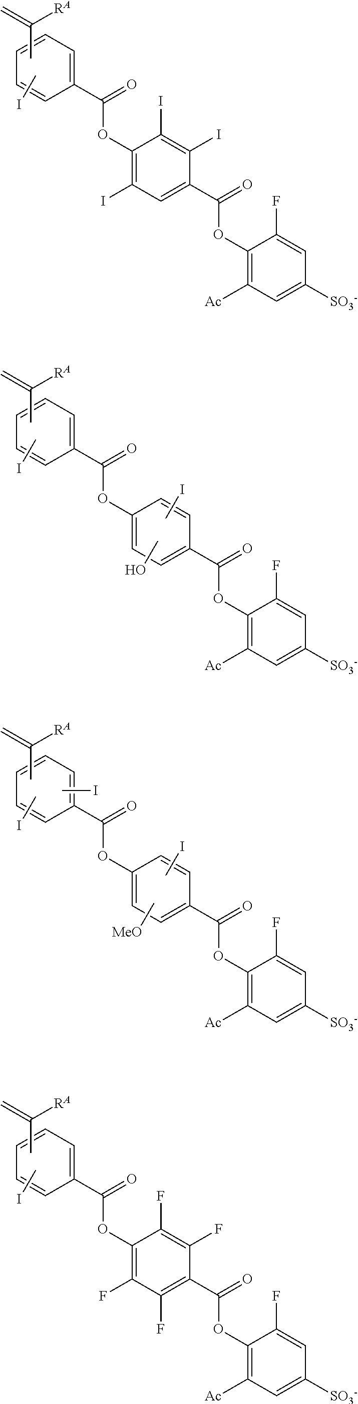

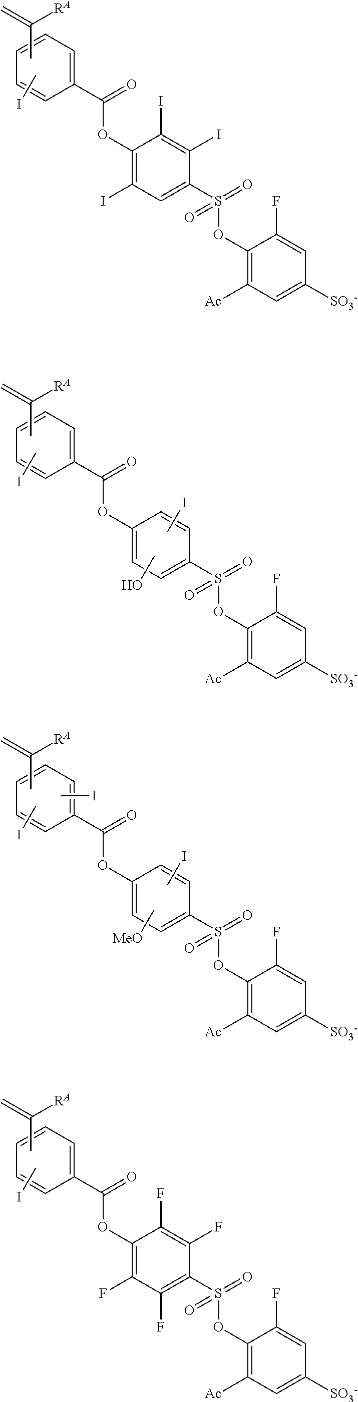

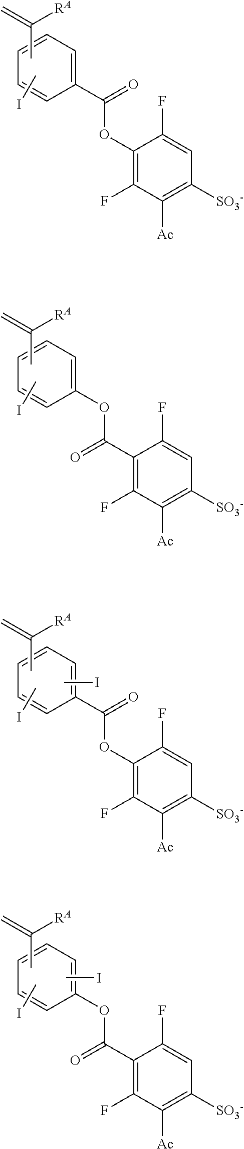

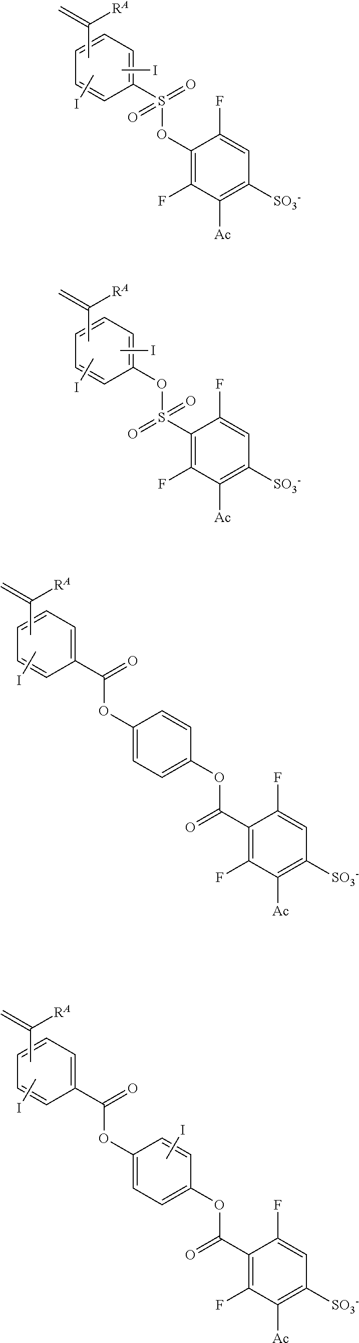

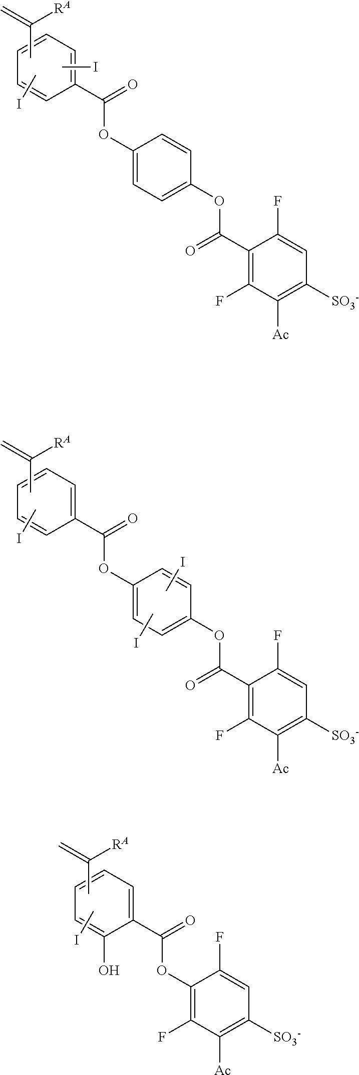

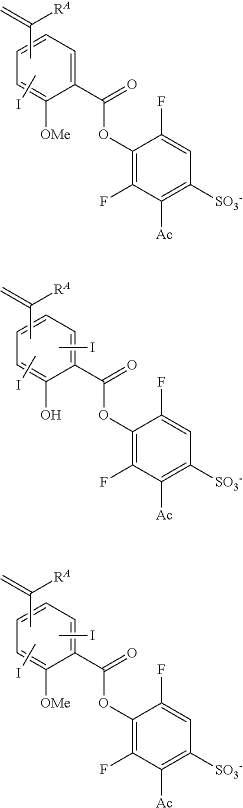

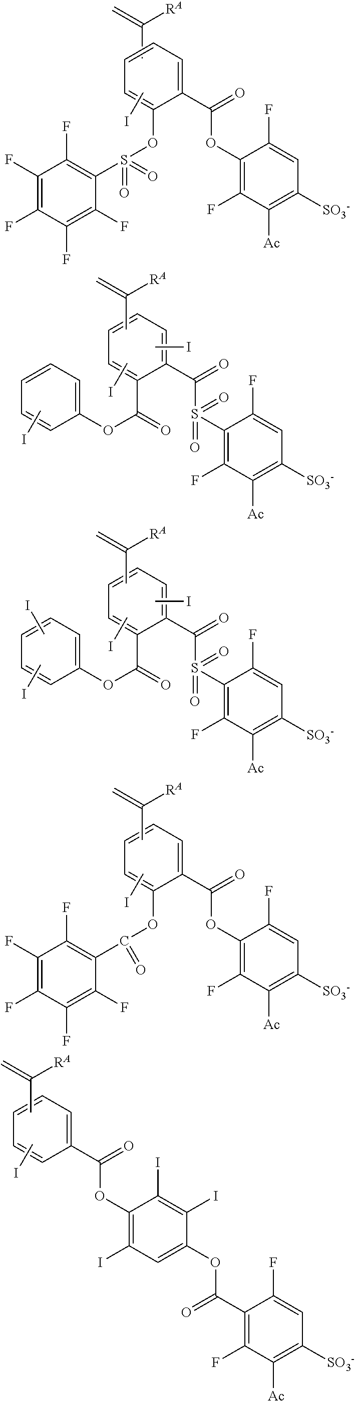

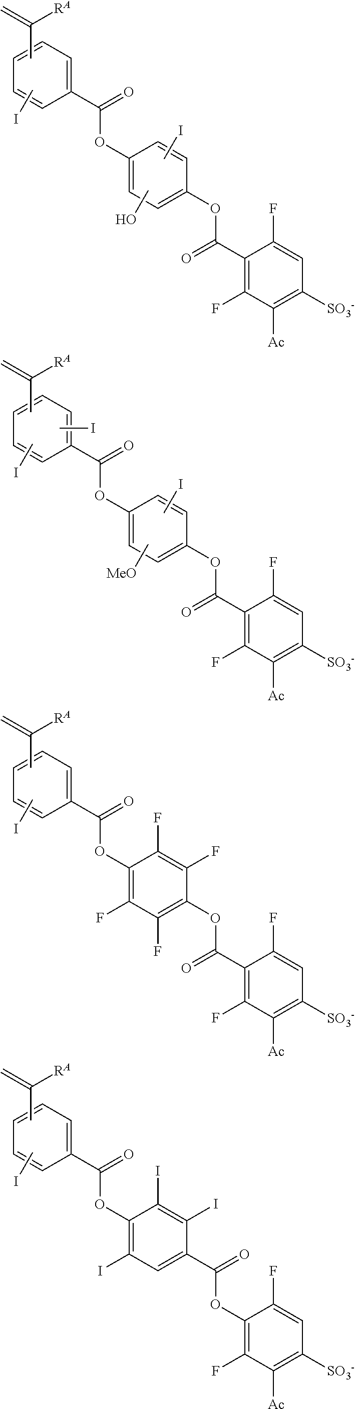

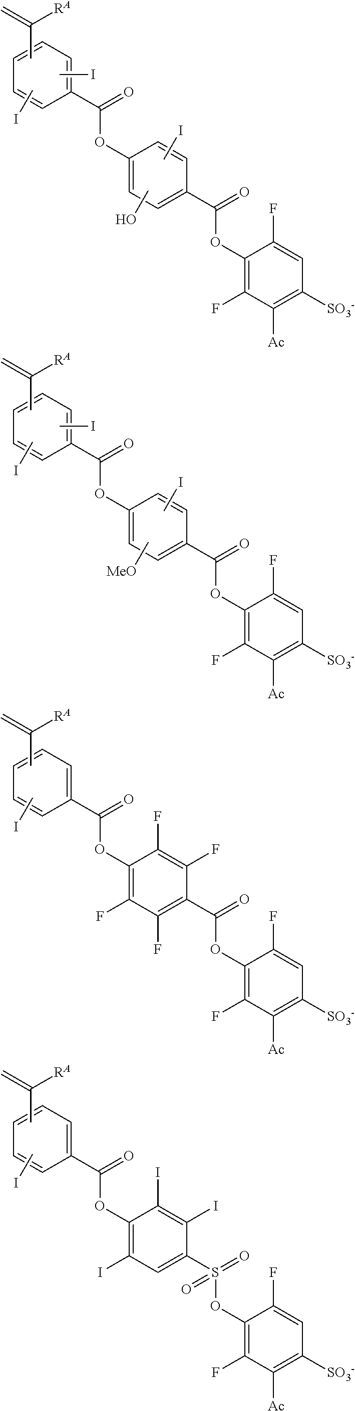

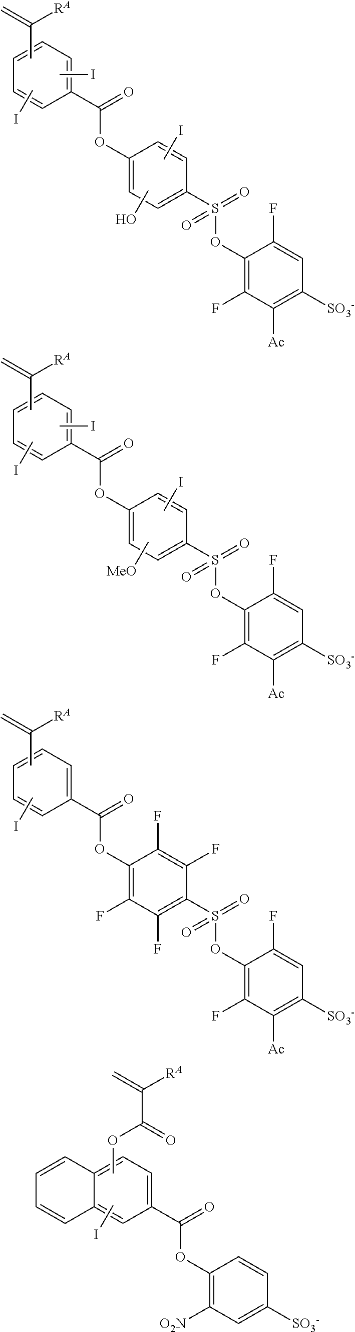

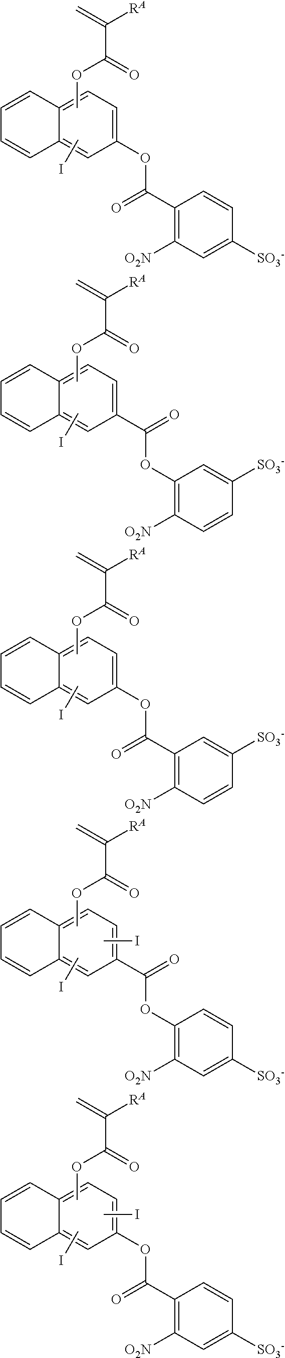

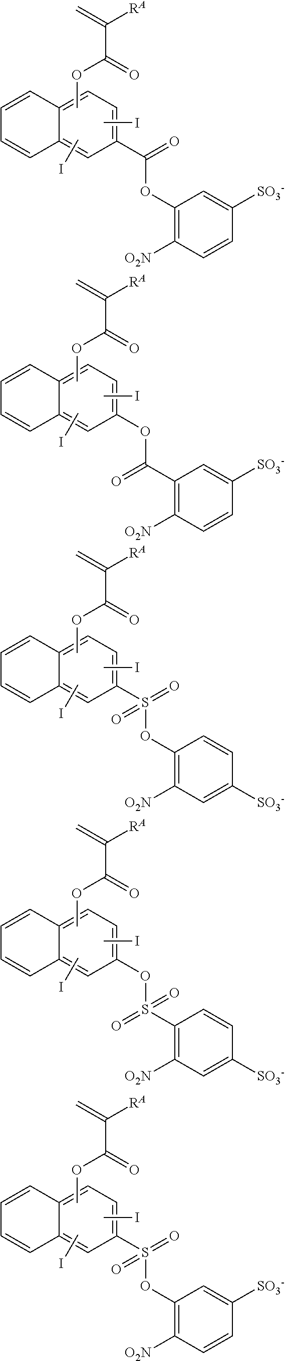

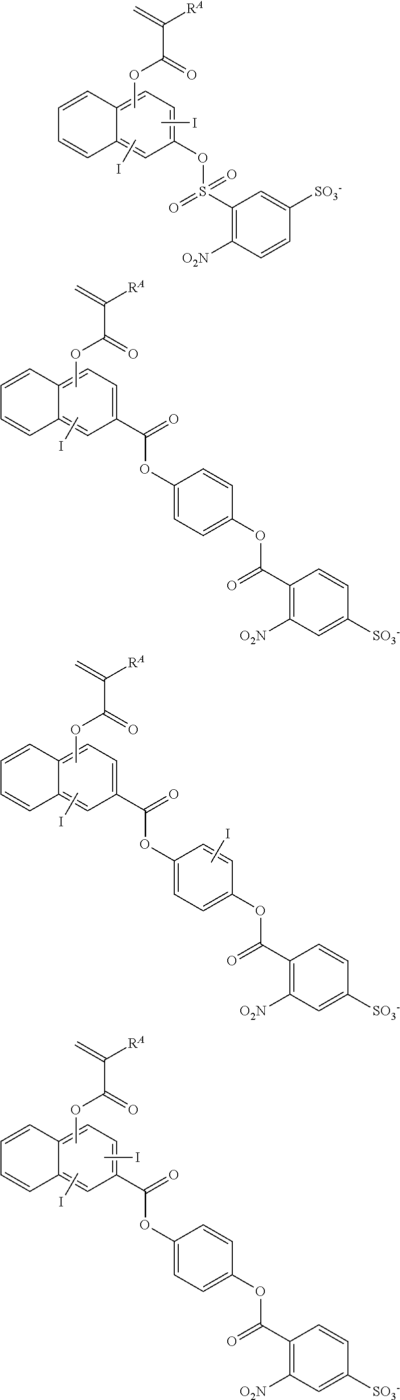

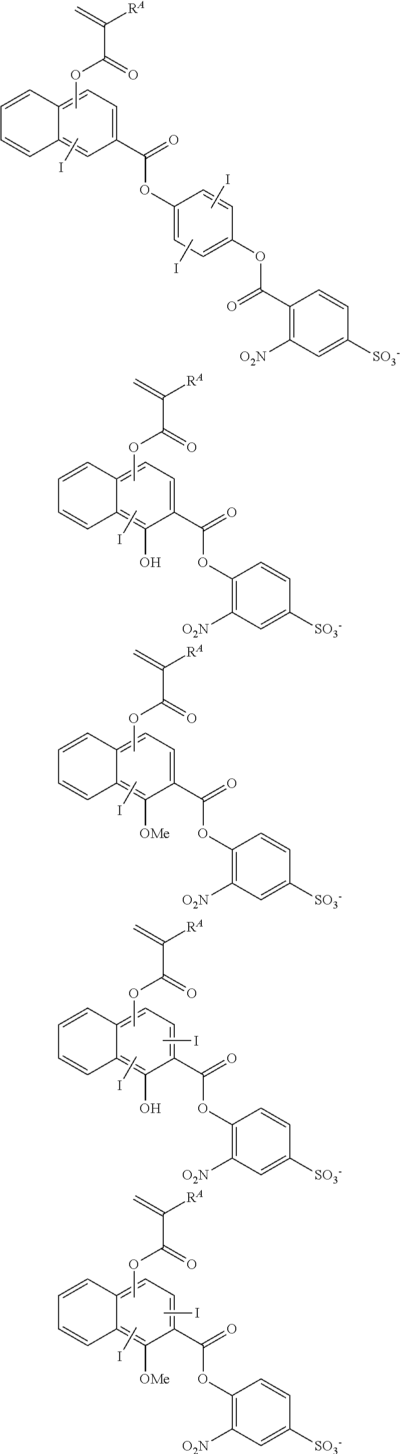

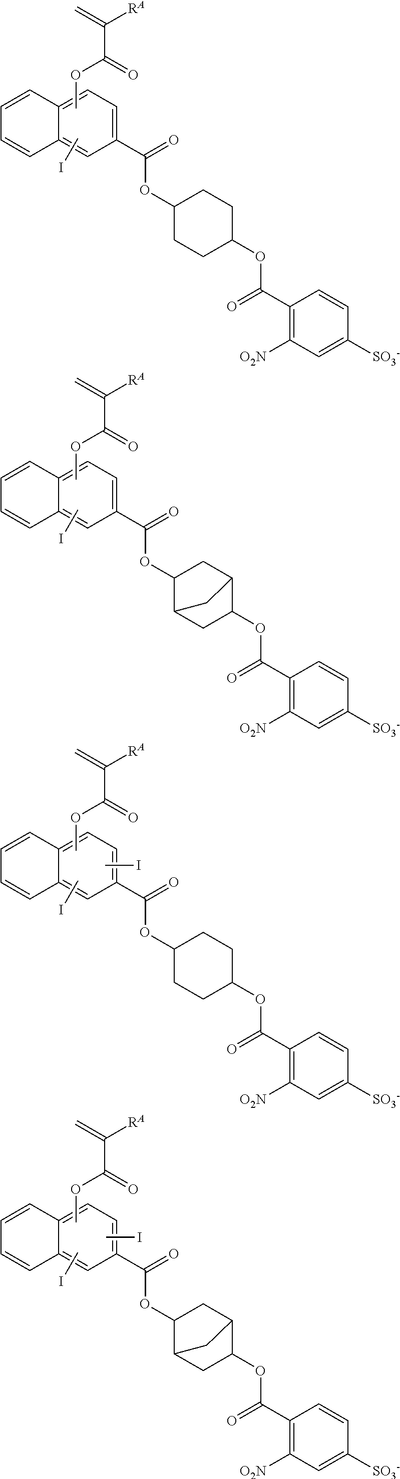

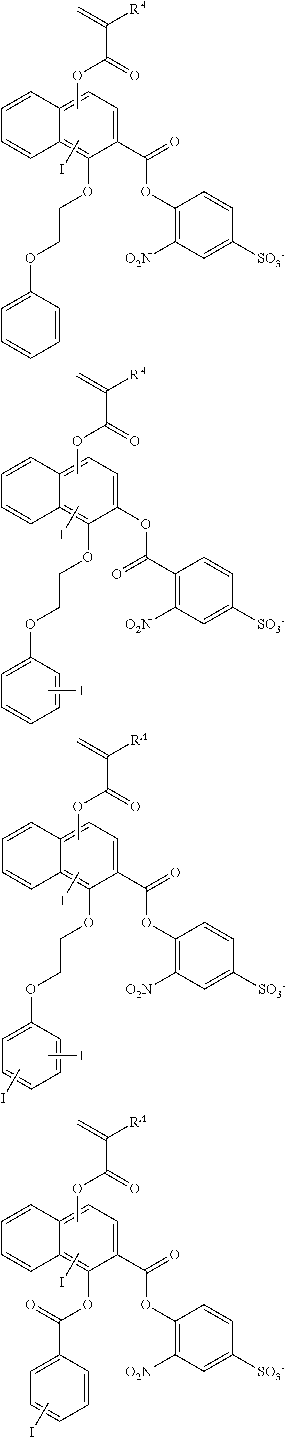

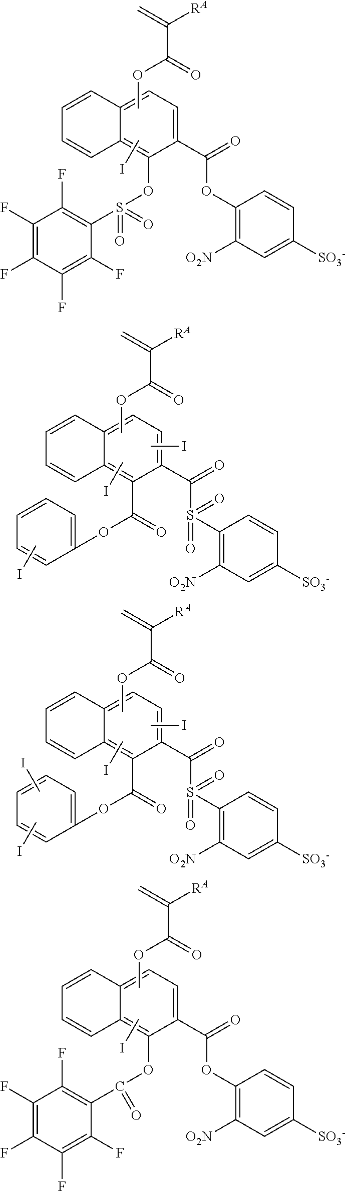

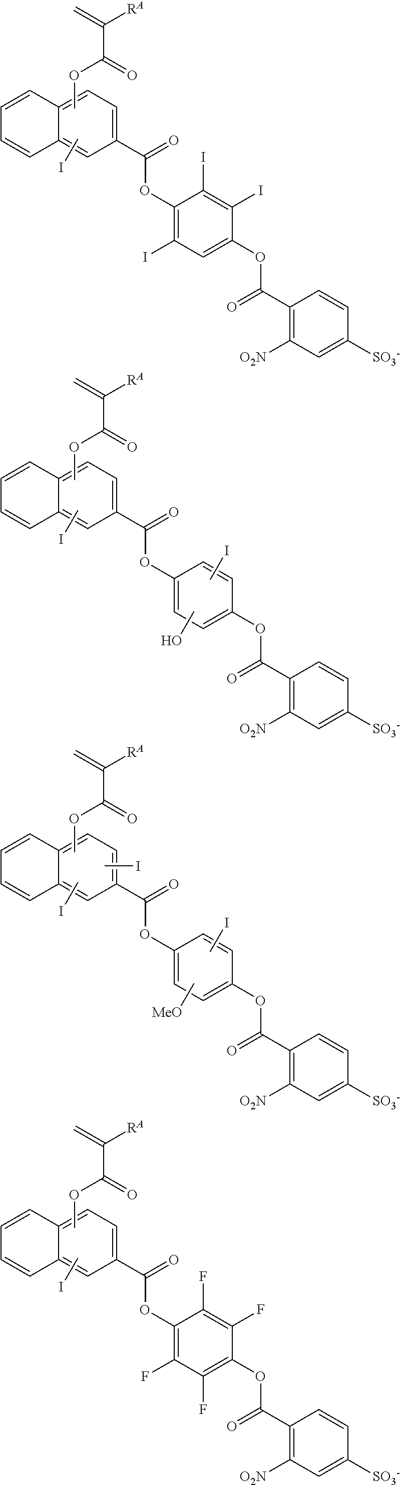

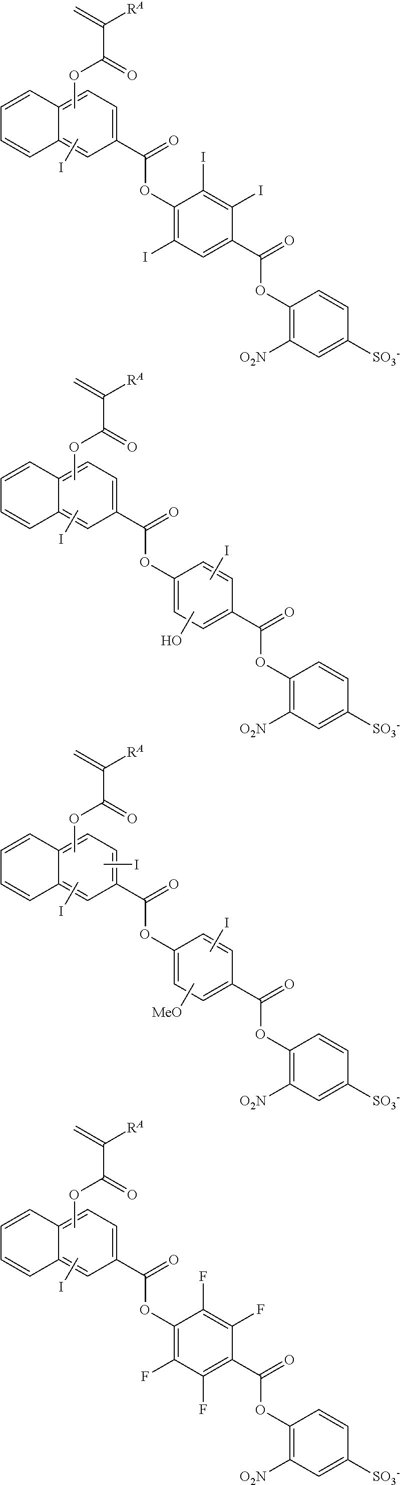

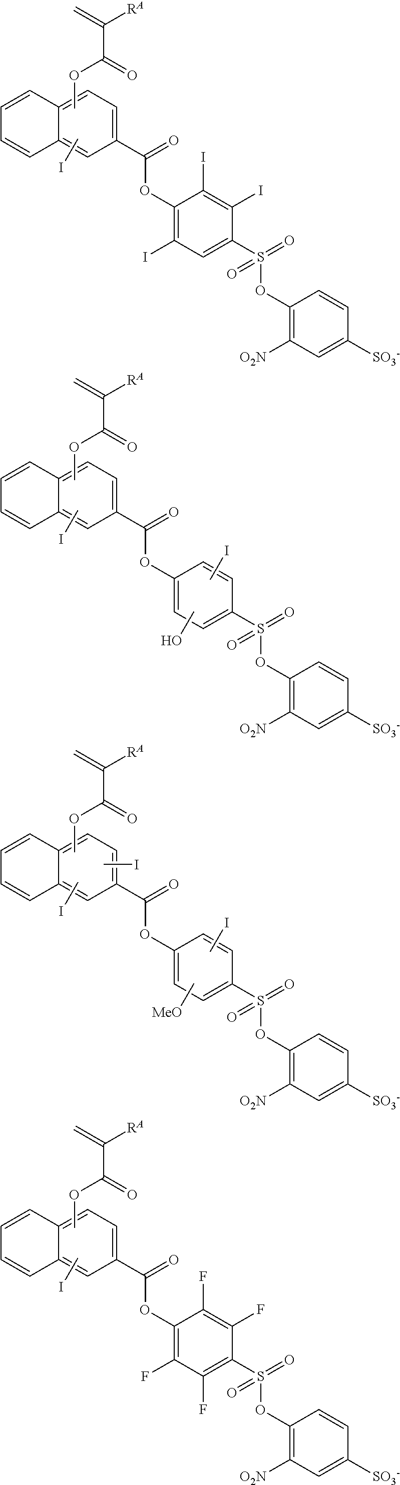

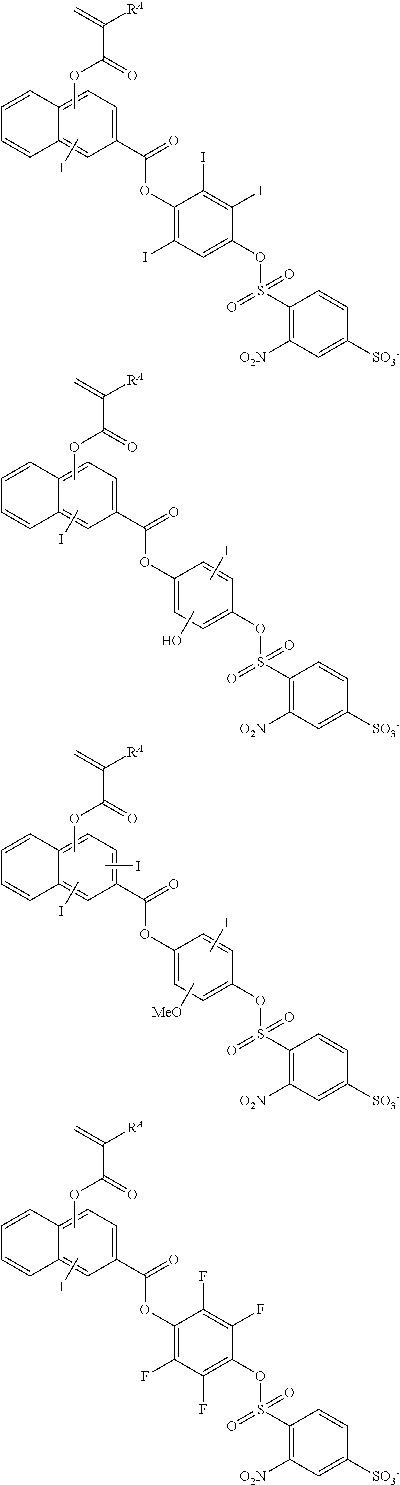

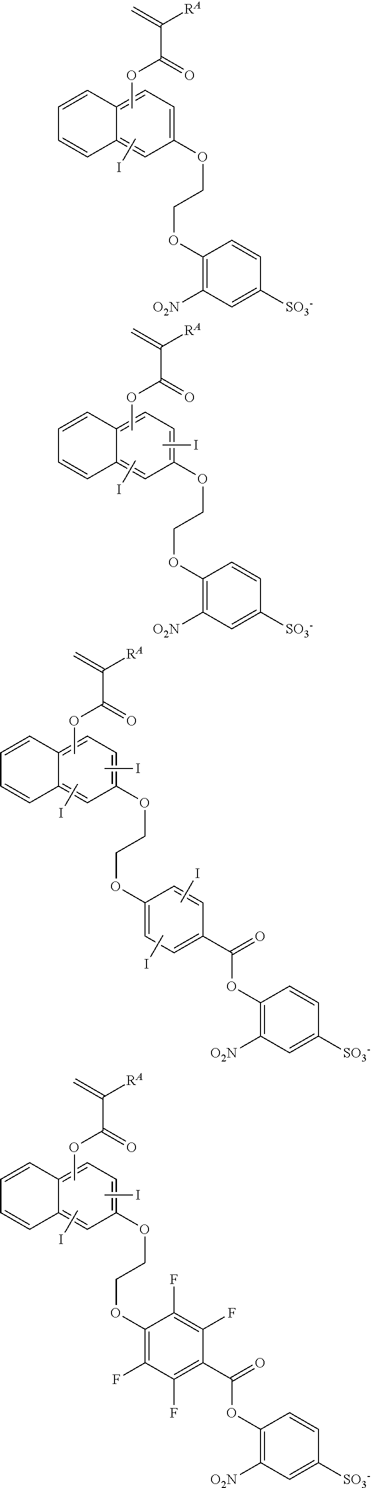

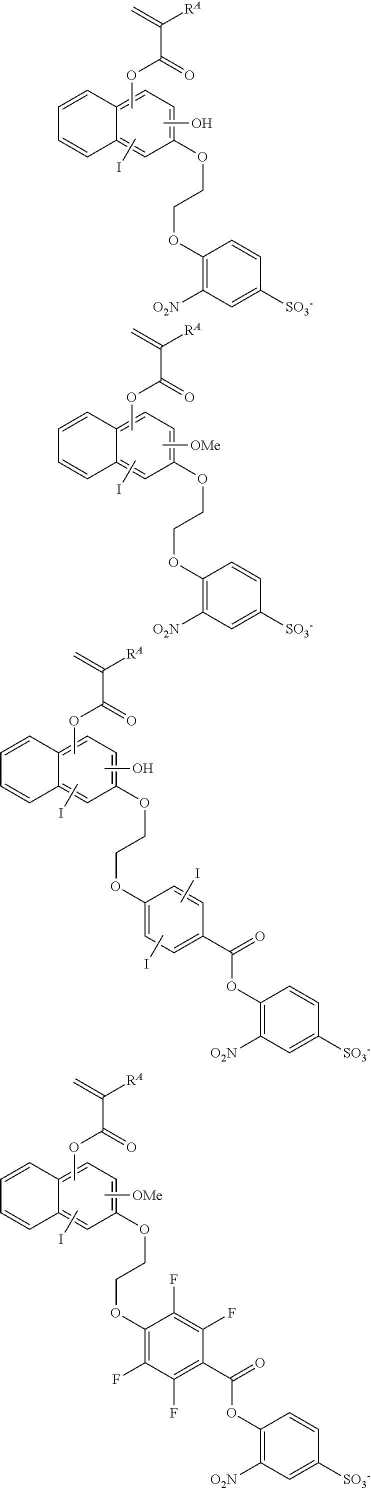

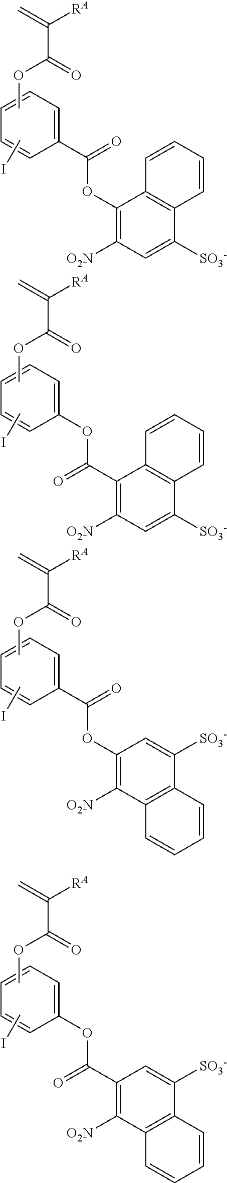

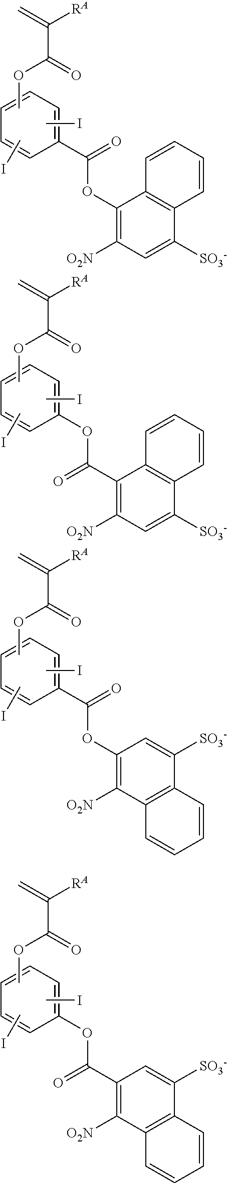

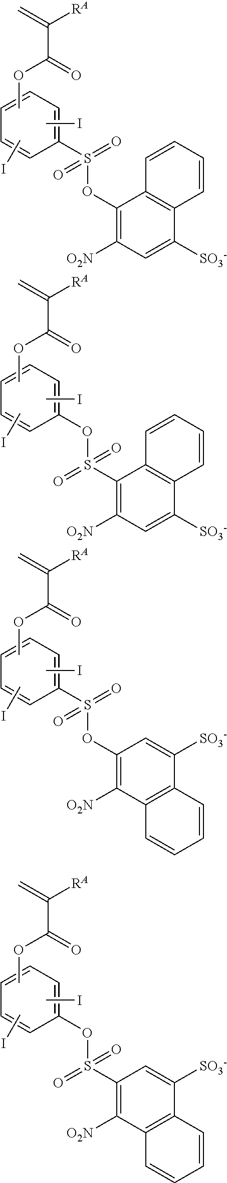

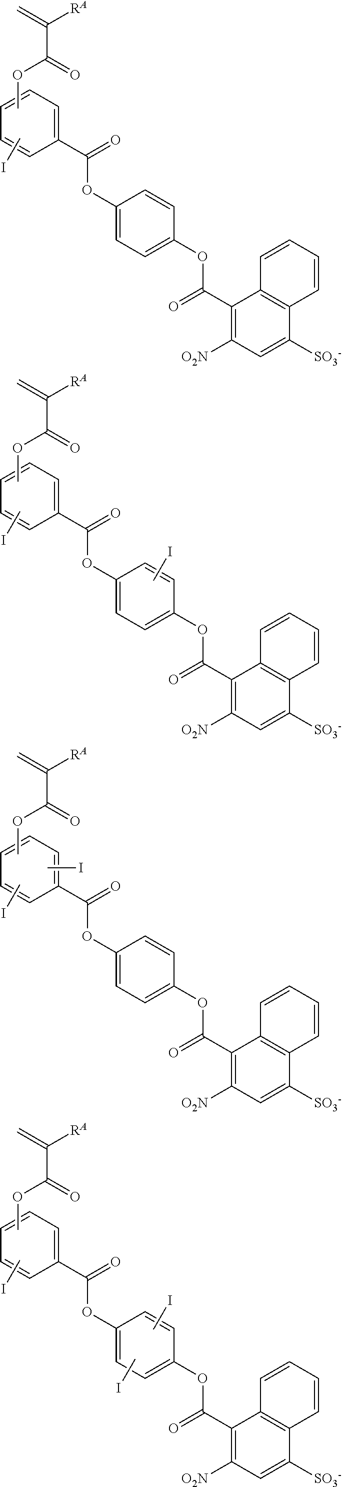

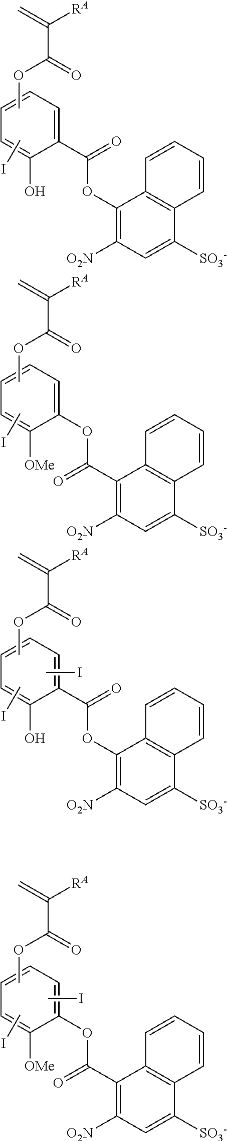

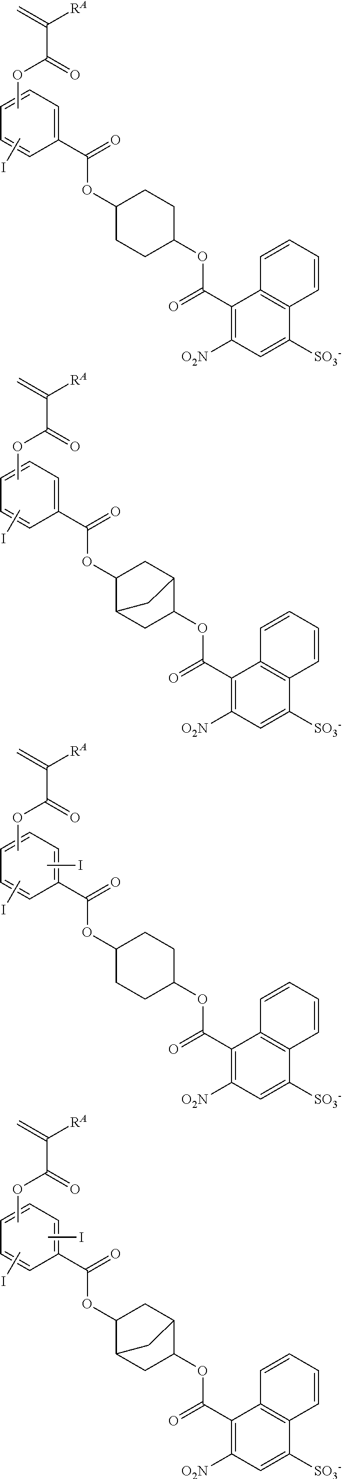

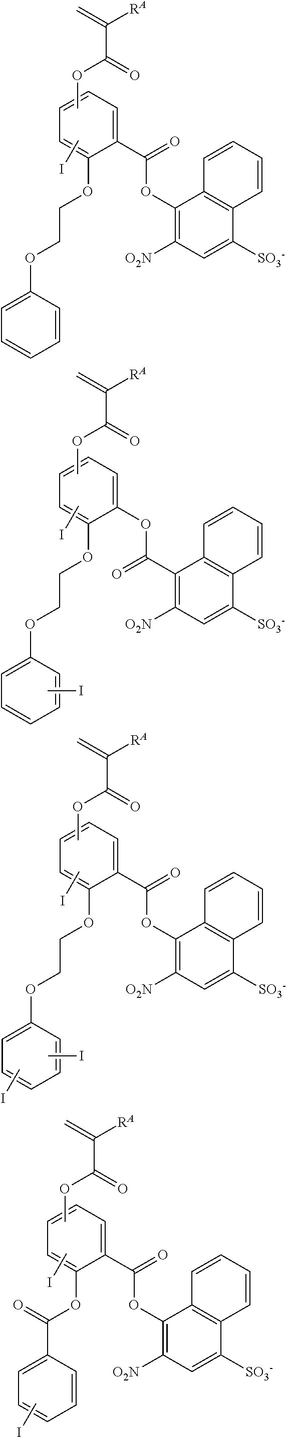

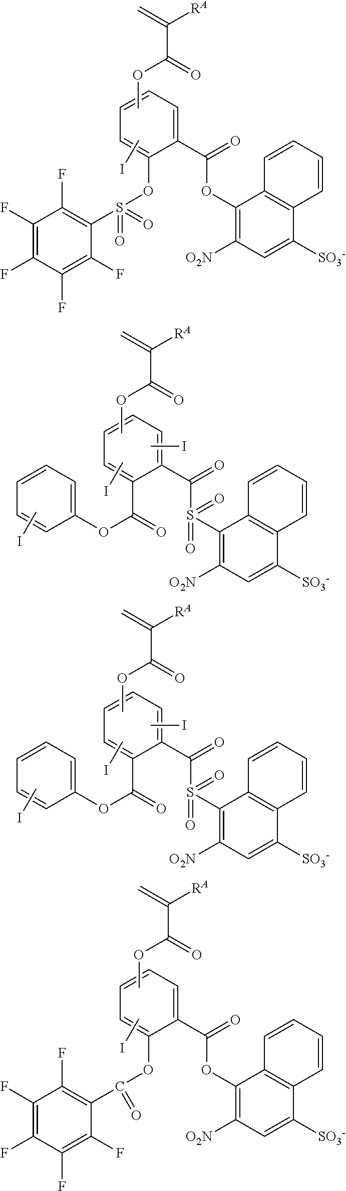

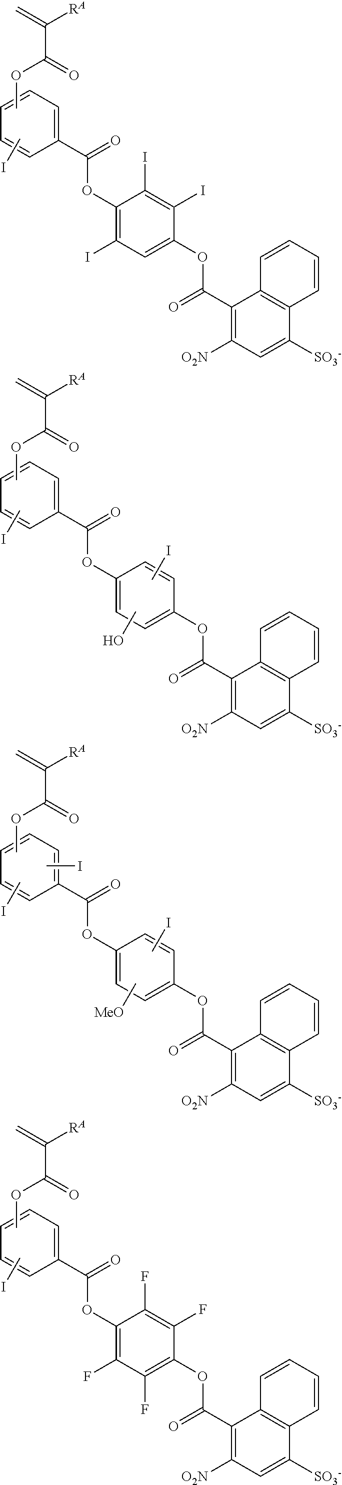

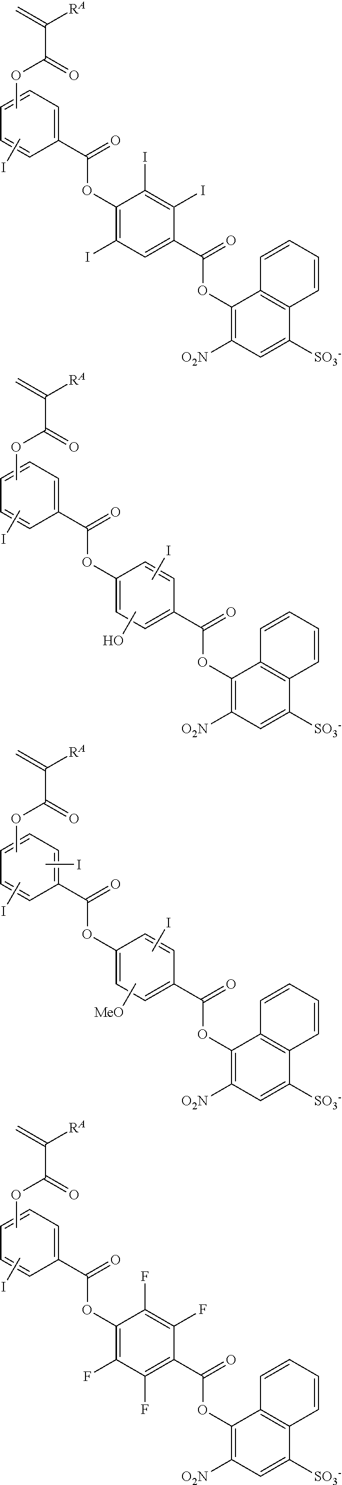

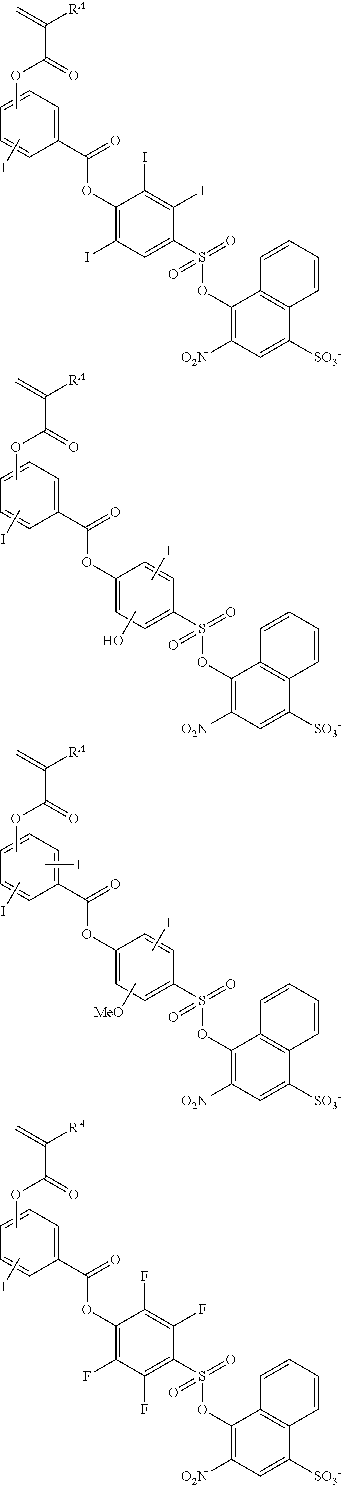

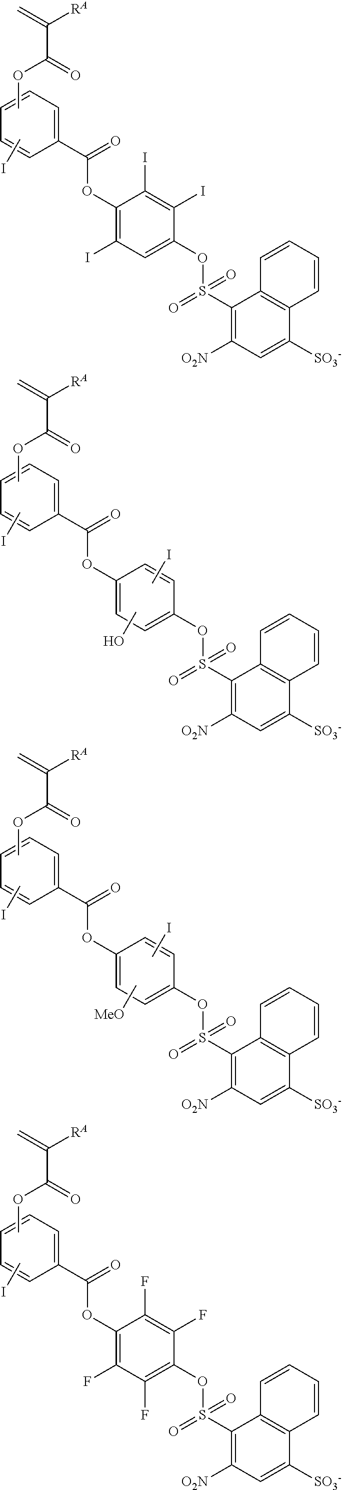

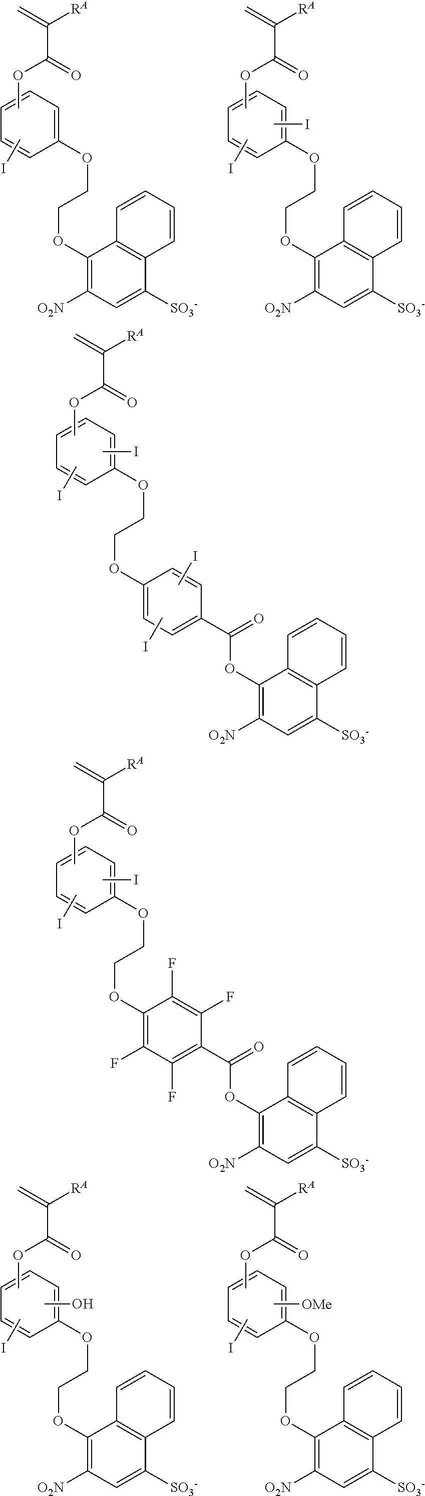

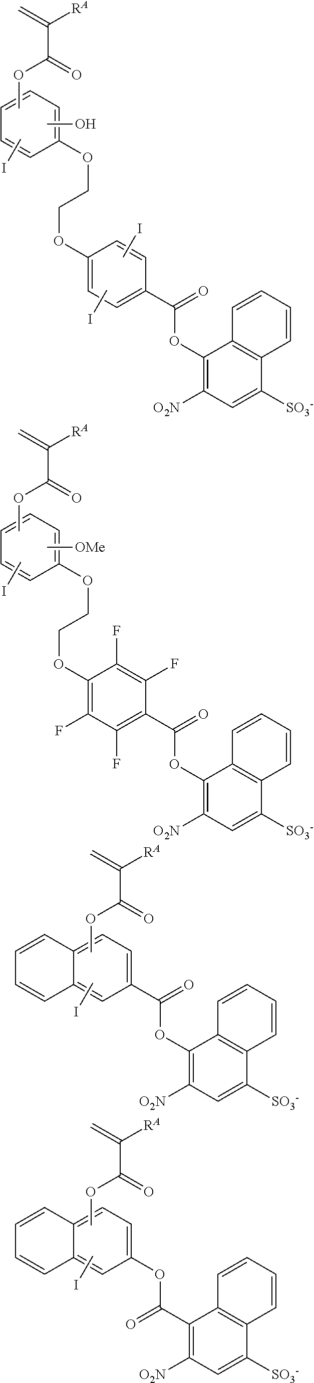

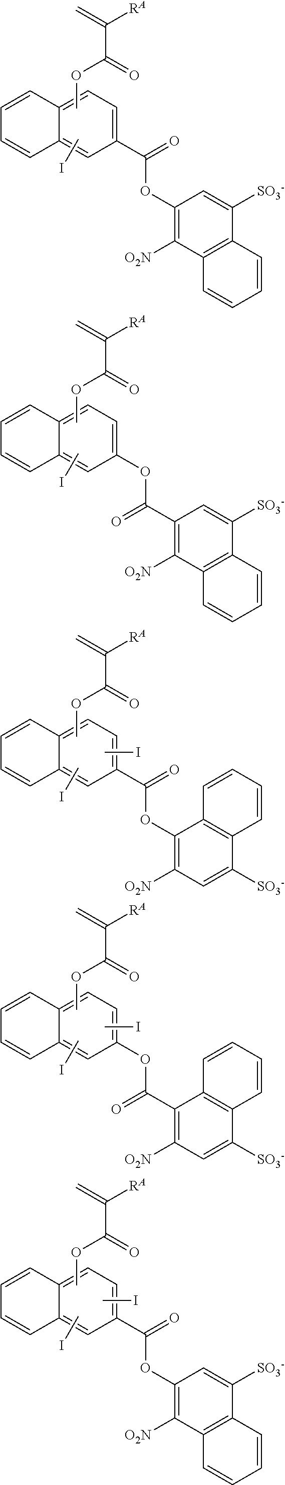

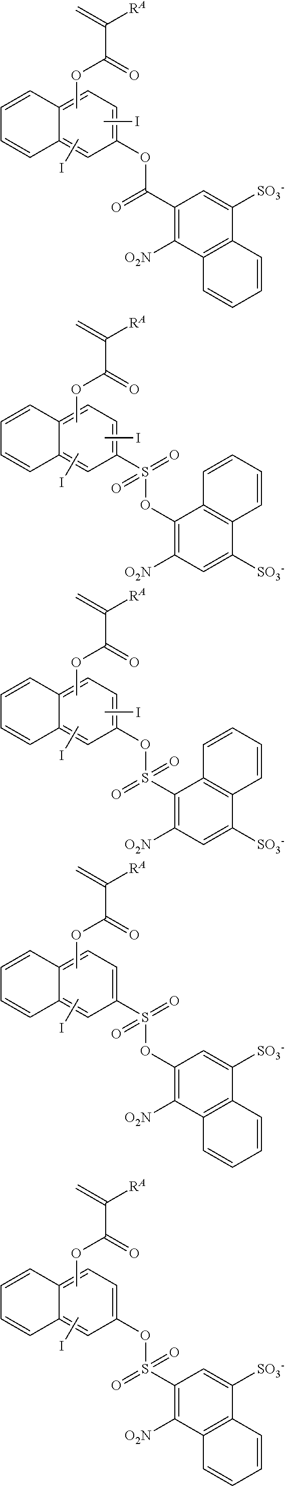

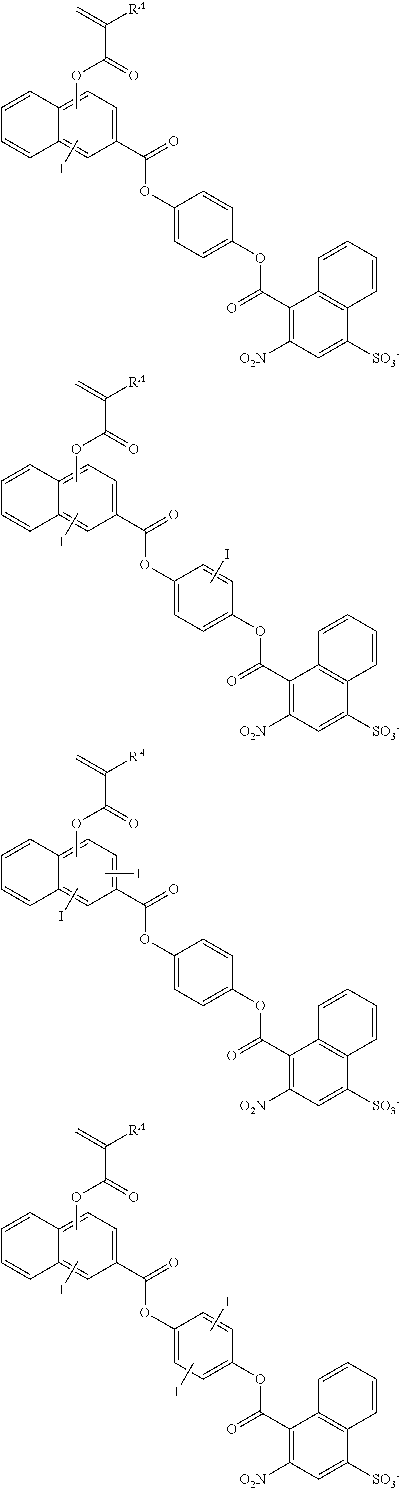

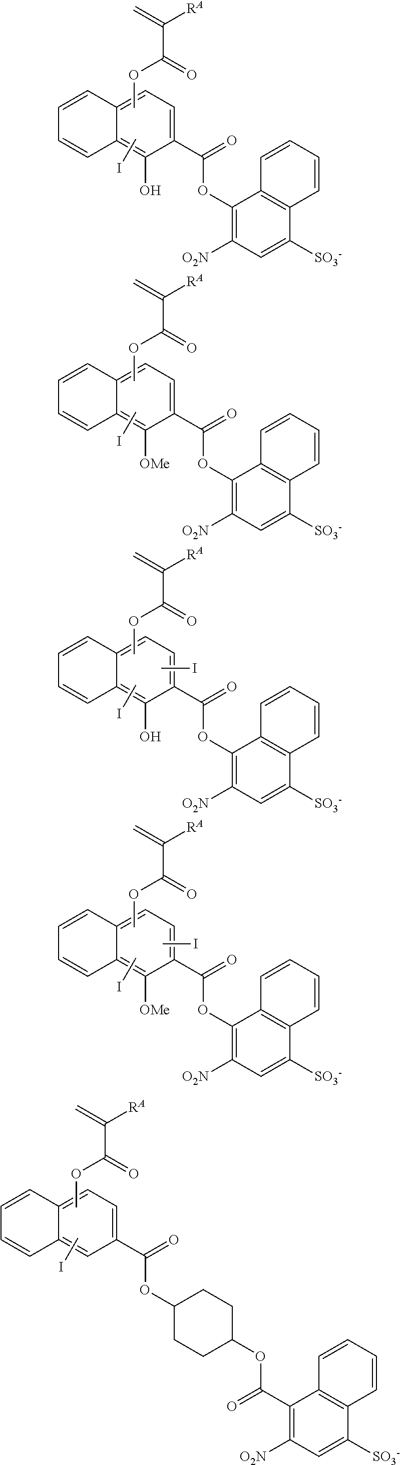

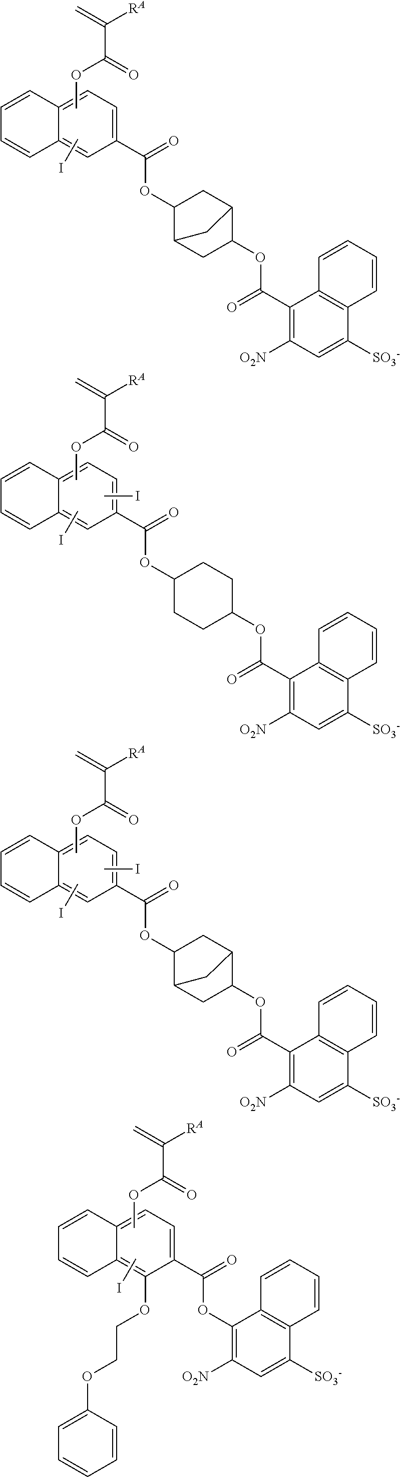

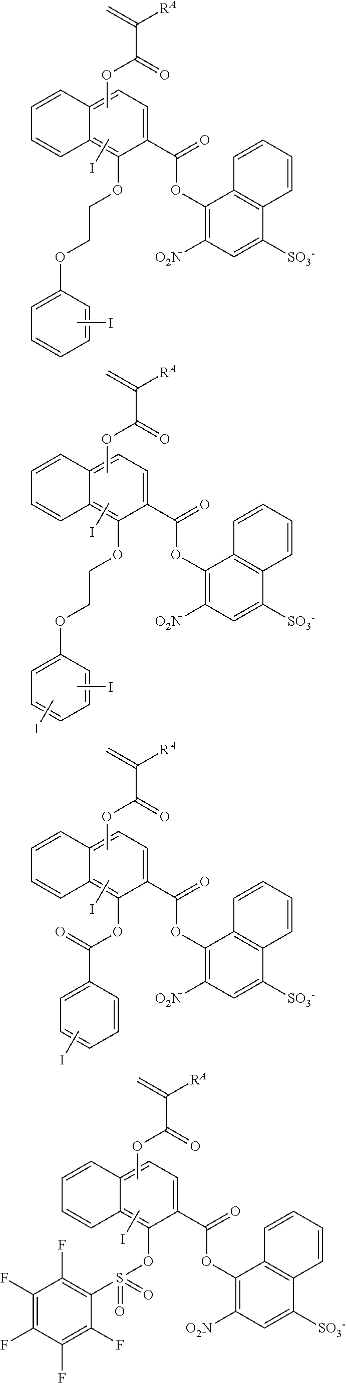

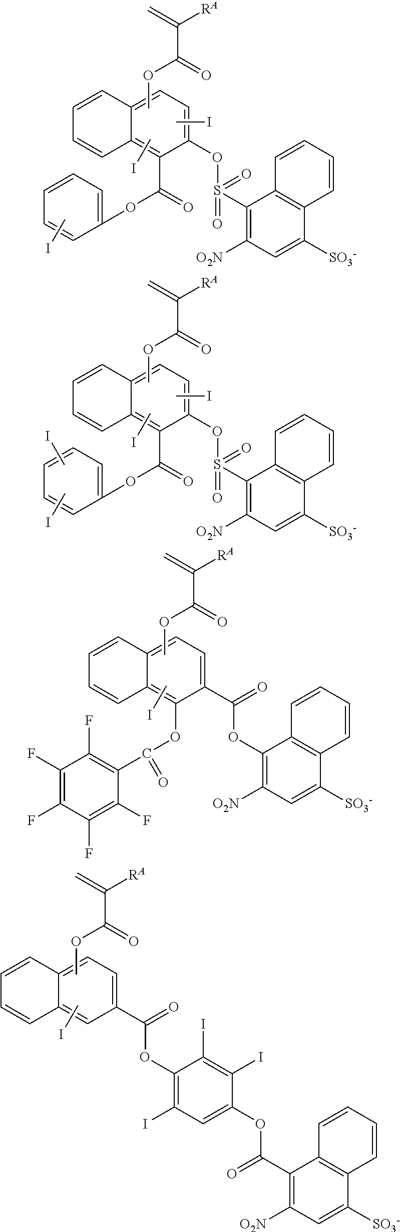

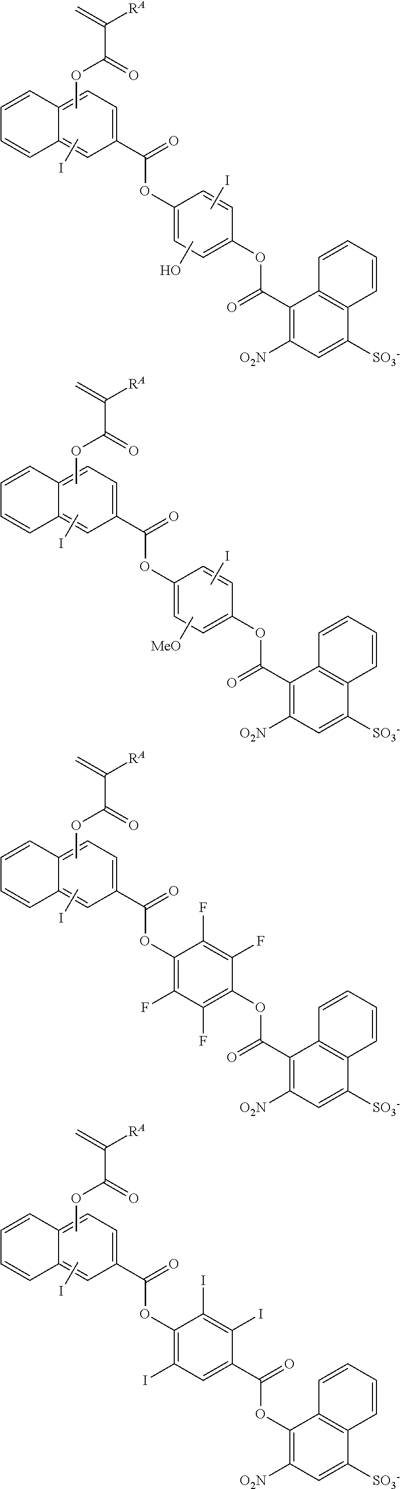

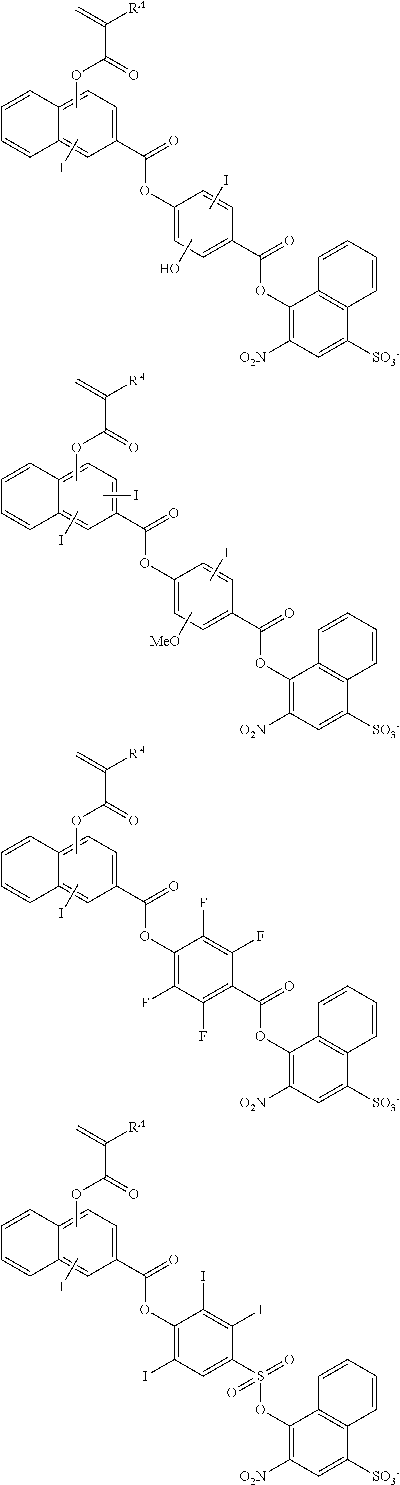

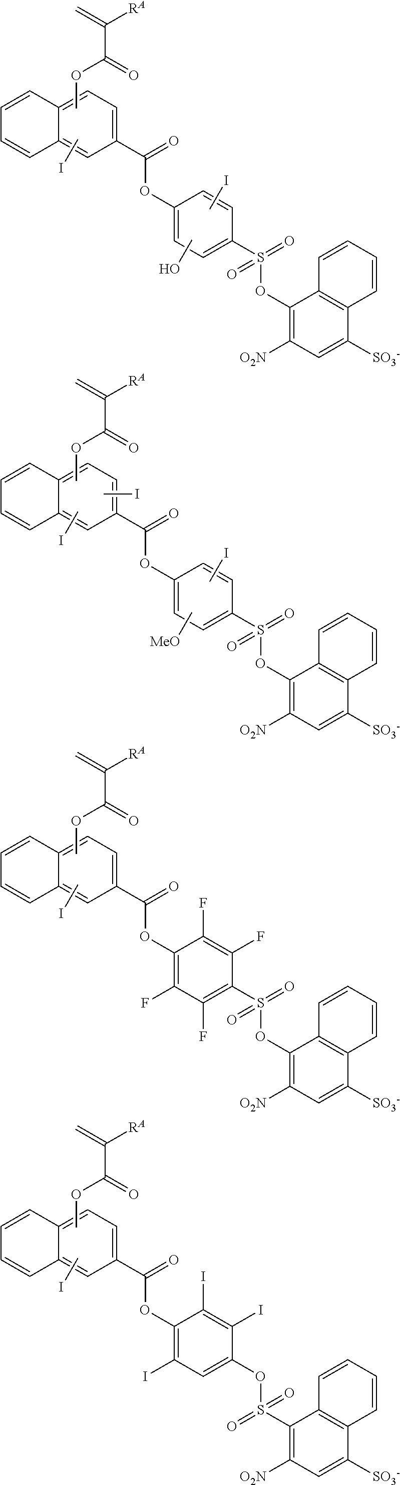

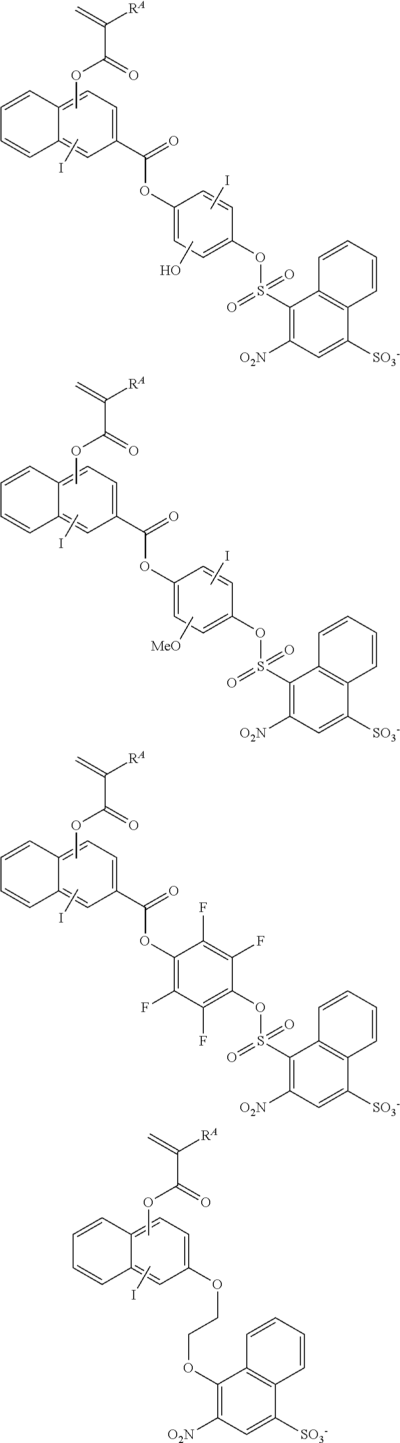

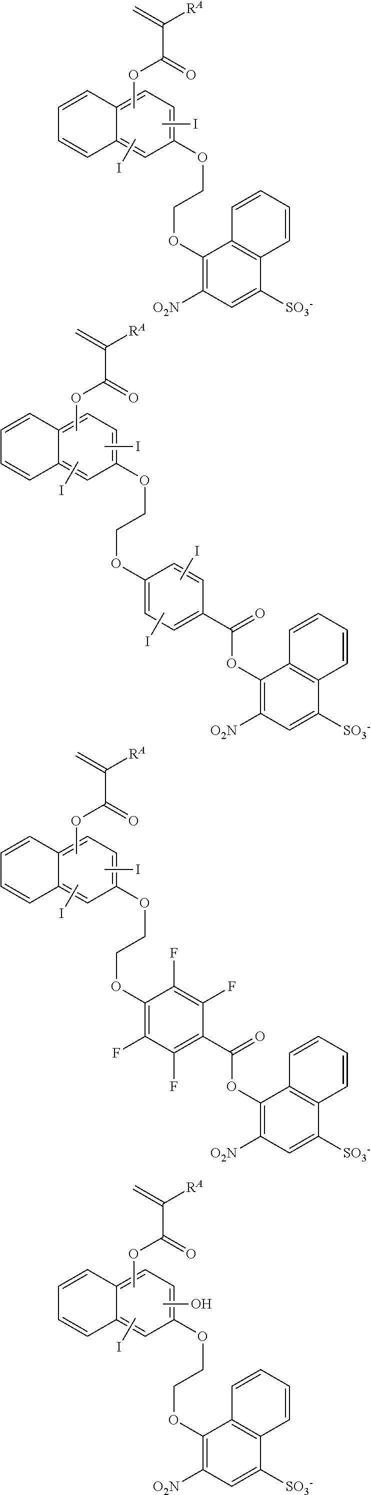

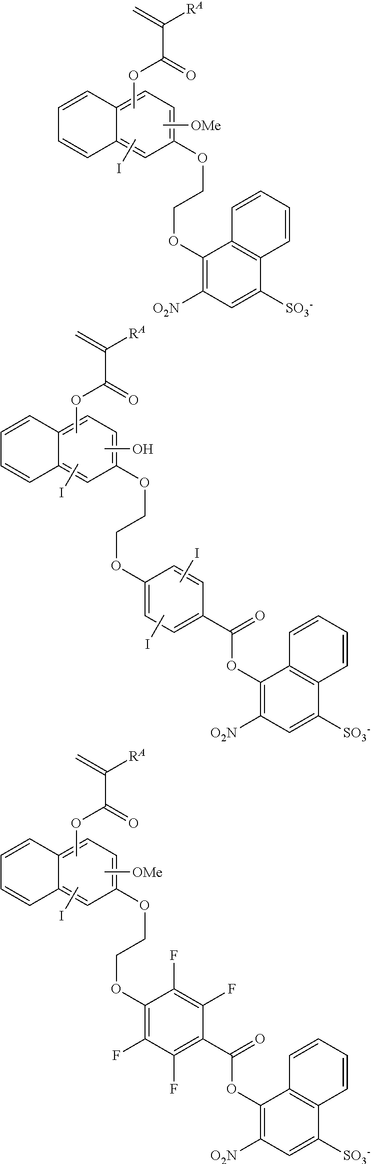

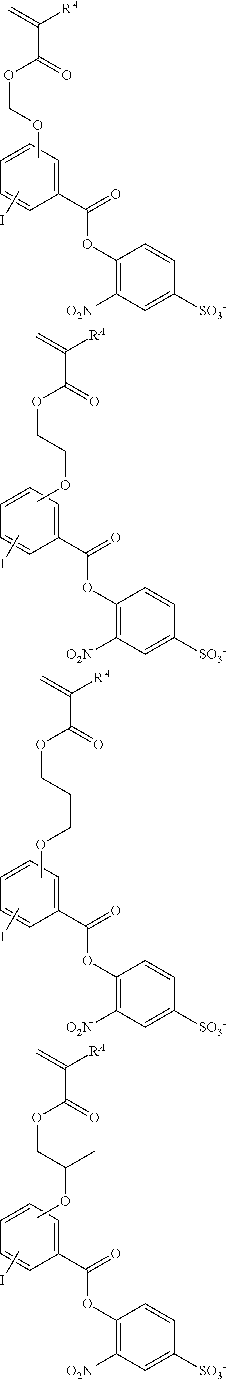

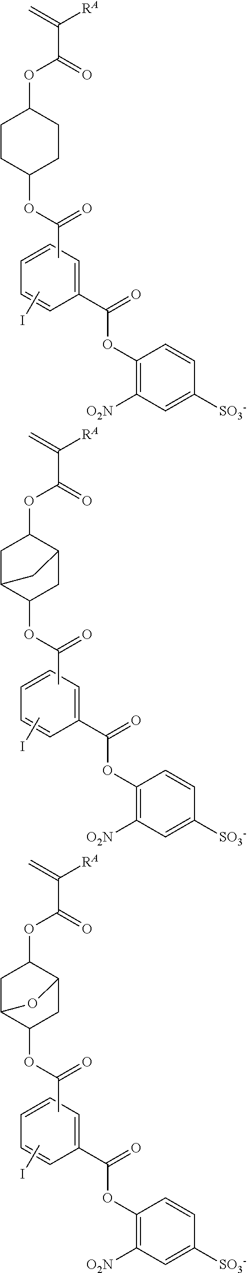

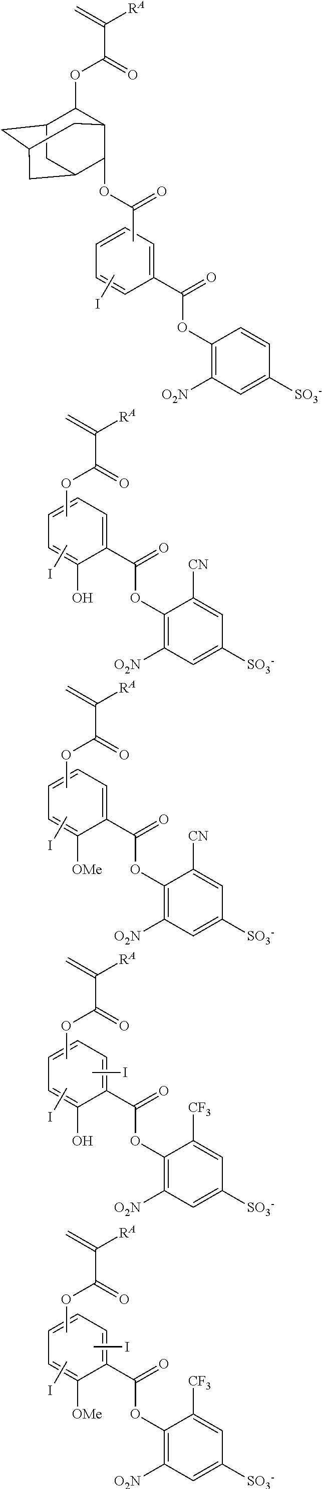

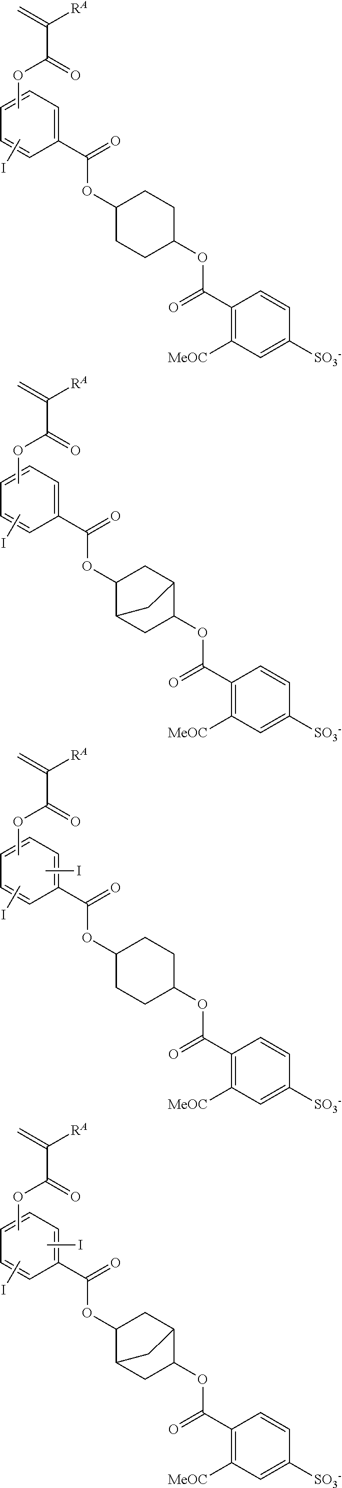

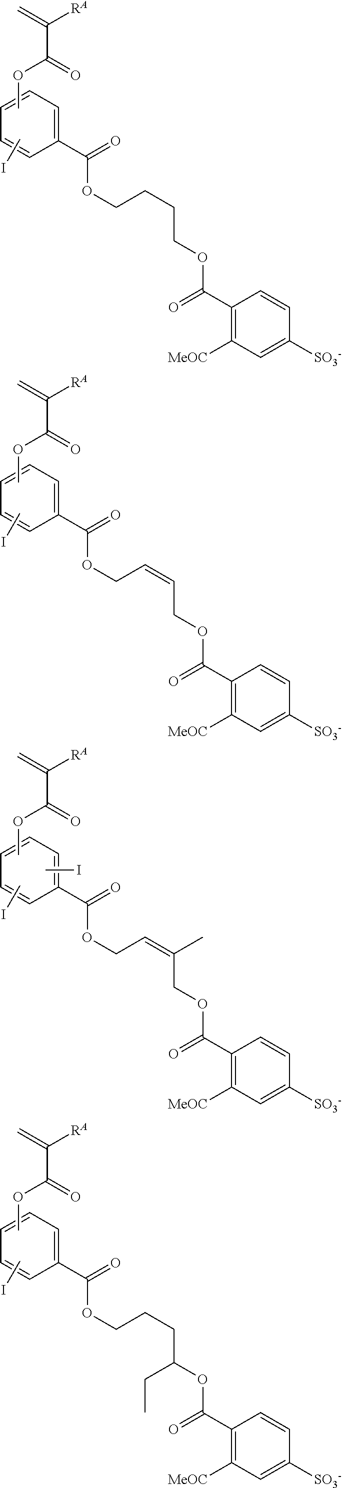

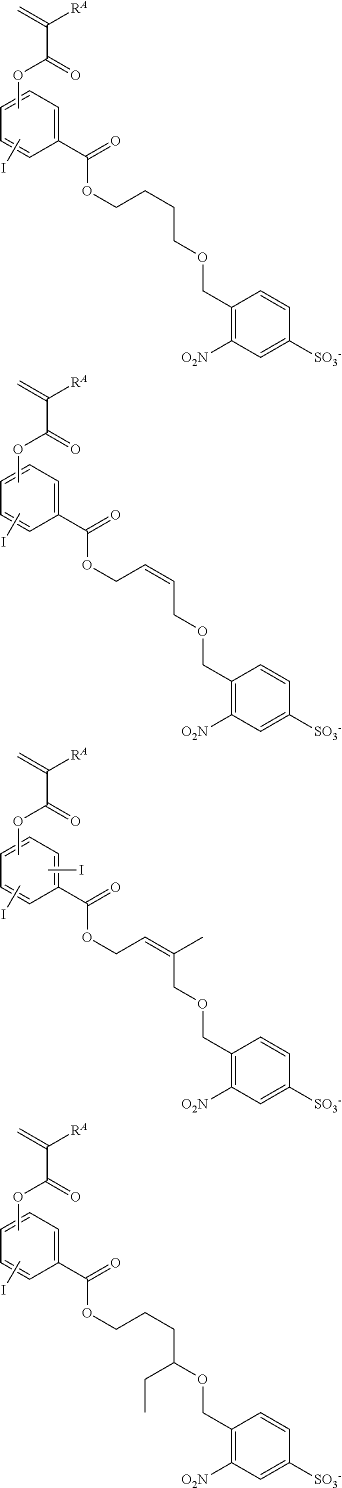

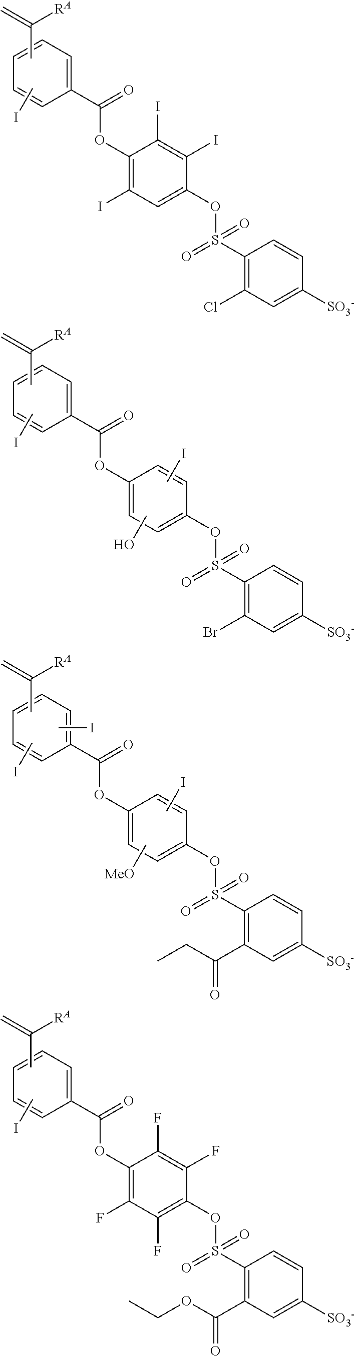

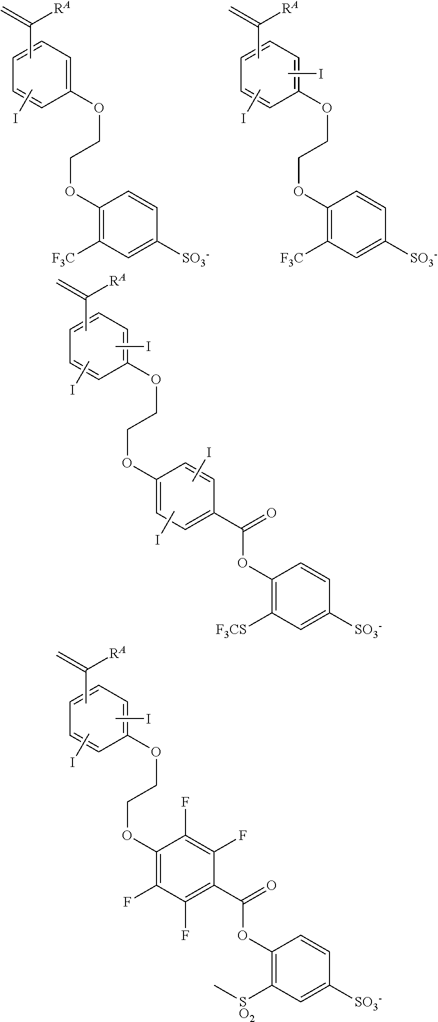

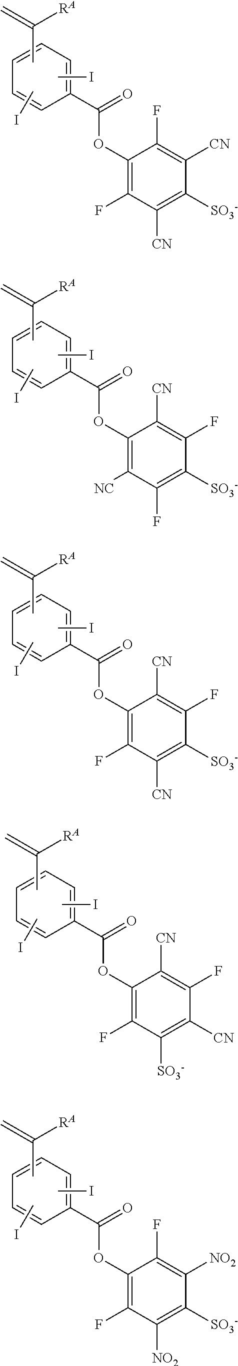

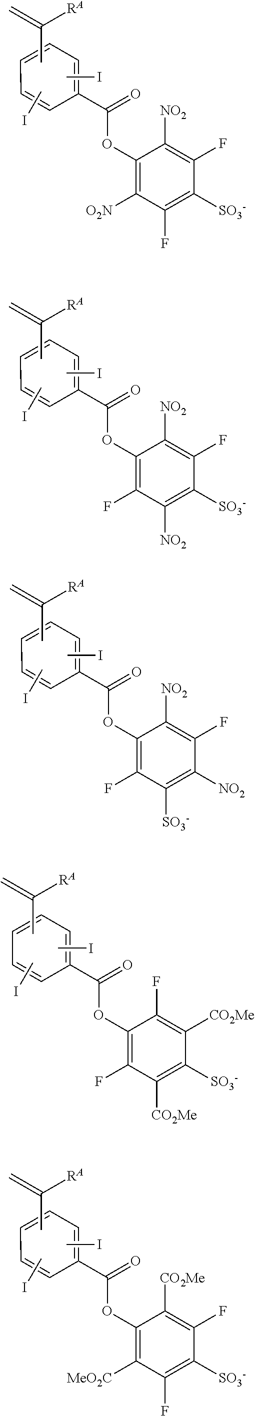

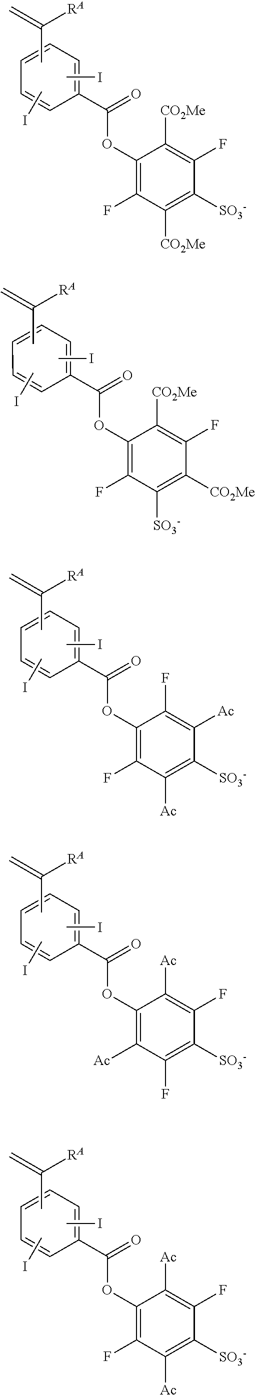

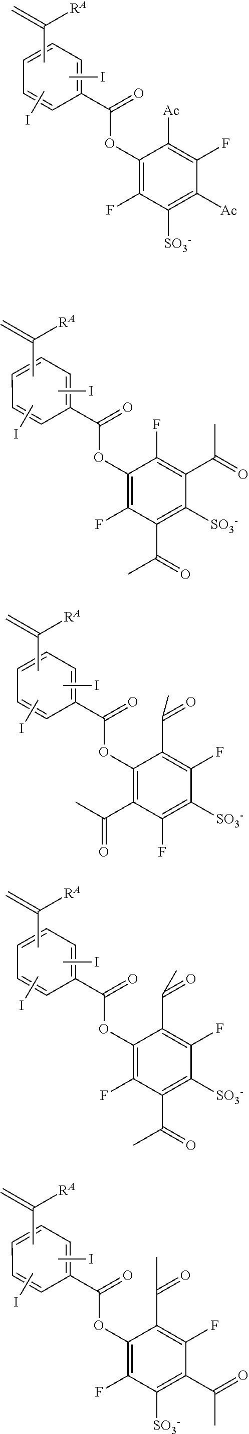

In the formula (a), Z⁺ is a sulfonium cation having the following formula (Z-1) or an iodonium cation having the following formula (Z-2).

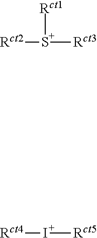

In formulae (Z-1) and (Z-2), R^{ct1} to R^{ct5} are each independently halogen, or a C₁-C₃₀ hydrocarbyl group which may contain a heteroatom.

Examples of the halogen atom represented by R^{ct1} to R^{ct5} include a fluorine atom, a chlorine atom, a bromine atom, and an iodine atom.

The hydrocarbyl group represented by R^{ct1} to R^{ct5} may be saturated or unsaturated, and may be straight, branched, or cyclic. Examples thereof include C₁-C₃₀ alkyl groups such as methyl, ethyl, n-propyl, isopropyl, n-butyl, isobutyl, sec-butyl and tert-butyl groups; C₃-C₃₀ cyclic saturated hydrocarbyl groups such as cyclopropyl, cyclopentyl, cyclohexyl, cyclopropylmethyl, 4-methylcyclohexyl, cyclohexylmethyl, norbornyl and adamantyl groups; C₂-C₃₀ alkenyl groups such as vinyl, allyl, propenyl, butenyl and hexenyl groups; C₃-C₃₀ cyclic unsaturated hydrocarbyl groups such as a cyclohexenyl group; C₆-C₃₀ aryl groups such as phenyl, naphthyl and thienyl groups; C₇-C₃₀ aralkyl groups such as benzyl, 1-phenylethyl and 2-phenylethyl groups; and combinations thereof. The aryl groups are preferred. In the hydrocarbyl group, some or all hydrogen may be replaced by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, nitro moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

R^{ct1} and R^{ct2} may bond together to form a ring with the sulfur atom to which they are attached. Examples of the structure of the ring include those represented by the following formula.

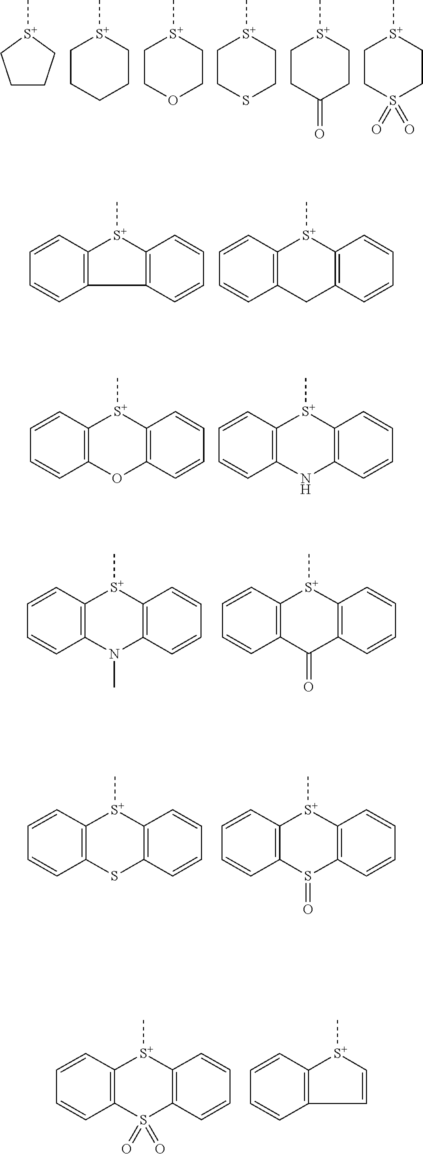

Herein the broken line designates a point of attachment to R^{ct3}.

Examples of the sulfonium cation of formula (Z-1) are shown below, but not limited thereto.

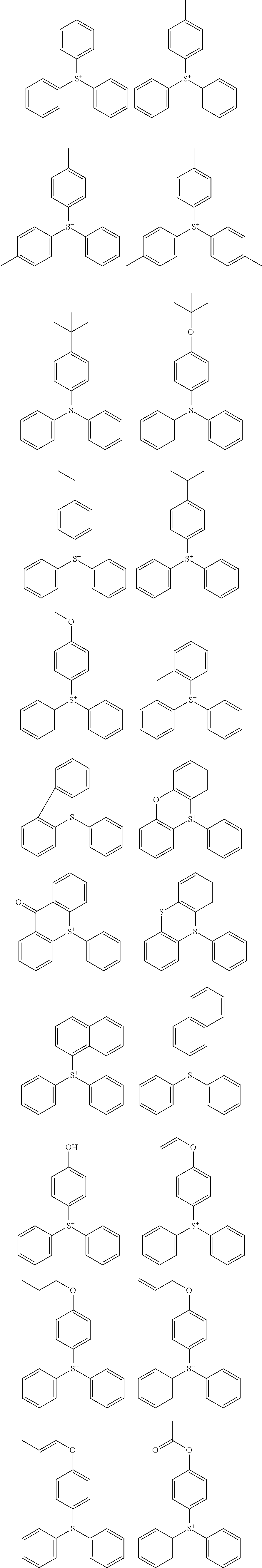

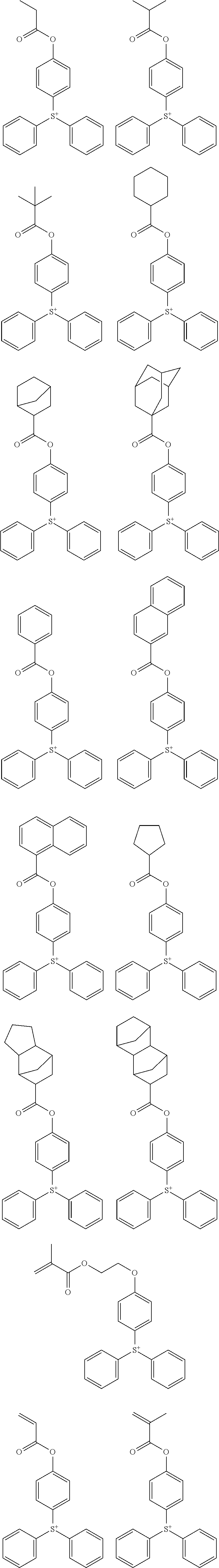

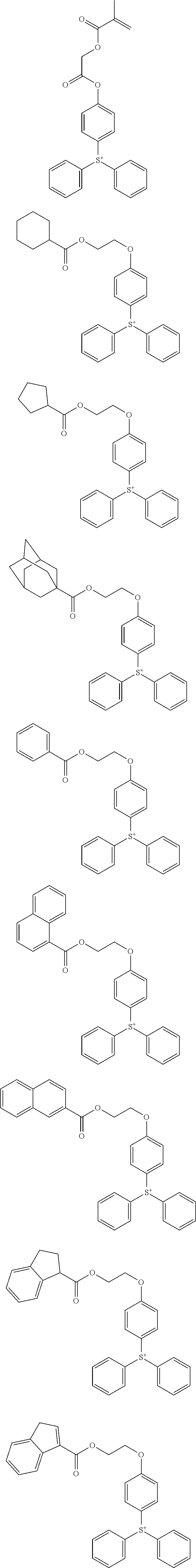

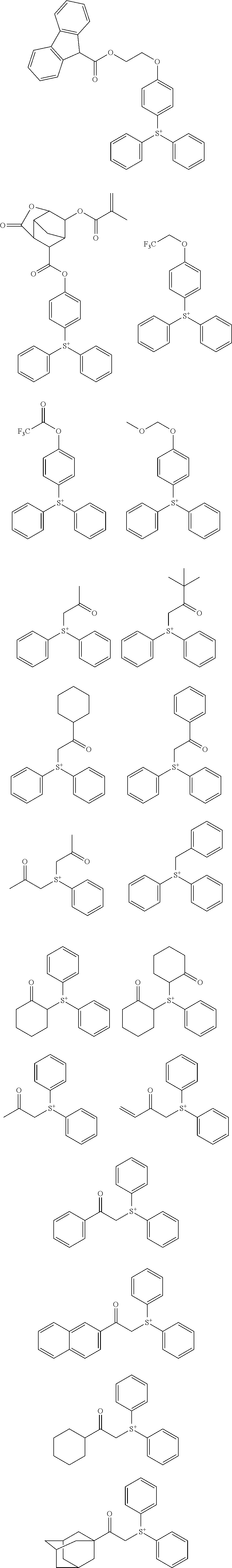

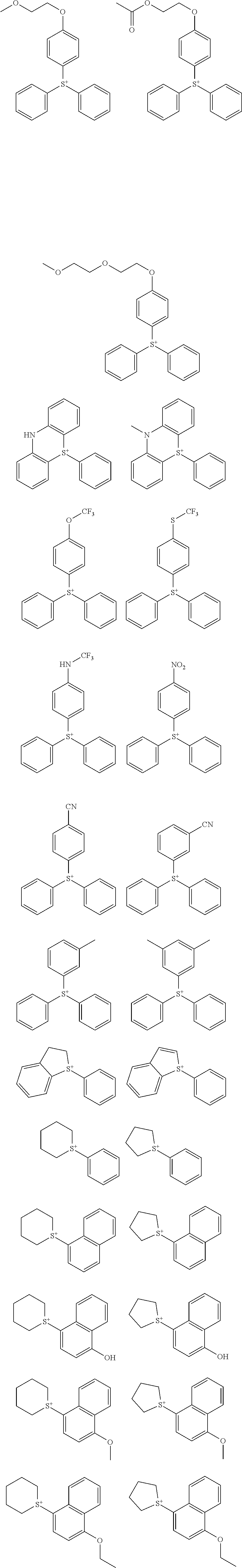

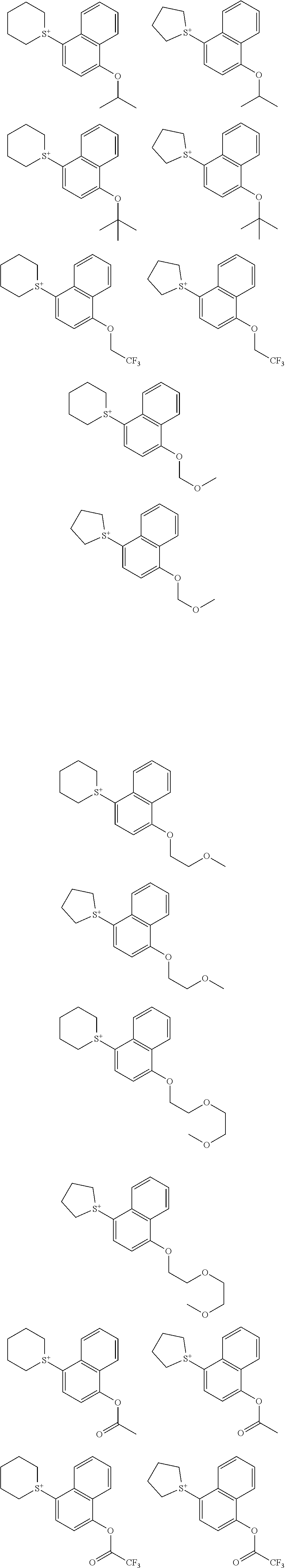

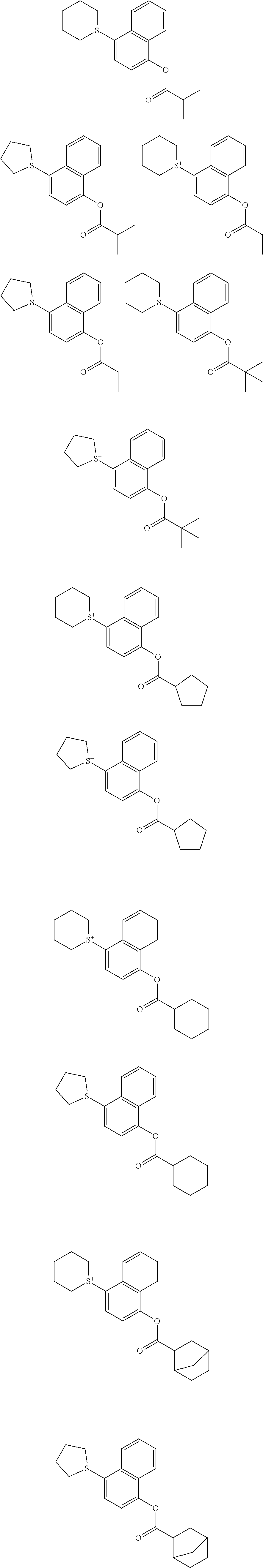

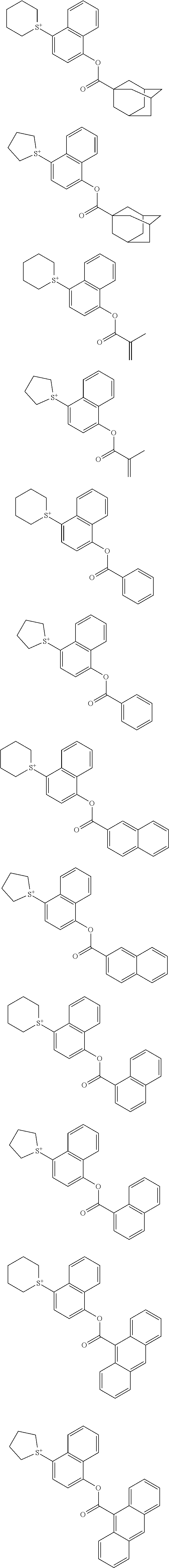

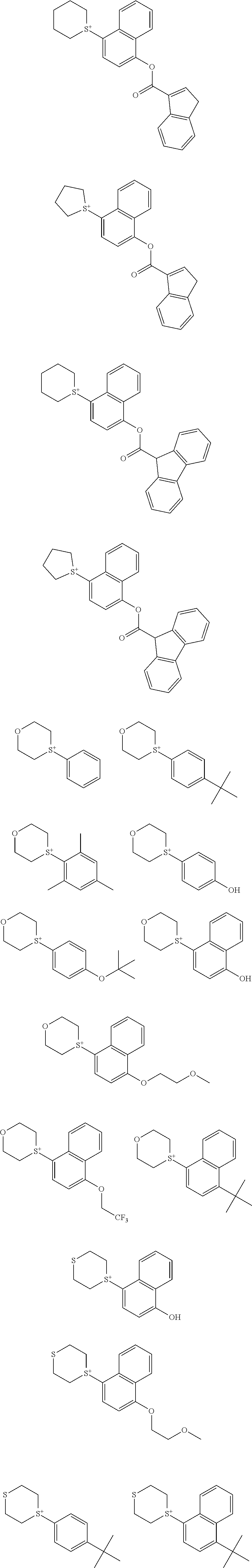

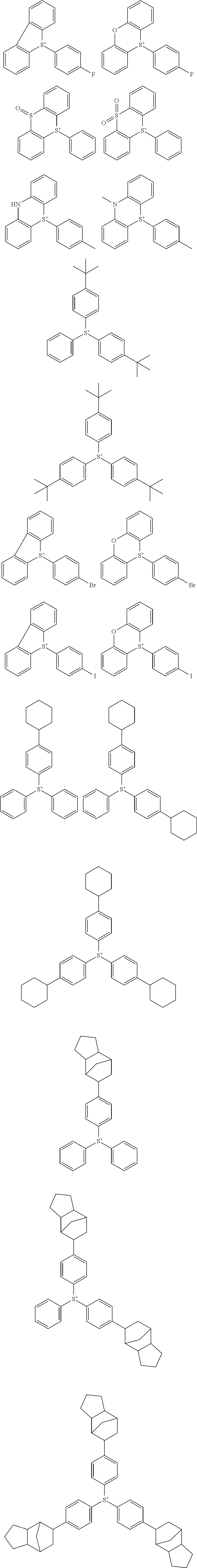

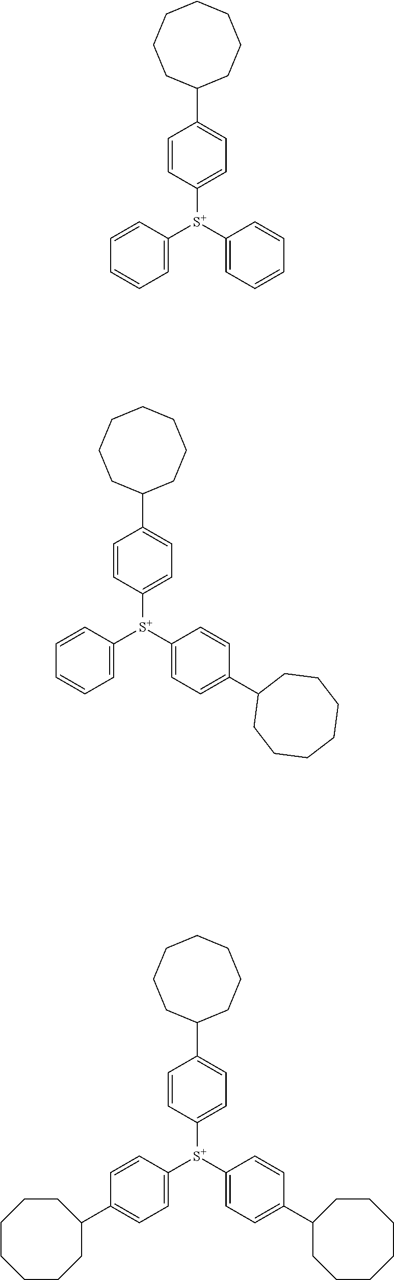 [SMILES: c1ccc([S+](c2ccccc2)c2ccc(C3CCCCCCC3)cc2)cc1 ; c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1]

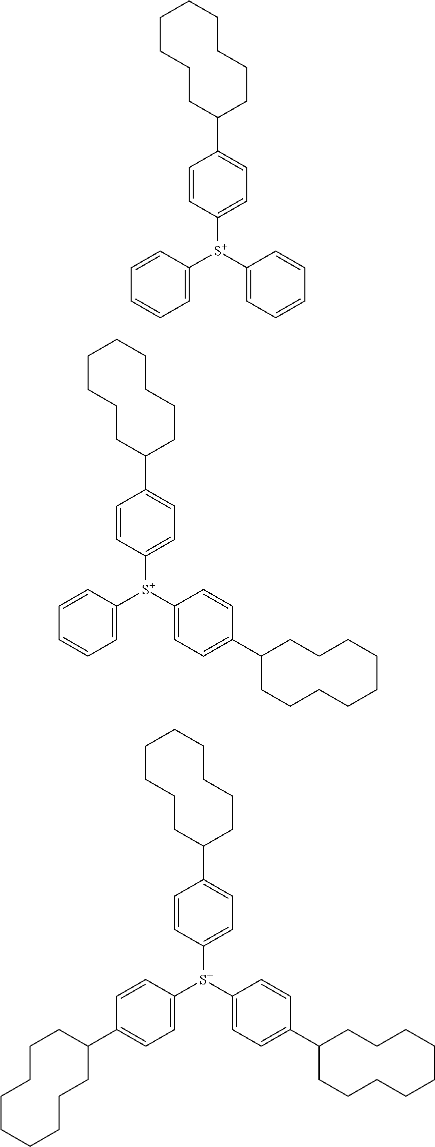 [SMILES: c1ccc([S+](c2ccccc2)c2ccc(C3CCCCCCCCC3)cc2)cc1 ; c1ccc([S+](c2ccc(C3CCCCCCCCC3)cc2)c2ccc(C3CCCCCCCCC3)cc2)cc1 ; c1cc([S+](c2ccc(C3CCCCCCCCC3)cc2)c2ccc(C3CCCCCCCCC3)cc2)ccc1C1CCCCCCCCC1]

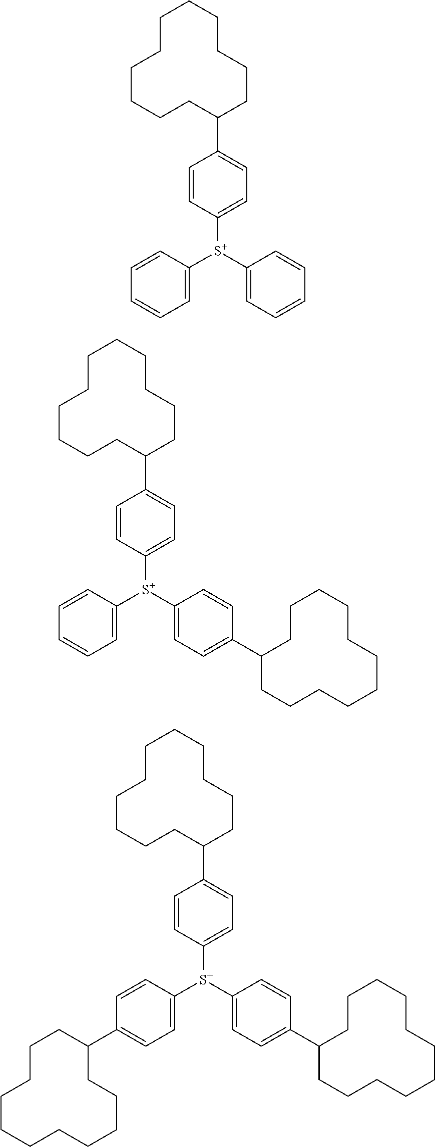 [SMILES: c1ccc([S+](c2ccccc2)c2ccc(C3CCCCCCCCCCC3)cc2)cc1 ; c1ccc([S+](c2ccc(C3CCCCCCCCCCC3)cc2)c2ccc(C3CCCCCCCCCCC3)cc2)cc1 ; c1cc([S+](c2ccc(C3CCCCCCCCCCC3)cc2)c2ccc(C3CCCCCCCCCCC3)cc2)ccc1C1CCCCCCCCCCC1]

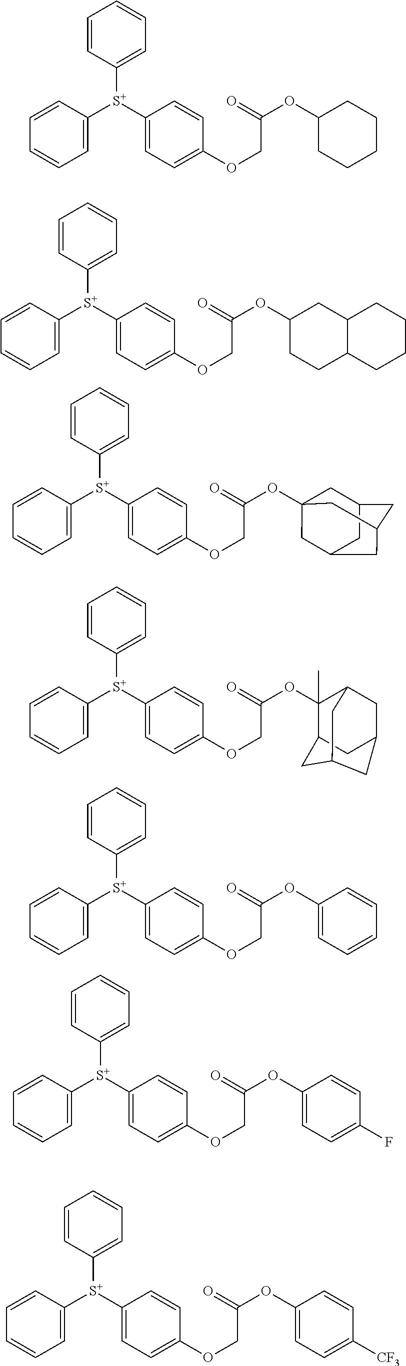

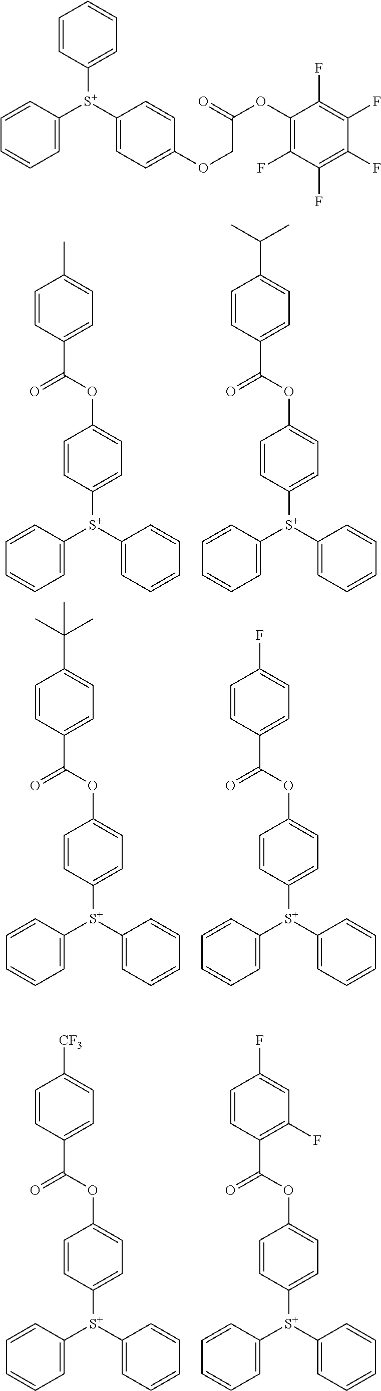

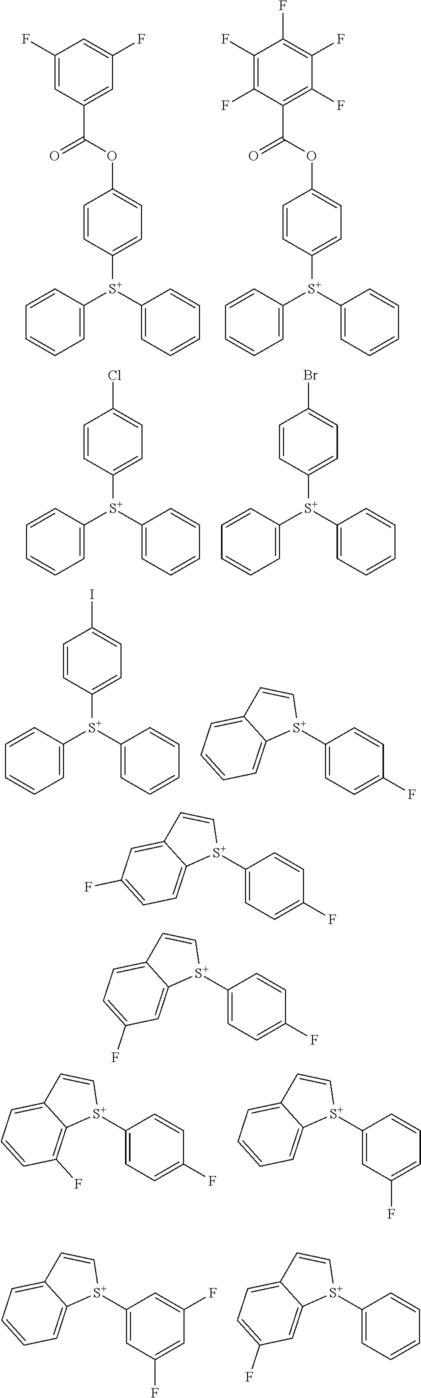

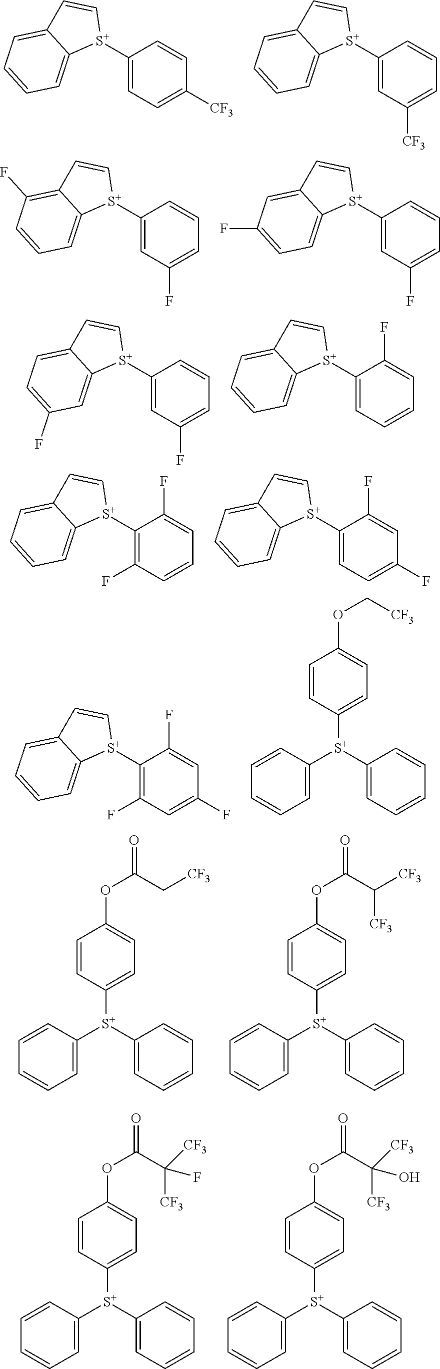

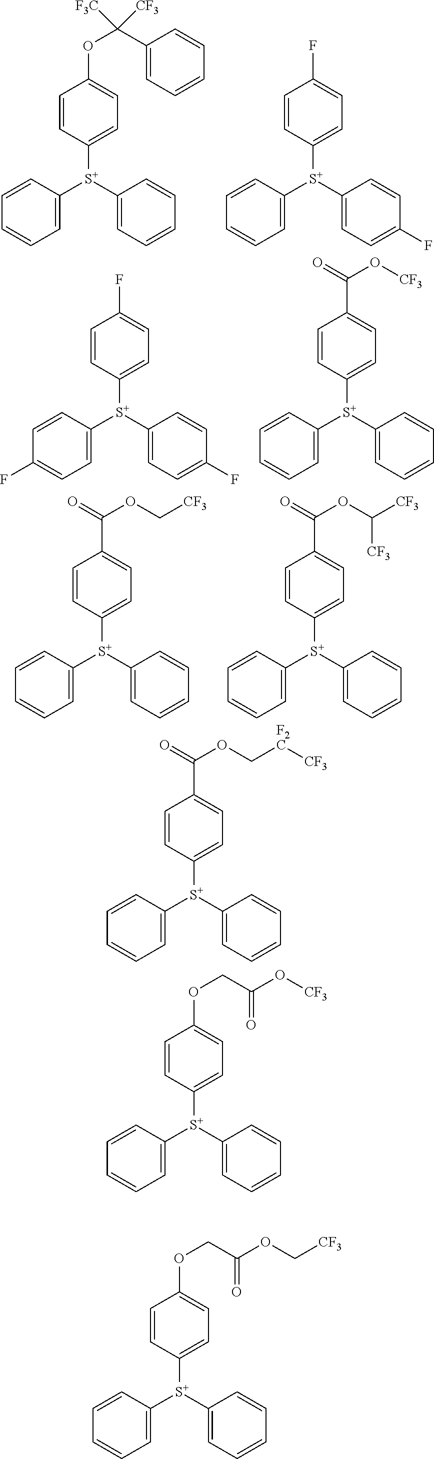

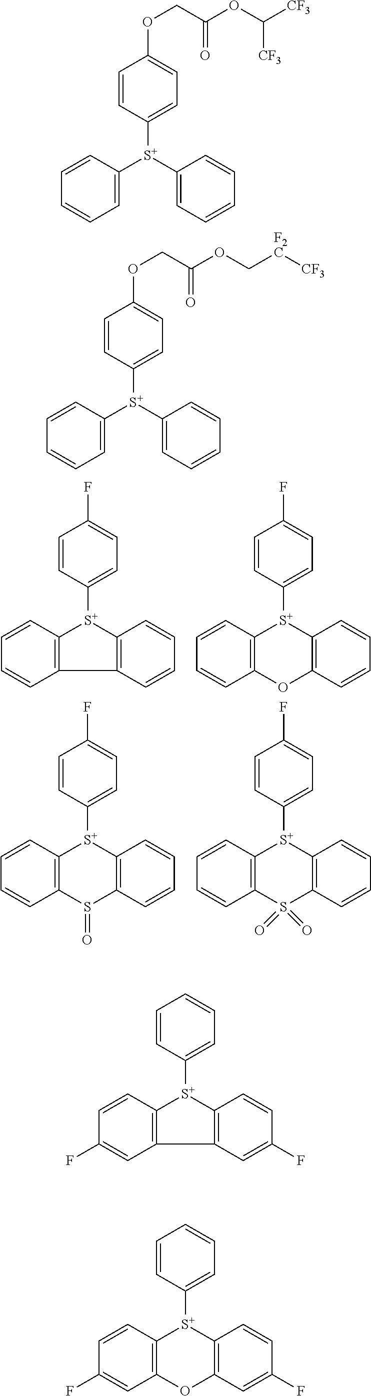

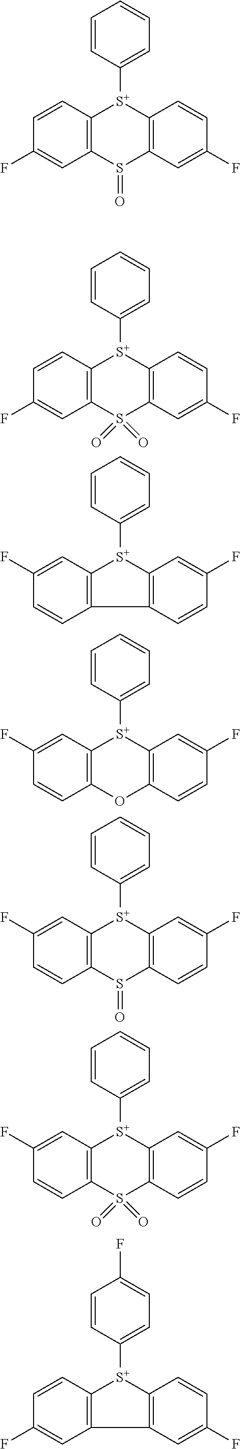

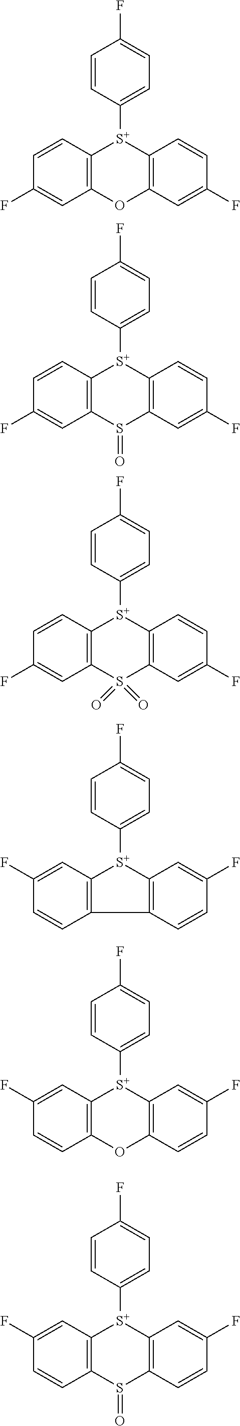 [SMILES: Fc1ccc([S+]2c3ccc(F)cc3Oc3cc(F)ccc32)cc1 ; O=S1c2cc(F)ccc2[S+](c2ccc(F)cc2)c2ccc(F)cc21 ; O=S1(=O)c2cc(F)ccc2[S+](c2ccc(F)cc2)c2ccc(F)cc21 ; Fc1ccc(-[s+]2c3cc(F)ccc3c3ccc(F)cc32)cc1 ; Fc1ccc([S+]2c3cc(F)ccc3Oc3ccc(F)cc32)cc1 ; O=S1c2ccc(F)cc2[S+](c2ccc(F)cc2)c2cc(F)ccc21]

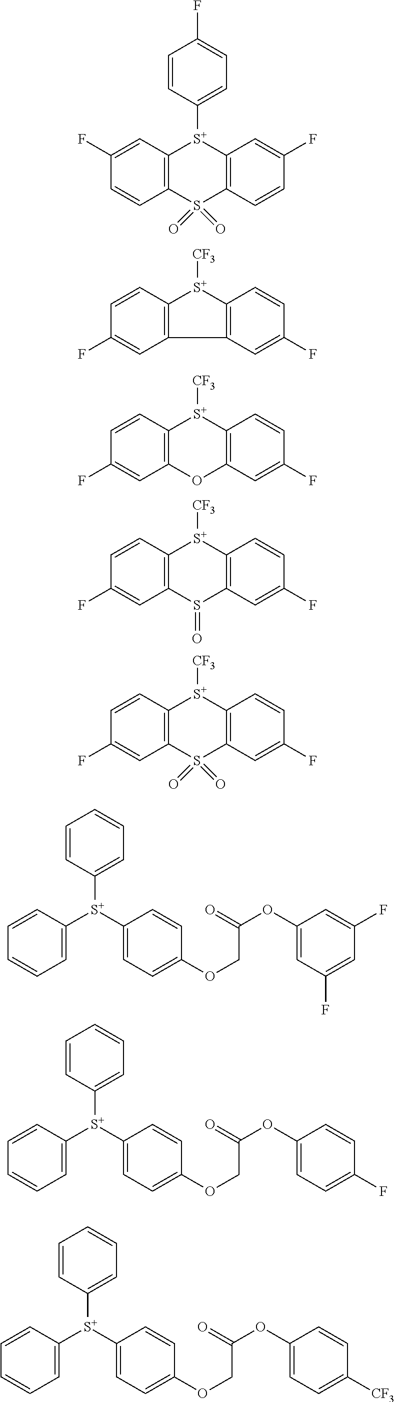

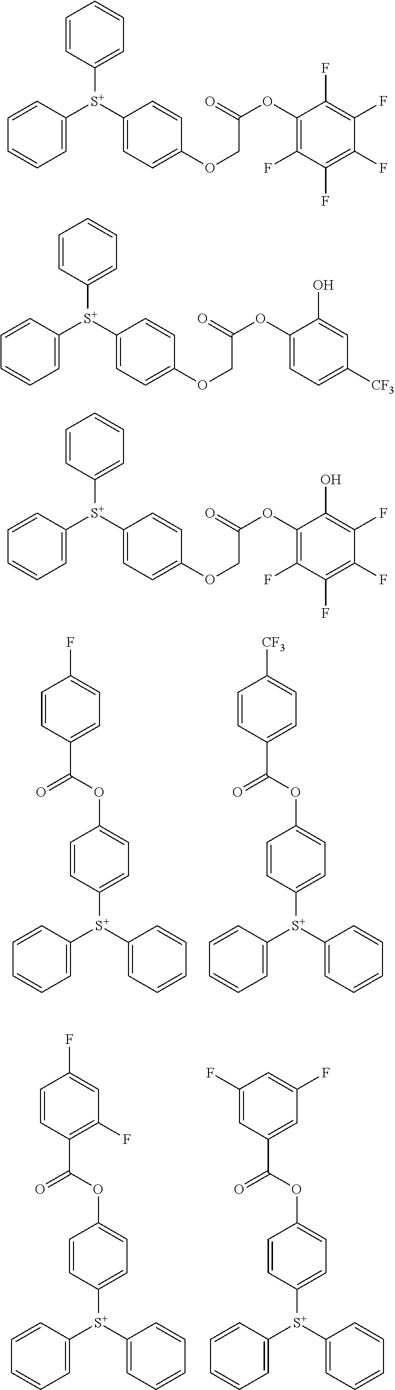

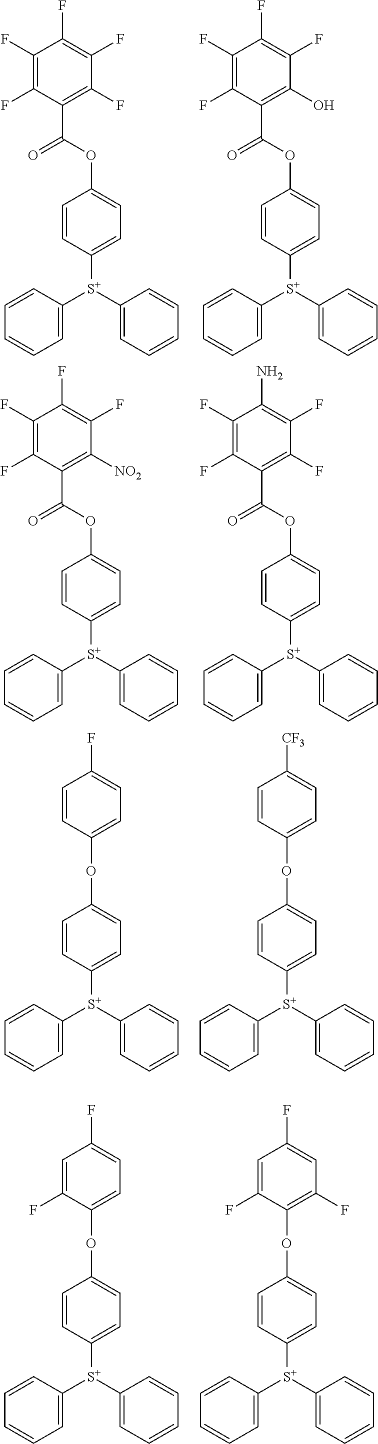

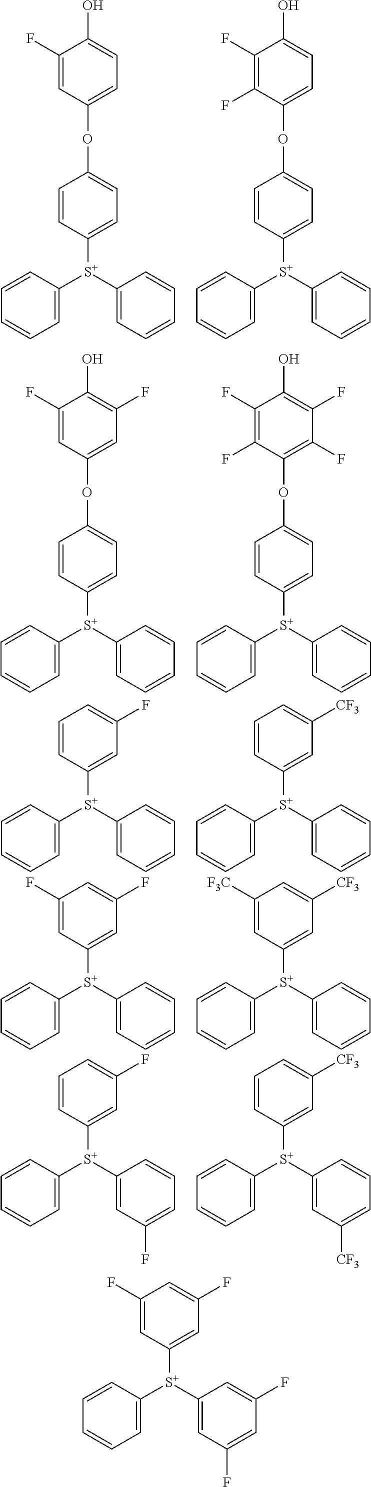

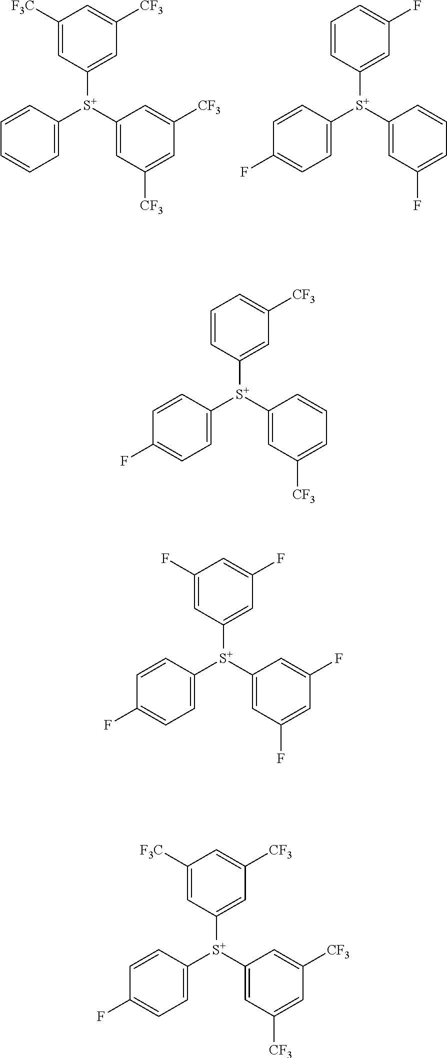 [SMILES: FC(F)(F)c1cc([S+](c2ccccc2)c2cc(C(F)(F)F)cc(C(F)(F)F)c2)cc(C(F)(F)F)c1 ; Fc1ccc([S+](c2cccc(F)c2)c2cccc(F)c2)cc1 ; Fc1ccc([S+](c2cccc(C(F)(F)F)c2)c2cccc(C(F)(F)F)c2)cc1 ; Fc1ccc([S+](c2cc(F)cc(F)c2)c2cc(F)cc(F)c2)cc1 ; Fc1ccc([S+](c2cc(C(F)(F)F)cc(C(F)(F)F)c2)c2cc(C(F)(F)F)cc(C(F)(F)F)c2)cc1]

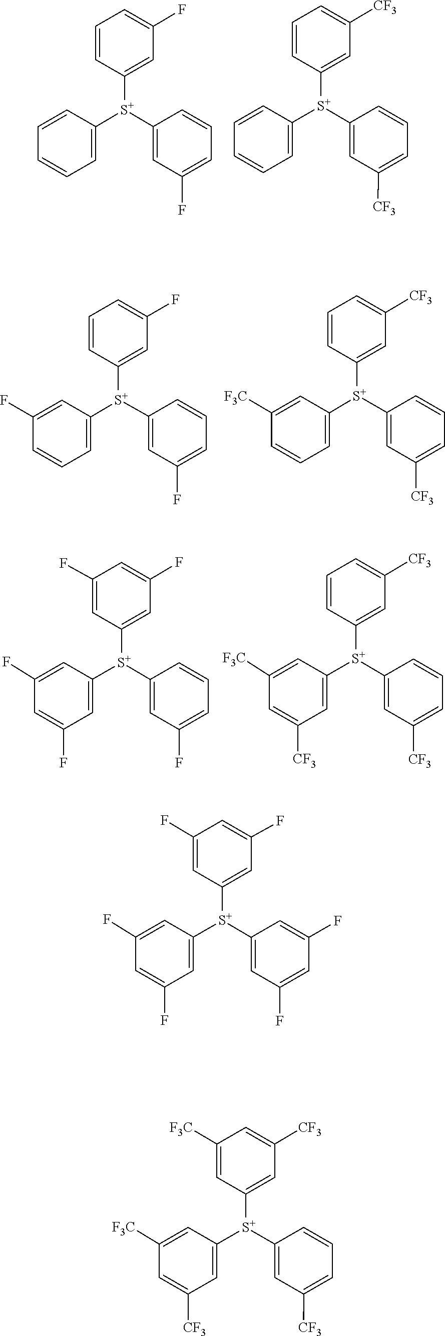

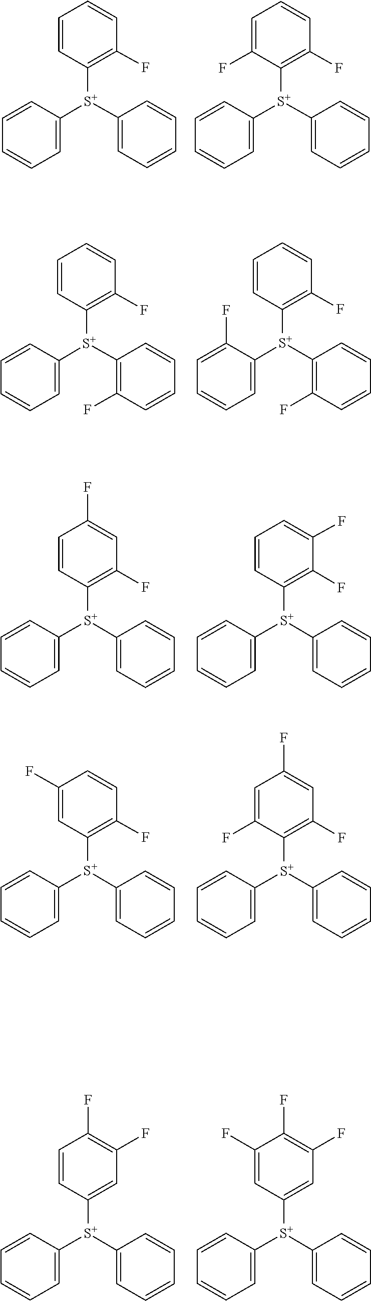

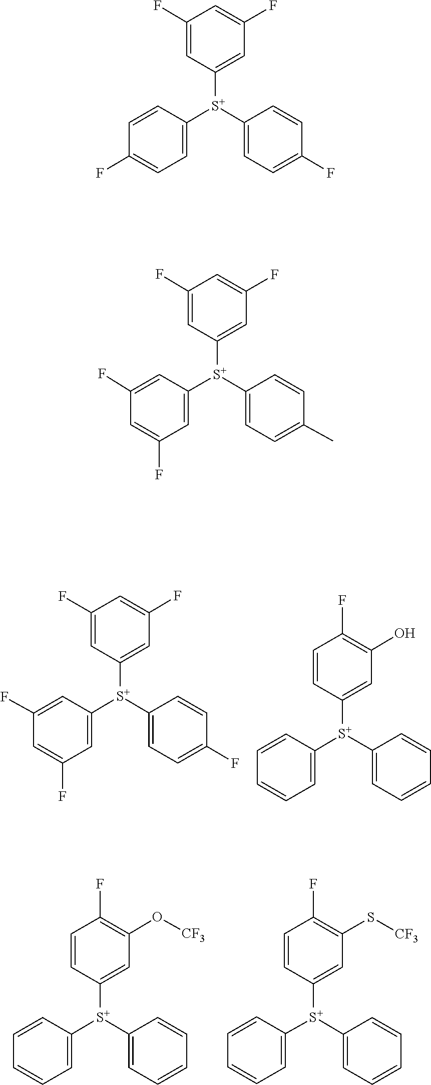 [SMILES: Fc1ccc([S+](c2ccc(F)cc2)c2cc(F)cc(F)c2)cc1 ; Cc1ccc([S+](c2cc(F)cc(F)c2)c2cc(F)cc(F)c2)cc1 ; Fc1ccc([S+](c2cc(F)cc(F)c2)c2cc(F)cc(F)c2)cc1 ; Oc1cc([S+](c2ccccc2)c2ccccc2)ccc1F ; Fc1ccc([S+](c2ccccc2)c2ccccc2)cc1OC(F)(F)F ; Fc1ccc([S+](c2ccccc2)c2ccccc2)cc1SC(F)(F)F]

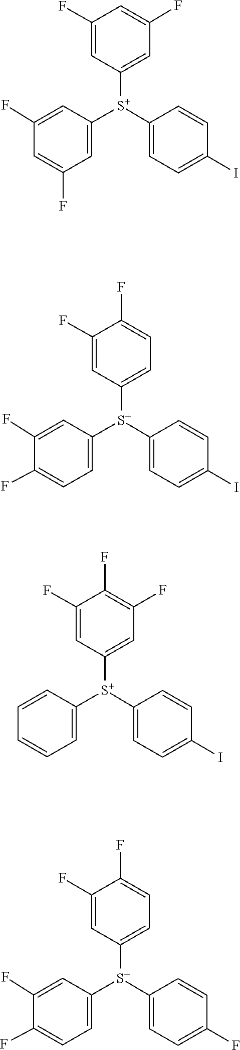 [SMILES: Fc1cc(F)cc([S+](c2ccc(I)cc2)c2cc(F)cc(F)c2)c1 ; Fc1ccc([S+](c2ccc(I)cc2)c2ccc(F)c(F)c2)cc1F ; Fc1cc([S+](c2ccccc2)c2ccc(I)cc2)cc(F)c1F ; Fc1ccc([S+](c2ccc(F)c(F)c2)c2ccc(F)c(F)c2)cc1]

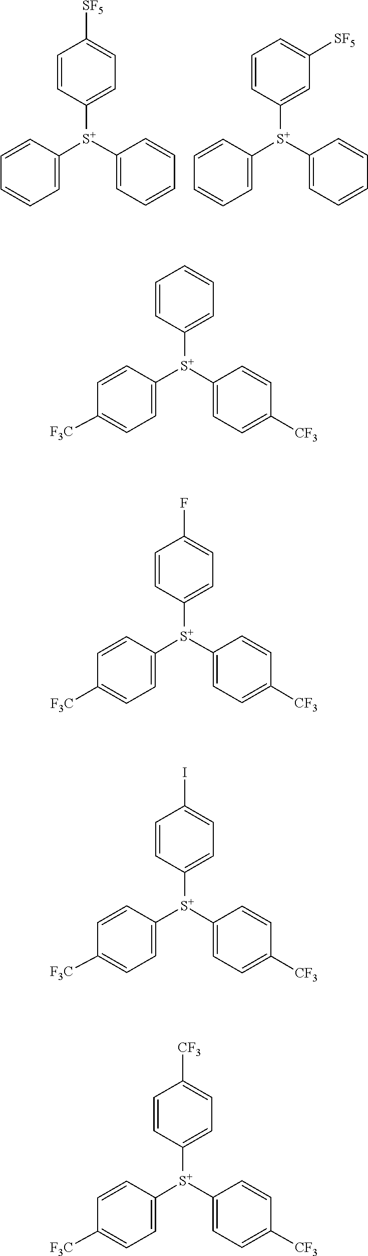

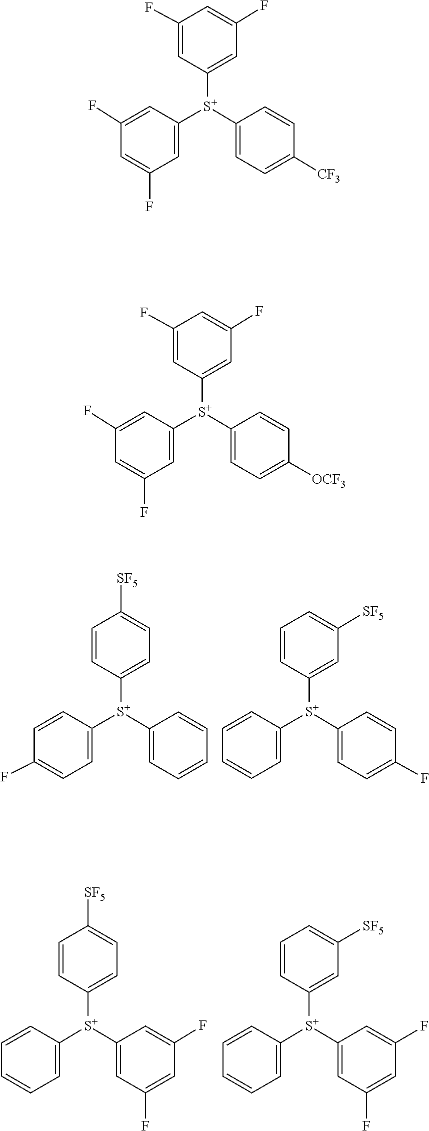

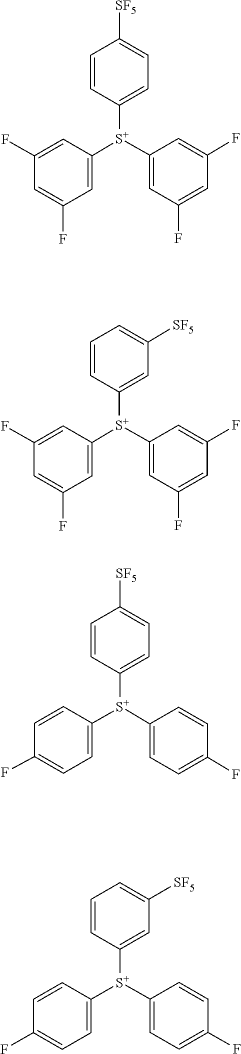

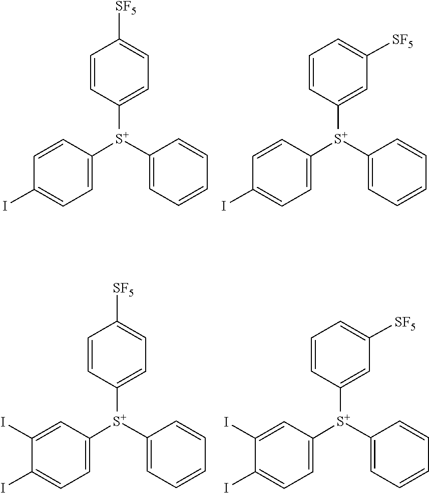

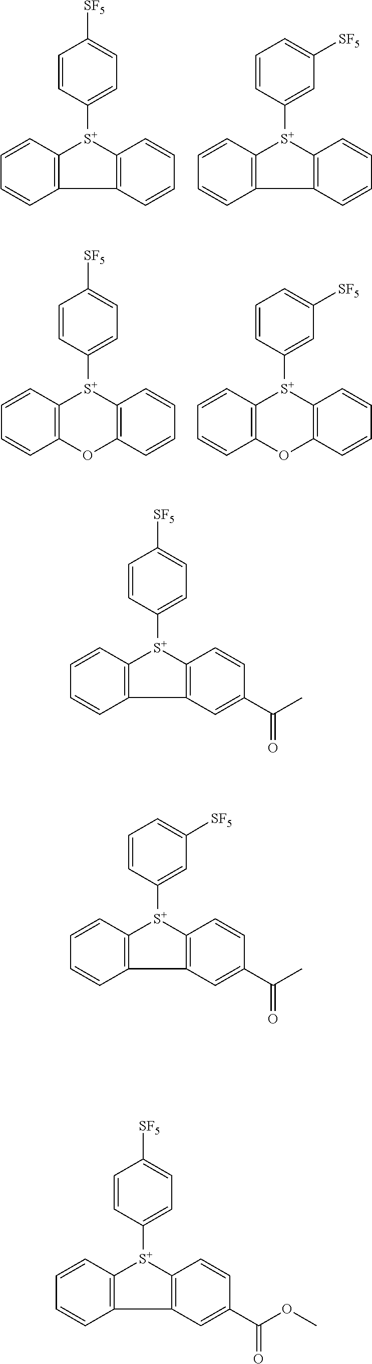

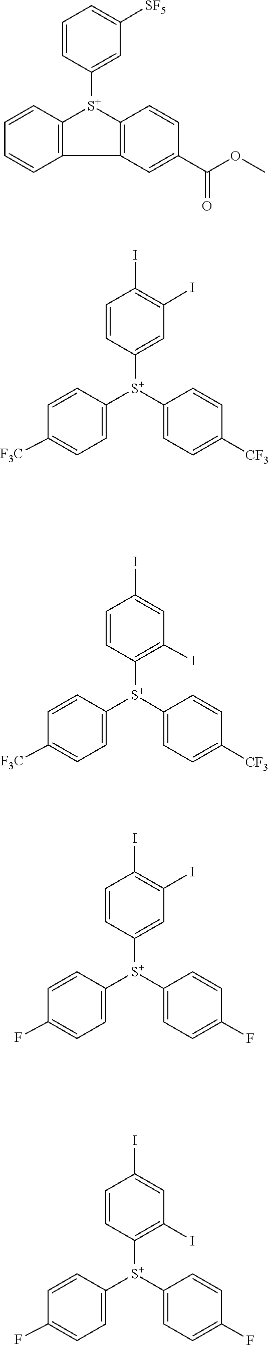

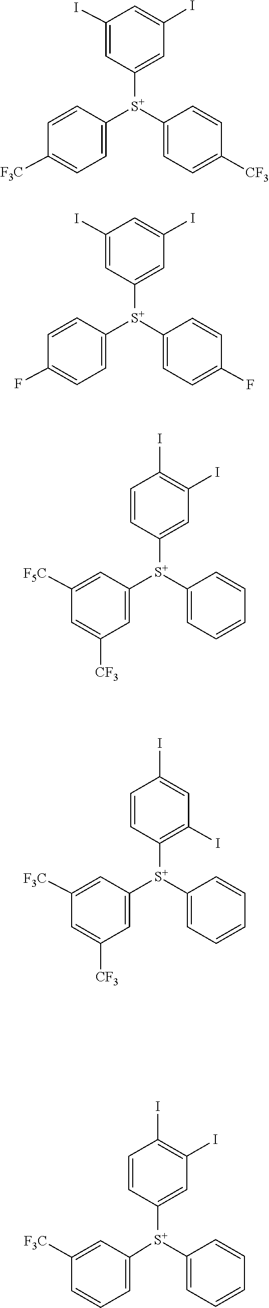

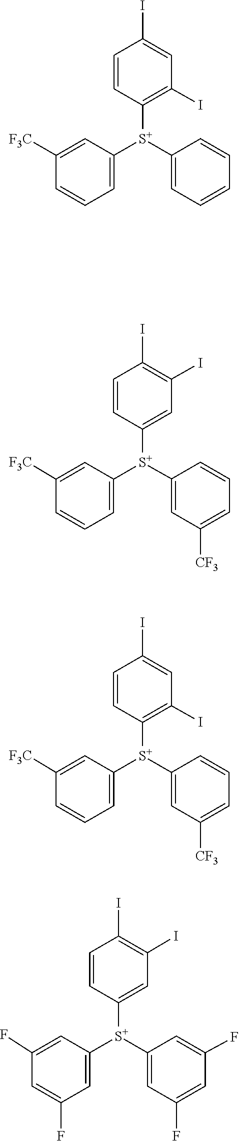 [SMILES: FC(F)(F)c1cccc([S+](c2ccccc2)c2ccc(I)cc2I)c1 ; FC(F)(F)c1cccc([S+](c2cccc(C(F)(F)F)c2)c2ccc(I)c(I)c2)c1 ; FC(F)(F)c1cccc([S+](c2cccc(C(F)(F)F)c2)c2ccc(I)cc2I)c1 ; Fc1cc(F)cc([S+](c2cc(F)cc(F)c2)c2ccc(I)c(I)c2)c1]

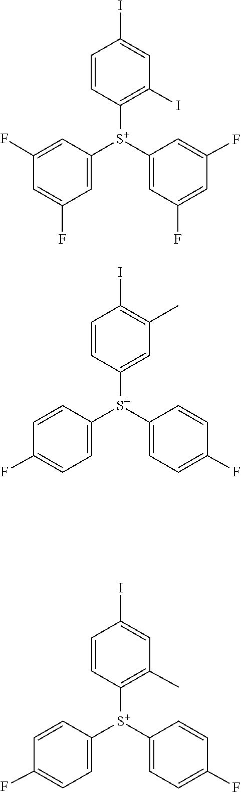 [SMILES: Fc1cc(F)cc([S+](c2cc(F)cc(F)c2)c2ccc(I)cc2I)c1 ; Cc1cc([S+](c2ccc(F)cc2)c2ccc(F)cc2)ccc1I ; Cc1cc(I)ccc1[S+](c1ccc(F)cc1)c1ccc(F)cc1]

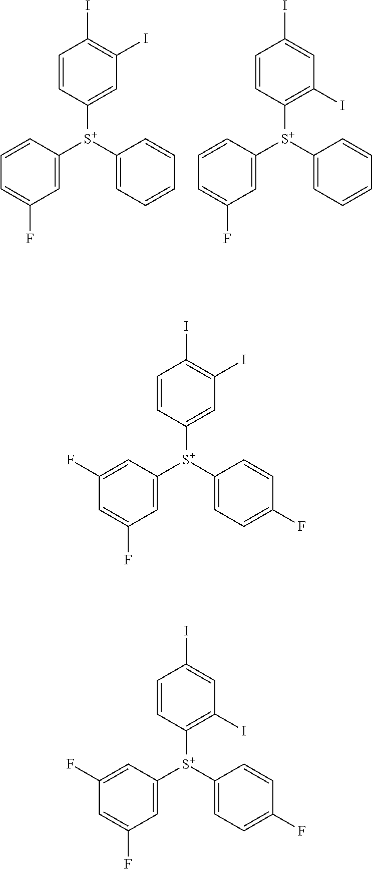 [SMILES: Fc1cccc([S+](c2ccccc2)c2ccc(I)c(I)c2)c1 ; Fc1cccc([S+](c2ccccc2)c2ccc(I)cc2I)c1 ; Fc1ccc([S+](c2cc(F)cc(F)c2)c2ccc(I)c(I)c2)cc1 ; Fc1ccc([S+](c2cc(F)cc(F)c2)c2ccc(I)cc2I)cc1]

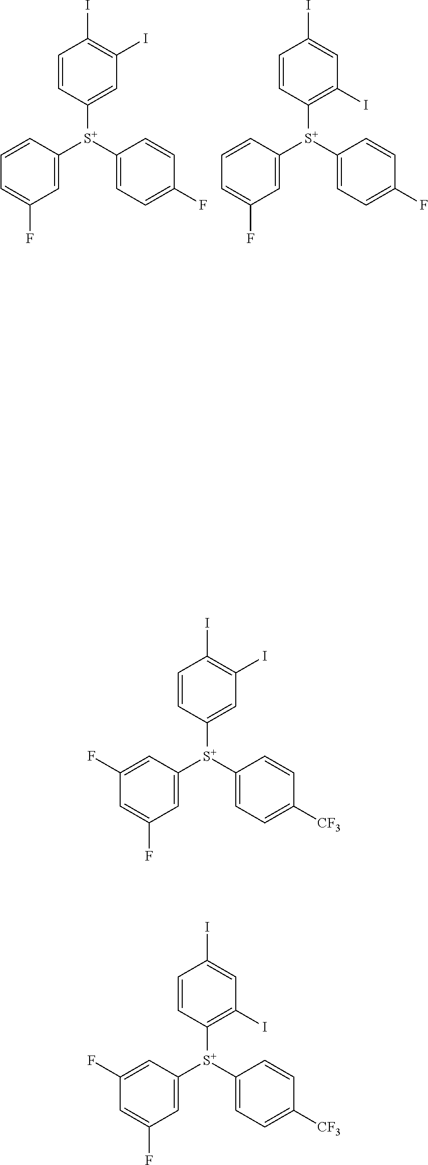 [SMILES: Fc1ccc([S+](c2cccc(F)c2)c2ccc(I)c(I)c2)cc1 ; Fc1ccc([S+](c2cccc(F)c2)c2ccc(I)cc2I)cc1 ; Fc1cc(F)cc([S+](c2ccc(C(F)(F)F)cc2)c2ccc(I)c(I)c2)c1 ; Fc1cc(F)cc([S+](c2ccc(C(F)(F)F)cc2)c2ccc(I)cc2I)c1]

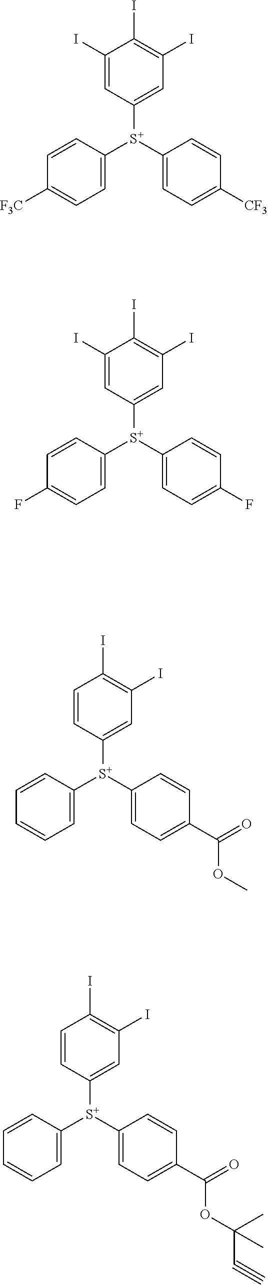 [SMILES: FC(F)(F)c1ccc([S+](c2ccc(C(F)(F)F)cc2)c2cc(I)c(I)c(I)c2)cc1 ; Fc1ccc([S+](c2ccc(F)cc2)c2cc(I)c(I)c(I)c2)cc1 ; COC(=O)c1ccc([S+](c2ccccc2)c2ccc(I)c(I)c2)cc1 ; C#CC(C)(C)OC(=O)c1ccc([S+](c2ccccc2)c2ccc(I)c(I)c2)cc1]

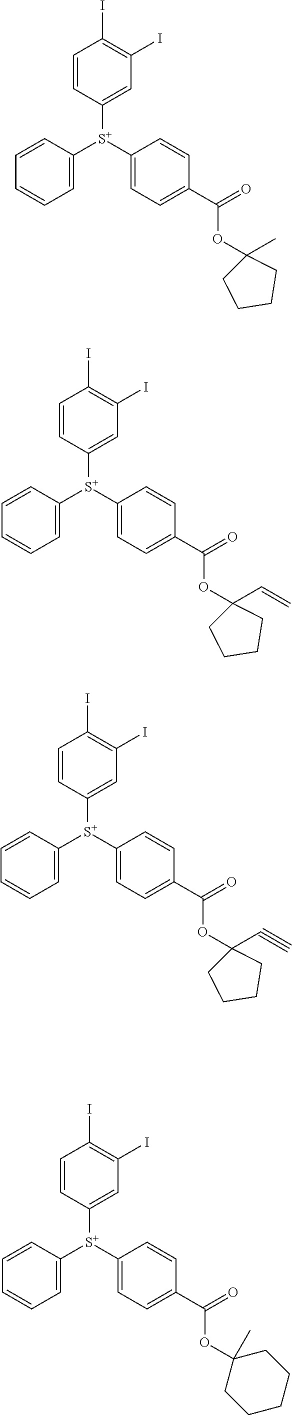 [SMILES: CC1(OC(=O)c2ccc([S+](c3ccccc3)c3ccc(I)c(I)c3)cc2)CCCC1 ; C=CC1(OC(=O)c2ccc([S+](c3ccccc3)c3ccc(I)c(I)c3)cc2)CCCC1 ; C#CC1(OC(=O)c2ccc([S+](c3ccccc3)c3ccc(I)c(I)c3)cc2)CCCC1 ; CC1(OC(=O)c2ccc([S+](c3ccccc3)c3ccc(I)c(I)c3)cc2)CCCCC1]

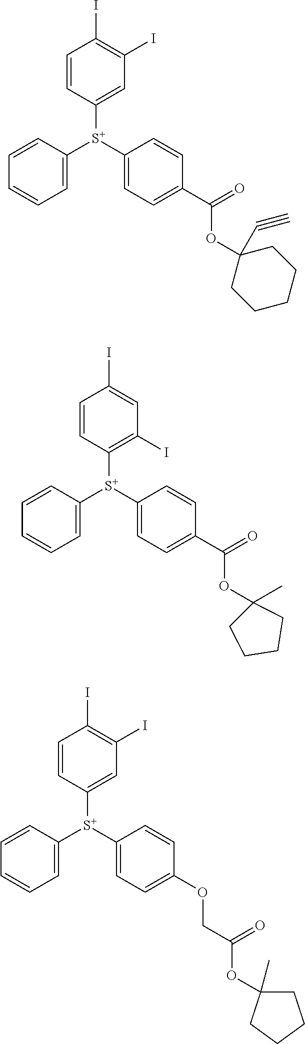 [SMILES: C#CC1(OC(=O)c2ccc([S+](c3ccccc3)c3ccc(I)c(I)c3)cc2)CCCCC1 ; CC1(OC(=O)c2ccc([S+](c3ccccc3)c3ccc(I)cc3I)cc2)CCCC1 ; CC1(OC(=O)COc2ccc([S+](c3ccccc3)c3ccc(I)c(I)c3)cc2)CCCC1]

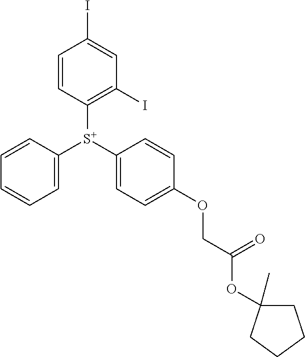 [SMILES: CC1(OC(=O)COc2ccc([S+](c3ccccc3)c3ccc(I)cc3I)cc2)CCCC1]

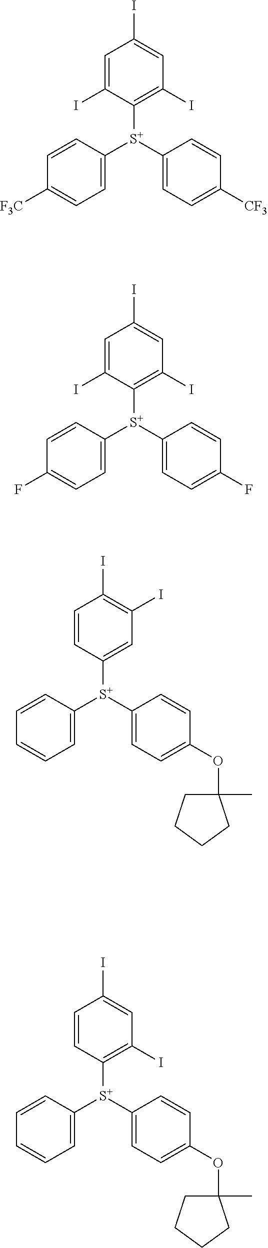 [SMILES: FC(F)(F)c1ccc([S+](c2ccc(C(F)(F)F)cc2)c2c(I)cc(I)cc2I)cc1 ; Fc1ccc([S+](c2ccc(F)cc2)c2c(I)cc(I)cc2I)cc1 ; CC1(Oc2ccc([S+](c3ccccc3)c3ccc(I)c(I)c3)cc2)CCCC1 ; CC1(Oc2ccc([S+](c3ccccc3)c3ccc(I)cc3I)cc2)CCCC1]

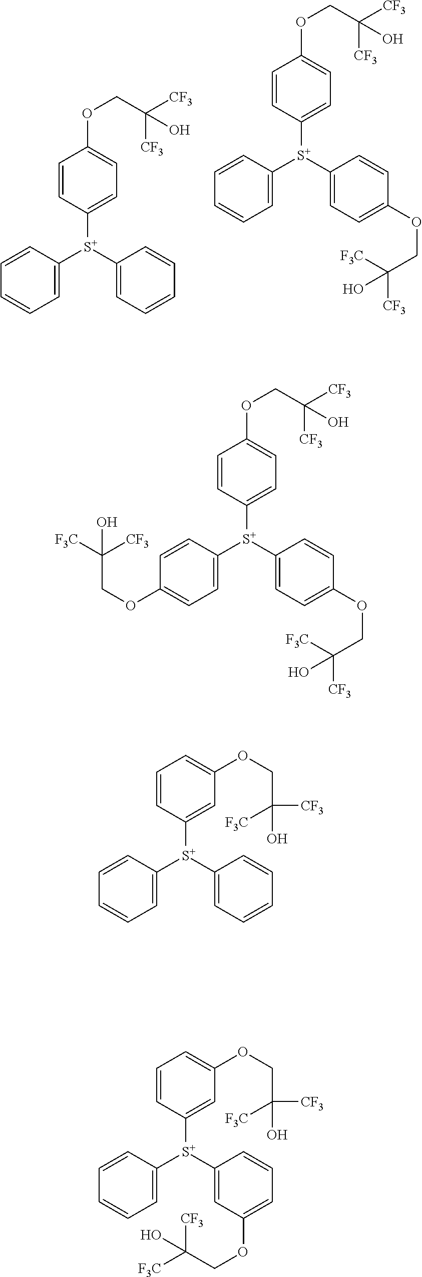 [SMILES: OC(COc1ccc([S+](c2ccccc2)c2ccccc2)cc1)(C(F)(F)F)C(F)(F)F ; OC(COc1ccc([S+](c2ccccc2)c2ccc(OCC(O)(C(F)(F)F)C(F)(F)F)cc2)cc1)(C(F)(F)F)C(F)(F)F ; OC(COc1ccc([S+](c2ccc(OCC(O)(C(F)(F)F)C(F)(F)F)cc2)c2ccc(OCC(O)(C(F)(F)F)C(F)(F)F)cc2)cc1)(C(F)(F)F)C(F)(F)F ; OC(COc1cccc([S+](c2ccccc2)c2ccccc2)c1)(C(F)(F)F)C(F)(F)F ; OC(COc1cccc([S+](c2ccccc2)c2cccc(OCC(O)(C(F)(F)F)C(F)(F)F)c2)c1)(C(F)(F)F)C(F)(F)F]

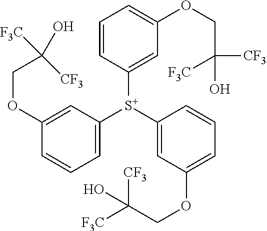 [SMILES: OC(COc1cccc([S+](c2cccc(OCC(O)(C(F)(F)F)C(F)(F)F)c2)c2cccc(OCC(O)(C(F)(F)F)C(F)(F)F)c2)c1)(C(F)(F)F)C(F)(F)F]

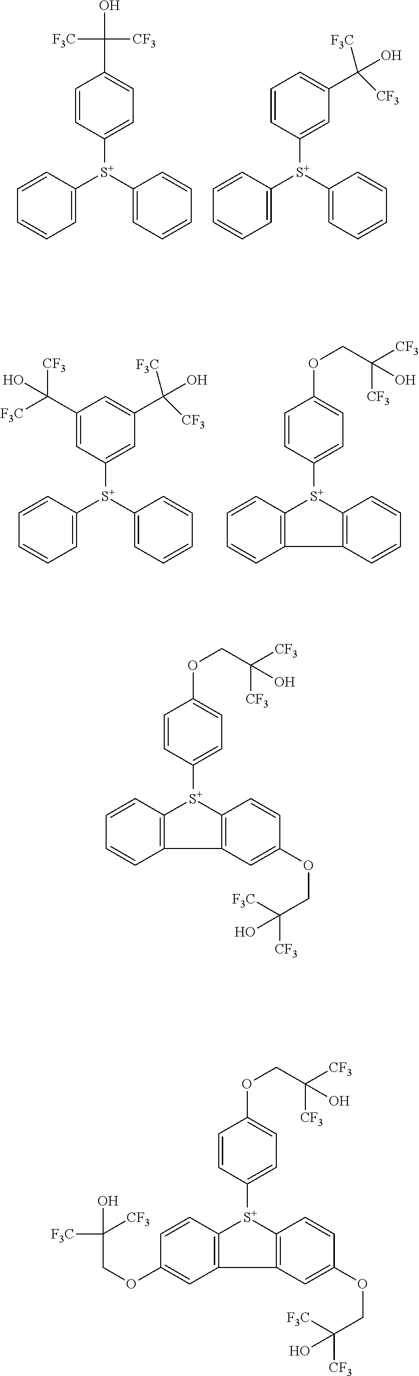 [SMILES: OC(c1ccc([S+](c2ccccc2)c2ccccc2)cc1)(C(F)(F)F)C(F)(F)F ; OC(c1cccc([S+](c2ccccc2)c2ccccc2)c1)(C(F)(F)F)C(F)(F)F ; OC(c1cc([S+](c2ccccc2)c2ccccc2)cc(C(O)(C(F)(F)F)C(F)(F)F)c1)(C(F)(F)F)C(F)(F)F ; OC(COc1ccc(-[s+]2c3ccccc3c3ccccc32)cc1)(C(F)(F)F)C(F)(F)F ; OC(COc1ccc(-[s+]2c3ccccc3c3cc(OCC(O)(C(F)(F)F)C(F)(F)F)ccc32)cc1)(C(F)(F)F)C(F)(F)F ; OC(COc1ccc(-[s+]2c3ccc(OCC(O)(C(F)(F)F)C(F)(F)F)cc3c3cc(OCC(O)(C(F)(F)F)C(F)(F)F)ccc32)cc1)(C(F)(F)F)C(F)(F)F]

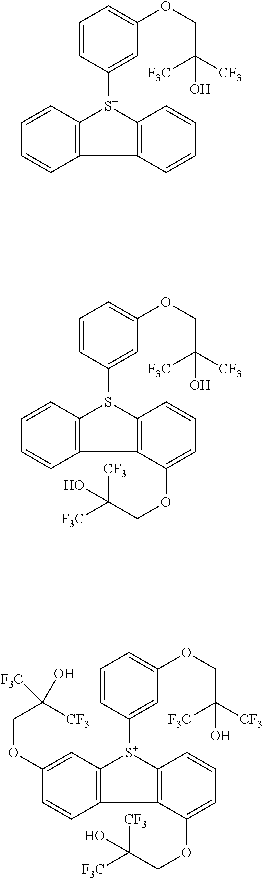 [SMILES: OC(COc1cccc(-[s+]2c3ccccc3c3ccccc32)c1)(C(F)(F)F)C(F)(F)F ; OC(COc1cccc(-[s+]2c3ccccc3c3c(OCC(O)(C(F)(F)F)C(F)(F)F)cccc32)c1)(C(F)(F)F)C(F)(F)F ; OC(COc1cccc(-[s+]2c3cc(OCC(O)(C(F)(F)F)C(F)(F)F)ccc3c3c(OCC(O)(C(F)(F)F)C(F)(F)F)cccc32)c1)(C(F)(F)F)C(F)(F)F]

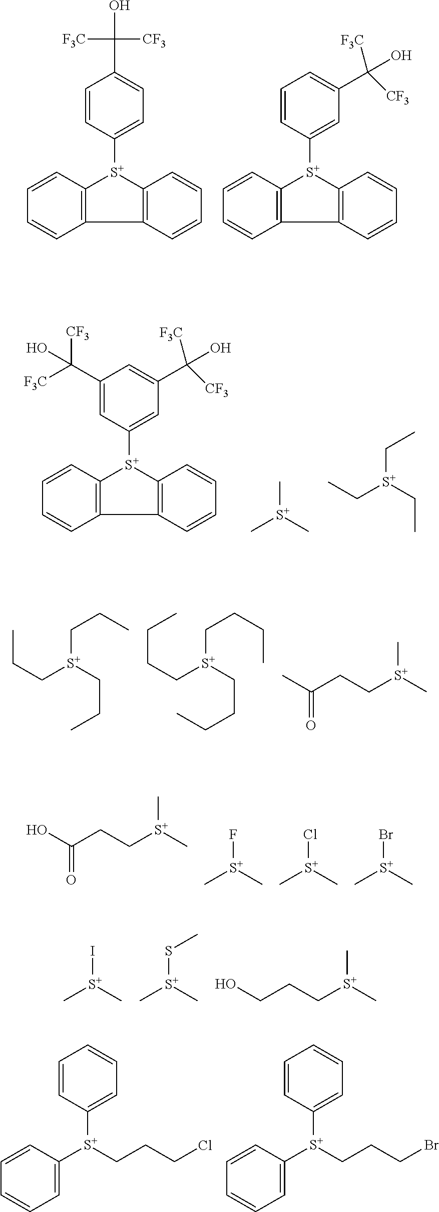

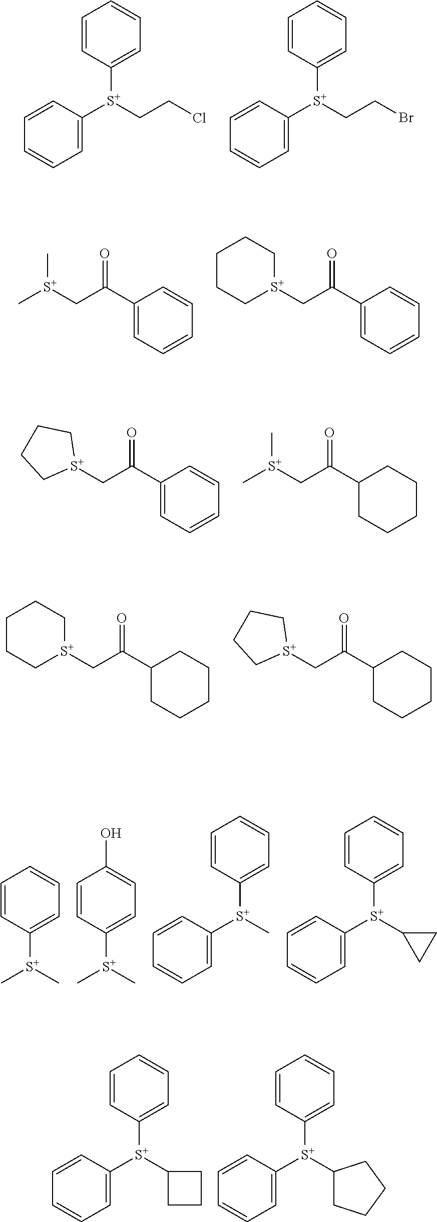

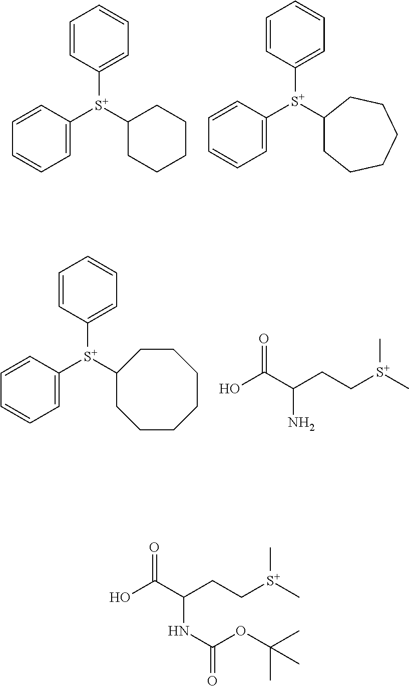

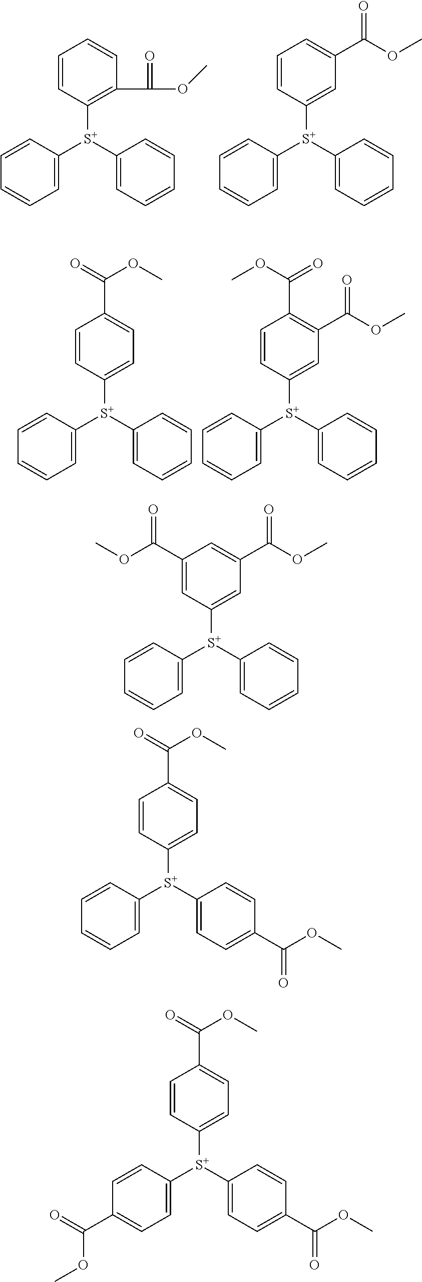

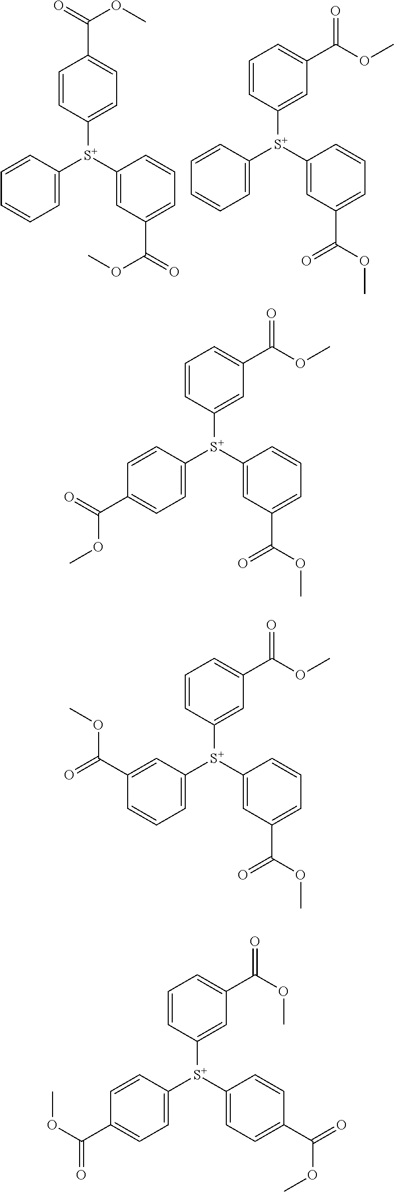 [SMILES: COC(=O)c1ccc([S+](c2ccccc2)c2cccc(C(=O)OC)c2)cc1 ; COC(=O)c1cccc([S+](c2ccccc2)c2cccc(C(=O)OC)c2)c1 ; COC(=O)c1ccc([S+](c2cccc(C(=O)OC)c2)c2cccc(C(=O)OC)c2)cc1 ; COC(=O)c1cccc([S+](c2cccc(C(=O)OC)c2)c2cccc(C(=O)OC)c2)c1 ; COC(=O)c1ccc([S+](c2ccc(C(=O)OC)cc2)c2cccc(C(=O)OC)c2)cc1]

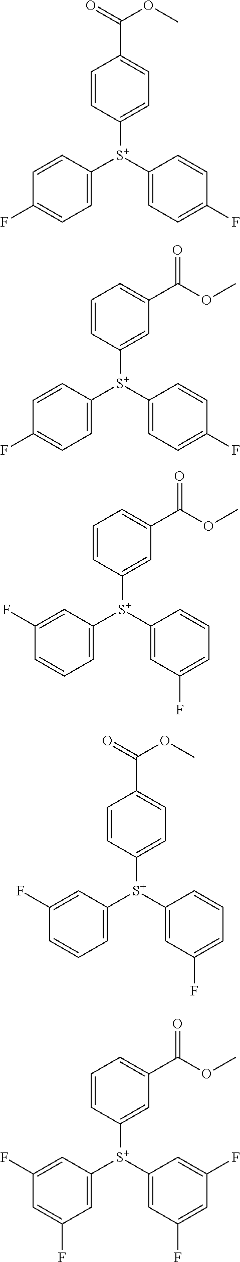 [SMILES: COC(=O)c1ccc([S+](c2ccc(F)cc2)c2ccc(F)cc2)cc1 ; COC(=O)c1cccc([S+](c2ccc(F)cc2)c2ccc(F)cc2)c1 ; COC(=O)c1cccc([S+](c2cccc(F)c2)c2cccc(F)c2)c1 ; COC(=O)c1ccc([S+](c2cccc(F)c2)c2cccc(F)c2)cc1 ; COC(=O)c1cccc([S+](c2cc(F)cc(F)c2)c2cc(F)cc(F)c2)c1]

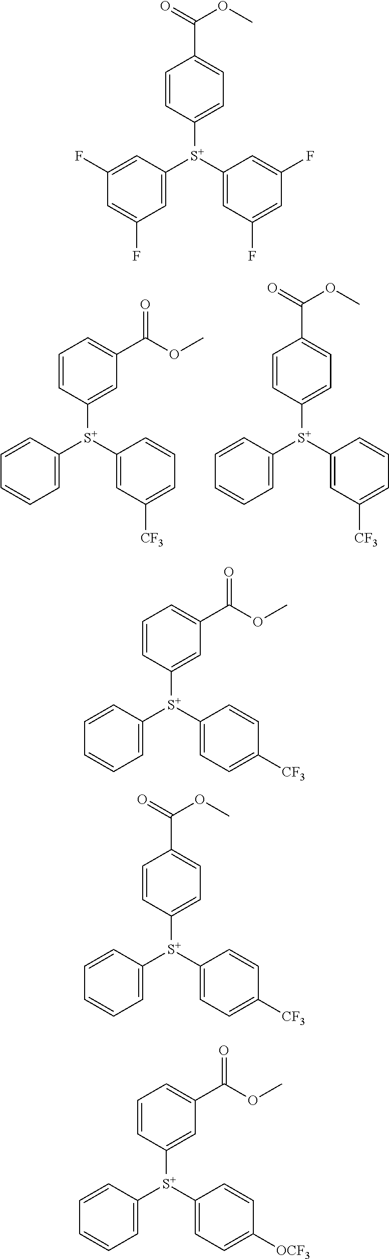 [SMILES: COC(=O)c1ccc([S+](c2cc(F)cc(F)c2)c2cc(F)cc(F)c2)cc1 ; COC(=O)c1cccc([S+](c2ccccc2)c2cccc(C(F)(F)F)c2)c1 ; COC(=O)c1ccc([S+](c2ccccc2)c2cccc(C(F)(F)F)c2)cc1 ; COC(=O)c1cccc([S+](c2ccccc2)c2ccc(C(F)(F)F)cc2)c1 ; COC(=O)c1ccc([S+](c2ccccc2)c2ccc(C(F)(F)F)cc2)cc1 ; COC(=O)c1cccc([S+](c2ccccc2)c2ccc(OC(F)(F)F)cc2)c1]

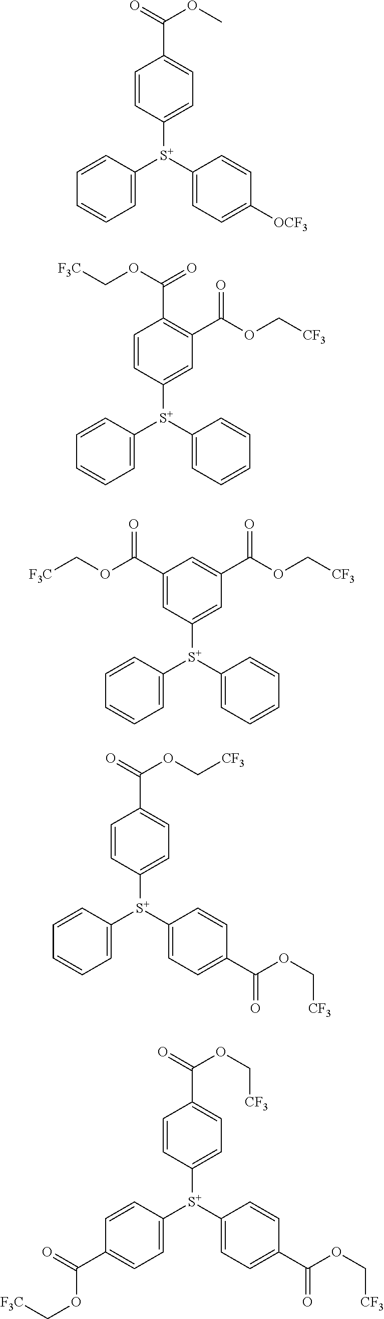 [SMILES: COC(=O)c1ccc([S+](c2ccccc2)c2ccc(OC(F)(F)F)cc2)cc1 ; O=C(OCC(F)(F)F)c1ccc([S+](c2ccccc2)c2ccccc2)cc1C(=O)OCC(F)(F)F ; O=C(OCC(F)(F)F)c1cc(C(=O)OCC(F)(F)F)cc([S+](c2ccccc2)c2ccccc2)c1 ; O=C(OCC(F)(F)F)c1ccc([S+](c2ccccc2)c2ccc(C(=O)OCC(F)(F)F)cc2)cc1 ; O=C(OCC(F)(F)F)c1ccc([S+](c2ccc(C(=O)OCC(F)(F)F)cc2)c2ccc(C(=O)OCC(F)(F)F)cc2)cc1]

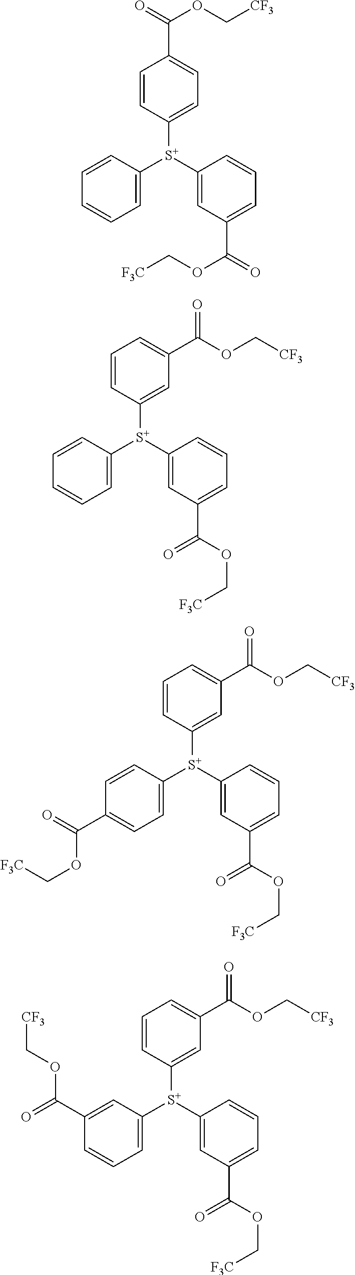 [SMILES: O=C(OCC(F)(F)F)c1ccc([S+](c2ccccc2)c2cccc(C(=O)OCC(F)(F)F)c2)cc1 ; O=C(OCC(F)(F)F)c1cccc([S+](c2ccccc2)c2cccc(C(=O)OCC(F)(F)F)c2)c1 ; O=C(OCC(F)(F)F)c1ccc([S+](c2cccc(C(=O)OCC(F)(F)F)c2)c2cccc(C(=O)OCC(F)(F)F)c2)cc1 ; O=C(OCC(F)(F)F)c1cccc([S+](c2cccc(C(=O)OCC(F)(F)F)c2)c2cccc(C(=O)OCC(F)(F)F)c2)c1]

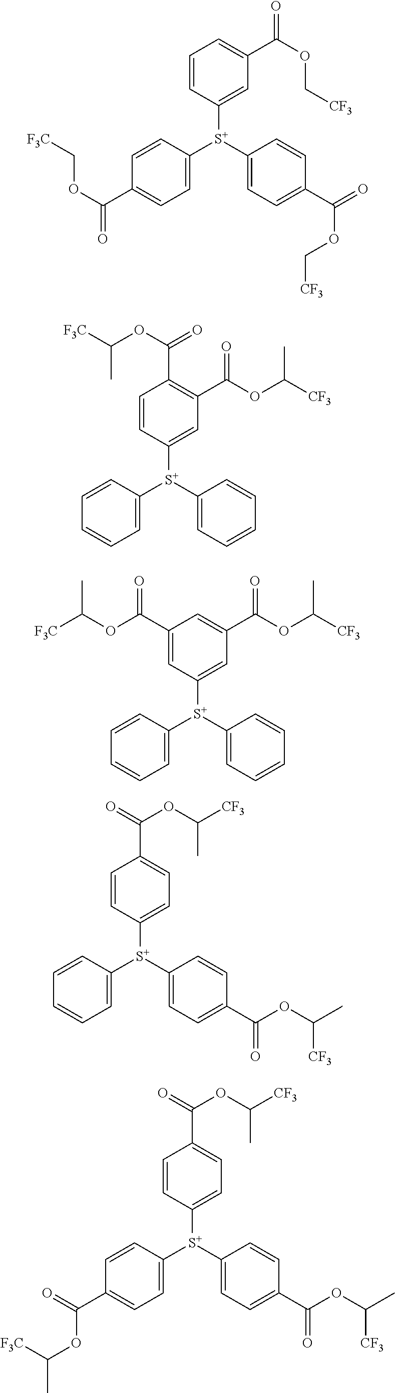 [SMILES: O=C(OCC(F)(F)F)c1ccc([S+](c2ccc(C(=O)OCC(F)(F)F)cc2)c2cccc(C(=O)OCC(F)(F)F)c2)cc1 ; CC(OC(=O)c1ccc([S+](c2ccccc2)c2ccccc2)cc1C(=O)OC(C)C(F)(F)F)C(F)(F)F ; CC(OC(=O)c1cc(C(=O)OC(C)C(F)(F)F)cc([S+](c2ccccc2)c2ccccc2)c1)C(F)(F)F ; CC(OC(=O)c1ccc([S+](c2ccccc2)c2ccc(C(=O)OC(C)C(F)(F)F)cc2)cc1)C(F)(F)F ; CC(OC(=O)c1ccc([S+](c2ccc(C(=O)OC(C)C(F)(F)F)cc2)c2ccc(C(=O)OC(C)C(F)(F)F)cc2)cc1)C(F)(F)F]

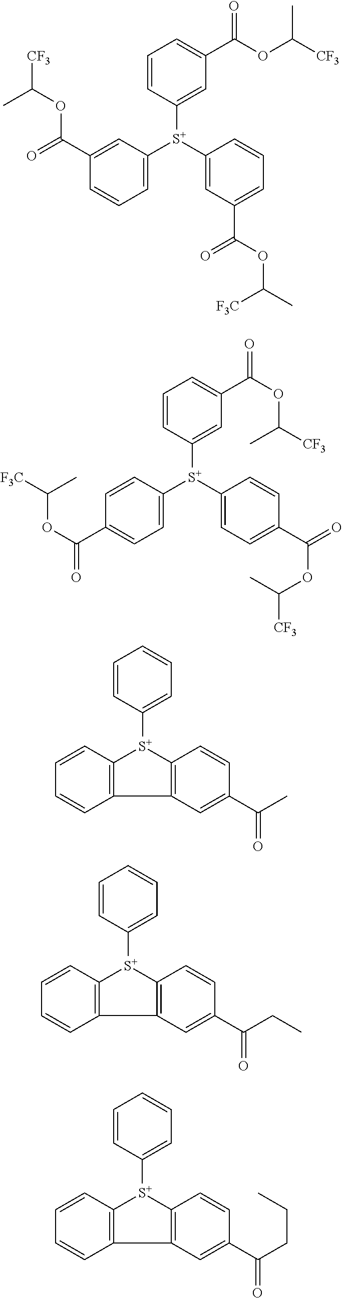 [SMILES: CC(OC(=O)c1cccc([S+](c2cccc(C(=O)OC(C)C(F)(F)F)c2)c2cccc(C(=O)OC(C)C(F)(F)F)c2)c1)C(F)(F)F ; CC(OC(=O)c1ccc([S+](c2ccc(C(=O)OC(C)C(F)(F)F)cc2)c2cccc(C(=O)OC(C)C(F)(F)F)c2)cc1)C(F)(F)F ; CC(=O)c1ccc2c(c1)c1ccccc1[s+]2-c1ccccc1 ; CCC(=O)c1ccc2c(c1)c1ccccc1[s+]2-c1ccccc1 ; CCCC(=O)c1ccc2c(c1)c1ccccc1[s+]2-c1ccccc1]

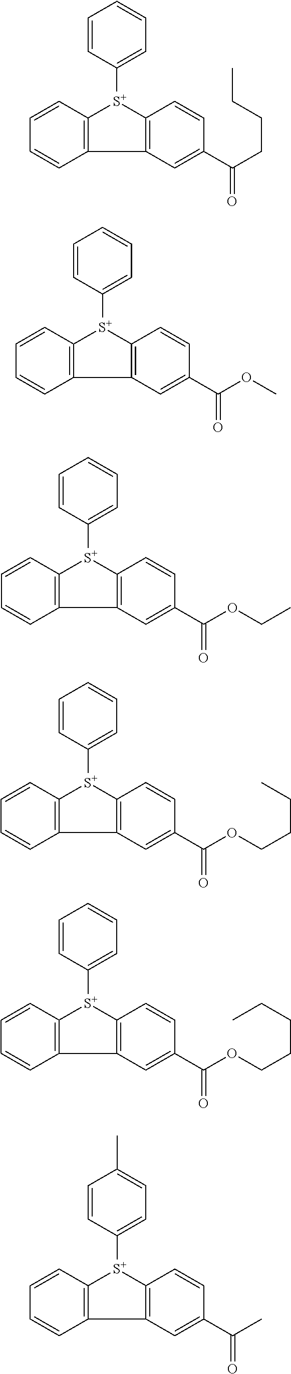 [SMILES: CCCCC(=O)c1ccc2c(c1)c1ccccc1[s+]2-c1ccccc1 ; COC(=O)c1ccc2c(c1)c1ccccc1[s+]2-c1ccccc1 ; CCOC(=O)c1ccc2c(c1)c1ccccc1[s+]2-c1ccccc1 ; CCCCOC(=O)c1ccc2c(c1)c1ccccc1[s+]2-c1ccccc1 ; CCCCCOC(=O)c1ccc2c(c1)c1ccccc1[s+]2-c1ccccc1 ; CC(=O)c1ccc2c(c1)c1ccccc1[s+]2-c1ccc(C)cc1]

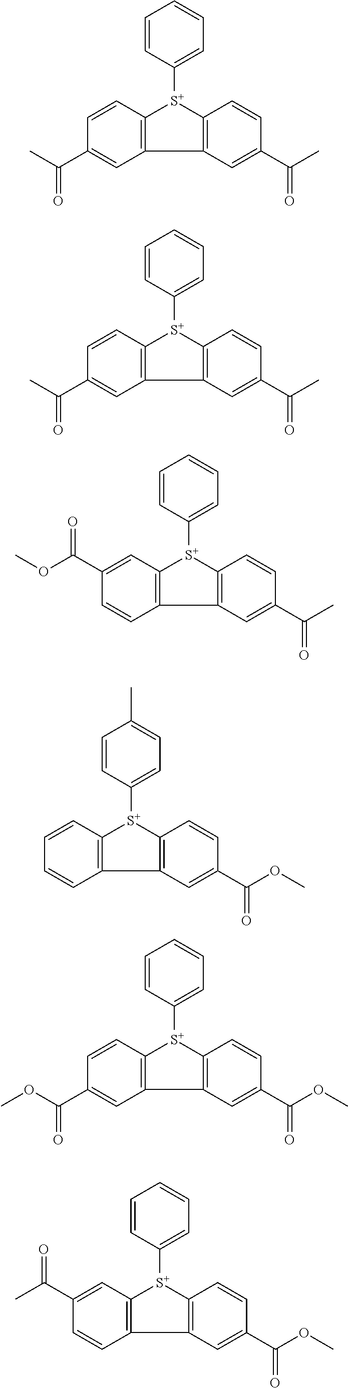

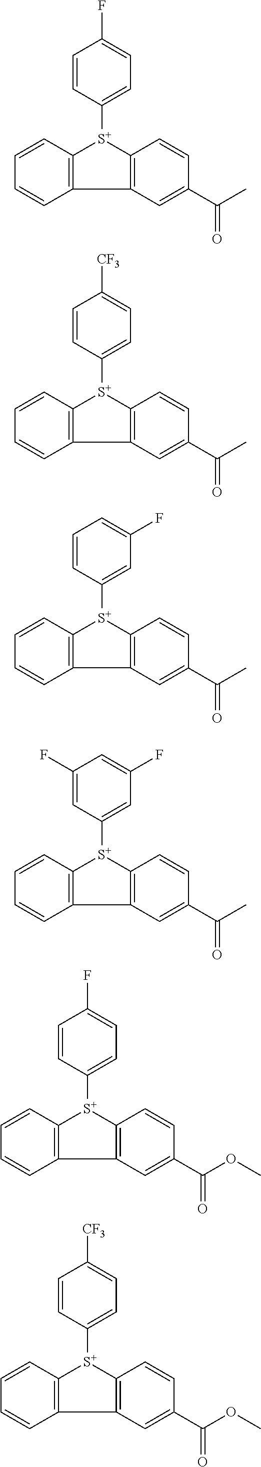 [SMILES: CC(=O)c1ccc2c(c1)c1ccccc1[s+]2-c1ccc(F)cc1 ; CC(=O)c1ccc2c(c1)c1ccccc1[s+]2-c1ccc(C(F)(F)F)cc1 ; CC(=O)c1ccc2c(c1)c1ccccc1[s+]2-c1cccc(F)c1 ; CC(=O)c1ccc2c(c1)c1ccccc1[s+]2-c1cc(F)cc(F)c1 ; COC(=O)c1ccc2c(c1)c1ccccc1[s+]2-c1ccc(F)cc1 ; COC(=O)c1ccc2c(c1)c1ccccc1[s+]2-c1ccc(C(F)(F)F)cc1]

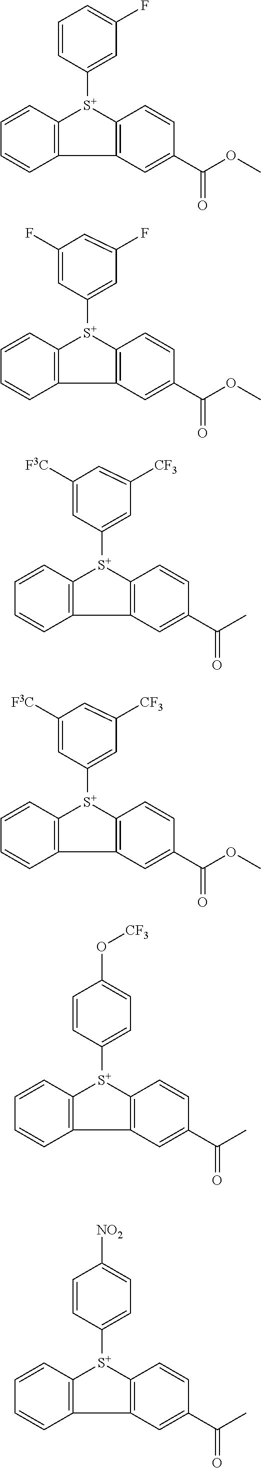 [SMILES: COC(=O)c1ccc2c(c1)c1ccccc1[s+]2-c1cccc(F)c1 ; COC(=O)c1ccc2c(c1)c1ccccc1[s+]2-c1cc(F)cc(F)c1 ; CC(=O)c1ccc2c(c1)c1ccccc1[s+]2-c1cc(C(F)(F)F)cc(C(F)(F)F)c1 ; COC(=O)c1ccc2c(c1)c1ccccc1[s+]2-c1cc(C(F)(F)F)cc(C(F)(F)F)c1 ; CC(=O)c1ccc2c(c1)c1ccccc1[s+]2-c1ccc(OC(F)(F)F)cc1 ; CC(=O)c1ccc2c(c1)c1ccccc1[s+]2-c1ccc([N+](=O)[O-])cc1]

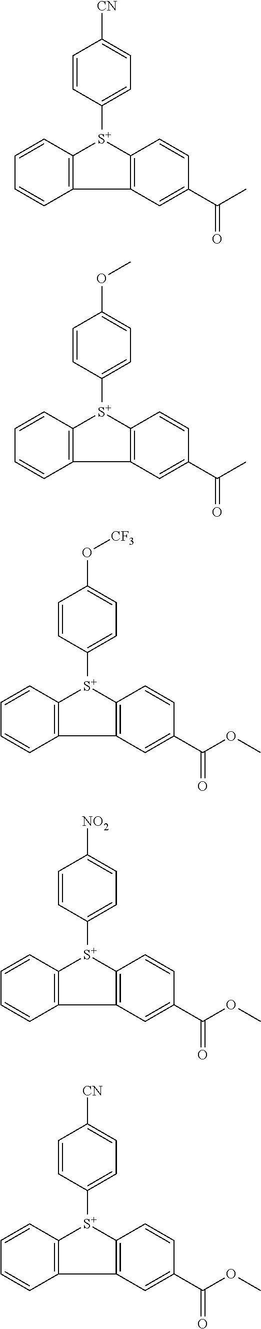 [SMILES: CC(=O)c1ccc2c(c1)c1ccccc1[s+]2-c1ccc(C#N)cc1 ; COc1ccc(-[s+]2c3ccccc3c3cc(C(C)=O)ccc32)cc1 ; COC(=O)c1ccc2c(c1)c1ccccc1[s+]2-c1ccc(OC(F)(F)F)cc1 ; COC(=O)c1ccc2c(c1)c1ccccc1[s+]2-c1ccc([N+](=O)[O-])cc1 ; COC(=O)c1ccc2c(c1)c1ccccc1[s+]2-c1ccc(C#N)cc1]

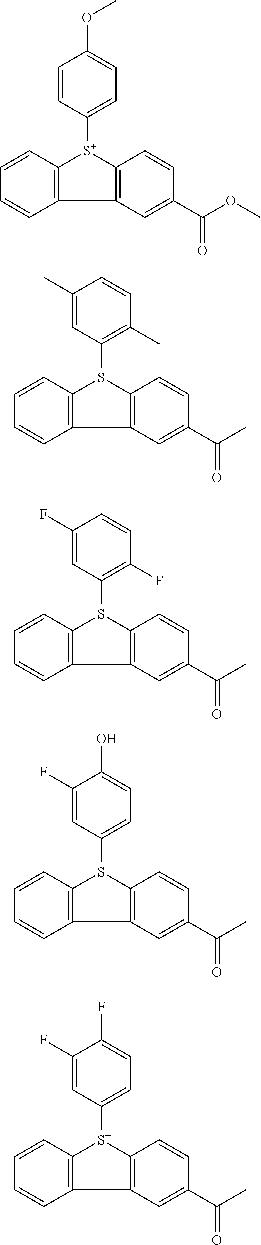 [SMILES: COC(=O)c1ccc2c(c1)c1ccccc1[s+]2-c1ccc(OC)cc1 ; CC(=O)c1ccc2c(c1)c1ccccc1[s+]2-c1cc(C)ccc1C ; CC(=O)c1ccc2c(c1)c1ccccc1[s+]2-c1cc(F)ccc1F ; CC(=O)c1ccc2c(c1)c1ccccc1[s+]2-c1ccc(O)c(F)c1 ; CC(=O)c1ccc2c(c1)c1ccccc1[s+]2-c1ccc(F)c(F)c1]

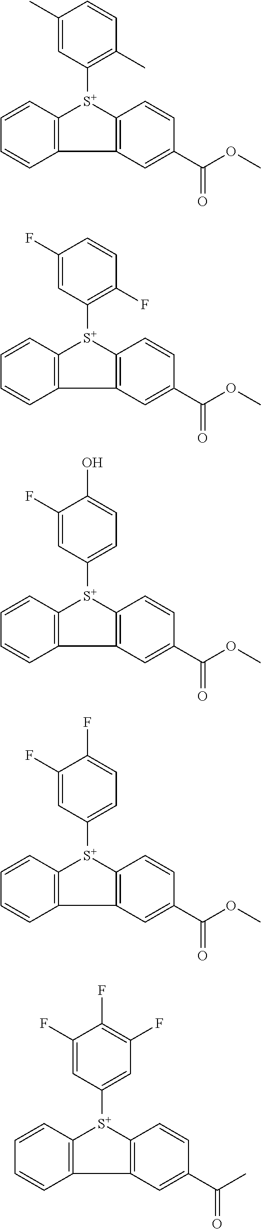 [SMILES: COC(=O)c1ccc2c(c1)c1ccccc1[s+]2-c1cc(C)ccc1C ; COC(=O)c1ccc2c(c1)c1ccccc1[s+]2-c1cc(F)ccc1F ; COC(=O)c1ccc2c(c1)c1ccccc1[s+]2-c1ccc(O)c(F)c1 ; COC(=O)c1ccc2c(c1)c1ccccc1[s+]2-c1ccc(F)c(F)c1 ; CC(=O)c1ccc2c(c1)c1ccccc1[s+]2-c1cc(F)c(F)c(F)c1]

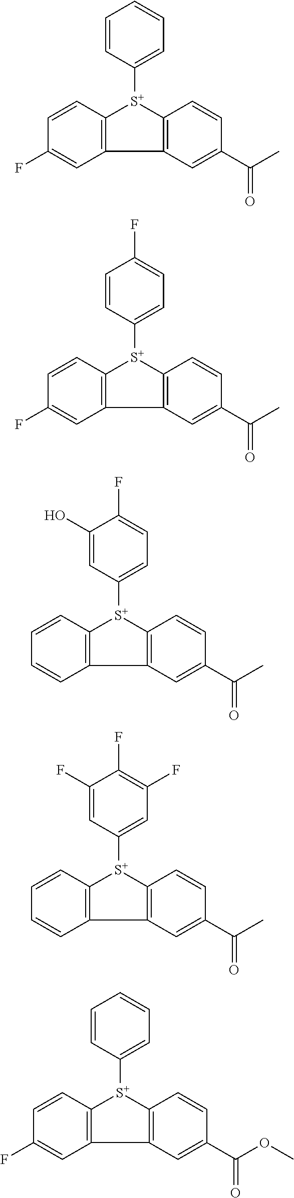 [SMILES: CC(=O)c1ccc2c(c1)c1cc(F)ccc1[s+]2-c1ccccc1 ; CC(=O)c1ccc2c(c1)c1cc(F)ccc1[s+]2-c1ccc(F)cc1 ; CC(=O)c1ccc2c(c1)c1ccccc1[s+]2-c1ccc(F)c(O)c1 ; CC(=O)c1ccc2c(c1)c1ccccc1[s+]2-c1cc(F)c(F)c(F)c1 ; COC(=O)c1ccc2c(c1)c1cc(F)ccc1[s+]2-c1ccccc1]

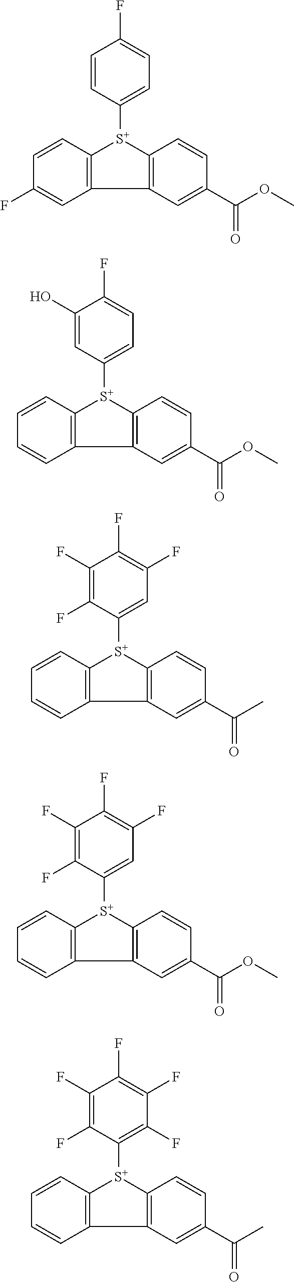 [SMILES: COC(=O)c1ccc2c(c1)c1cc(F)ccc1[s+]2-c1ccc(F)cc1 ; COC(=O)c1ccc2c(c1)c1ccccc1[s+]2-c1ccc(F)c(O)c1 ; CC(=O)c1ccc2c(c1)c1ccccc1[s+]2-c1cc(F)c(F)c(F)c1F ; COC(=O)c1ccc2c(c1)c1ccccc1[s+]2-c1cc(F)c(F)c(F)c1F ; CC(=O)c1ccc2c(c1)c1ccccc1[s+]2-c1c(F)c(F)c(F)c(F)c1F]

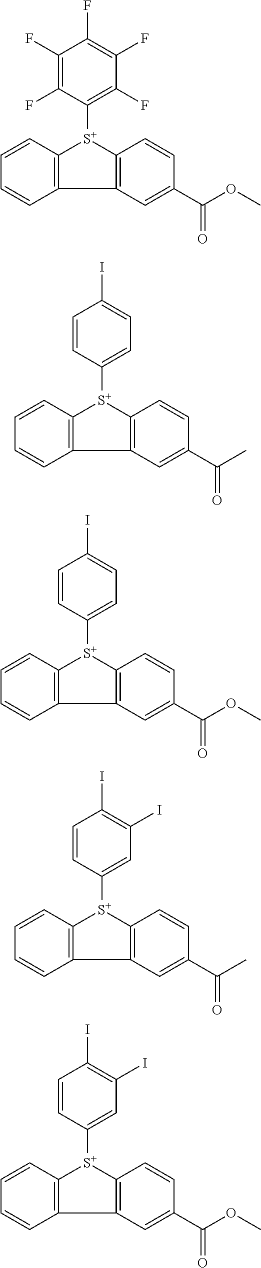 [SMILES: COC(=O)c1ccc2c(c1)c1ccccc1[s+]2-c1c(F)c(F)c(F)c(F)c1F ; CC(=O)c1ccc2c(c1)c1ccccc1[s+]2-c1ccc(I)cc1 ; COC(=O)c1ccc2c(c1)c1ccccc1[s+]2-c1ccc(I)cc1 ; CC(=O)c1ccc2c(c1)c1ccccc1[s+]2-c1ccc(I)c(I)c1 ; COC(=O)c1ccc2c(c1)c1ccccc1[s+]2-c1ccc(I)c(I)c1]

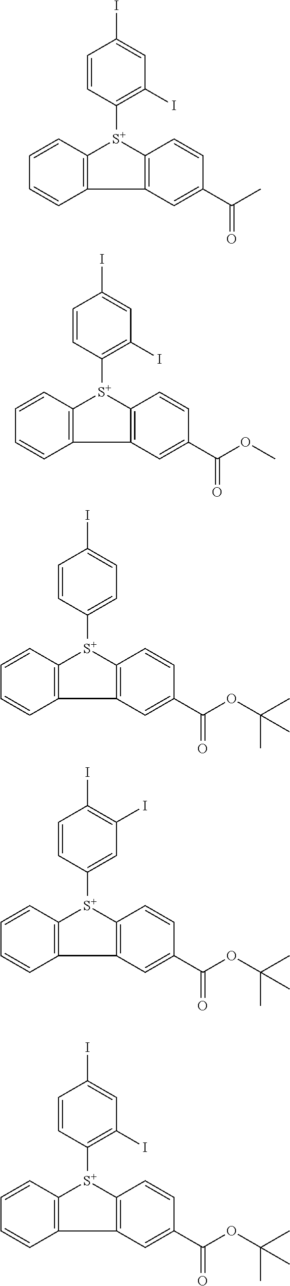 [SMILES: CC(=O)c1ccc2c(c1)c1ccccc1[s+]2-c1ccc(I)cc1I ; COC(=O)c1ccc2c(c1)c1ccccc1[s+]2-c1ccc(I)cc1I ; CC(C)(C)OC(=O)c1ccc2c(c1)c1ccccc1[s+]2-c1ccc(I)cc1 ; CC(C)(C)OC(=O)c1ccc2c(c1)c1ccccc1[s+]2-c1ccc(I)c(I)c1 ; CC(C)(C)OC(=O)c1ccc2c(c1)c1ccccc1[s+]2-c1ccc(I)cc1I]

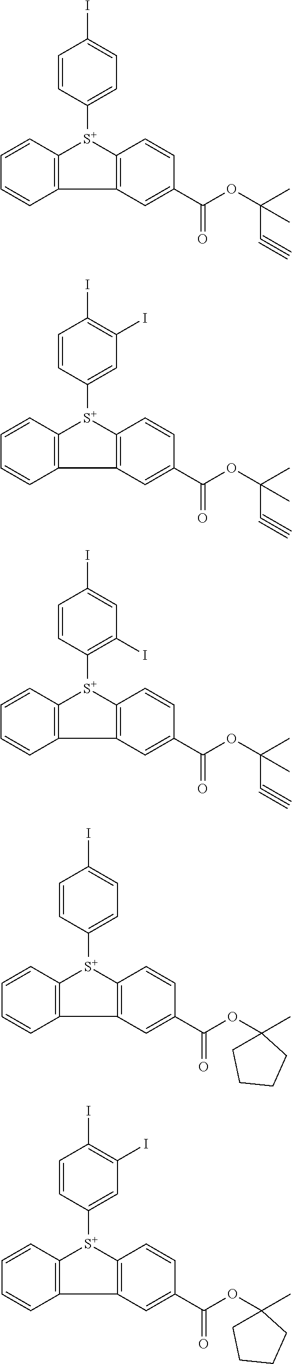 [SMILES: C#CC(C)(C)OC(=O)c1ccc2c(c1)c1ccccc1[s+]2-c1ccc(I)cc1 ; C#CC(C)(C)OC(=O)c1ccc2c(c1)c1ccccc1[s+]2-c1ccc(I)c(I)c1 ; C#CC(C)(C)OC(=O)c1ccc2c(c1)c1ccccc1[s+]2-c1ccc(I)cc1I ; CC1(OC(=O)c2ccc3c(c2)c2ccccc2[s+]3-c2ccc(I)cc2)CCCC1 ; CC1(OC(=O)c2ccc3c(c2)c2ccccc2[s+]3-c2ccc(I)c(I)c2)CCCC1]

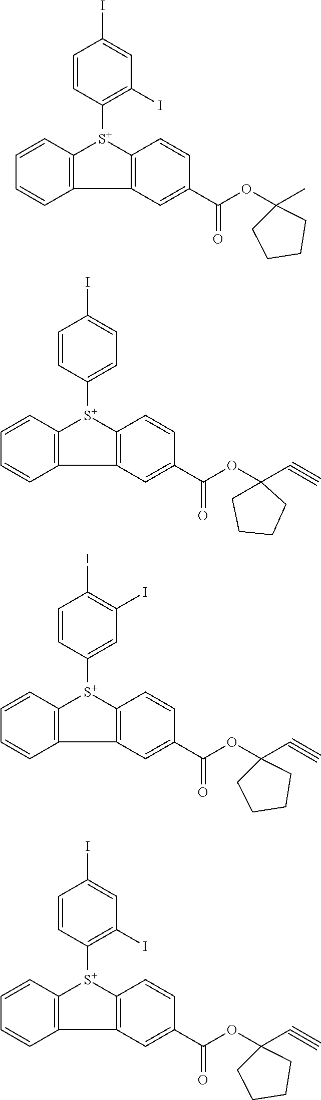 [SMILES: CC1(OC(=O)c2ccc3c(c2)c2ccccc2[s+]3-c2ccc(I)cc2I)CCCC1 ; C#CC1(OC(=O)c2ccc3c(c2)c2ccccc2[s+]3-c2ccc(I)cc2)CCCC1 ; C#CC1(OC(=O)c2ccc3c(c2)c2ccccc2[s+]3-c2ccc(I)c(I)c2)CCCC1 ; C#CC1(OC(=O)c2ccc3c(c2)c2ccccc2[s+]3-c2ccc(I)cc2I)CCCC1]

Examples of the iodonium cation represented by formula (Z-2) are shown below, but not limited thereto.

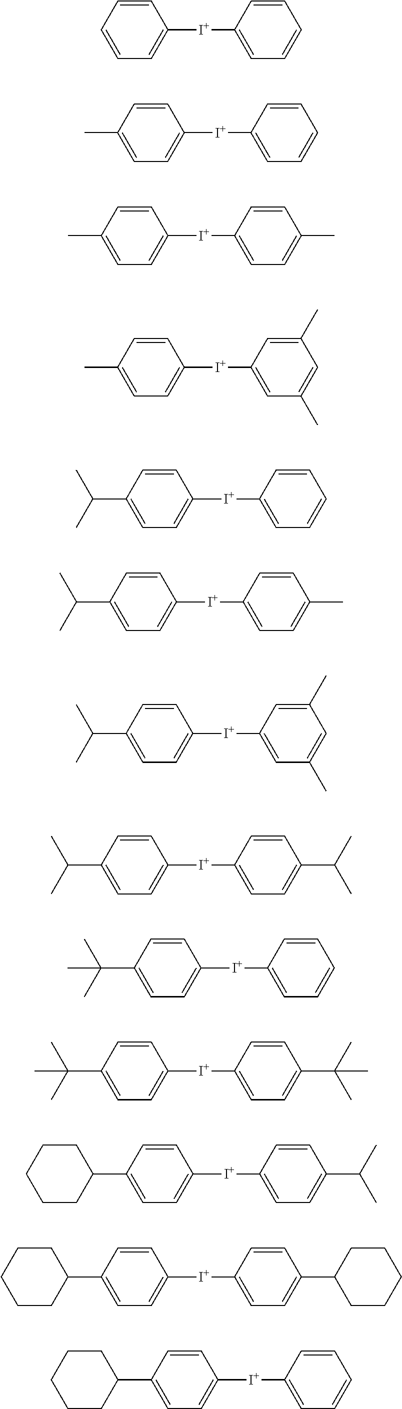

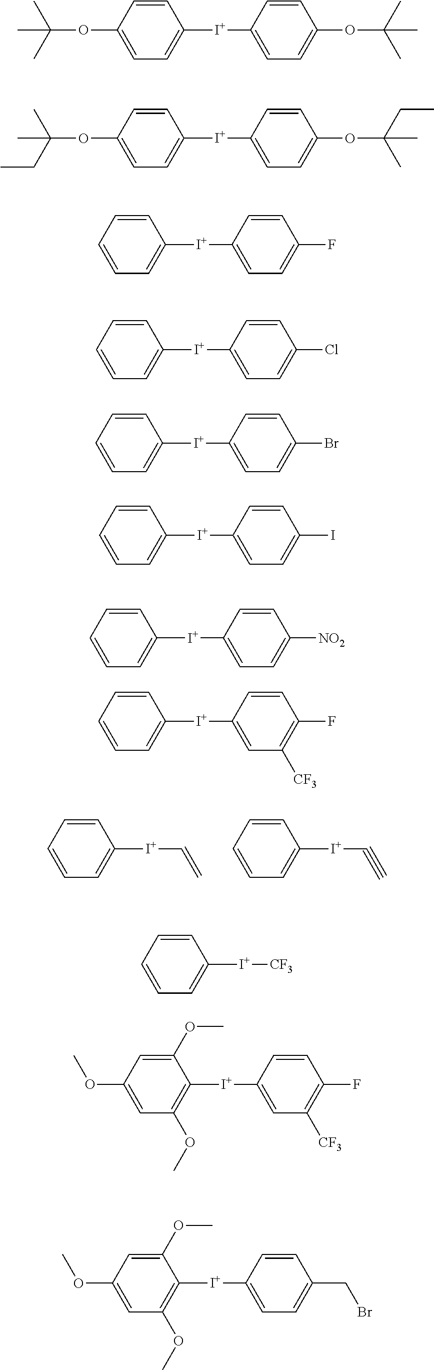

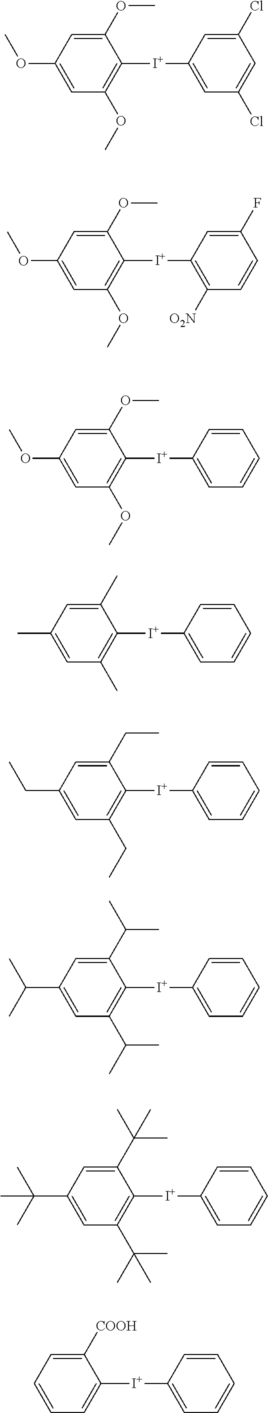

Examples of the inventive onium salt monomer include arbitrary combinations of the anion with the cation.

The inventive onium salt monomer may be synthesized in the same manner as in synthesis of a sulfonium salt having a polymerizable anion in JP 5201363, but the synthesis method is not limited thereto.

### [Polymer]

The inventive polymer comprises repeat units derived from the onium salt monomer having formula (a) (hereinafter, also referred to as repeat units (a)).

The inventive polymer is a polymer-bound photoacid generator that functions as a photoacid generator and also as a base polymer in the chemically amplified resist composition. A structural characteristic of the inventive polymer is that the polymer comprises repeat units derived from an onium salt monomer having an electron withdrawing group-substituted benzenesulfonic acid structure. The benzenesulfonate anion substituted with an electron withdrawing group is capable of increasing the acid strength of the sulfonic acid, and exerts not only an inductive effect but also a resonance effect to make the acid strength higher as compared to a case of being substituted with only fluorine. LWR, CDU, EL and DOF are improved, and a pattern resistant to pattern collapse can be formed. The inventive polymer is particularly suitable as a material for chemically amplified resist compositions.

The resist composition comprises a base polymer. When the resist composition is of positive tone, a base polymer comprising repeat units having an acid labile group is used. The repeat units having an acid labile group are preferably repeat units having the formula (b1) or repeat units having formula (b2). These repeat units are also referred to as repeat units (b1) or (b2).

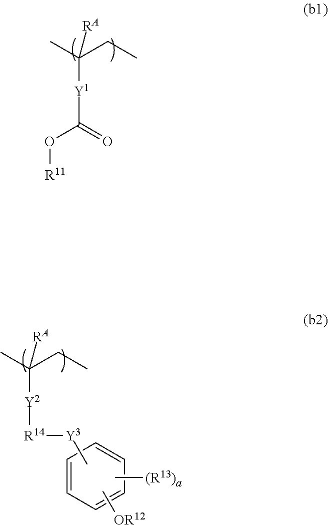

In formulae (b1) and (b2), R^{A} is each independently hydrogen or methyl. Y¹ is a single bond, a phenylene group, a naphthylene group, or a C₁-C₁₂ linking group containing at least one selected from an ester bond, an ether bond and a lactone ring, the phenylene group, the naphthylene group and the linking group may have at least one selected from a hydroxy group, a C₁-C₈ saturated hydrocarbyloxy group and a C₂-C₈ saturated hydrocarbylcarbonyloxy group. Y² is a single bond or an ester bond. Y³ is a single bond, an ether bond or an ester bond. R¹¹ and R¹² are each independently an acid labile group. R¹³ is a C₁-C₄ saturated hydrocarbyl group, a halogen atom, a C₂-C₅ saturated hydrocarbylcarbonyl group, a cyano group, or C₂-C₅ saturated hydrocarbyloxycarbonyl group. R¹⁴ is a single bond or C₁-C₆ alkanediyl group, and the alkanediyl group may contain at least one selected from a hydroxy group, a C₁-C₈ saturated hydrocarbyloxy group, a C₂-C₈ saturated hydrocarbylcarbonyloxy group, an ether bond and an ester bond. The subscript a is 0, 1, 2, 3 or 4.

Examples of the monomer from which repeat units (b1) are derived are shown below, but not limited thereto. Herein R^{A} and R¹¹ are as defined above.

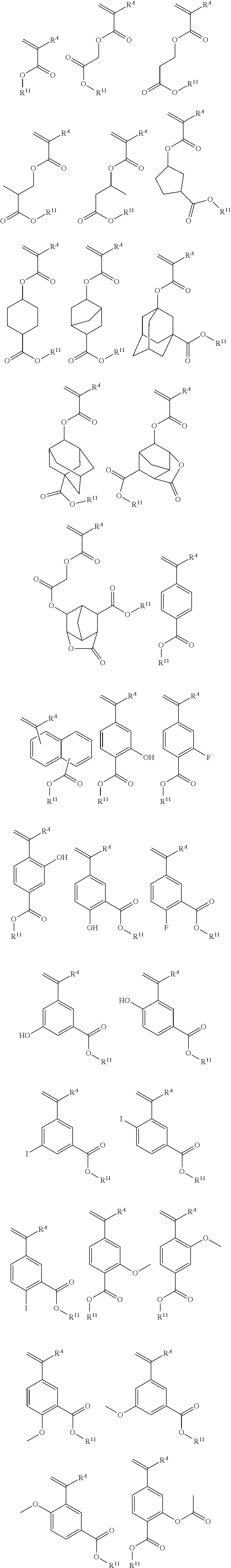

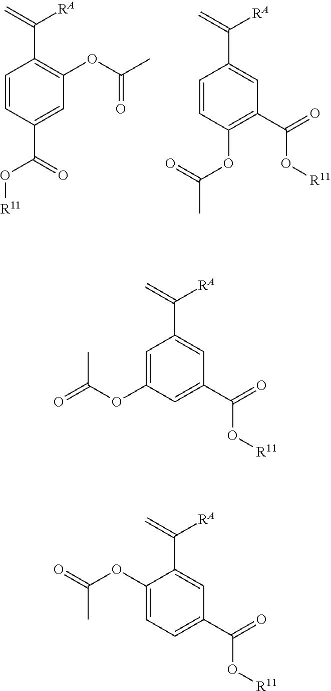

Examples of the monomer from which repeat units (b2) are derived are shown below, but not limited thereto. Herein R^{A} and R¹² are as defined above.

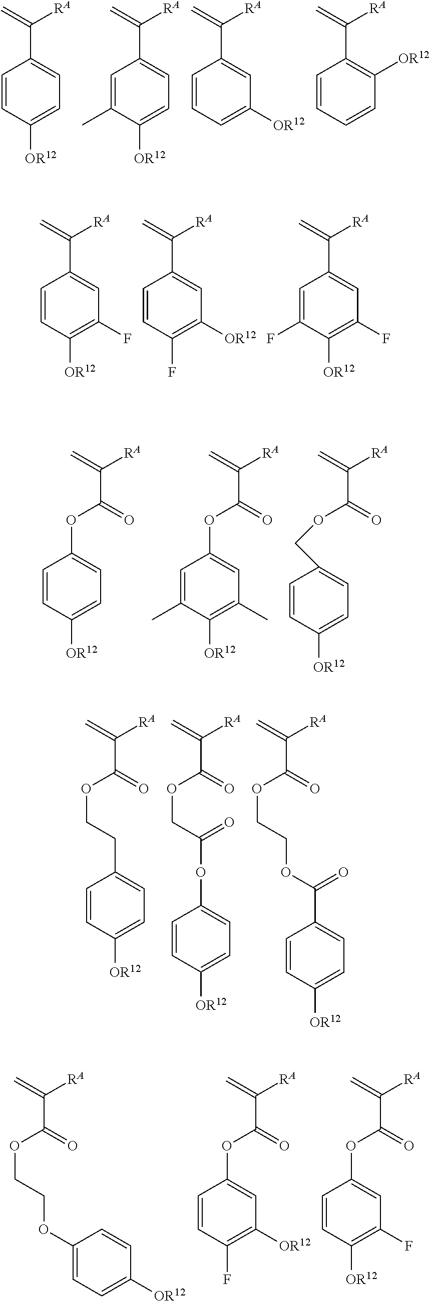

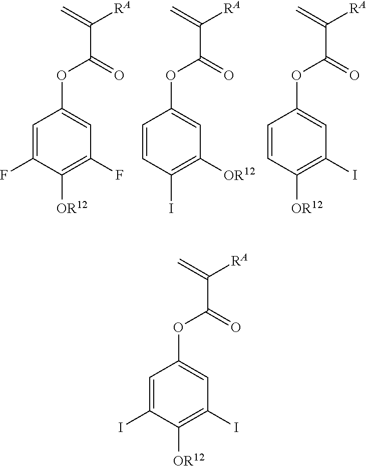

The acid labile group represented by R¹¹ or R¹² may be selected from a variety of such groups, for example, groups having the following formulae (AL-1) to (AL-3).

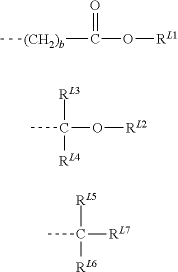

Herein the broken line designates a point of attachment.

In formula (AL-1), b is 0, 1, 2, 3, 4, 5 or 6. R^{L1} is a C₄-C₂₀, preferably C₄-C₁₅ tertiary hydrocarbyl group, a trihydrocarbylsilyl group in which each of the hydrocarbyl groups is a C₁-C₆ saturated hydrocarbyl group, a C₄-C₂₀ saturated hydrocarbyl group containing a carbonyl group, ether bond or ester bond, or a group having the formula (AL-3). The tertiary hydrocarbyl group means a group obtained by eliminating hydrogen from a tertiary carbon atom of a hydrocarbon.

The tertiary hydrocarbyl group R^{L1} may be saturated or unsaturated, and may be straight, branched or cyclic. Examples thereof include tert-butyl, tert-pentyl, 1,1-diethylpropyl, 1-ethylcyclopentyl, 1-butylcyclopentyl, 1-ethylcyclohexyl, 1-butylcyclohexyl, 1-ethyl-2-cyclopentenyl, 1-ethyl-2-cyclohexenyl, and 2-methyl-2-adamantyl groups. Specific examples of the trihydrocarbylsilyl group include trimethylsilyl, triethylsilyl, and dimethyl-tert-butylsilyl groups. The saturated hydrocarbyl group containing a carbonyl group, ether bond or ester bond may be straight, branched or cyclic, and is preferably cyclic, and examples thereof include 3-oxocyclohexyl, 4-methyl-2-oxooxan-4-yl, 5-methyl-2-oxooxolan-5-yl, 2-tetrahydropyranyl, and 2-tetrahydrofuranyl groups.

Examples of the acid labile group having the formula (AL-1) include tert-butoxycarbonyl, tert-butoxycarbonylmethyl, tert-pentyloxycarbonyl, tert-pentyloxycarbonylmethyl, 1,1-diethylpropyloxycarbonyl, 1,1-diethylpropyloxycarbonylmethyl, 1-ethylcyclopentyloxycarbonyl, 1-ethylcyclopentyloxycarbonylmethyl, 1-ethyl-2-cyclopentenyloxycarbonyl, 1-ethyl-2-cyclopentenyloxycarbonylmethyl, 1-ethoxyethoxycarbonylmethyl, 2-tetrahydropyranyloxycarbonylmethyl, and 2-tetrahydrofuranyloxycarbonylmethyl groups.

Other examples of the acid labile group having the formula (AL-1) include groups having the formulae (AL-1)-1 to (AL-1)-10.

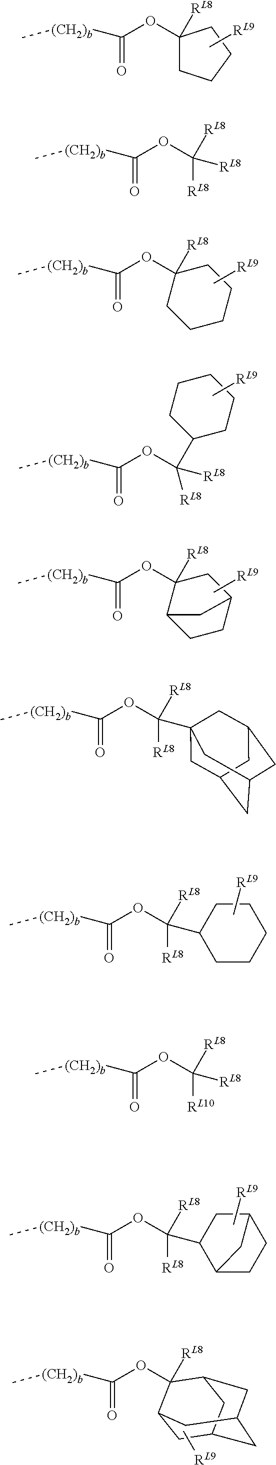

Herein the broken line designates a point of attachment.

In the formulae (AL-1)-1 to (AL-1)-10, b is as defined above. R^{L8} is each independently a C₁-C₁₀ saturated hydrocarbyl group or C₆-C₂₀ aryl group. R^{L9} is hydrogen or a C₁-C₁₀ saturated hydrocarbyl group. R^{L10} is each independently a C₂-C₁₀ saturated hydrocarbyl group or C₆-C₂₀ aryl group. The saturated hydrocarbyl group may be straight, branched or cyclic.

In the formula (AL-2), R^{L2} and R^{L3} are each independently hydrogen, or a C₁-C₁₈, preferably C₁-C₁₀ saturated hydrocarbyl group. The saturated hydrocarbyl group may be straight, branched or cyclic, and examples thereof include methyl, ethyl, n-propyl, isopropyl, n-butyl, isobutyl, sec-butyl, tert-butyl, cyclopentyl, cyclohexyl, 2-ethylhexyl and n-octyl groups.

In the formula (AL-2), R^{L4} is a C₁-C₁₈, preferably C₁-C₁₀ hydrocarbyl group which may contain a heteroatom. The hydrocarbyl group may be saturated or unsaturated and may be straight, branched or cyclic. Examples of the hydrocarbyl group include a C₁-C₁₈ saturated hydrocarbyl group, in which some hydrogen may be substituted by hydroxy, alkoxy, oxo, amino, or alkylamino. Examples of the substituted saturated hydrocarbyl groups are shown below.

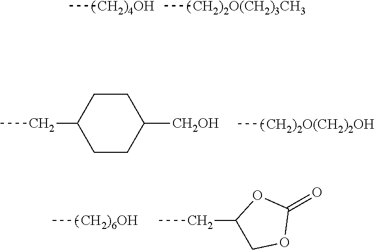

Herein the broken line designates a point of attachment.

A pair of R^{L2} and R^{L3}, R^{L2} and R^{L4}, or R^{L3} and R^{L4} may bond together to form a ring with the carbon atom or carbon and oxygen atoms to which they are attached. R^{L2} and R^{L3}, R^{L2} and R^{L4}, or R^{L3} and R^{L4} which form a ring are each independently a C₁-C₁₈, preferably C₁-C₁₀ alkanediyl group. The ring thus formed is preferably of 3 to 10, more preferably 4 to 10 carbon atoms.

Of the acid labile groups having formula (AL-2), suitable straight or branched groups include those having formulae (AL-2)-1 to (AL-2)-69, but are not limited thereto. In the following formulae, the broken line designates a point of attachment.

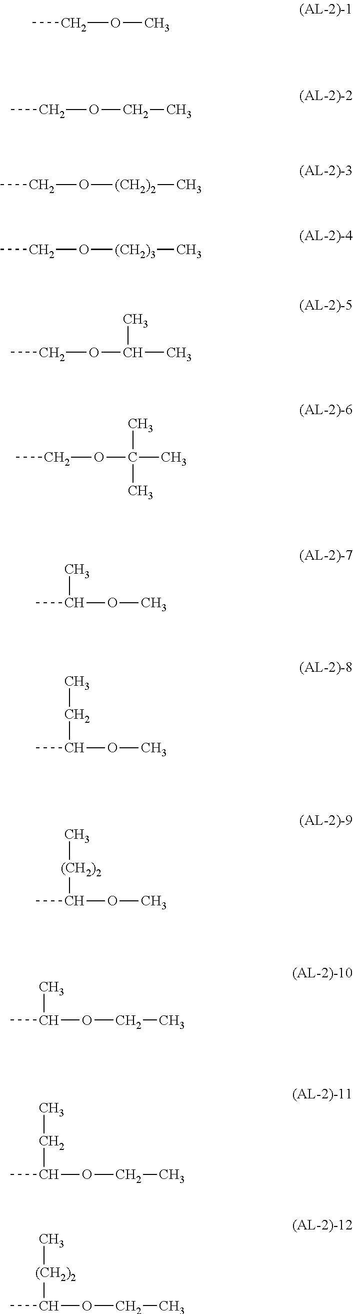

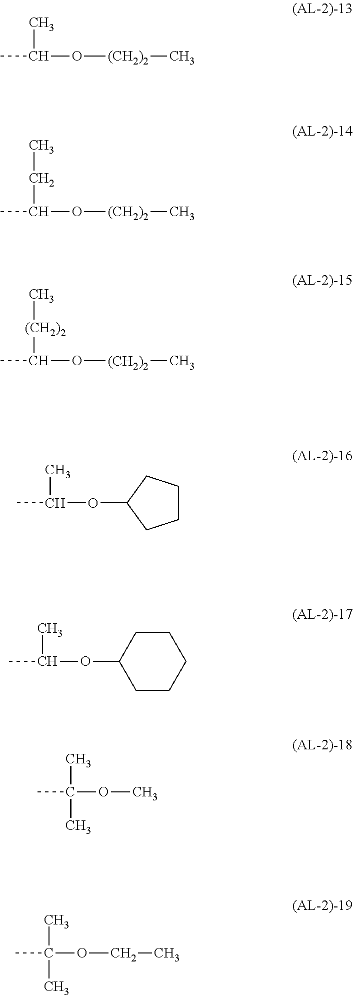

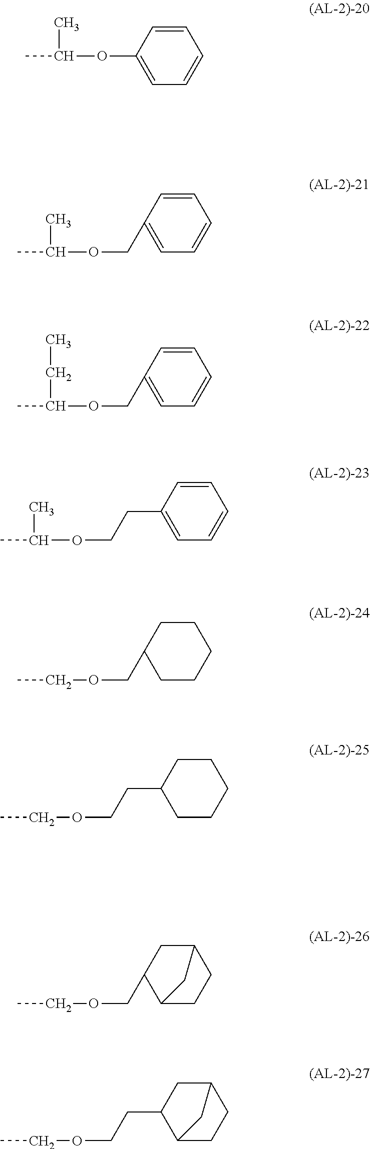

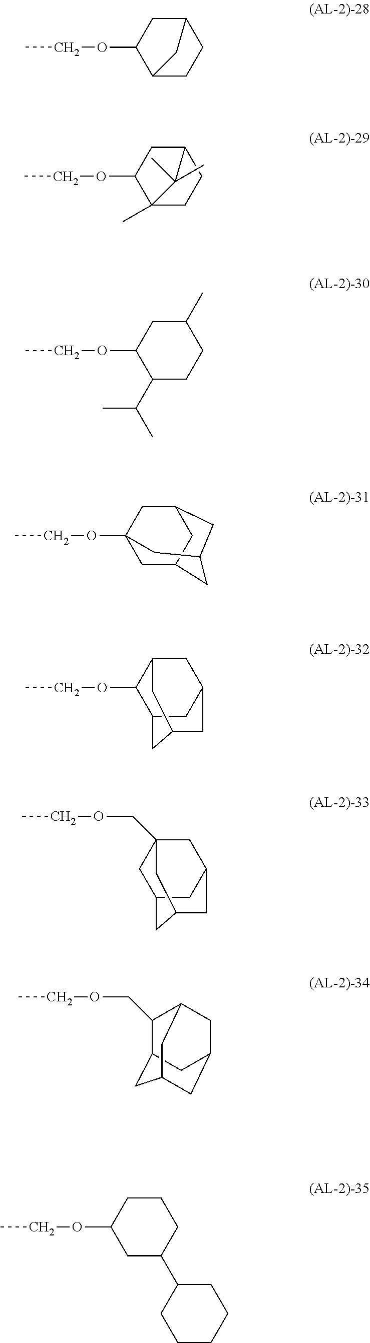

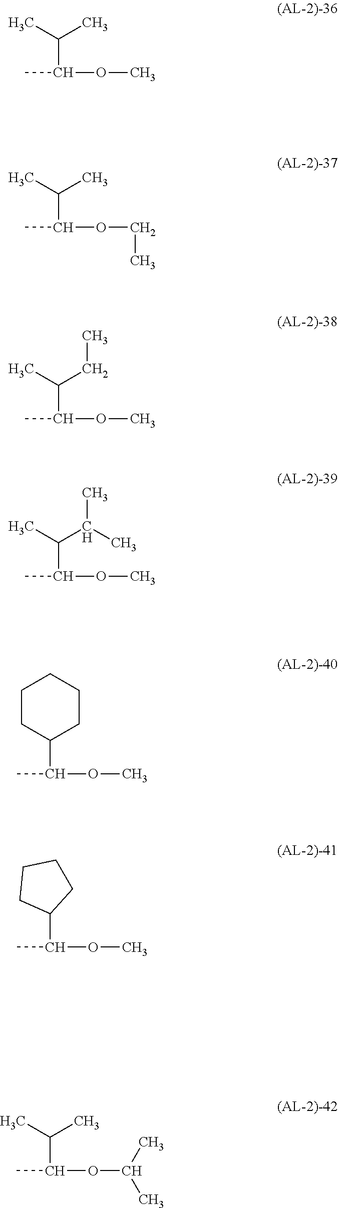

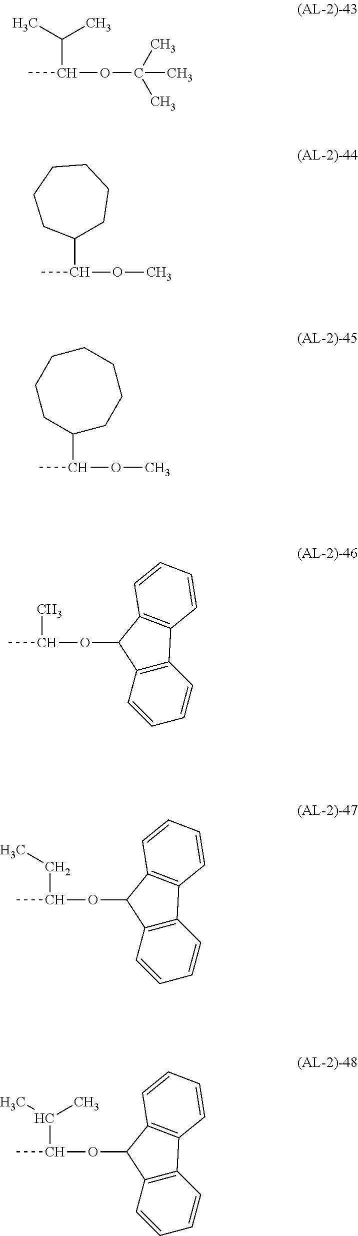

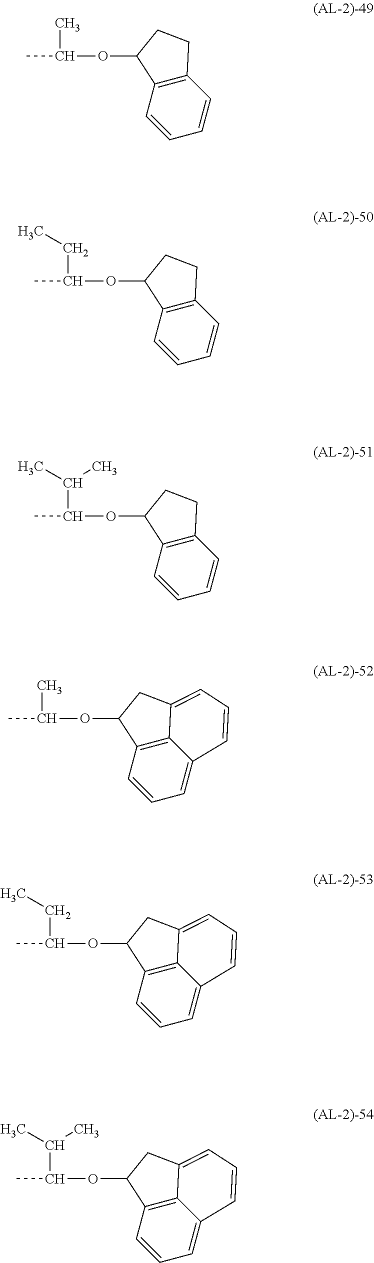

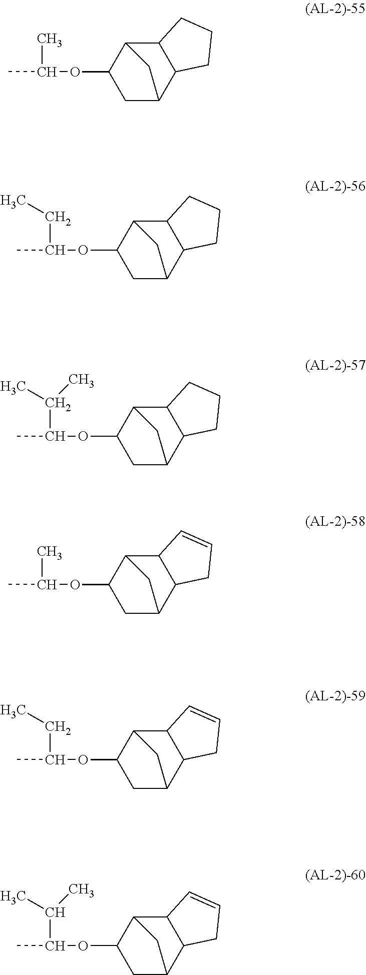

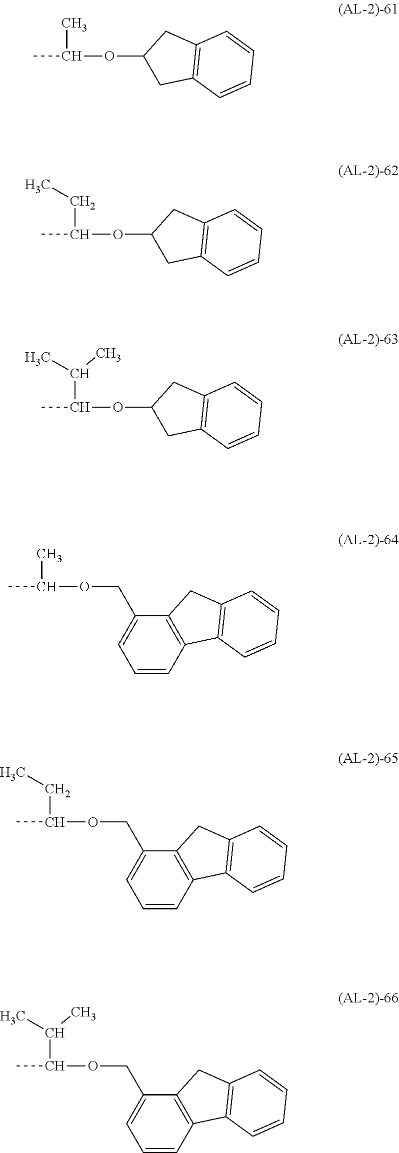

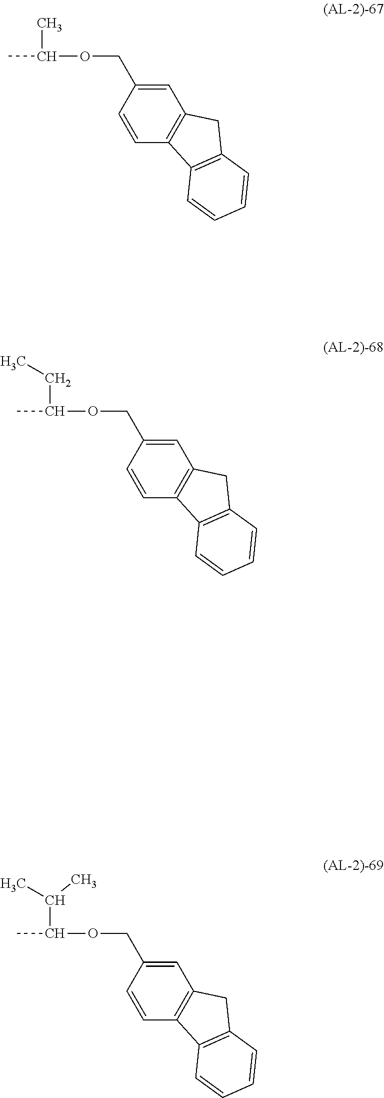

Of the acid labile groups having formula (AL-2), suitable cyclic groups include tetrahydrofuran-2-yl, 2-methyltetrahydrofuran-2-yl, tetrahydropyran-2-yl, and 2-methyltetrahydropyran-2-yl.

Also included are acid labile groups having the following formulae (AL-2a) and (AL-2b). With these acid labile groups, the base polymer may be crosslinked within the molecule or between molecules.

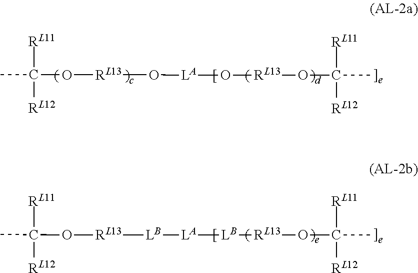

Herein the broken line designates a point of attachment.

In the formulae (AL-2a) and (AL-2b), R^{L11} and R^{L12} are each independently hydrogen or a C₁-C₈ saturated hydrocarbyl group which may be straight, branched or cyclic. The saturated hydrocarbyl group may be straight, branched or cyclic. Also, R^{L11} and R^{L12} may bond together to form a ring with the carbon atom to which they are attached, and in this case, R^{L11} and R^{L12} are each independently a C₁-C₈ alkanediyl group. R^{L13} is each independently a C₁-C₁₀ saturated hydrocarbylene group. The saturated hydrocarbylene group may be straight, branched or cyclic. The subscripts c and d are each independently an integer of 0 to 10, preferably 0, 1, 2, 3, 4 or 5, and e is an integer of 1 to 7, preferably 1, 2 or 3.

In the formulae (AL-2a) and (AL-2b), L^{A} is a C₁-C₅₀ (e+1)-valent aliphatic saturated hydrocarbon group, C₃-C₅₀ (e+1)-valent alicyclic saturated hydrocarbon group, C₆-C₅₀ (e+1)-valent aromatic hydrocarbon group or C₃-C₅₀ (e+1)-valent heterocyclic group. In these groups, some constituent —CH₂— may be replaced by a heteroatom-containing moiety, or some carbon-bonded hydrogen may be substituted by a hydroxy, carboxy, acyl moiety or fluorine. L^{A} is preferably a C₁-C₂₀ saturated hydrocarbon group (e.g., saturated hydrocarbylene group, trivalent saturated hydrocarbon group or tetravalent saturated hydrocarbon group), or C₆-C₃₀ arylene group. The saturated hydrocarbon group may be straight, branched or cyclic. L^{B} is —C(═O)—O—, —NH—C(═O)—O— or —NH—C(═O)—NH—.

Examples of the crosslinking acetal groups having the formulae (AL-2a) and (AL-2b) include groups having the following formulae (AL-2)-70 to (AL-2)-77.

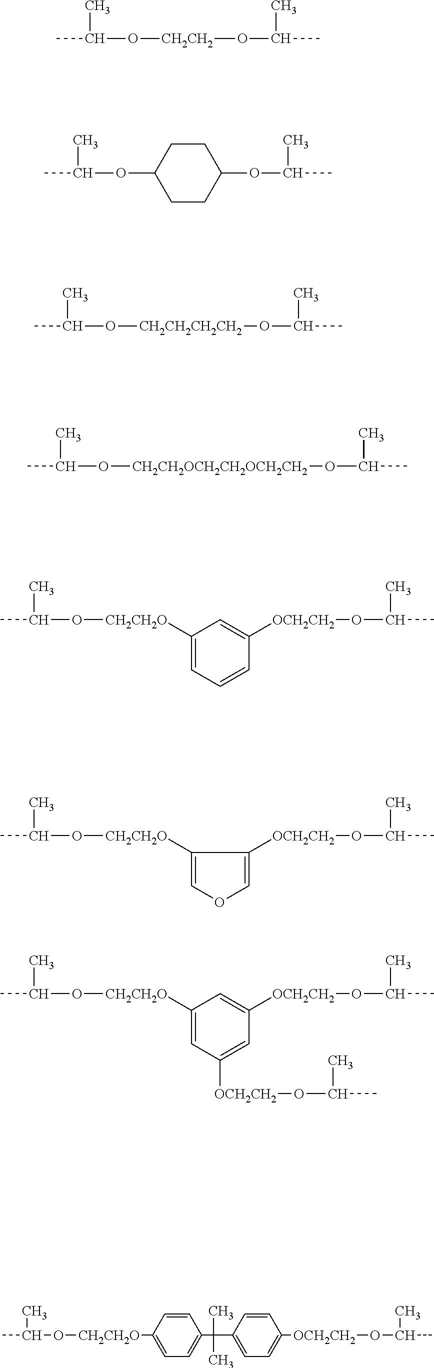

Herein the broken line designates a point of attachment.

In the formula (AL-3), R^{L5} is hydrogen or a C₁-C₂₀ hydrocarbyl group, and the hydrocarbyl group may contain a heteroatom such as oxygen, sulfur, nitrogen, or fluorine. R^{L6} and R^{L7} are each independently a C₁-C₂₀ hydrocarbyl group which may contain a heteroatom such as oxygen, sulfur, nitrogen, or fluorine. The hydrocarbyl group may be saturated or unsaturated and straight, branched or cyclic. Examples thereof include C₁-C₂₀ alkyl groups, C₃-C₂₀ cyclic saturated hydrocarbyl groups, C₂-C₂₀ alkenyl groups, C₃-C₂₀ cyclic unsaturated hydrocarbyl groups, and C₆-C₁₀ aryl groups. A pair of R^{L5} and R^{L6}, R^{L5} and R^{L7}, or R^{L6} and R^{L7} may bond together to form a C₃-C₂₀ aliphatic ring with the carbon atom to which they are attached.

Examples of the group having the formula (AL-3) include tert-butyl, 1,1-diethylpropyl, 1-ethylnorbornyl, 1-methylcyclopentyl, 1-ethylcyclopentyl, 1-isopropylcyclopentyl, 1-methylcyclohexyl, 2-(2-methyl)adamantyl, 2-(2-ethyl)adamantyl, and tert-pentyl.

Examples of the group having formula (AL-3) also include groups having the formulae (AL-3)-1 to (AL-3)-22.

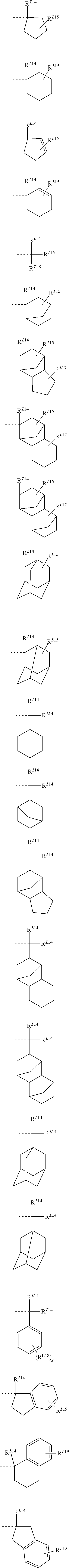

Herein the broken line designates a point of attachment.

In the formulae (AL-3)-1 to (AL-3)-22, R^{L14} is each independently hydrogen, a C₁-C₈ aliphatic hydrocarbyl group or C₆-C₂₀ aryl group. R^{L15} and R^{L17} are each independently hydrogen, or a C₁-C₂₀ saturated hydrocarbyl group. R^{L16} is a C₆-C₂₀ aryl group. The saturated hydrocarbyl group may be straight, branched or cyclic. Typical of the aryl group is phenyl. R^{L18} is fluorine, iodine, a nitro group or a trifluoromethyl group. R^{L19} is each independently hydrogen, fluorine, iodine, a nitro group, a C₁-C₈ saturated hydrocarbyl group, or a C₁-C₈ hydrocarbyloxy group. The subscript f is 1, 2, 3, 4 or 5.

Other examples of the acid labile group include groups having the formulae (AL-3)-23 and (AL-3)-24. With these acid labile groups, the base polymer may be crosslinked within the molecule or between molecules.

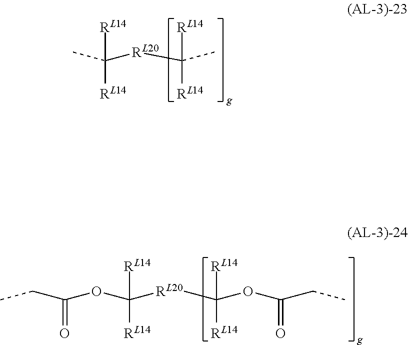

Herein the broken line designates a point of attachment.

In the formulae (AL-3)-23 and (AL-3)-24, R^{L14} is as defined above. R^{L20} is a C₁-C₂₀ (g+1)-valent saturated or unsaturated hydrocarbylene group or C₆-C₂₀ (g+1)-valent arylene group, which may contain a heteroatom such as oxygen, sulfur or nitrogen. The saturated or unsaturated hydrocarbylene group may be straight, branched or cyclic. The subscript g is 1, 2 or 3.

Not only these acid labile groups, but also aromatic group-containing acid labile groups described in JP 5565293, JP 5434983, JP 5407941, JP 5655756, and JP 5655755 may be used.

The base polymer may further comprise repeat units (c) having a phenolic hydroxy group as an adhesive group. Examples of the monomer from which repeat units (c) are derived are shown below, but not limited thereto. Herein R^{A} is as defined above.

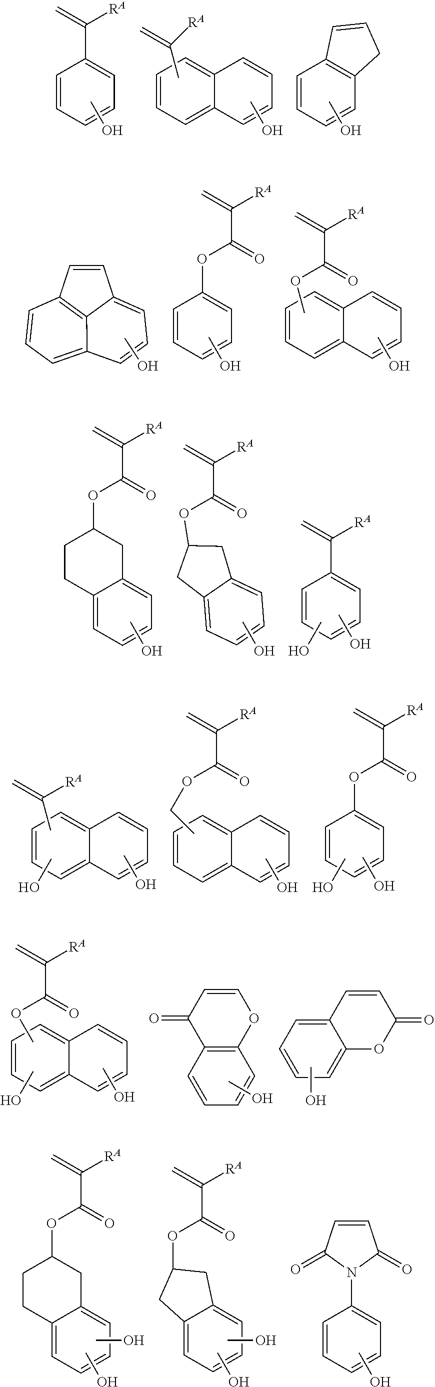

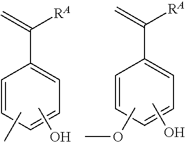

Further, repeat units (d) having another adhesive group selected from hydroxy (other than the foregoing phenolic hydroxy), lactone ring, sultone ring, ether bond, ester bond, sulfonic ester bond, carbonyl, sulfonyl, cyano and carboxy groups may also be incorporated in the base polymer. Examples of the monomer from which the repeat units (d) are derived are shown below, but not limited thereto. Herein R^{A} is as defined above.

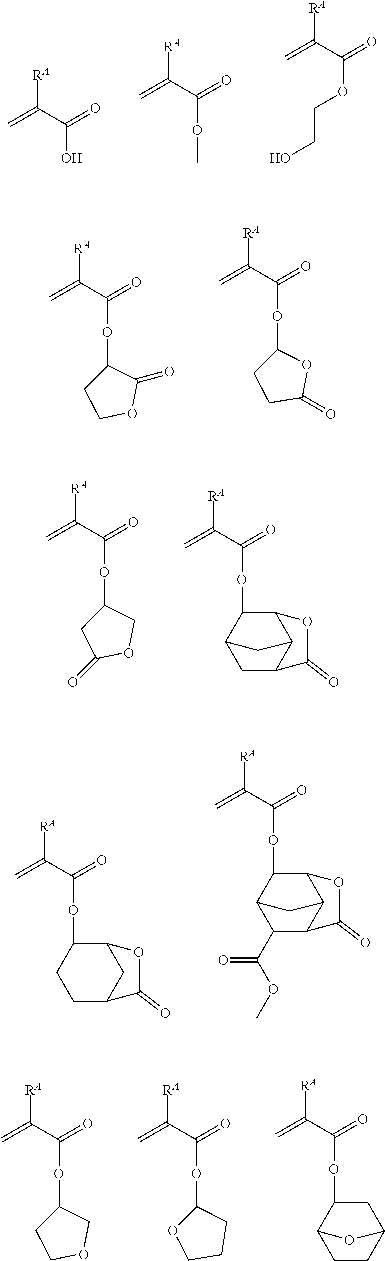

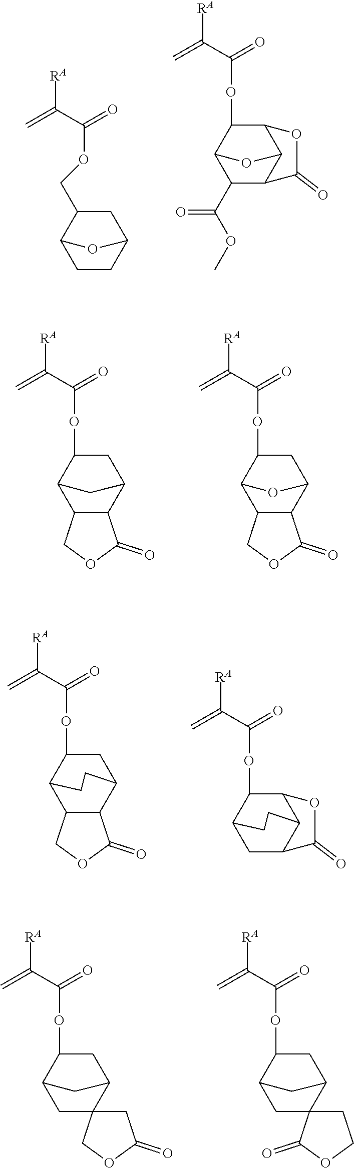

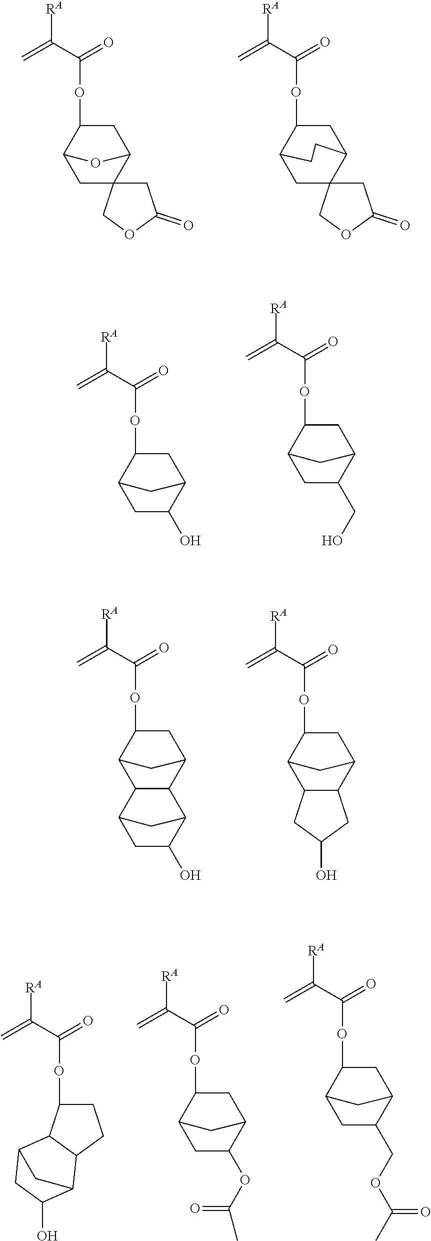

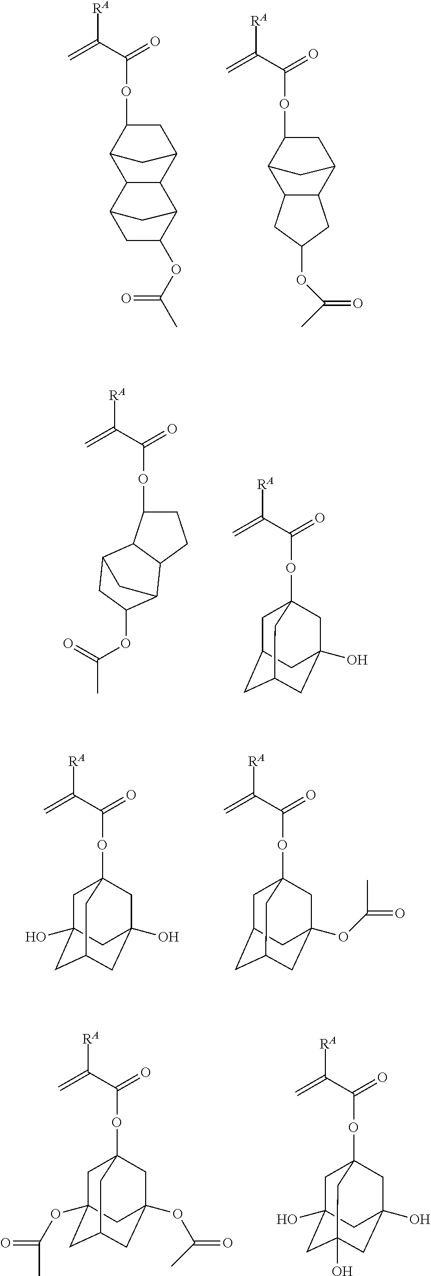

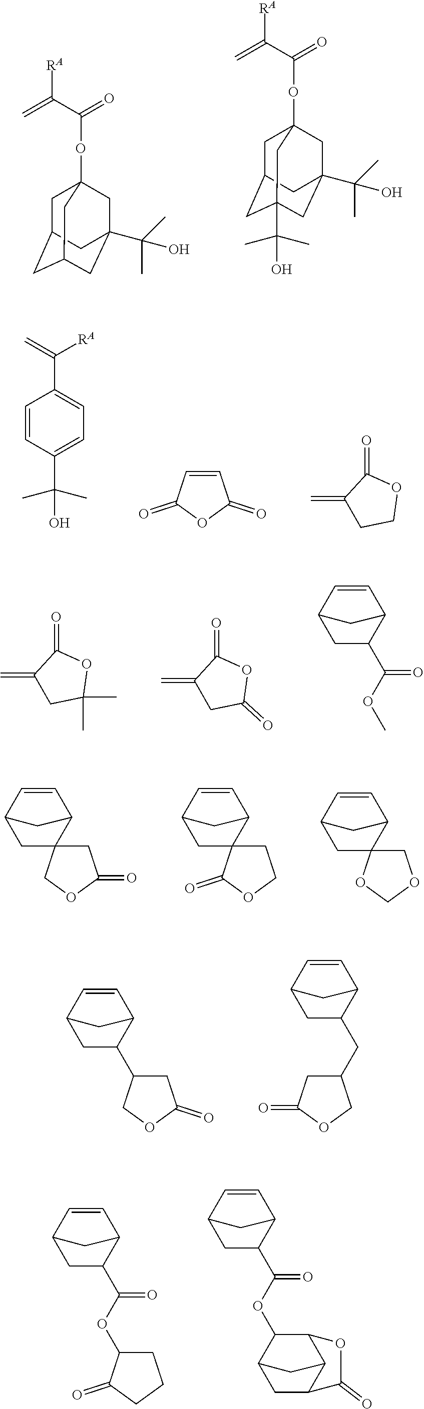

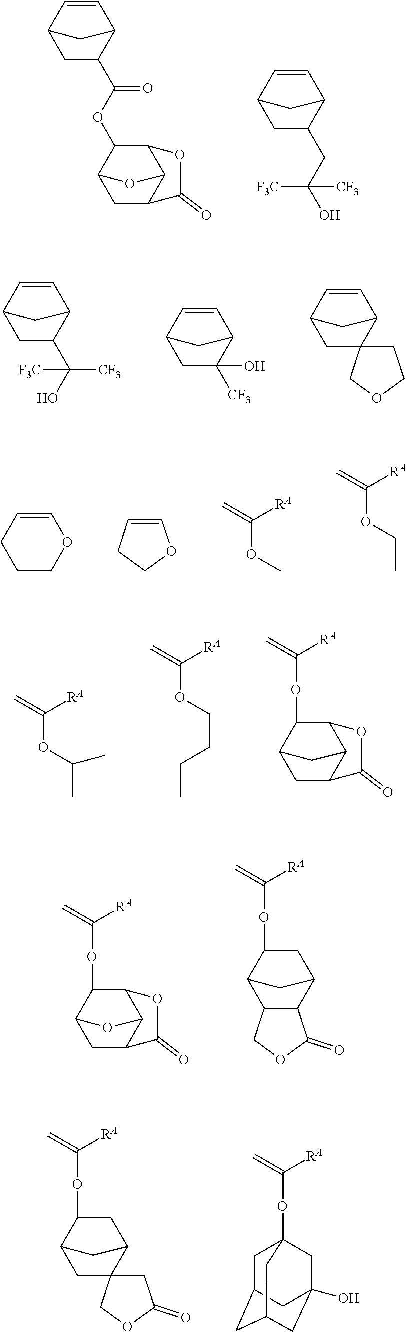

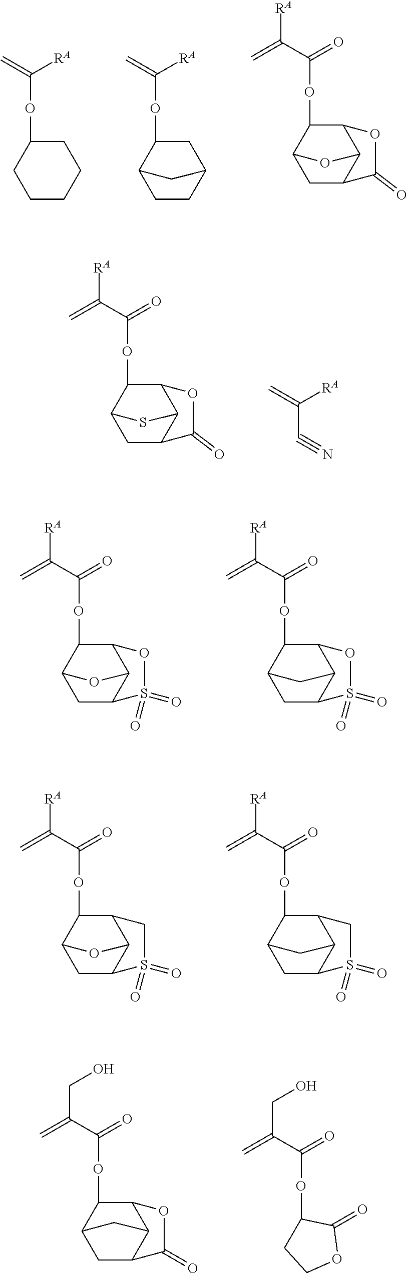

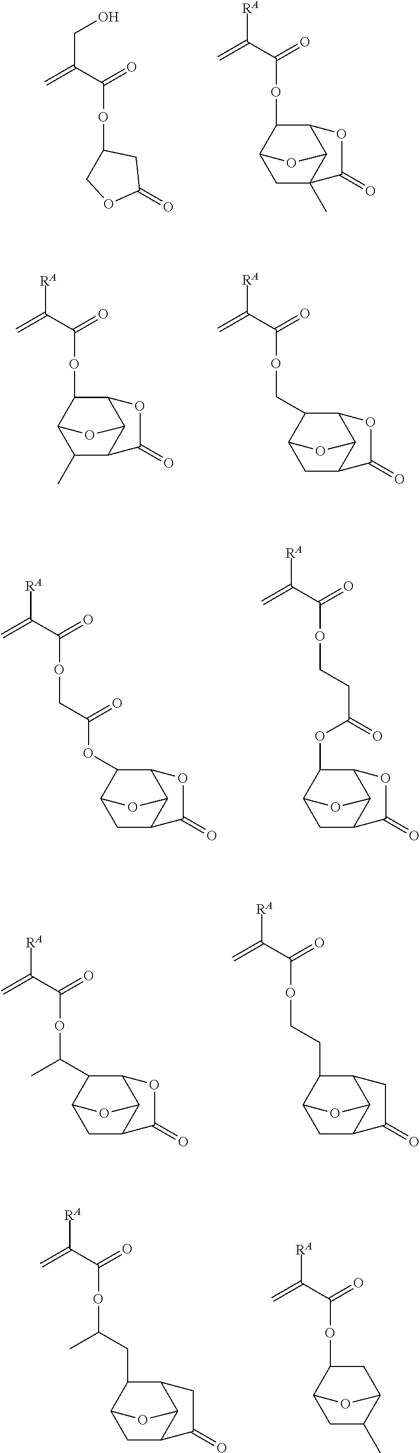

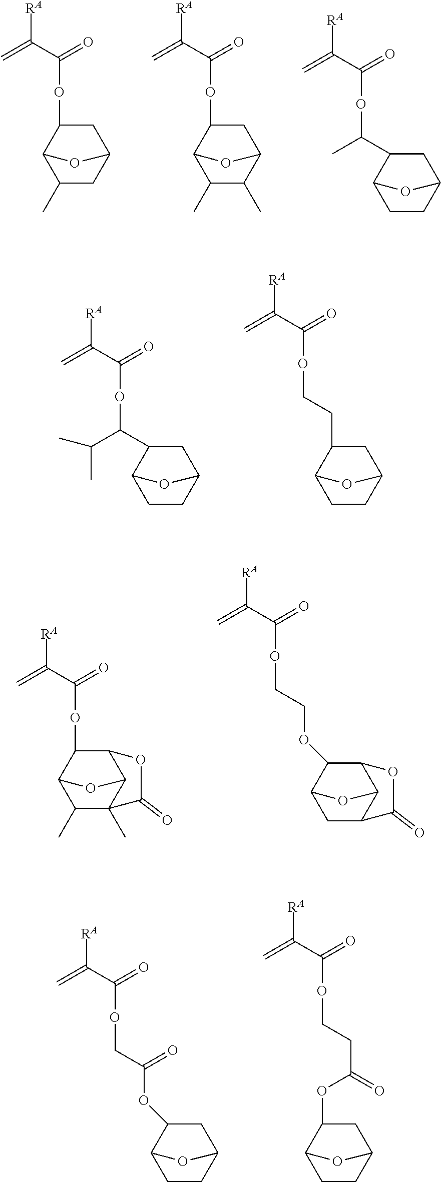

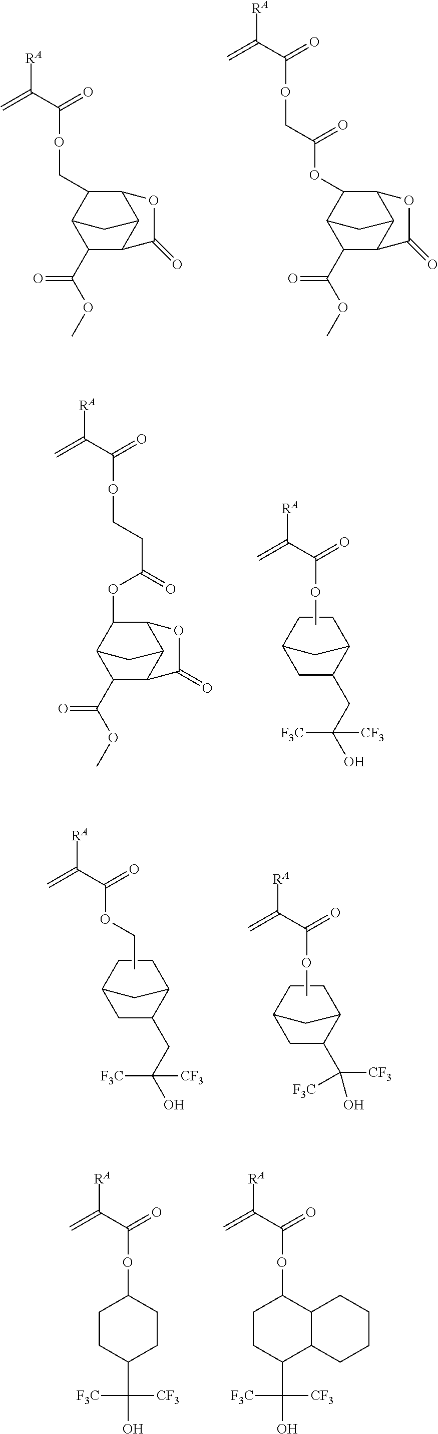

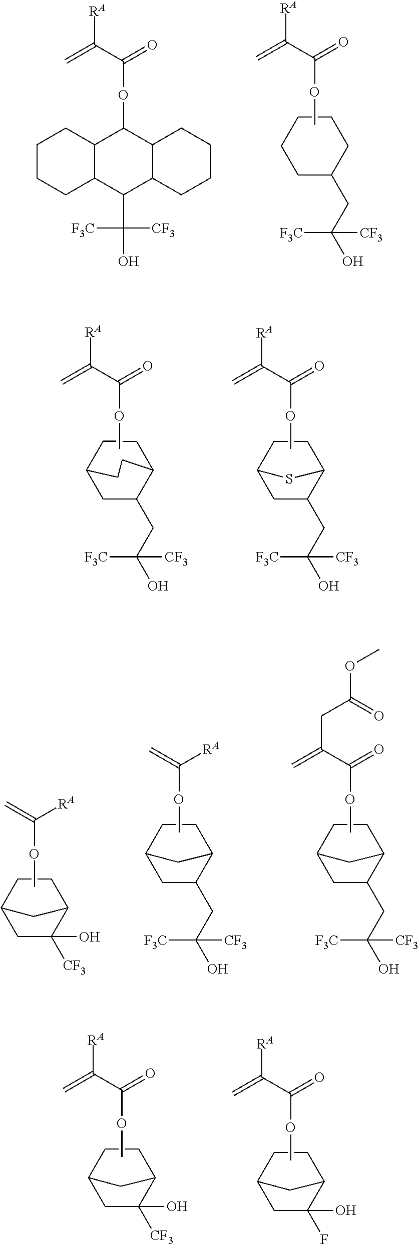

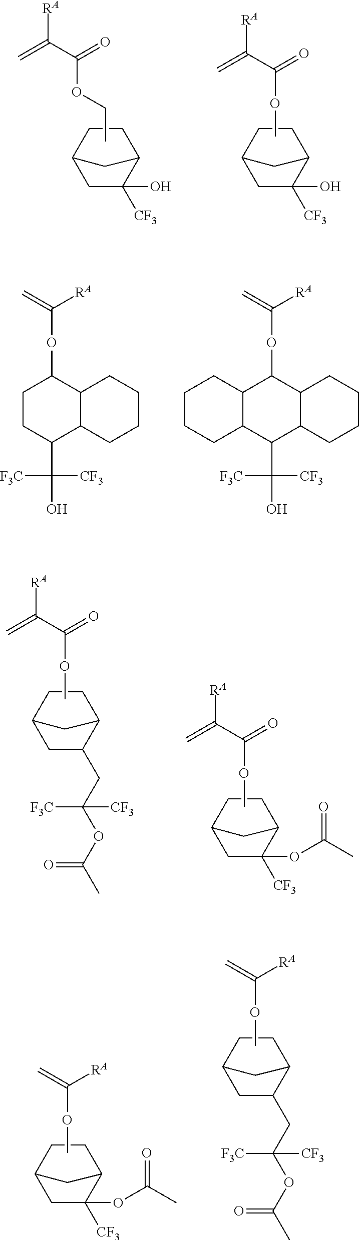

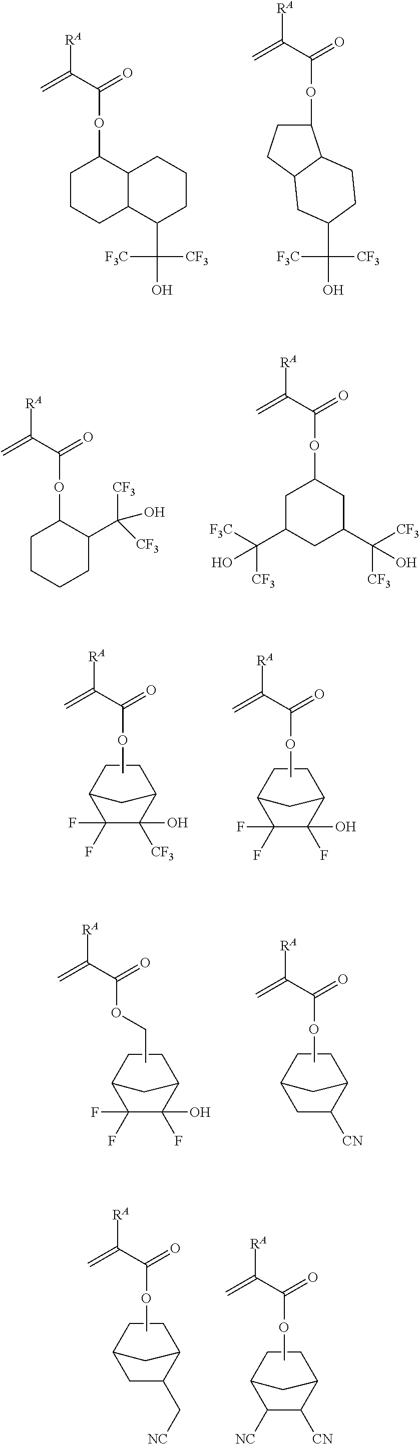

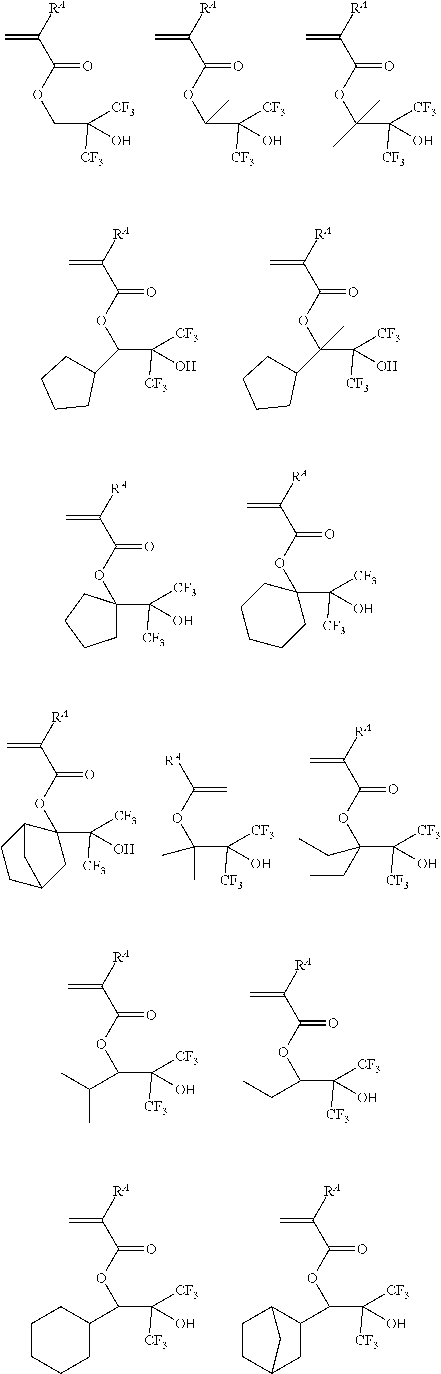

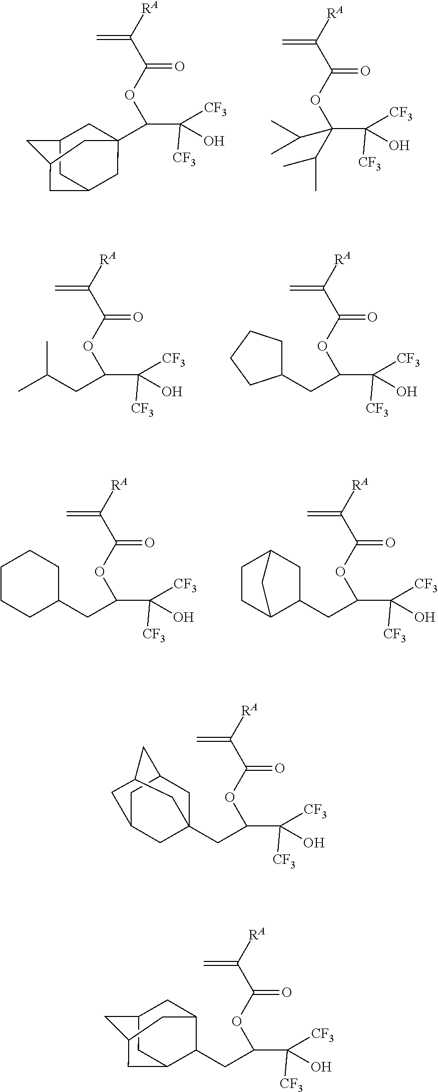

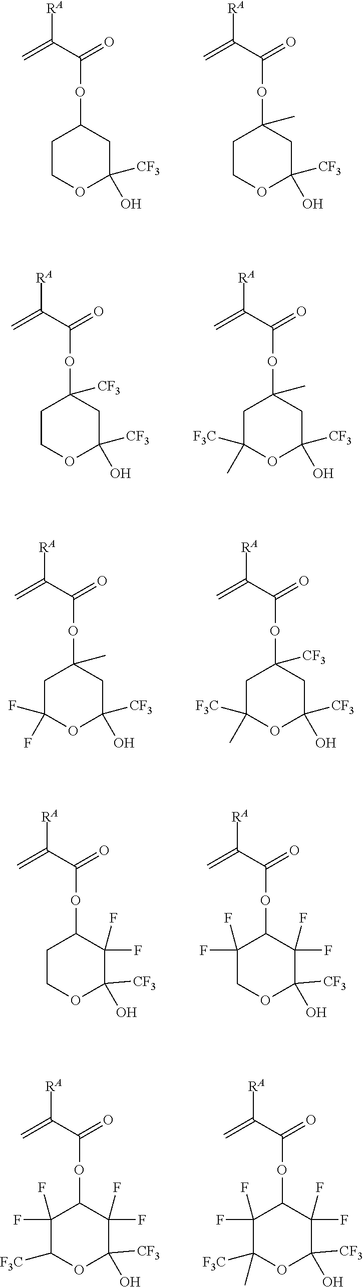

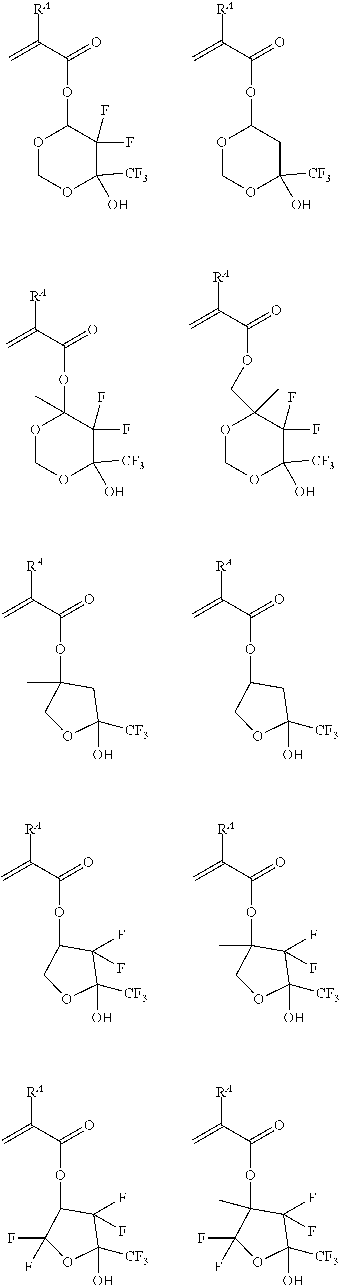

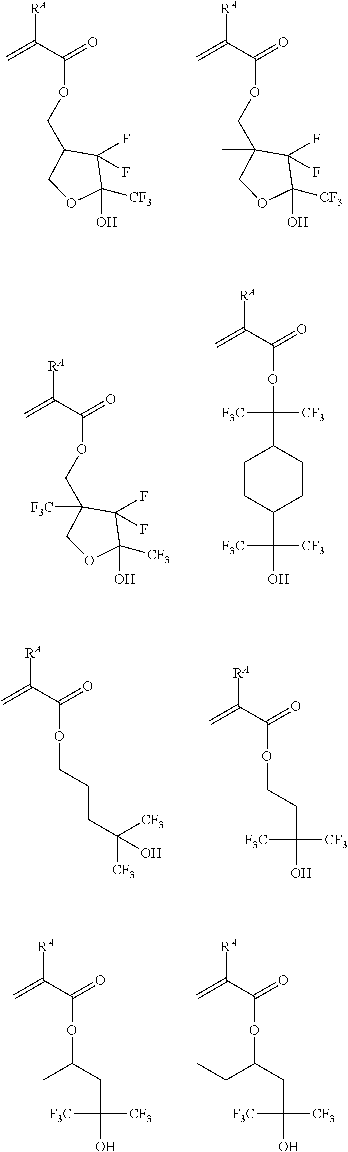

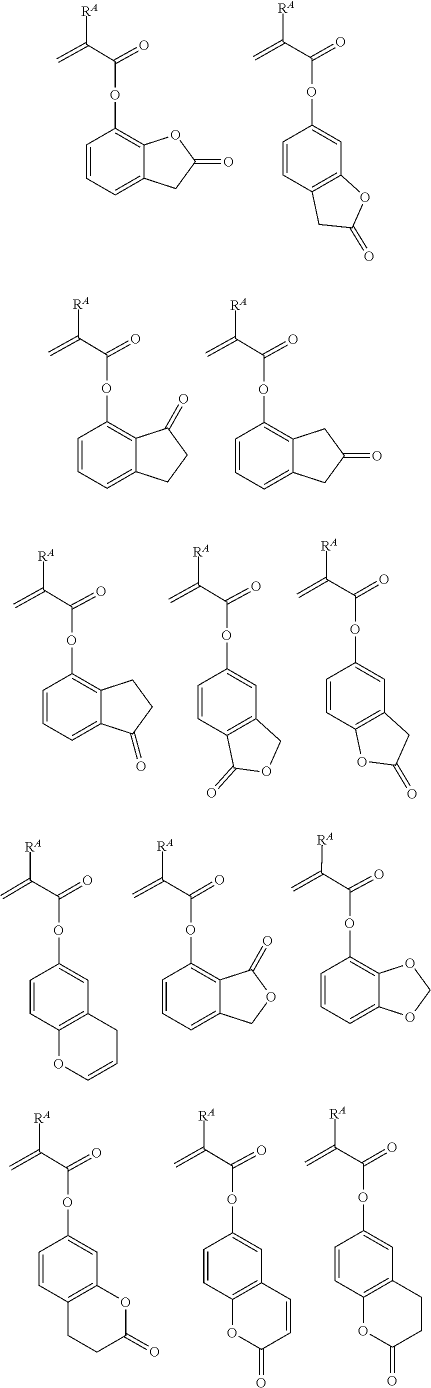

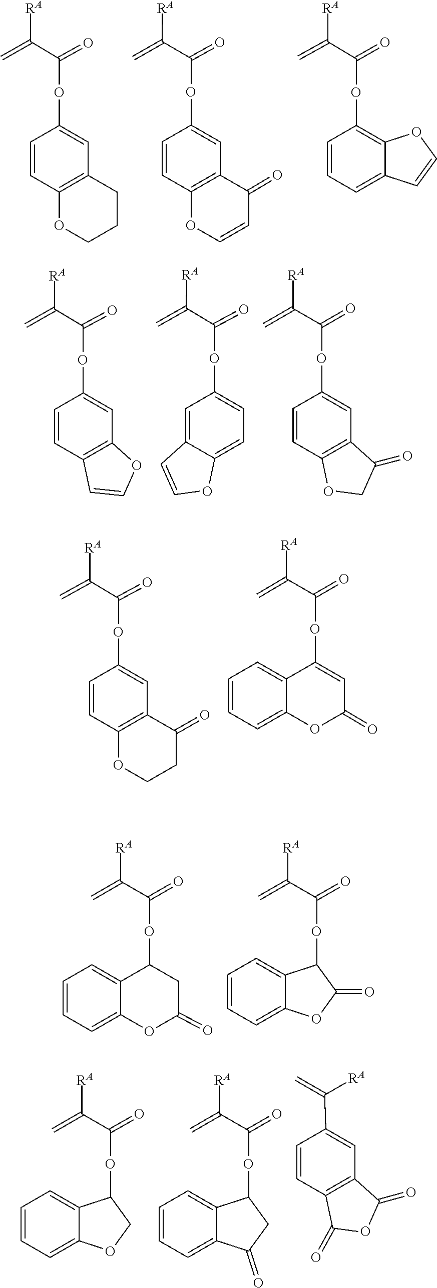

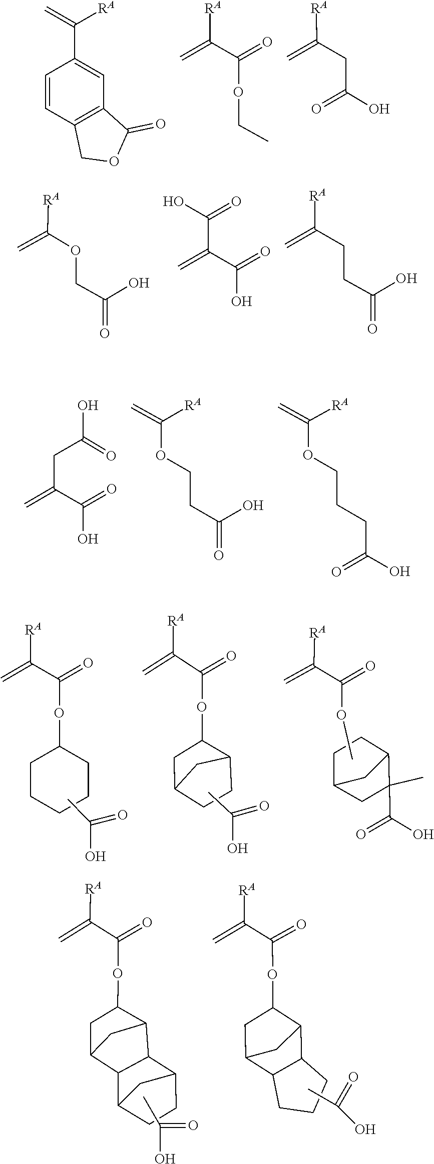

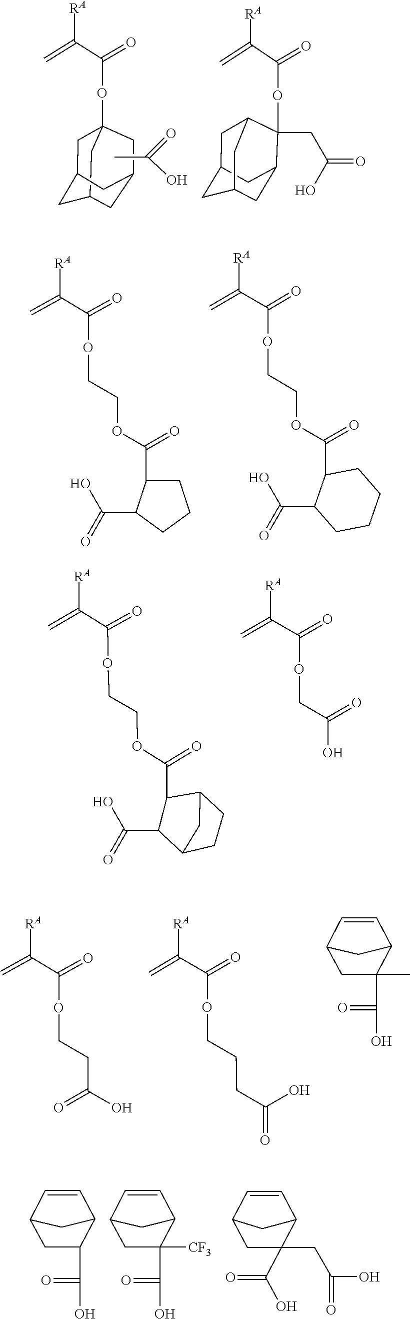

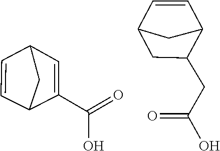

The base polymer may further comprise repeat units (e) derived from indene, benzofuran, benzothiophene, acenaphthylene, chromone, coumarin, and norbornadiene, or derivatives thereof. Examples of the monomer from which repeat units (e) are derived are shown below, but not limited thereto.

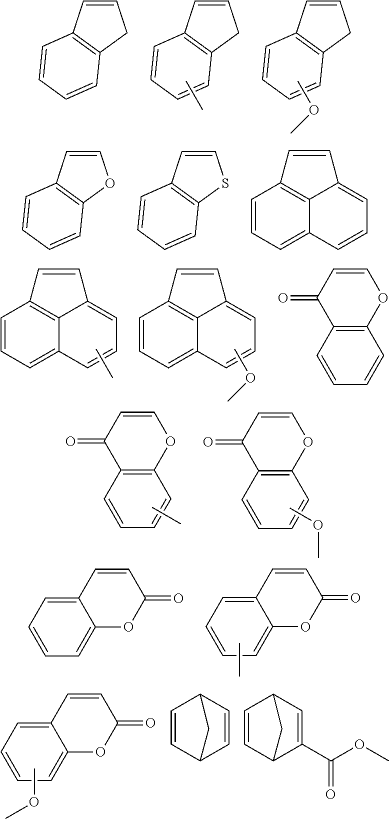

The base polymer may further include repeat units (f) which are derived from styrene, vinylnaphthalene, vinylanthracene, vinylpyrene, methyleneindane, vinylpyridine, vinylcarbazole.

The base polymer for formulating positive resist compositions comprises repeat units (a), as well as repeat units (b1) or (b2) containing an acid labile group. The base polymer contains repeat units (a), (b1), (b2), (c), (d), (e), and (f) in the following fraction:
preferably 0<a≤0.5, 0≤b1<1.0, 0≤b2<1.0, 0<b1+b2<1.0, 0≤c≤0.9, 0≤d≤0.9, 0≤e≤0.8, and 0≤f≤0.8;
more preferably 0.02≤a≤0.4, 0≤b1≤0.9, 0≤b2≤0.9, 0.1≤b1+b2≤0.9, 0≤c≤0.8, 0≤d≤0.8, 0≤e≤0.7, and 0≤f≤0.7;
even more preferably 0.05≤a≤0.35, 0≤b1≤0.8, 0≤b2≤0.8, 0.1≤b1+b2≤0.8, 0≤c≤0.75, 0≤d≤0.75, 0≤e≤0.6, and 0≤f≤0.6. In addition, a+b1+b2+c+d+e+f=1.0.

For the base polymer for formulating the negative resist composition, an acid labile group is not necessarily essential. The base polymer comprises repeat units (a), as well as repeat units (c), and optionally repeat units (d), (e), and/or (f). The base polymer contains these repeat units in the following fraction:
preferably 0<a≤0.5, 0<c≤1.0, 0≤d≤0.9, 0≤e≤0.8, and 0≤f≤0.8;
more preferably 0.02≤a≤0.4, 0.2≤c≤1.0, 0≤d≤0.8, 0≤e≤0.7, and 0≤f≤0.7;
even more preferably 0.05≤a≤0.35, 0.3≤c≤1.0, 0≤d≤0.75, 0≤e≤0.6 and 0≤f≤0.6. In addition, a+c+d+e+f=1.0.

Examples of the method for synthesizing the base polymer include a method in which in an organic solvent, a radical polymerization initiator is added to a monomer from which the repeat units are derived, and the mixture is heated for polymerization.

Examples of the organic solvent used during polymerization include toluene, benzene, tetrahydrofuran (THF), methyl ethyl ketone (MEK), diethyl ether, and dioxane. Examples of the polymerization initiator used herein include 2,2′-azobisisobutyronitrile (AIBN), 2,2′-azobis(2,4-dimethylvaleronitrile), dimethyl 2,2-azobis(2-methylpropionate), benzoyl peroxide, and lauroyl peroxide. The temperature during polymerization is preferably 50 to 80° C. The reaction time is preferably 2 to 100 hours, and more preferably 5 to 20 hours.

When a monomer having a hydroxy group is copolymerized, the hydroxy group may be replaced by an acetal group susceptible to deprotection with acid, typically ethoxyethoxy, prior to polymerization, and the polymerization be followed by deprotection with weak acid and water. Alternatively, the hydroxy group may be replaced by an acetyl, formyl, pivaloyl or similar group prior to polymerization, and the polymerization be followed by alkaline hydrolysis.

When hydroxystyrene or hydroxyvinylnaphthalene is copolymerized, an alternative method is possible. Specifically, acetoxystyrene or acetoxyvinylnaphthalene is used instead of hydroxystyrene or hydroxyvinylnaphthalene, and after polymerization, the acetoxy group is deprotected by alkaline hydrolysis, for thereby converting the polymer product to hydroxystyrene or hydroxyvinylnaphthalene.

For alkaline hydrolysis, a base such as aqueous ammonia or triethylamine may be used. Preferably the reaction temperature is −20° C. to 100° C., more preferably 0° C. to 60° C. The reaction time is 0.2 to 100 hours, more preferably 0.5 to 20 hours.

The base polymer preferably has a weight average molecular weight (Mw) in the range of 1,000 to 500,000, and more preferably 2,000 to 30,000, as measured by gel permeation chromatography (GPC) versus polystyrene standards using a THF solvent. A Mw in the range ensures that the resist film has heat resistance and high solubility in alkaline developer.

If a base polymer has a wide molecular weight distribution or dispersity (Mw/Mn), which indicates the presence of lower and higher molecular weight polymer fractions, there is a possibility that foreign matter is left on the pattern or the pattern profile is degraded. The influences of Mw and Mw/Mn become stronger as the pattern rule becomes finer. Therefore, the base polymer should preferably have a narrow dispersity (Mw/Mn) of 1.0 to 2.0, especially 1.0 to 1.5, in order to provide a resist composition suitable for micropatterning to a small feature size.

The base polymer may be a blend of two or more polymers which differ in compositional ratio, Mw or Mw/Mn.

### [Organic Solvent]

An organic solvent may be added to the resist composition. The organic solvent used herein is not particularly limited as long as the foregoing and other components are soluble therein. Examples of the organic solvent are described in JP-A 2008-111103, paragraphs [0144]-[0145](U.S. Pat. No. 7,537,880). Exemplary solvents include ketones such as cyclohexanone, cyclopentanone, methyl-2-n-pentyl ketone and 2-heptanone; alcohols such as 3-methoxybutanol, 3-methyl-3-methoxybutanol, 1-methoxy-2-propanol, 1-ethoxy-2-propanol and diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate and propylene glycol mono-tert-butyl ether acetate; and lactones such as γ-butyrolactone.

The organic solvent is preferably added to the resist composition of the present invention in an amount of 100 to 10,000 parts by weight, and more preferably 200 to 8,000 parts by weight per 100 parts by weight of the base polymer. The organic solvents may be used alone or in mixture of two or more thereof.

### [Quencher]

The inventive chemically amplified resist composition may comprise a quencher if necessary. As used herein, the “quencher” refers to a compound capable of trapping the acid generated from the acid generator for thereby preventing the acid from diffusing to the unexposed region.

Examples of the quencher include amine compounds, sulfonic salts, and carboxylic salts. Suitable amine compounds include primary, secondary and tertiary amine compounds, specifically amine compounds having any of a hydroxy group, ether bond, ester bond, lactone ring, cyano group or sulfonate ester bond, as described in JP-A 2008-111103, paragraphs [0146]-[0164]. Also suitable are primary or secondary amine compounds protected with a carbamate group, as described in JP 3790649. The protected amine compounds are effective when there are base labile components in the resist composition.

Examples of the sulfonic salt include compounds having the following formula (1). Examples of the carboxylic salt include compounds having the following formula (2).

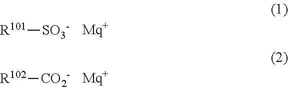

In formula (1), R¹⁰¹ is hydrogen or a C₁-C₄₀ hydrocarbyl group which may contain a heteroatom, exclusive of the hydrocarbyl group in which the hydrogen bonded to the carbon atom at α-position of the sulfo group is substituted by fluorine or fluoroalkyl moiety.

The C₁-C₄₀ hydrocarbyl group R¹⁰¹ may be saturated or unsaturated and straight, branched or cyclic. Examples thereof include C₁-C₄₀ alkyl groups such as methyl, ethyl, propyl, isopropyl, n-butyl, sec-butyl, tert-butyl, tert-pentyl, n-pentyl, n-hexyl, n-octyl, 2-ethylhexyl, n-nonyl, n-decyl; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decanyl, adamantyl, and adamantylmethyl; C₂-C₄₀ alkenyl groups such as vinyl, 1-propenyl, 2-propenyl, butenyl and hexenyl; C₃-C₄₀ unsaturated cyclic aliphatic hydrocarbyl groups such as cyclohexenyl; C₆-C₄₀ aryl groups such as phenyl, naphthyl, alkylphenyl groups (e.g., 2-methylphenyl, 3-methylphenyl, 4-methylphenyl, 4-ethylphenyl, 4-tert-butylphenyl, 4-n-butylphenyl), dialkylphenyl groups (e.g., 2,4-dimethylphenyl and 2,4,6-triisopropylphenyl), alkylnaphthyl groups (e.g., methylnaphthyl and ethylnaphthyl), dialkylnaphthyl groups (e.g., dimethylnaphthyl and diethylnaphthyl); and C₇-C₄₀ aralkyl groups such as benzyl, 1-phenylethyl and 2-phenylethyl.

In the hydrocarbyl group, some or all hydrogen may be replaced by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, carbonyl moiety, ether bond, thioether bond, ester bond, sulfonate ester bond, carbonate bond, lactone ring, sultone ring, carboxylic anhydride (—C(═O)—O—C(═O)—) or haloalkyl moiety. Examples of heteroatom-containing hydrocarbyl groups include heteroaryl groups such as thienyl, alkoxyphenyl groups such as 4-hydroxyphenyl, 4-methoxyphenyl, 3-methoxyphenyl, 2-methoxyphenyl, 4-ethoxyphenyl, 4-tert-butoxyphenyl and 3-tert-butoxyphenyl; alkoxynaphthyl groups such as methoxynaphthyl, ethoxynaphthyl, n-propoxynaphthyl and n-butoxynaphthyl; dialkoxynaphthyl groups such as dimethoxynaphthyl and diethoxynaphthyl; and aryloxoalkyl groups, typically 2-aryl-2-oxoethyl groups such as 2-phenyl-2-oxoethyl, 2-(1-naphthyl)-2-oxoethyl and 2-(2-naphthyl)-2-oxoethyl.

R¹⁰² is a C₁-C₄₀ hydrocarbyl group which may contain a heteroatom. Examples of the hydrocarbyl group R¹⁰² are as exemplified above for the hydrocarbyl group R¹⁰¹. Also included are fluorine-containing alkyl groups such as trifluoromethyl, trifluoroethyl, 2,2,2-trifluoro-1-methyl-1-hydroxyethyl, 2,2,2-trifluoro-1-(trifluoromethyl)-1-hydroxyethyl, and fluorine-containing aryl groups such as pentafluorophenyl and 4-trifluoromethylphenyl.

Examples of the anion in the sulfonic salt having the formula (1) are shown below, but not limited thereto.

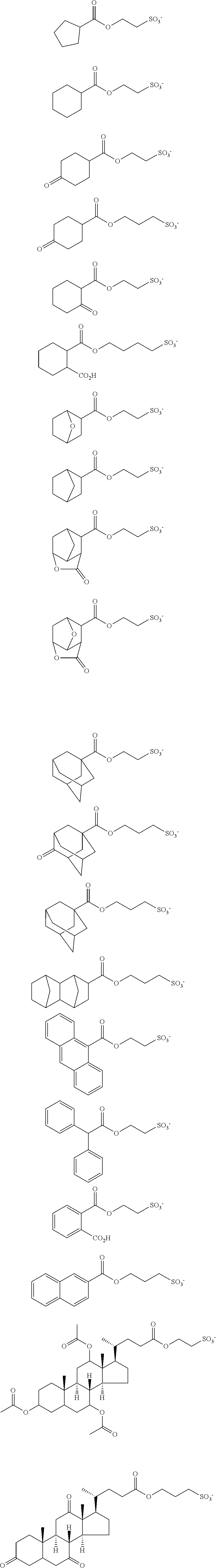

Examples of the anion in the carboxylic salt having the formula (2) are shown below, but not limited thereto. In the following formula, Me is methyl.

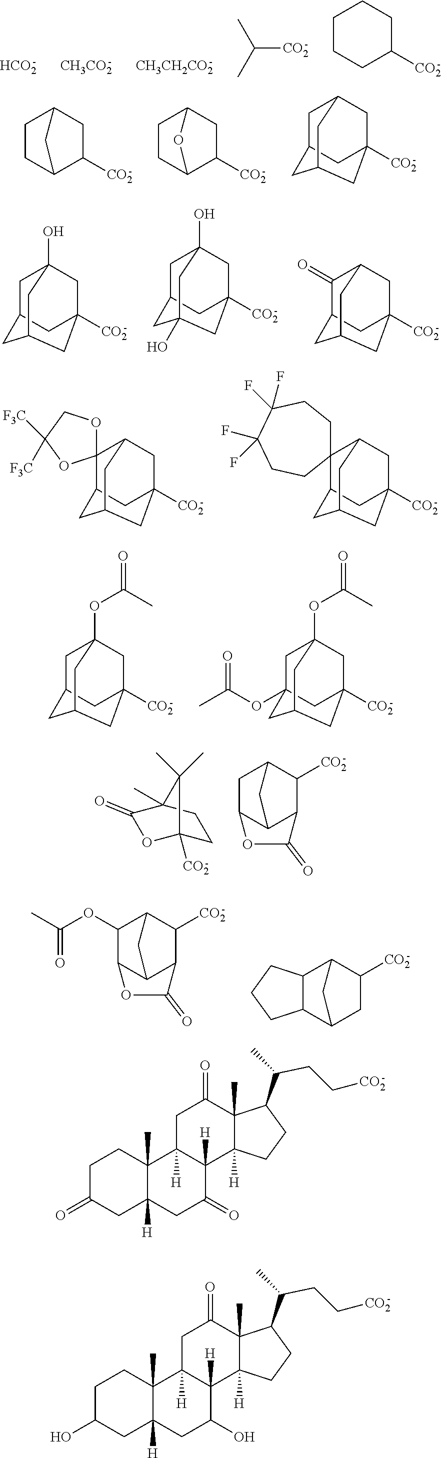

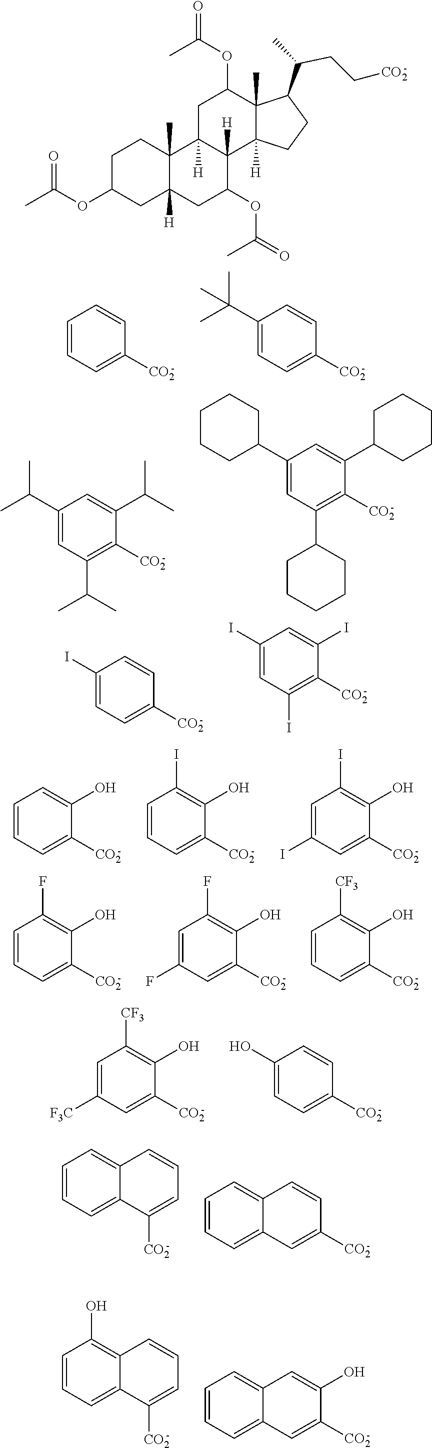

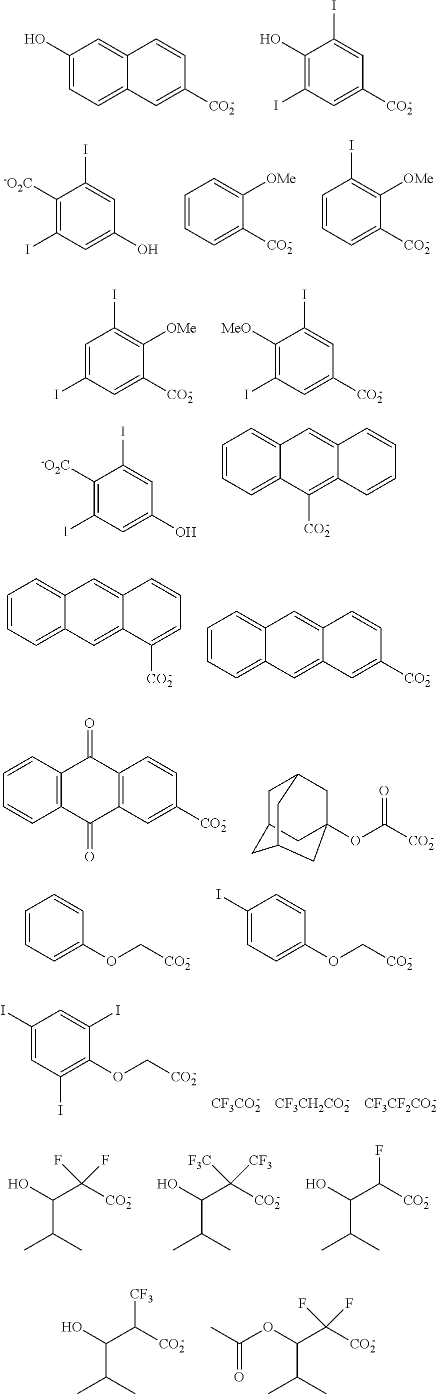

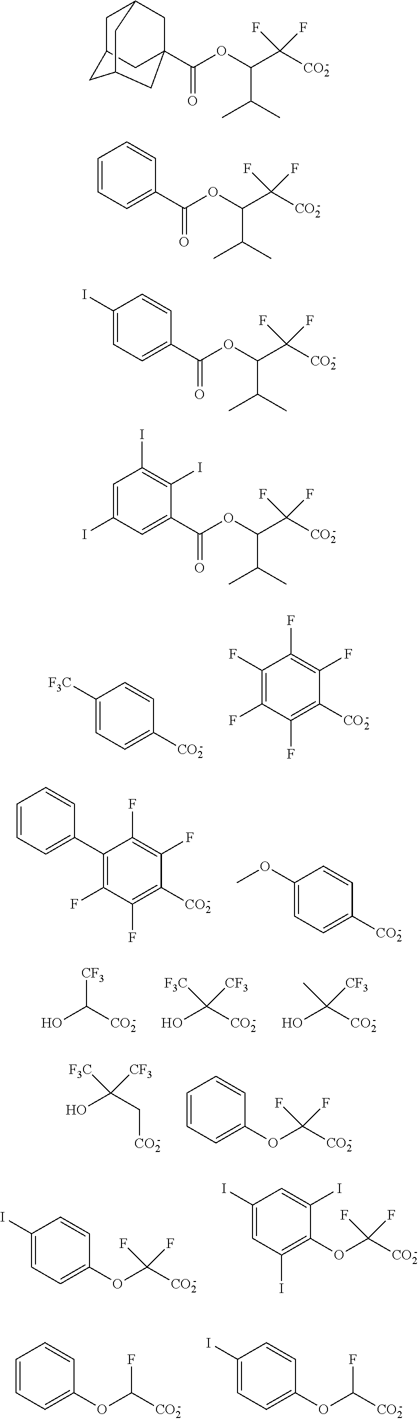

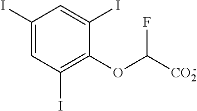

In the formulae (1) and (2), Mq⁺ is an onium cation. The onium cation is preferably a sulfonium, iodonium or ammonium cation. As the sulfonium ion, sulfonium cations having the formula (Z-1) are preferred. Examples thereof are as exemplified above for the sulfonium cations of the formulae (Z-1). As the iodonium cation, iodonium ions having the formula (Z-2) are preferred. Examples thereof are as exemplified above for the iodonium cations of the formulae (Z-2).

Also useful are quenchers of polymer type as described in U.S. Pat. No. 7,598,016 (JP-A 2008-239918). The polymeric quencher segregates at the resist surface and thus enhances the rectangularity of resist pattern. When a protective film is applied as is often the case in the immersion lithography, the polymeric quencher is also effective for preventing a film thickness loss of resist pattern or rounding of pattern top.

JP 6848776 discloses an onium salt having sulfonium cation and phenoxide anion sites in the same molecule, JP 6583136 and JP-A 2020-200311 disclose an onium salt having sulfonium cation and carboxylate anion sites in the same molecule, and JP 6274755 discloses an onium salt having iodonium cation and carboxylate anion sites in the same molecule. These onium salts may also be used as the quencher.

When the inventive chemically amplified resist composition comprises the quencher, the content of the quencher is preferably 0.001 to 12 parts by weight, more preferably 0.01 to 8 parts by weight per 80 parts by weight of the base polymer. By blending the quencher, the sensitivity of the resist film is easily adjusted, the diffusion of the acid in the resist film is suppressed to improve the resolution, and a sensitivity change after the exposure is suppressed, or the dependency on the substrate and the environment is reduced, so that the exposure latitude and the pattern profile can be improved. Also, adhesion to the substrate can be improved by adding the quencher. The quenchers may be used alone, or in combination of two or more thereof.

### [Other Components]

In addition to the foregoing components, the inventive chemically amplified resist composition may contain other components such as an acid generator, surfactant, dissolution inhibitor, crosslinker, water repellency improver and acetylene alcohol.

The acid generator is typically a compound (photoacid generator) capable of generating an acid upon exposure to actinic ray or radiation. Although the PAG used herein may be any compound capable of generating an acid upon exposure to high-energy radiation, those compounds capable of generating sulfonic acid, imide acid (imidic acid) or methide acid are preferred. Suitable PAGs include sulfonium salts, iodonium salts, sulfonyldiazomethane, N-sulfonyloxyimide, and oxime-O-sulfonate acid generators. Exemplary PAGs are described in JP-A 2008-111103, paragraphs [0122]-[0142](U.S. Pat. No. 7,537,880), JP-A 2018-5224, and JP-A 2018-25789. The acid generator is preferably used in an amount of 0 to 200 parts, more preferably 0.1 to 100 parts by weight per 100 parts by weight of the base polymer.

Exemplary surfactants are described in JP-A 2008-111103, paragraphs [0165]-[0166]. Inclusion of a surfactant may improve or control the coating characteristics of the resist composition. The surfactant is preferably added in an amount of 0.0001 to 10 parts by weight per 100 parts by weight of the base polymer. The surfactants may be used alone or in admixture of two or more thereof.

The inclusion of a dissolution inhibitor in the positive resist composition of the present invention may lead to an increased difference in dissolution rate between exposed and unexposed areas and a further improvement in resolution. Examples of the dissolution inhibitor include a compound having at least two phenolic hydroxy groups on the molecule, in which an average of from 0 to 100 mol % of all the hydrogen atoms on the phenolic hydroxy groups are replaced by acid labile groups or a compound having at least one carboxy group on the molecule, in which an average of 50 to 100 mol % of all the hydrogen atoms on the carboxy groups are replaced by acid labile groups, both the compounds having a molecular weight of 100 to 1,000, and preferably 150 to 800. Typical are bisphenol A, trisphenol, phenolphthalein, cresol novolac, naphthalenecarboxylic acid, adamantanecarboxylic acid, and cholic acid derivatives in which the hydrogen atom on the hydroxy or carboxy group is replaced by an acid labile group, as described in U.S. Pat. No. 7,771,914 (JP-A 2008-122932, paragraphs [0155]-[0178]).

When the inventive resist material is positive, and contains the dissolution inhibitor, the dissolution inhibitor is preferably added in an amount of 0 to 50 parts, more preferably 5 to 40 parts by weight per 100 parts by weight of the base polymer. The dissolution inhibitors may be used alone or in combination of two or more thereof.

In the case of negative resist compositions, a negative pattern may be formed by adding a crosslinker to reduce the dissolution rate of exposed area. Examples of the crosslinker include epoxy compounds, melamine compounds, guanamine compounds, glycoluril compounds and urea compounds having substituted thereon at least one group selected from among methylol, alkoxymethyl and acyloxymethyl groups, isocyanate compounds, azide compounds, and compounds having a double bond such as an alkenyloxy group. These compounds may be used as an additive or introduced into a polymer side chain as a pendant. Hydroxy-containing compounds may also be used as the crosslinker.

Examples of the epoxy compound include tris(2,3-epoxypropyl) isocyanurate, trimethylolmethane triglycidyl ether, trimethylolpropane triglycidyl ether, and triethylolethane triglycidyl ether.

Examples of the melamine compound include hexamethylol melamine, hexamethoxymethyl melamine, hexamethylol melamine compounds having 1 to 6 methylol groups methoxymethylated and mixtures thereof, hexamethoxyethyl melamine, hexaacyloxymethyl melamine, hexamethylol melamine compounds having 1 to 6 methylol groups acyloxymethylated and mixtures thereof.

Examples of the guanamine compound include tetramethylol guanamine, tetramethoxymethyl guanamine, tetramethylol guanamine compounds having 1 to 4 methylol groups methoxymethylated and mixtures thereof, tetramethoxyethyl guanamine, tetraacyloxyguanamine, tetramethylol guanamine compounds having 1 to 4 methylol groups acyloxymethylated and mixtures thereof.

Examples of the glycoluril compound include tetramethylol glycoluril, tetramethoxyglycoluril, tetramethoxymethyl glycoluril, tetramethylol glycoluril compounds having 1 to 4 methylol groups methoxymethylated and mixtures thereof, tetramethylol glycoluril compounds having 1 to 4 methylol groups acyloxymethylated and mixtures thereof. Examples of the urea compound include tetramethylol urea, tetramethoxymethyl urea, tetramethylol urea compounds having 1 to 4 methylol groups methoxymethylated and mixtures thereof, and tetramethoxyethyl urea.

Examples of the isocyanate compound include tolylene diisocyanate, diphenylmethane diisocyanate, hexamethylene diisocyanate and cyclohexane diisocyanate.

Examples of the azide compound include 1,1′-biphenyl-4,4′-bisazide, 4,4′-methylidenebisazide, and 4,4′-oxybisazide.

Examples of the alkenyl ether group-containing compound include ethylene glycol divinyl ether, triethylene glycol divinyl ether, 1,2-propanediol divinyl ether, 1,4-butanediol divinyl ether, tetramethylene glycol divinyl ether, neopentyl glycol divinyl ether, trimethylol propane trivinyl ether, hexanediol divinyl ether, 1,4-cyclohexanediol divinyl ether, pentaerythritol trivinyl ether, pentaerythritol tetravinyl ether, sorbitol tetravinyl ether, sorbitol pentavinyl ether, and trimethylol propane trivinyl ether.

In the negative resist composition, the crosslinker is preferably added in an amount of 0.1 to 50 parts, more preferably 1 to 40 parts by weight per 100 parts by weight of the base polymer. The crosslinkers may be used alone or in admixture of two or more thereof.

The water repellency improver improves the water repellency of the resist film surface, and may be used in the topcoatless immersion lithography. Suitable water repellency improvers include polymers having a fluoroalkyl group and polymers having a specific structure with a 1,1,1,3,3,3-hexafluoro-2-propanol residue and are described in JP-A 2007-297590 and JP-A 2008-111103, for example. The water repellency improver should be soluble in alkaline developers and organic solvent developers. The water repellency improver of specific structure with a 1,1,1,3,3,3-hexafluoro-2-propanol residue is well soluble in the developer. A polymer having an amino group or amine salt copolymerized as repeat units may serve as the water repellency improver and is effective for preventing evaporation of acid during PEB, thus preventing any hole pattern opening failure after development. An appropriate amount of the water repellency improver is 0 to 20 parts, preferably 0.5 to 10 parts by weight per 100 parts by weight of the base polymer. The water repellency improver may be used alone, or in combination of two or more thereof.

Specific examples of the acetylene alcohol include those described in paragraphs [0179]-[0182] of JP-A 2008-122932. The acetylene alcohol is preferably added to the resist composition of the present invention in an amount of 0 to 5 parts by weight per 100 parts by weight of the base polymer. The acetylene alcohols may be used alone or in combination of two or more thereof.

### [Pattern Forming Process]

The resist composition is used in the fabrication of various integrated circuits. Examples of the pattern forming process include a process including the steps of: applying the above-described resist composition onto a substrate to form a resist film on the substrate, exposing the resist film to high-energy radiation, and developing the exposed resist film in a developer.

The inventive resist composition is first applied onto a substrate on which an integrated circuit is to be formed (e.g., Si, SiO₂, SiN, SiON, TiN, WSi, BPSG, SOG, or organic antireflective coating) or a substrate on which a mask circuit is to be formed (e.g., Cr, CrO, CrON, MoSi₂, or SiO₂) by a suitable coating technique such as spin coating, roll coating, flow coating, dipping, spraying or doctor coating. The resulting resist film is generally 0.01 to 2 μm thick. The coating is prebaked on a hotplate preferably at a temperature of 60 to 150° C. for 10 seconds to 30 minutes, and more preferably at 80 to 120° C. for 30 seconds to 20 minutes to form a resist film.

Then the resist film is exposed to high-energy radiation. Specific examples of the high-energy radiation include UV, deep-UV, EB, EUV of wavelength 3-15 nm, i-line, x-ray, soft x-ray, excimer laser light, γ-ray or synchrotron radiation. When UV, deep-UV, EUV, X-rays, soft X-rays, excimer laser light, γ-rays, or synchrotron radiation is used as the high-energy radiation, the resist film is exposed thereto directly or through a mask having a desired pattern preferably at a dose of about 1 to 200 mJ/cm², and more preferably about 10 to 100 mJ/cm². When EB is used as the high-energy radiation, the resist film is exposed thereto directly or through a mask having a desired pattern preferably at a dose of about 0.1 to 300 μC/cm², and more preferably about 0.5 to 200 μC/cm². It is appreciated that the inventive resist composition is suited in micropatterning using KrF excimer laser light, ArF excimer laser, EB, EUV, x-ray, soft x-ray, γ-ray or synchrotron radiation, especially in micropatterning using EB or EUV.

After the exposure, the resist film may be baked (PEB) on a hotplate or in an oven preferably at 30 to 150° C. for 10 seconds to 30 minutes, more preferably at 50 to 120° C. for 30 seconds to 20 minutes.

After the exposure or PEB, the resist film is developed in a developer in the form of an aqueous base solution for 3 seconds to 3 minutes, preferably 5 seconds to 2 minutes by conventional techniques such as dip, puddle and spray techniques. A typical developer is preferably a 0.1 to 10 wt %, more preferably 2 to 5 wt % aqueous solution of tetramethylammonium hydroxide (TMAH), tetraethylammonium hydroxide, tetrapropylammonium hydroxide, or tetrabutylammonium hydroxide. In the case of positive tone, the resist film in the exposed area is dissolved in the developer whereas the resist film in the unexposed area is not dissolved. In this way, the desired positive pattern is formed on the substrate. In the case of negative tone, inversely the resist film in the exposed area is insolubilized whereas the resist film in the unexposed area is dissolved away.

In an alternative embodiment, a negative pattern can be obtained from the positive resist composition comprising a base polymer containing acid labile groups by effecting organic solvent development. Examples of the developer used herein include 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate. The organic solvents may be used alone or in admixture.

At the end of development, the resist film is rinsed. As the rinsing liquid, a solvent which is miscible with the developer and does not dissolve the resist film is preferred. Suitable solvents include alcohols of 3 to 10 carbon atoms, ether compounds of 8 to 12 carbon atoms, alkanes, alkenes, and alkynes of 6 to 12 carbon atoms, and aromatic solvents.

Specifically, suitable alcohols of 3 to 10 carbon atoms include n-propyl alcohol, isopropyl alcohol, 1-butyl alcohol, 2-butyl alcohol, isobutyl alcohol, tert-butyl alcohol, 1-pentanol, 2-pentanol, 3-pentanol, tert-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, and 1-octanol.

Suitable ether compounds of 8 to 12 carbon atoms include di-n-butyl ether, diisobutyl ether, di-sec-butyl ether, di-n-pentyl ether, diisopentyl ether, di-sec-pentyl ether, di-tert-pentyl ether, and di-n-hexyl ether.

Suitable alkanes of 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, and cyclononane. Suitable alkenes of 6 to 12 carbon atoms include hexene, heptene, octene, cyclohexene, methylcyclohexene, dimethylcyclohexene, cycloheptene, and cyclooctene. Suitable alkynes of 6 to 12 carbon atoms include hexyne, heptyne, and octyne.

Suitable aromatic solvents include toluene, xylene, ethylbenzene, isopropylbenzene, tert-butylbenzene and mesitylene.

Rinsing is effective for minimizing the risks of resist pattern collapse and defect formation. However, rinsing is not essential. If rinsing is omitted, the amount of solvent used may be reduced.

A hole or trench pattern after development may be shrunk by the thermal flow, RELACS® or DSA process. A hole pattern is shrunk by coating a shrink agent thereto, and baking such that the shrink agent may undergo crosslinking at the resist surface as a result of the acid catalyst diffusing from the resist layer during bake, and the shrink agent may attach to the sidewall of the hole pattern. The bake is preferably at a temperature of 70 to 180° C., more preferably 80 to 170° C., for a time of 10 to 300 seconds. The extra shrink agent is stripped and the hole pattern is shrunk.

### EXAMPLES

Synthesis Examples, Examples and Comparative Examples are given below by way of illustration and not by way of limitation. Analysis is made by time-of-flight mass spectrometry using the instrument,
MALDI TOF-MS: S3000 manufactured by JEOL Ltd.

### [1] Synthesis of Onium Salt Monomers

### Example 1-1

### Synthesis of Onium Salt Monomer a-1

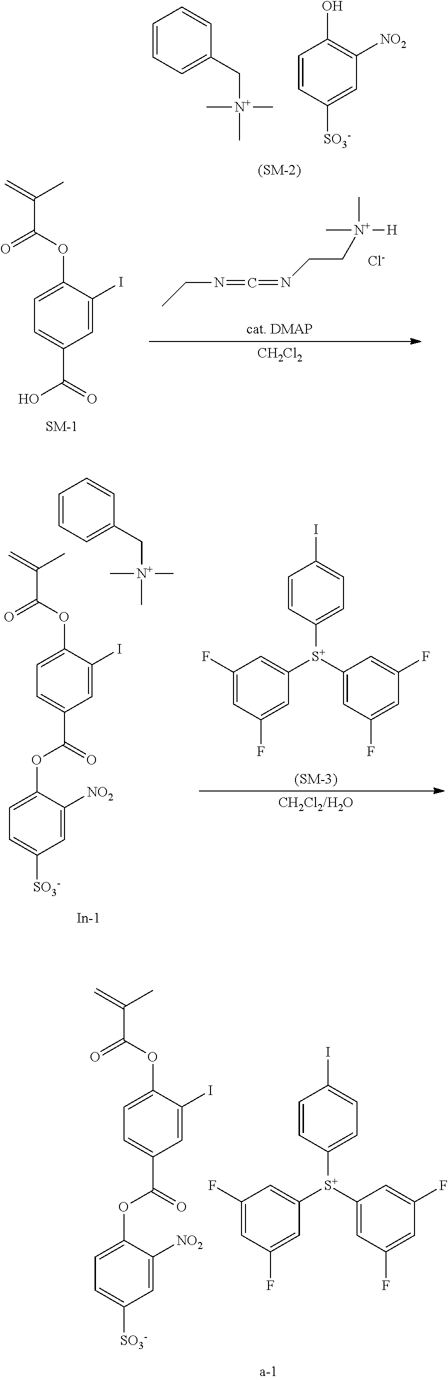

(1) Synthesis of Intermediate In-1

In nitrogen atmosphere, a reactor was charged with 16.6 g of reactant SM-1, 20.3 g of reactant SM-2, 0.75 g of DMAP, and 300 g of methylene chloride and cooled in an ice bath. While the internal temperature of the reactor was kept below 20° C., 13.3 g of 1-ethyl-3-(3-dimethylaminopropyl)carbodiimide hydrochloride in powder form was added. Thereafter, the reactor was allowed to reach room temperature, at which the reaction solution was aged for 12 hours. At the end of aging, water was added to quench the reaction, followed by ordinary aqueous work-up. The subsequent steps of distilling off the solvent and washing the residue with diisopropyl ether gave 33.8 g of Intermediate In-1 as a brown solid (yield 99%).

### (2) Synthesis of Onium Salt Monomer a-1

Under nitrogen atmosphere, a reactor was charged with 33.8 g of Intermediate In-1, 23.2 g of reactant SM-3, 200 g of methylene chloride and 150 g of water, and the mixture was stirred for 30 minutes. Subsequently, the organic layer was taken out, washed with water, and then concentrated under reduced pressure. The concentrate was purified by silica gel chromatography, and recrystallized by addition of diisopropyl ether to give 44.3 g (yield 90%) of the titled onium salt monomer a-1 as a brown crystal.

### MALDI TOF-MS:

POSITIVE M⁺461 (corresponding to C₁₈H₁₀F₄IS⁺)
NEGATIVE M⁻532 (corresponding to C₁₇H₁₁INO₉S⁻)

### Examples 1-2 to 1-13

### Synthesis of Onium Salt Type Monomers a-2 to a-8

Onium Salt Type Monomers a-2 to a-8 of the following formulae were synthesized using the corresponding reactants and well-known organic chemistry reaction.

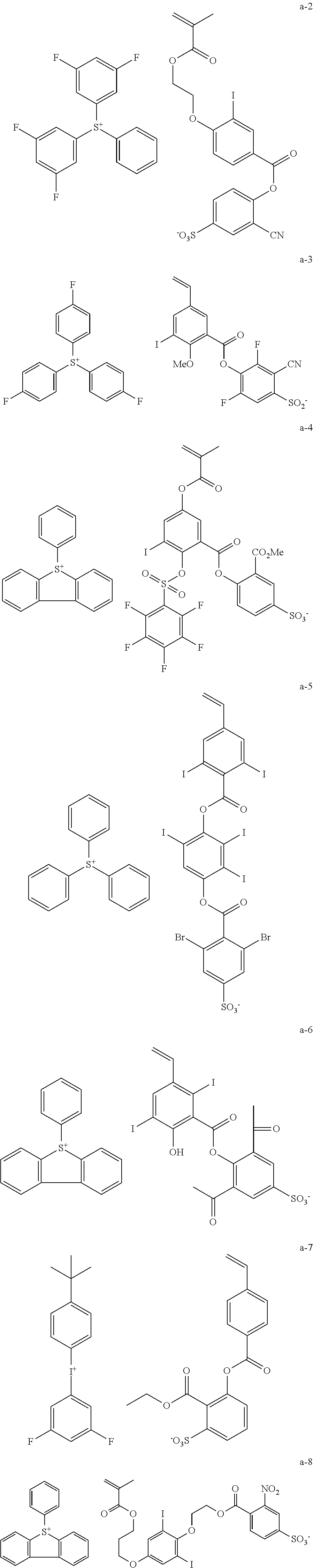

### Comparative Examples 1-1 to 1-4

Synthesis of Comparative Onium Salt Type Monomers ca-1 to ca-4

Comparative onium salt type monomers ca-1 to ca-4 of the following formulae were synthesized using the corresponding reactants and well-known organic chemistry reaction.

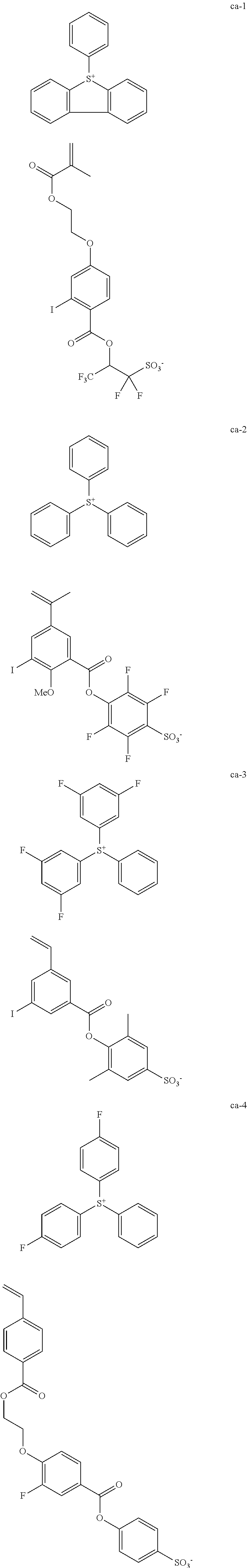

### [2] Synthesis of Base Polymer

Monomers a-1 to a-8, comparative monomers ca-1 to ca-4, and the monomers shown below were used in the synthesis of base polymers.

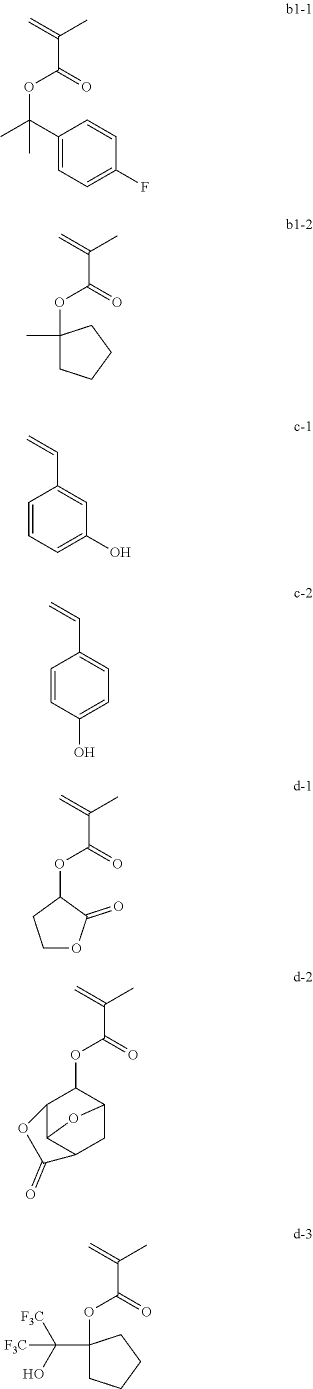

### Example 2-1

### Synthesis of Polymer P-1

In nitrogen atmosphere, a flask was charged with 43.0 g of Monomer a-1, 35.6 g of Monomer b1-1, 10.4 g of Monomer c-1, 3.34 g of V-601 (manufactured by Fujifilm Wako Pure Chemical Corp.), and 139 g of MEK to prepare a monomer/initiator solution. Another flask under nitrogen atmosphere was charged with 46 g of MEK, which was heated to 80° C. with stirring. The monomer/initiator solution was added dropwise to the MEK over 4 hours. At the end of dropwise addition, the polymerization solution was continuously stirred for 2 hours while maintaining the temperature of 80° C. The polymerization solution was cooled to room temperature. The obtained polymerization solution was added dropwise to 3,000 g of hexane with vigorous stirring. The precipitate was collected by filtration. The precipitate was washed twice with 600 g of hexane and vacuum dried at 50° C. for 20 hours to obtain Polymer P-1 as white powder (amount 85.2 g, yield 96%). The polymer P-1 had a value of Mw of 9,200, and a value of Mw/Mn of 1.73. It is noted that Mw is measured by GPC versus polystyrene standards using DMF solvent.

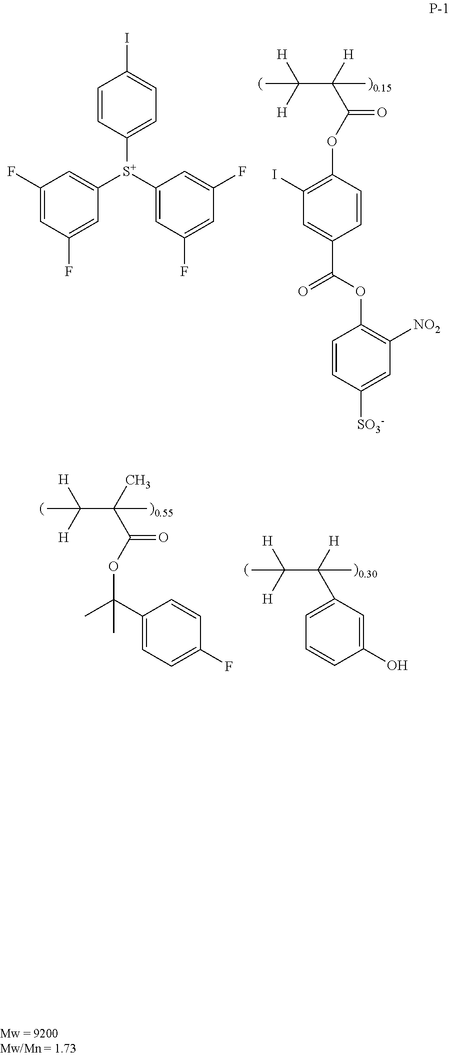

### Examples 2-2 to 2-15 and Comparative Examples 2-1 to 2-10

### Synthesis of Polymers P-2 to P-15 and Comparative Polymers CP-1 to CP-10

Polymers shown in Tables 1 and 2 were prepared by the same procedure as in Example 2-1 except that the type and blending ratio of monomers were changed.

TABLE 1

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| P-1 | a-1 | 15 | b1-1 | 55 | c-1 | 30 | — | — | — | — | 9,200 | 1.73 |
| P-2 | a-2 | 15 | b1-1 | 55 | c-1 | 30 | — | — | — | — | 9,200 | 1.76 |
| P-3 | a-3 | 15 | b1-1 | 55 | c-1 | 30 | — | — | — | — | 9,200 | 1.75 |
| P-4 | a-4 | 15 | b1-1 | 55 | c-1 | 30 | — | — | — | — | 9,100 | 1.75 |
| P-5 | a-5 | 15 | b1-1 | 55 | c-1 | 30 | — | — | — | — | 9,200 | 1.72 |
| P-6 | a-6 | 15 | b1-1 | 55 | c-1 | 30 | — | — | — | — | 9,100 | 1.75 |
| P-7 | a-7 | 15 | b1-1 | 55 | c-1 | 30 | — | — | — | — | 9,300 | 1.73 |
| P-8 | a-8 | 15 | b1-1 | 55 | c-1 | 30 | — | — | — | — | 9,100 | 1.74 |
| P-9 | a-1 | 15 | b1-2 | 55 | c-1 | 30 | — | — | — | — | 9,000 | 1.71 |
| P-10 | a-2 | 15 | b1-2 | 55 | c-1 | 30 | — | — | — | — | 9,100 | 1.74 |
| P-11 | a-1 | 15 | b1-1 | 55 | c-2 | 30 | — | — | — | — | 9,200 | 1.72 |
| P-12 | a-1 | 10 | b1-1 | 50 | c-1 | 30 | d-1 | 10 | — | — | 9,100 | 1.76 |
| P-13 | a-1 | 10 | b1-1 | 50 | c-1 | 30 | d-2 | 10 | — | — | 9,100 | 1.77 |
| P-14 | a-1 | 10 | b1-2 | 50 | c-1 | 30 | d-3 | 10 | — | — | 9,200 | 1.71 |
| P-15 | a-3 | 12 | b1-1 | 55 | c-1 | 38 | — | — | — | — | 8,900 | 1.76 |

TABLE 2

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| CP-1 | ca-1 | 15 | b1-1 | 55 | c-1 | 30 | — | — | — | — | 9,400 | 1.74 |
| CP-2 | ca-2 | 15 | b1-1 | 55 | c-1 | 30 | — | — | — | — | 9,200 | 1.74 |
| CP-3 | ca-3 | 15 | b1-1 | 55 | c-1 | 30 | — | — | — | — | 9,200 | 1.75 |
| CP-4 | ca-4 | 15 | b1-1 | 55 | c-1 | 30 | — | — | — | — | 8,800 | 1.74 |
| CP-5 | ca-1 | 15 | b1-2 | 55 | c-1 | 30 | — | — | — | — | 9,400 | 1.73 |
| CP-6 | ca-2 | 15 | b1-1 | 55 | c-2 | 30 | — | — | — | — | 9,100 | 1.71 |
| CP-7 | ca-3 | 10 | b1-1 | 50 | c-1 | 30 | d-1 | 10 | — | — | 9,000 | 1.74 |
| CP-8 | ca-4 | 10 | b1-1 | 50 | c-1 | 30 | d-2 | 10 | — | — | 9,000 | 1.73 |
| CP-9 | ca-1 | 10 | b1-1 | 50 | c-1 | 20 | d-2 | 10 | d-3 | 10 | 9,100 | 1.76 |
| CP-10 | ca-2 | 12 | b1-1 | 55 | c-1 | 38 | — | — | — | — | 9,200 | 1.75 |

### [3] Preparation of Chemically Amplified Resist Compositions

### Examples 3-1 to 3-15 and Comparative Examples 3-1 to 3-10

A chemically amplified resist composition (R-1 to R-15 and CR-1 to CR-10) was prepared by dissolving a component selected from the inventive base polymers (P-1 to P-15), a component selected from comparative base polymers (CP-1 to CP-10), a component selected from acid generators (PAG-1 and PAG-2) and a component selected from quenchers (SQ-1 to SQ-3 and AQ-1) in a solvent containing 0.01 wt % of surfactant FC-4430 (3M Company) in accordance with the formulation shown in Tables 3 and 4 below, and filtering the solution through a Teflon® filter with a pore size of 0.2 μm.

TABLE 3

| | Resist | Base polymer | Quencher | Acid generator | Solvent 1 | Solvent 2 |
|---|---|---|---|---|---|---|
| Example | composition | (pbw) | (pbw) | (pbw) | (pbw) | (pbw) |
| 3-1 | R-1 | P-1 (80) | SQ-1 (8.0) | — | PGMEA (2,200) | DAA (900) |
| 3-2 | R-2 | P-2 (80) | SQ-1 (7.8) | — | PGMEA (2,200) | DAA (900) |
| 3-3 | R-3 | P-3 (80) | SQ-1 (7.4) | — | PGMEA (2,200) | DAA (900) |
| 3-4 | R-4 | P-4 (80) | SQ-1 (8.0) | — | PGMEA (2,200) | DAA (900) |
| 3-5 | R-5 | P-5 (80) | SQ-1 (8.0) | — | PGMEA (2,200) | DAA (900) |
| 3-6 | R-6 | P-6 (80) | SQ-1 (8.0) | — | PGMEA (2,200) | DAA (900) |
| 3-7 | R-7 | P-7 (80) | SQ-1 (4.0) | — | PGMEA (2,200) | DAA (900) |
| | | | AQ-1 (4.0) | | | |
| 3-8 | R-8 | P-8 (80) | SQ-3 (7.8) | — | PGMEA (2,200) | DAA (900) |
| 3-9 | R-9 | P-9 (80) | SQ-1 (8.0) | — | PGMEA (2,200) | DAA (900) |
| 3-10 | R-10 | P-10 (80) | SQ-1 (8.0) | PAG-1 (10) | PGMEA (2,200) | DAA (900) |
| 3-11 | R-11 | P-11 (80) | SQ-1 (8.0) | PAG-2 (15) | PGMEA (2,200) | DAA (900) |
| 3-12 | R-12 | P-12 (80) | SQ-1 (8.0) | — | PGMEA (2,200) | DAA (900) |
| 3-13 | R-13 | P-13 (80) | SQ-1 (8.0) | — | PGMEA (2,200) | DAA (900) |
| 3-14 | R-14 | P-14 (80) | SQ-2 (8.0) | — | PGMEA (2,200) | DAA (900) |
| 3-15 | R-15 | P-15 (80) | SQ-2 (8.0) | — | PGMEA (2,200) | DAA (900) |

TABLE 4

| Comparative | Resist | Base polymer | Quencher | Acid generator | Solvent 1 | Solvent 2 |
|---|---|---|---|---|---|---|
| Example | composition | (pbw) | (pbw) | (pbw) | (pbw) | (pbw) |
| 3-1 | CR-1 | CP-1 (80) | SQ-1 (8.0) | — | PGMEA (2,200) | DAA (900) |
| 3-2 | CR-2 | CP-2 (80) | SQ-1 (7.8) | — | PGMEA (2,200) | DAA (900) |
| 3-3 | CR-3 | CP-3 (80) | SQ-1 (7.8) | — | PGMEA (2,200) | DAA (900) |
| 3-4 | CR-4 | CP-4 (80) | SQ-1 (8.2) | — | PGMEA (2,200) | DAA (900) |
| 3-5 | CR-5 | CP-5 (80) | SQ-3 (7.8) | — | PGMEA (2,200) | DAA (900) |
| 3-6 | CR-6 | CP-6 (80) | SQ-2 (4.0) | PAG-1 (10) | PGMEA (2,200) | DAA (900) |
| | | | AQ-1 (4.0) | | | |
| 3-7 | CR-7 | CP-7 (80) | SQ-1 (8.2) | — | PGMEA (2,200) | DAA (900) |
| 3-8 | CR-8 | CP-8 (80) | SQ-2 (7.8) | — | PGMEA (2,200) | DAA (900) |
| 3-9 | CR-9 | CP-9 (80) | SQ-3 (4.0) | PAG-2 (15) | PGMEA (2,200) | DAA (900) |
| | | | AQ-1 (4.0) | | | |
| 3-10 | CR-10 | CP-10 (80) | SQ-3 (7.8) | — | PGMEA (2,200) | DAA (900) |

The solvents, quenchers (SQ-1 to SQ-3, AQ-1) and acid generators (PAG-1, PAG-2) in Tables 3 and 4 are as identified below.

### Solvent:

PGMEA (propylene glycol monomethyl ether acetate)
DAA (diacetone alcohol)

### Quenchers: SQ-1 to SQ-3, AQ-1

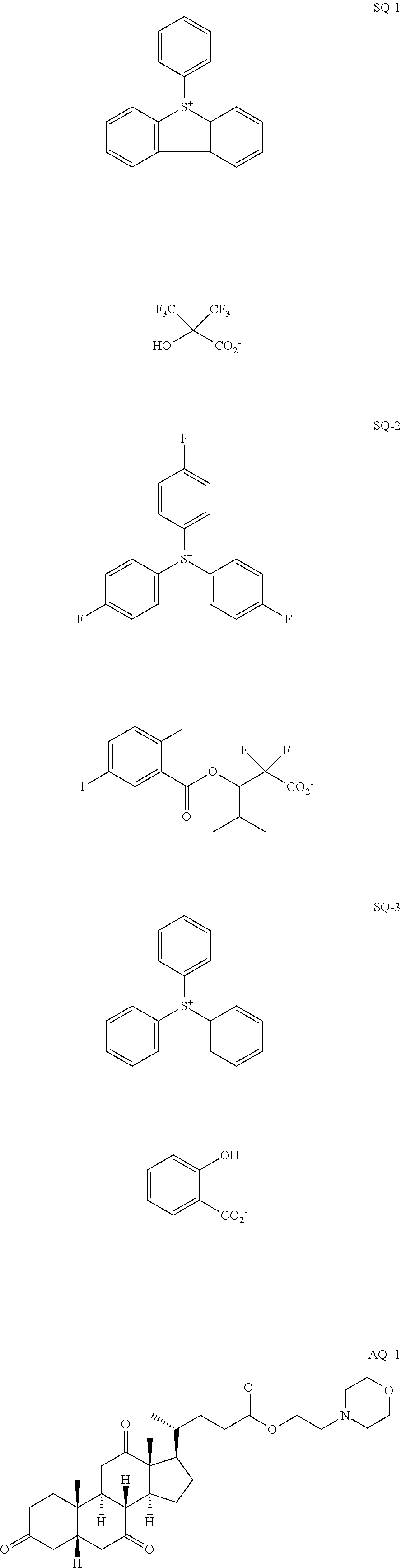

Acid generators: PAG-1, PAG-2

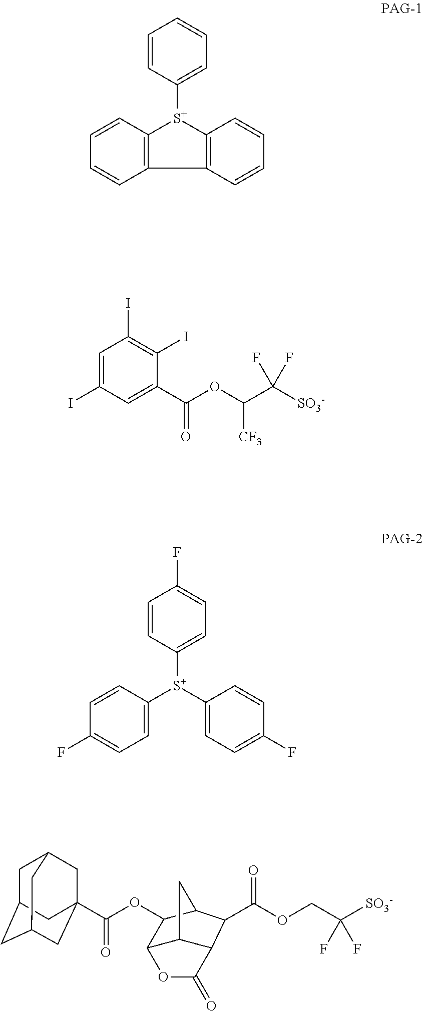

### [4] EUV Lithography Test (1)

### Examples 4-1 to 4-15 and Comparative Examples 4-1 to 4-10

Each of the chemically amplified resist compositions (R-1 to R-15, CR-1 to CR-10) shown in Tables 3 and 4 was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3300 manufactured by ASML (NA 0.33, a 0.9/0.6, dipole illumination), the resist film was exposed to EUV through a mask bearing a line-and-space (LS) pattern having a width of 18 nm (on-wafer size) and a pitch of 36 nm while changing the dose at a pitch of 1 mJ/cm² and the focus at a pitch of 0.020 μm. The resist film was baked (PEB) at the temperature shown in Tables 5 and 6 for 60 seconds. Thereafter, the film was puddle developed in a 2.38 wt % TMAH aqueous solution for 30 seconds, rinsed with a rinse fluid containing surfactant, and spin dried to form a positive LS pattern.

The obtained LS pattern was observed under CD-SEM (CG6300, Hitachi High-Technologies Corp.) and evaluated for sensitivity, exposure latitude (EL), LWR, depth of focus (DOF), and collapse limit by the following methods. The results are shown in Tables 5 and 6.

### [Evaluation of Sensitivity]

The optimum exposure dose Eop (mJ/cm²) which provided a LS pattern with a line width of 18 nm and a pitch of 36 nm was determined as an index of sensitivity. A smaller value indicates a higher sensitivity.

### [Evaluation of EL]

The exposure dose which provided a LS pattern with a space width of 18 nm±10% (i.e., 16.2 to 19.8 nm) was determined. EL (%) is calculated from the exposure doses according to the following equation. A greater value indicates better performance.

$$EL ( \% ) = ( \vert {E}_{1} - {E}_{2} \vert / Eop ) \times 100$$

Herein E₁ is an optimum exposure dose which provides a LS pattern with a line width of 16.2 nm and a pitch of 36 nm,
E₂ is an optimum exposure dose which provides a LS pattern with a line width of 19.8 nm and a pitch of 36 nm, and
Eop is an optimum exposure dose which provides a LS pattern with a line width of 18 nm and a pitch of 36 nm.

### [Evaluation of LWR]

For the LS pattern formed by exposure at the optimum dose Eop, the line width was measured at 10 longitudinally spaced apart points, from which a 3-fold value (3σ) of standard deviation (σ) was determined and reported as LWR. A smaller value of 3σ indicates a pattern having small roughness and uniform line width.

### [Evaluation of DOF]

As an index of DOF, a range of focus which provided a LS pattern with a size of 18 nm±10% (i.e., 16.2 to 19.8 nm) was determined. A greater value indicates a wider DOF.

### [Evaluation of Collapse Limit of Line Pattern]

For the LS pattern formed by exposure at the dose corresponding to the optimum focus, the line size was measured at 10 longitudinally spaced apart points. The minimum line size above which lines could be resolved without collapse was determined and reported as collapse limit. A smaller value indicates better collapse limit.

TABLE 5

| | | | Optimum | | | | |
|---|---|---|---|---|---|---|---|
| | Resist | PEB temp. | exposure dose | EL | LWR | DOF | Collapse limit |
| Example | composition | (° C.) | (mJ/cm²) | (%) | (nm) | (nm) | (nm) |
| 4-1 | R-1 | 100 | 35 | 19 | 2.4 | 120 | 10.3 |
| 4-2 | R-2 | 100 | 35 | 18 | 2.6 | 120 | 10.5 |
| 4-3 | R-3 | 100 | 36 | 20 | 2.4 | 110 | 10.8 |
| 4-4 | R-4 | 105 | 34 | 18 | 2.4 | 105 | 10.7 |
| 4-5 | R-5 | 105 | 35 | 18 | 2.5 | 120 | 11.3 |
| 4-6 | R-6 | 100 | 35 | 18 | 2.6 | 120 | 10.9 |
| 4-7 | R-7 | 100 | 36 | 18 | 2.7 | 110 | 11.2 |
| 4-8 | R-8 | 100 | 33 | 18 | 2.4 | 120 | 10.4 |
| 4-9 | R-9 | 100 | 31 | 19 | 2.5 | 120 | 10.5 |
| 4-10 | R-10 | 100 | 35 | 18 | 2.7 | 115 | 11.1 |
| 4-11 | R-11 | 100 | 36 | 17 | 2.6 | 110 | 10.5 |
| 4-12 | R-12 | 100 | 33 | 18 | 2.5 | 120 | 11.3 |
| 4-13 | R-13 | 100 | 32 | 18 | 2.7 | 120 | 10.4 |
| 4-14 | R-14 | 95 | 35 | 19 | 2.4 | 120 | 10.5 |
| 4-15 | R-15 | 100 | 34 | 18 | 2.7 | 120 | 11.3 |

TABLE 6

| | | | Optimum | | | | |
|---|---|---|---|---|---|---|---|
| Comparative | Resist | PEB temp. | exposure dose | EL | LWR | DOF | Collapse limit |
| Example | composition | (° C.) | (mJ/cm²) | (%) | (nm) | (nm) | (nm) |
| 4-1 | CR-1 | 100 | 38 | 12 | 3.4 | 80 | 13.0 |
| 4-2 | CR-2 | 100 | 38 | 13 | 3.4 | 90 | 13.0 |
| 4-3 | CR-3 | 100 | 47 | 13 | 3.0 | 80 | 13.1 |
| 4-4 | CR-4 | 105 | 45 | 13 | 3.2 | 80 | 12.5 |
| 4-5 | CR-5 | 95 | 38 | 13 | 3.1 | 80 | 12.9 |
| 4-6 | CR-6 | 100 | 38 | 14 | 3.2 | 90 | 12.7 |
| 4-7 | CR-7 | 105 | 45 | 14 | 3.1 | 80 | 12.4 |
| 4-8 | CR-8 | 100 | 46 | 14 | 3.0 | 80 | 12.5 |
| 4-9 | CR-9 | 100 | 40 | 15 | 3.4 | 90 | 12.4 |
| 4-10 | CR-10 | 100 | 42 | 14 | 3.1 | 80 | 12.5 |

From the results shown in Tables 5 and 6, it is revealed that the inventive chemically amplified resist composition comprising a base polymer containing repeat units derived from an onium salt monomer has a high sensitivity, and excellent EL, LWR and DOF. The resist composition is also confirmed to have a low collapse limit value, and resistance to pattern collapse in fine pattern formation. This demonstrates that chemically amplified resist compositions are suitable as materials for EUV lithography.

### [5] EUV Lithography Test (2)

### Examples 5-1 to 5-15 and Comparative Examples 5-1 to 5-10

Each of the chemically amplified resist compositions (R-1 to R-15, CR-1 to CR-10) shown in Tables 3 and 4 was spin coated on a silicon substrate having a 20-nm coating of silicon-containing spin-on hard mask SHB-A940 (Shin-Etsu Chemical Co., Ltd., silicon content 43 wt %) and prebaked on a hotplate at 105° C. for 60 seconds to form a resist film of 50 nm thick. Using an EUV scanner NXE3400 manufactured by ASML (NA 0.33, a 0.9/0.6, quadrupole illumination), the resist film was exposed to EUV through a mask bearing a hole pattern having a pitch (on-wafer size) of 46 nm+20% bias. The resist film was baked (PEB) on a hotplate at the temperature shown in Tables 7 and 8 for 60 seconds and developed in a 2.38 wt % TMAH aqueous solution for 30 seconds to form a hole pattern having a size of 23 nm.

The pattern as developed was observed under CD-SEM (CG6300, Hitachi High-Technologies Corp.). The dose (mJ/cm²) at which a pattern with a hole size of 23 nm was printed was determined as an index of sensitivity. The size of 50 holes was measured, from which a 3-fold value (3σ) of the standard deviation (σ) was determined as a dimensional variation (or CDU). The results are shown in Tables 7 and 8.

TABLE 7

| | | Resist | PEB temp. | Optimum exposure | CDU |
|---|---|---|---|---|---|
| | | composition | (° C.) | dose (mJ/cm²) | (nm) |
| Example | 5-1 | R-1 | 95 | 25 | 2.3 |
| | 5-2 | R-2 | 95 | 24 | 2.3 |
| | 5-3 | R-3 | 90 | 25 | 2.5 |
| | 5-4 | R-4 | 90 | 25 | 2.5 |
| | 5-5 | R-5 | 90 | 25 | 2.5 |
| | 5-6 | R-6 | 95 | 24 | 2.4 |
| | 5-7 | R-7 | 95 | 26 | 2.3 |
| | 5-8 | R-8 | 90 | 25 | 2.2 |
| | 5-9 | R-9 | 90 | 24 | 2.3 |
| | 5-10 | R-10 | 90 | 22 | 2.5 |
| | 5-11 | R-11 | 90 | 24 | 2.4 |
| | 5-12 | R-12 | 90 | 25 | 2.4 |
| | 5-13 | R-13 | 90 | 24 | 2.4 |
| | 5-14 | R-14 | 90 | 25 | 2.5 |
| | 5-15 | R-15 | 95 | 24 | 2.3 |

TABLE 8

| | | | | Optimum | |
|---|---|---|---|---|---|
| | | Resist | PEB temp. | exposure | CDU |
| | | composition | (° C.) | dose (mJ/cm²) | (nm) |
| Comparative | 5-1 | CR-1 | 95 | 29 | 2.9 |
| Example | 5-2 | CR-2 | 95 | 29 | 2.9 |
| | 5-3 | CR-3 | 95 | 35 | 3.1 |
| | 5-4 | CR-4 | 90 | 36 | 3.0 |
| | 5-5 | CR-5 | 90 | 29 | 3.1 |
| | 5-6 | CR-6 | 95 | 29 | 3.2 |
| | 5-7 | CR-7 | 90 | 34 | 3.2 |
| | 5-8 | CR-8 | 90 | 34 | 3.2 |
| | 5-9 | CR-9 | 90 | 31 | 2.8 |
| | 5-10 | CR-10 | 95 | 29 | 3.2 |

It is demonstrated in Tables 7 and 8 that chemically amplified resist compositions within the scope of the invention exhibit a high sensitivity and improved CDU.

Japanese Patent Application No. 2025-051348 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. An onium salt monomer having the following formula (a): 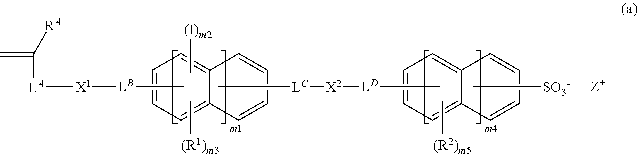
wherein m1 is 0 or 1, m2 is 0, 1, 2, 3 or 4 in case of m1=0, and 0, 1, 2, 3, 4, 5 or 6 in case of m1=1, m3 is 0, 1, 2, 3 or 4 in case of m1=0, and 0, 1, 2, 3, 4, 5 or 6 in case of m1=1, m2+m3 is from 0 to 4 when m1=0 and m2+m3 is from 0 to 6 when m1=1, m4 is 0 or 1, m5 is 1, 2, 3 or 4 when m4=0, and 1, 2, 3, 4, 5 or 6 when m4=1,
R^{A} is hydrogen atom or methyl,
R¹ is halogen exclusive of iodine, nitro group, hydroxy group, cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, a plurality of R¹ may be identical or different and a plurality of R¹ may bond together to form a ring with the carbon atoms to which they are attached, when m3 is 2 or more,
R² is halogen exclusive of iodine, or an electron withdrawing group, a plurality of R² may be identical or different and a plurality of R² may bond together to form a ring with the carbon atom to which they are attached, when m5 is 2 or more, except that all R² is fluorine,
L^{A}, L^{B}, L^{C} and L^{D} are each independently a single bond, ester bond, ether bond, amide bond, sulfonic ester bond, sulfonic amide bond, carbonate bond or carbamate bond,
X¹ and X² are each independently a single bond, or a C₁-C₄₀ hydrocarbylene group which may contain a heteroatom,
Z⁺ is a sulfonium cation having the following formula (Z-1) or an iodonium cation having the following formula (Z-2); 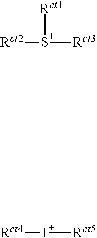
wherein R^{ct1} to R^{ct5} are each independently halogen, or a C₁-C₃₀ hydrocarbyl group which may contain a heteroatom, and R^{ct1} and R^{ct2} may bond together to form a ring with the sulfur atom to which they are attached.

2. The onium salt monomer of claim 1 which has the following formula (a1): 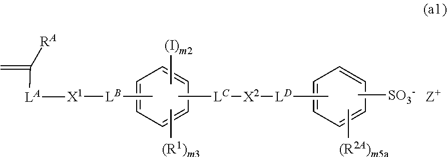
wherein m2, m3, R^{A}, R¹, R², X¹, X², L^{A} to L^{D} and Z⁺ are as defined above, and m5a is 1, 2, 3 or 4.

3. The onium salt monomer of claim 2 which has the following formula (a2): 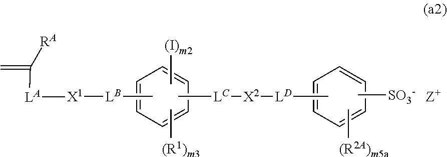
wherein m2, m3, m5a, R^{A}, R¹, X¹, X², L^{A} to L^{D} and Z⁺ are as defined above, and R^{2A} is an electron withdrawing group exclusive of halogen.

4. A monomeric photoacid generator comprising the onium salt monomer of claim 1.

5. A polymer comprising repeat units derived from the monomeric photoacid generator of claim 4.

6. The polymer of claim 5 comprising repeat units of at least one type selected from repeat units of the formulae (b1) and (b2): 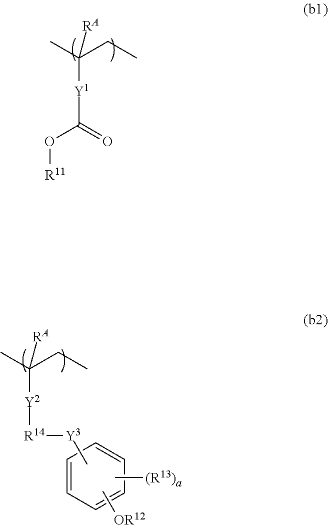
wherein R^{A} is each independently hydrogen or methyl,
Y¹ is a single bond, a phenylene group, a naphthylene group, or a C₁-C₁₂ linking group containing at least one selected from an ester bond, an ether bond and a lactone ring, the phenylene group, the naphthylene group and the linking group may have at least one selected from a hydroxy group, a C₁-C₈ saturated hydrocarbyloxy group and a C₂-C₈ saturated hydrocarbylcarbonyloxy group,
Y² is a single bond or ester bond,
Y³ is a single bond, ether bond or ester bond,
R¹¹ and R¹² are each independently an acid labile group,
R¹³ is a C₁-C₄ saturated hydrocarbyl group, halogen, C₂-C₅ saturated hydrocarbylcarbonyl group, cyano group, or C₂-C₅ saturated hydrocarbyloxycarbonyl group,
R¹⁴ is a single bond or C₁-C₆ alkanediyl group, and the alkanediyl group may contain at least one selected from a hydroxy group, a C₁-C₈ saturated hydrocarbyloxy group, a C₂-C₈ saturated hydrocarbylcarbonyloxy group, an ether bond and an ester bond, and
a is 0, 1, 2, 3 or 4.

7. A chemically amplified resist composition comprising a base polymer containing the polymer of claim 6.

8. The chemically amplified resist composition of claim 7, further comprising an organic solvent.

9. The chemically amplified resist composition of claim 7, further comprising a quencher.

10. The chemically amplified resist composition of claim 7, further comprising an acid generator.

11. The chemically amplified resist composition of claim 7, further comprising a surfactant.

12. A pattern forming process comprising the steps of applying the chemically amplified resist composition of claim 7 onto a substrate to form a resist film thereon, exposing the resist film to high-energy radiation, and developing the exposed resist film in a developer.

13. The pattern forming process of claim 12, wherein the high-energy radiation is ArF excimer laser having a wavelength 193 nm, KrF excimer laser having a wavelength 248 nm, an electron beam, or an extreme ultraviolet ray having a wavelength 3 to 15 nm.
